(19) United States
(12) Patent Application Publication
(10) Pub. No.: US 2019/0264287 A1
(43) Pub. Date: Aug. 29, 2019

(54) DNA METHYLATION SIGNATURES ASSOCIATED WITH CHILDHOOD TRAUMA AND METHODS INVOLVING SAME

(71) Applicants: Elaine L. BEARER, Albuquerque, NM (US); STC.UNM, Albuquerque, NM (US)

(72) Inventor: Elaine L. Bearer, Albuquerque, NM (US)

(21) Appl. No.: 16/317,923

(22) PCT Filed: Jul. 14, 2017
(86) PCT No.: PCT/US2017/042201

§ 371 Date: Jan. 15, 2019

Related U.S. Application Data
(60) Provisional application No. 62/362,903, filed on Jul. 15, 2016.

(51) Int. Cl. C12Q 1/6883

(52) U.S. Cl. CPC C12Q 1/6883; C12Q 2600/154

(57) ABSTRACT

A method includes obtaining a biological sample from an individual, extracting DNA from cells in the sample, measuring the degree of methylation of the extracted DNA at one or more specified loci, and identifying if at least one locus exhibits an alteration in methylation or a degree of methylation that is outside of a normal limit. The method may further include determining whether an individual was exposed to abuse and/or trauma. In some cases, the method further includes treating the individual with a therapy effective to reverse the differential methylation of the DNA.

### CONTINUING APPLICATION DATA

This application claims the benefit of U.S. Provisional Application Ser. No. 62/362,903, filed Jul. 15, 2016, which is incorporated by reference herein.

### GOVERNMENT FUNDING

This invention was made with government support under MH096093 awarded by the National Institutes of Health. The government has certain rights in the invention.

### SEQUENCE LISTING

This application contains a Sequence Listing electronically submitted to the United States Patent and Trademark Office via EFS-Web as an ASCII text file entitled “310-01130201 ST25.txt” having a size of 168 kilobytes and created on Jul. 12, 2017. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### SUMMARY

This disclosure describes epigenetic changes, particularly DNA methylation, that may occur in a traumatized individual and methods of detecting those changes. In one aspect, this disclosure describes a method that includes obtaining a biological sample from an individual, extracting DNA from cells in the sample, and measuring the degree of methylation of the extracted DNA at one or more specified loci. In some embodiments, the method includes determining whether the degree of methylation at one or more of the specified loci is outside of a normal limit. In some embodiments, the method includes identifying the individual as having been subjected to a traumatic event. The individual may be identified as having been subjected to a traumatic event if one or more of the specified loci exhibits a degree of methylation outside of a normal limit. In some embodiments, the individual is a child.

In some cases, the method may be used to follow an individual over time. In such embodiments, the method may be repeated at a second time point. For example, the method may include identifying the individual as having been subjected to trauma between collection of a first biological sample and collection of a second biological sample if the second biological sample exhibits an alteration in the degree of DNA methylation compared to the first biological sample. Additionally or alternatively, the method may include treating the individual with a therapy between collection of a first biological sample and collection of a second biological sample. In some embodiments, the therapy is effective to reverse methylation at one or more loci.

In some embodiments, the method includes measuring the degree of methylation at a plurality of loci. In some embodiments, the one or more loci include one or more CpG sites.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains drawings executed in color. Copies of this patent or patent application publication with color drawings will be provided by the Office upon request and payment of the necessary fee.

FIG. 1. An exemplary experimental design.

FIG. 2. Exemplary subject enrollment. For subjects in rows 1 to 6, cortisol measurements were taken, and methylation was determined (because the subjects exhibited the most reliable cortisol measurements). For rows 7-10, only cortisol measurements were taken for the subject.

FIG. 3. Exemplary behavioral data. Subjects 2, 4, and 5 exhibited no difference from intake to follow-up in traumatic event; Subjects 1, 3, and 6 exhibited difference in trauma from intake to follow-up; Boxes outlined with dotted lines=either no trauma or traumatic event was not directed toward individual; Boxes including circles=trauma included the individual.

FIG. 4(A-B). Cortisol levels at intake and after therapy (FIG. 4A) and methylation patterns for the same subjects (FIG. 4B). Cortisol was extracted from 3 cm of hair at each time point and measured by ELISA. A heat map of methylation sites from a dataset of 850,000 sites showing the 1000 most significantly different (between methylated and non-methylated) sites in promoter/enhancer regions. Hierarchical clustering is by similarity between these sites across samples (dendrogram above hear map). Note the subject 6: follow-up, when cortisol is high, is on the left of the heat map, showing strong difference between all other samples, including intake (lane 6 from the left on the heat map) for this same subject. This difference indicates that cortisol alone is not responsible for the flip in methylation of these promoter sites, since subjects 4 and 5 had high cortisol at intake but did not display this pattern.

FIG. 5 shows exemplary data comparing DNA methylation profiles of abuse. Left panel: Methylation values for all 850,000 sites demonstrate minor variability across all individuals. Red indicates low methylation, grey is intermediate, and blue is representative of higher methylation. Middle panel: Methylation values for the 1000 most variable promoter-associated sites including two more subjects at two time points. Three samples demonstrate a similar flip in the methylation of these sites (left three lanes). These samples are exemplary of individuals with higher instance of abuse at that time point. Right panel: Heat map comparing only the three individuals with the most drastic change in methylation for the most variable promoter-associated sites.

FIG. 6. Gene ontology enrichment for the top 2000 promoter-associated sites. Ten categories reached significance. X-axis indicates number of sites involved in this pathway.

FIG. 7. Pathway analysis of enriched gene ontology (GO) terms. Percent change in this instance is referring to delta beta (difference between beta values, compared between subjects). Green indicates hypomethylation in better group (hypermethylation in worse group). Red indicates hypermethylation in better group (hypomethylation in worse group). Statistically significant differences between delta beta were compared across 850,000 sites the top 2000 sites with the highest probability of having a significant difference between unmethylated and methylated sites for each sample were selected. Six subjects were measured before and after therapy in duplicate, and two subjects were measured at two different time points also run in duplicate for a total of 32 measurements of 850,000 sites each. Genes with highest probability of being methylated and highest probability of being unmethylated in each individual were ranked across samples producing the heat map.

FIG. 8 (A-B). Exemplary micrographs of saliva smears. To determine the types of cells in saliva, saliva was collected from 6 healthy volunteers either directly onto a glass slide (FIG. 8A) or first into ORAGENE DNA collection cups which contain a buffer to preserve the DNA and then onto a glass slide (FIG. 8B). Slides were stained with hematoxylin and eosin according to normal pathology procedures. Slides were reviewed and types of cells counted. As shown in FIG. 8A, some saliva had abundant keratinocytes (solid arrow) and few WBCs (dashed arrow); other samples displayed the opposite ratio of cells. As shown in FIG. 8B, after treatment with the buffer, keratinocytes appeared ghosted and lacked nuclei. Some small round blue dots remaining represent un-extracted nuclei.

### DETAILED DESCRIPTION OF ILLUSTRATIVE EMBODIMENTS

This disclosure describes epigenetic changes, particularly DNA methylation, that occur in traumatized individuals and methods of detecting those changes. Accordingly, this disclosure describes a method of measuring the DNA methylation in a biological sample obtained from an individual at one or more specified loci. In some embodiments, the method includes identifying individuals who have been exposed to a traumatic experience (including, for example abuse) and/or treating individuals who have been exposed to a traumatic experience.

Childhood maltreatment is a major risk factor for poor physical outcomes in adulthood such as, for example, diabetes, cardiovascular disease, depression, and/or other cognitive dysfunction. Epigenetic modifications have become an increasingly popular mechanism to explain the relationship between adverse childhood experiences (ACE) and poor health outcomes later in life. DNA methylation is a commonly studied epigenetic modification since DNA methylation influences gene expression and is involved in cell differentiation during development. DNA methylation may alter gene expression without changing the genetic sequence, providing a potential therapeutic target for reversible modifications. The human genome is predicted to have over five million dynamic methylation sites, indicating that 20% of the methylation sites present in the genome may be altered by environmental and developmental influences. These dynamic sites are of particular interest in understanding the mechanism underlying the association between childhood maltreatment and poor health in adulthood because they may be potential therapeutic targets since methylation changes of these sites could be reversed.

Therapies that target methylation sites include drugs that alter DNA methylation in immune cells and/or in the brain. Some drugs may be already approved for use in treating other diseases. Exemplary drugs include, for example, valproic acid, 5-azocyidine, propranolol, inhibitors/activators of the norepinephrine system (for example, an alpha-1 blocker such as, for example, prazosin), or drugs approved for treating post-traumatic stress disorder (PTSD) and/or anxiety disorders in adults.

It may be that no single methylation site is responsible for driving all of the negative phenotypes seen in children that have been exposed to a traumatic experience. However, it may be that expression of many related genes is changed in tandem, leading to the disruption seen later in life in these individuals. A change in expression of a gene may be the result of a change in the methylation of a regulatory sequence that is operably linked to the gene so that the methylation status of the regulatory sequence influences expression of the gene. If so, then global methylation patterns may reveal a signature that reflects a history that includes exposure to a traumatic experience. The methylation sites that are associated with the signature—for example, the sites that are altered by childhood trauma—may be re-altered using appropriate therapy.

Significant changes in the methylation levels of 2000 promoter sites occurred in samples obtained from individuals subjected to abuse as reported in the PTSD Eyberg survey. Similar methylation data from archived saliva samples from children with documented abuse validated the initial data. So, too, did a comparison of the methylation patterns described herein with published findings in adults reporting abuse in childhood. All of these analyses produced the same signature pattern: Elevated methylation of 1000 sites, and decreased methylation in another 1000 sites. An ontology analysis revealed that the more significantly changed sites are in genes for hemostasis and immunological processes. This subset of sites may be used to design tests to detect the impact of early life abuse, and to develop and follow therapeutic interventions.

The methylation changes were analyzed using a whole genome approach implemented by the Illumina MethylationEPIC 850K BeadChip that interrogates over 850,000 methylation sites across the genome. The array-based approach provides single-base resolution and allows one to recognize patterns across the genome in sites that have already been shown to be involved in regulation of gene expression such as promoters and enhancers. This array also allows one to identify intergenic regions that are involved in psychiatric disorders such as bipolar and schizophrenia.

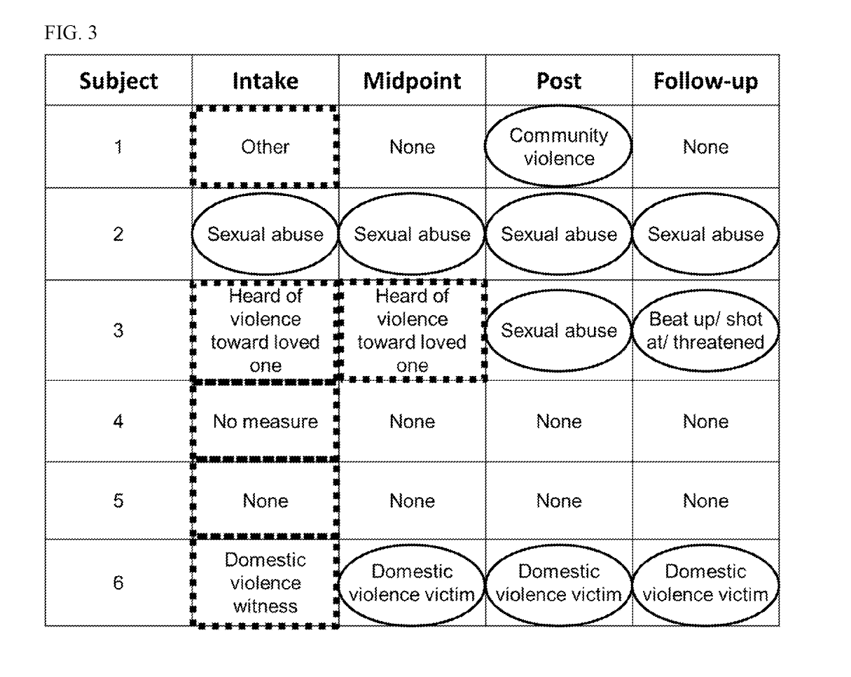

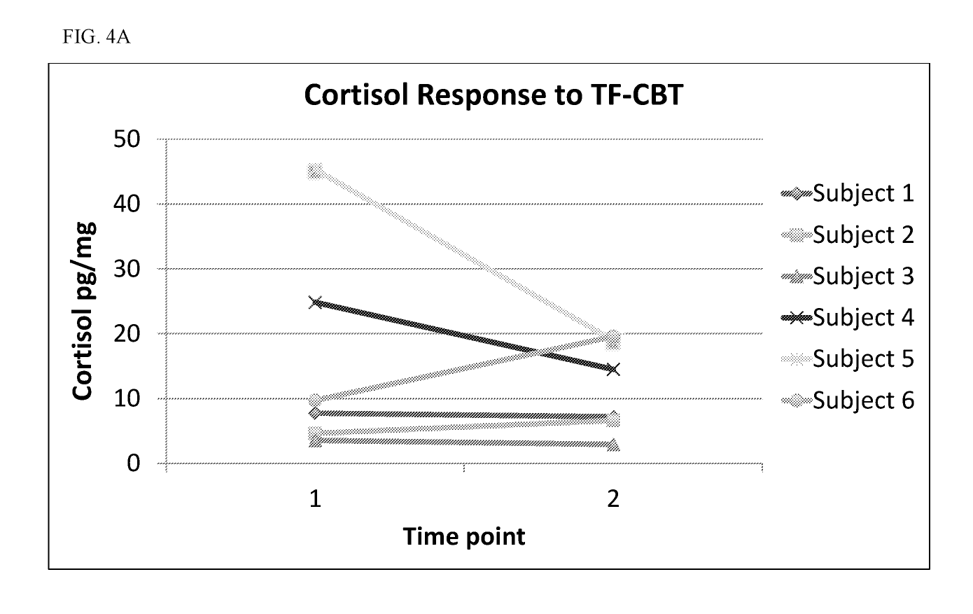

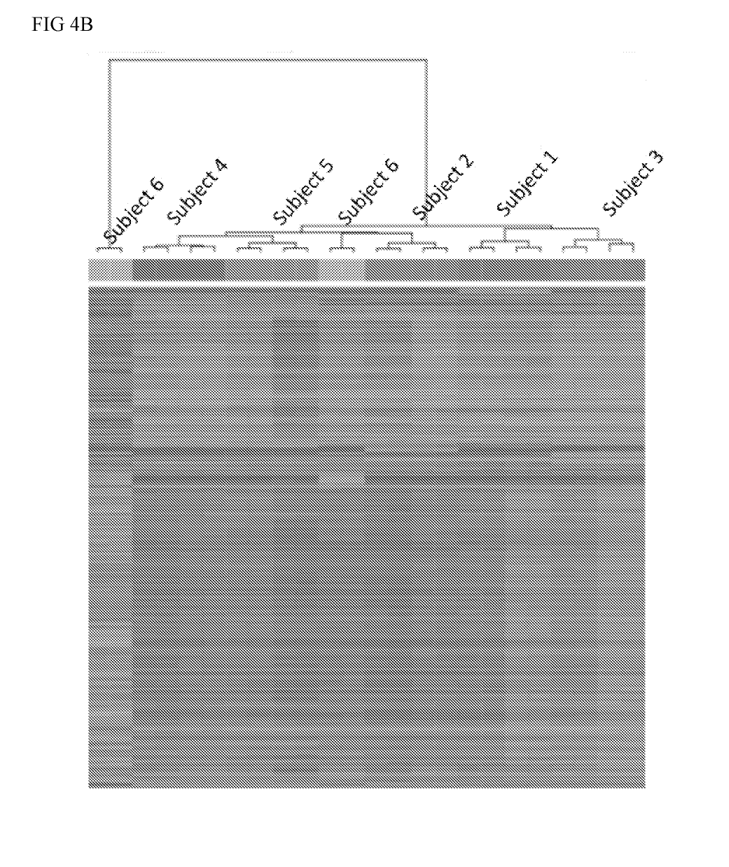

Since a traumatic experience may include abuse including, for example, a wide variety of maltreatment types such as physical, sexual, and emotional abuse, the analysis adopted an unbiased data-driven approach to allow the data to explain differences in children with different trauma histories (FIG. 3). The cortisol patterns appeared to be most related to service planning area (FIG. 4). Although cortisol had some correlation with behavioral measures, they were moderate and insignificant.

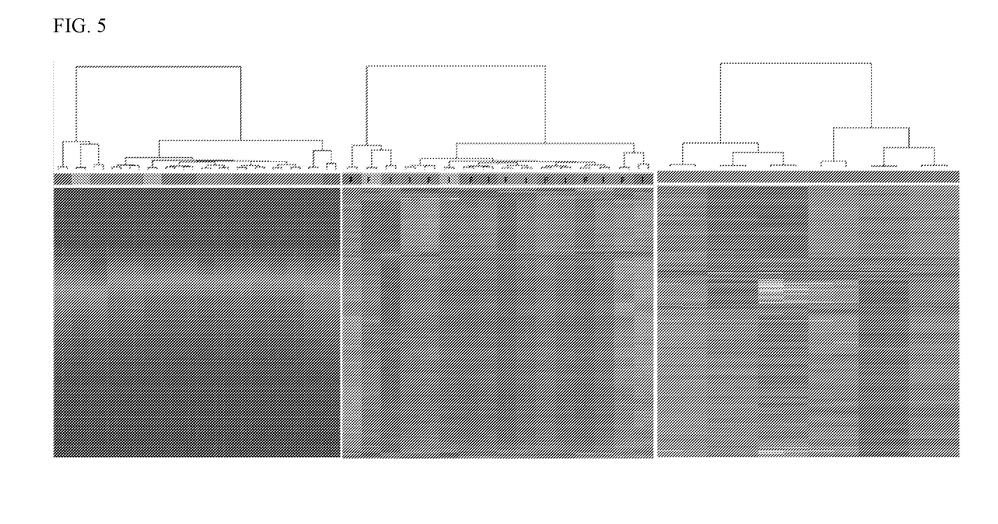

Methylation, however, was more informative of trauma effects. Methylation patterns for one individual in particular were opposite for a subset of 2,000 promoter-associated CpG sites. Methylation sites were enriched for genes involved in homeostasis as well as platelet activation and other inter-related pathways. While the overall methylation pattern was not drastically different from others, the methylation differences of these sites were extreme, demonstrating an almost complete reversal in methylation—that is, an increase in sites that had low methylation and a decrease in sites that had high methylation (FIG. 5). Samples from this child exhibited altered methylation at follow-up was the only child that also exhibited a dramatic increase in cortisol at follow-up. Behavioral analysis confirmed that this child had experienced worsening trauma throughout her time in therapy. At intake, she was a witness to domestic violence, but at all time points following, she was a victim of domestic violence. Two other children, while less extreme, demonstrated differing patterns of methylation from intake to follow-up at similar sites. Neither of these children reported trauma at intake on their behavioral records, but did report trauma for at least one later time point during therapy.

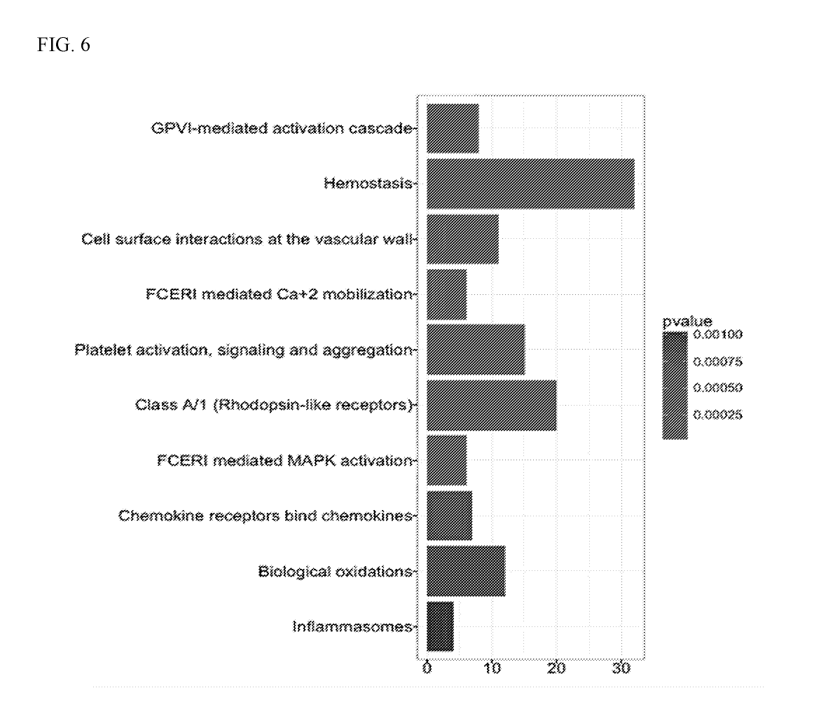

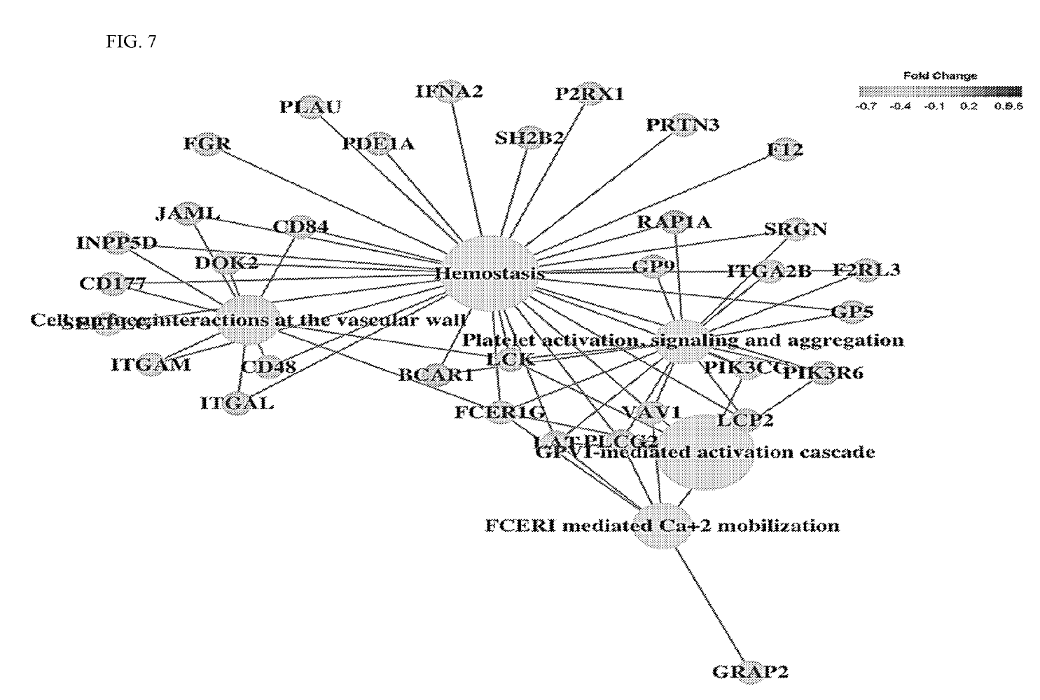

Gene ontology (GO) enrichment analysis was computed for the 2000 most variable promoter sites. Of these 2000 sites, 817 mapped to specific genes based on EntrezID. These sites were significantly (p<0.05 BH corrected) enriched in 10 categories (FIG. 6 and FIG. 7).

To validate this observed methylation patterns, results with previous publications on methylation changes in victims of child abuse. Suderman et al. (Proceedings of the National Academy of Sciences of the U.S. Pat. No. 2,013,110 (14), E1246) report a similar pattern in methylation for a subset of sites for adult males who were victims of abuse as children. Since this pattern appears to prevail into adulthood, saliva samples from a longitudinal study that followed children over a 15-year period from childhood into early adulthood were compared to the pilot study data described above. The pattern for the two individuals was nearly identical for the most variable sites seen in the pilot study.

In one aspect, the methods described herein include obtaining or having obtained at least one biological sample from an individual. In some embodiments, the individual is preferably a human. In some embodiments, the individual is preferably a child. As used herein, a child includes an individual under the age of 18. In some embodiments, the child is preferably between the ages of 2 and 18. In some embodiments, an additional (for example, second, third, fourth, etc.) biological sample may be obtained from the same individual. In some embodiments, the additional biological sample may be obtained at an additional (for example, second, third, fourth, etc.) time point. For example, the additional time point(s) may be a day, a plurality of days, a month, a plurality of months, a year, or a plurality of years after the first biological sample is collected. In some embodiments, when the individual is suspected of having been subject to a traumatic experience after the collection of the first sample, the additional biological sample may be collected after the traumatic experience has occurred. A traumatic experience may include, abuse including, for example, physical abuse, emotional abuse, sexual abuse, and neglect. A traumatic experience may additionally or alternatively include accidental trauma (for example, car accidents or other accidental injuries) or witnessing another individual undergoing abuse (for example, witnessing domestic violence).

A biological sample may include any suitable sample that includes cells from an individual. For example, a biological sample may include whole blood, semen, saliva, tears, urine, fecal material, sweat, buccal smears, skin, hair, and biopsy samples of organs and muscle.

The biological sample may be obtained by any suitable means. In some embodiments, the biological sample may be stored in a collection device. Collection devices suitable for use in the present invention include devices known in the art for collecting and/or storing a biological sample of an individual from which nucleic acids and/or polypeptides (including, for example DNA) may be isolated. Suitable collection devices include, for example, specimen cups, swabs, glass slides, test tubes, lancets, ORAGENE DNA collection cups, VACUTAINER tubes, and kits.

The methods described herein may include extracting or having extracted DNA from at least a portion of the cells included in the biological sample. The DNA may be extracted (including, for example, isolated and purified) by any suitable means known to those having skill in the art. Extracting DNA preferably preserves the methylation status of the DNA present in the cells of the biological sample.

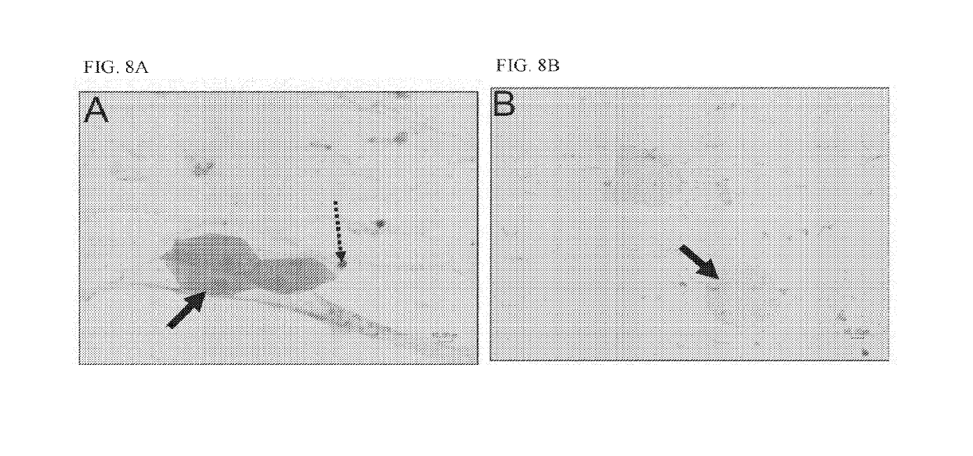

In some embodiments, including, for example, when the biological sample includes saliva, the biological sample may include at least one of a keratinocyte and a white blood cell. In some embodiments, the keratinocyte may include a buccal keratinocyte. Saliva contains both white blood cells and buccal keratinocytes, in different proportions in different samples (see FIG. 8). Moreover, saliva may be more easily obtained from children than blood. Saliva collection is more acceptable to many children than blood collection. Further, it is easier for investigators to obtain permission from offices for protection of human subjects from risk, such as Institutional Review Boards (IRB), to collect saliva for experimental procedures.

The methods described herein may include measuring or having measured the degree of methylation of the extracted DNA at one or more loci. In some embodiments, the one or more loci include one or more CpG sites (also referred to as CG sites or CpG methylation sites). The cytosine in the CpG dinucleotide may preferably be methylated to form 5-methylcytosine. In some embodiments, each of the one or more loci includes one or more CpG sites. In some embodiments, each of the one or more loci includes one CpG sites.

For example, in some embodiments, the degree of methylation of the DNA extracted from cells present in saliva may be measured. In some embodiments, methylation patterns obtained from saliva may need to be corrected for relative numbers of and/or DNA of keratinocytes versus white blood cells. Stressed children may have scant saliva and thus produce more keratinocytes than non-stressed children, since fear and anxiety in children produce a severe dry mouth which alters the cellular composition of saliva. Hence the cell composition of the saliva may need to be accounted for in the analysis of methylation sites, since keratinocytes and white blood cells may have widely different methylation patterns.

In some embodiments, the one or more loci exhibiting methylation patterns may preferably be selected such that no calculation of keratinocyte versus white blood cell numbers and/or DNA is required.

In some embodiments, measuring or having measured the degree of methylation of the extracted DNA preferably includes measuring the degree of methylation at one or more loci listed in Table 1. Without wishing to be bound by theory, it is believed that using the methylation patterns exhibited by the loci of Table 1 does not require a calculation of keratinocyte numbers versus white blood cell numbers.

DNA methylation—including the degree of methylation—may be measured by any suitable means. For example, DNA methylation may be identified using methyl-(MeDIP) pull-downs, bisulfite conversion, reduced representation bisulfite sequencing (RBBS) followed by DNA sequencing and/or high-density array hybridization. In some embodiments, a high-density array may be used. A high-density array may include, for example, an Infinium HumanMethylation450 BeadChip Kit (Illumina, San Diego, Calif.), an Infinium MethylationEPIC Kit (Illumina, San Diego, Calif.), etc. DNA methylation data may be analyzed using any suitable methodology known to those having skill in the art. For example, in some embodiments, DNA methylation data may be analyzed using RnBeads (available from the Max Planck Institut Informatik on the world wide web at rnbeads.mpi-infmpg.de/).

In some embodiments, the method includes determining if one or more loci exhibit a degree of methylation is outside of a normal limit. Whether a degree of methylation is outside of a normal limit for each loci may be calculated by a person having ordinary skill in the art based on a reference range. The reference range may be determined by the level of methylation most prevalent in a reference group taken from the general (for example, total) population. In some embodiments, the reference range may be defined as the interval between which 95% of values of a reference group fall into.

In some embodiments, whether the degree of methylation of a loci is outside of the normal limit may be calculated with reference to the mean difference (“mean.diff”) values provided in Table 1. For example, a degree of methylation at a locus may be outside of a normal limit when the absolute value of the degree of methylation at the locus is at least as great as the absolute value of the mean difference for the same locus listed in Table 1. The absolute values of mean difference provided in Table 1 range from 0.007 to 0.63, and negative values in this column of Table 1 indicate loci in which more methylation was observed (on average) in individuals with increased cortisol compared to control individuals, as further discussed in the Examples.

In some embodiments, the normal limit for a locus may be calculated with reference to a normal control. In some embodiments, a normal control may include an individual having similar characteristics to the individual (e.g., age, sex, etc.) from which the biological sample was obtained but known not to have been subject to a traumatic experience. In some embodiments, a normal control may preferably include a population of individuals taken from the general (for example, total) population.

In some embodiments, the degree of methylation at the locus may be altered from the degree of methylation for the same locus in a normal control. In some embodiments, the degree of methylation may be altered by at least 1%, at least 2%, at least 3%, at least 4%, at least 5%, at least 10%, at least 15%, at least 20%, at least 25%, at least 30%, at least 35%, or at least 40% from the degree of methylation for the same locus in a normal control.

In some embodiments, if one or more loci exhibit a degree of methylation outside of a normal limit an individual may be identified as having been subjected to a traumatic experience.

In some embodiments, the method preferably includes determining if a plurality of loci exhibit a degree of methylation outside of a normal limit. For example, the method may include measuring the degree of methylation at 2 or more loci, at least 10 loci, at least 20 loci, at least 30 loci, at least 40 loci, at least 50 loci, at least 100 loci, at least 150 loci, at least 200 loci, at least 225 loci, or at least 250 loci. In some embodiments, the loci are preferably selected from the loci listed in Table 1. In some embodiments, the degree of methylation may preferably be measured at 2 or more loci, at least 10 loci, at least 20 loci, at least 30 loci, at least 40 loci, at least 50 loci, at least 100 loci, at least 150 loci, at least 200 loci, at least 225 loci, or at each or the loci listed in Table 1.

In some embodiments, an individual may be identified as having been subjected to a traumatic experience if at least 10 loci, at least 20 loci, at least 30 loci, at least 40 loci, at least 50 loci, at least 100 loci, at least 150 loci, at least 200 loci, at least 225 loci, or each of the loci of Table 1 exhibit a degree of methylation outside of a normal limit.

In some embodiments, the method may include measuring the degree of methylation at loci from at least ten genes, at least 20 genes, at least 30 genes, at least 40 genes, or at least 50 genes.

In some embodiments, the method may include measuring the degree of methylation at loci having an absolute value of the mean difference (“mean.diff” column) as listed in Table 1 of at least 0.01, at least 0.02, at least 0.03, at least 0.04, at least 0.05, at least 0.1, at least 0.15, at least 0.2, at least 0.25, at least 0.3, at least 0.35, at least 0.4, at least 0.45, or at least 0.5.

In some embodiments, the method may include measuring the degree of methylation at loci having a degree of methylation that is altered by at least 1%, at least 2%, at least 3%, at least 4%, at least 5%, at least 10%, at least 15%, at least 20%, at least 25%, at least 30%, at least 35%, or at least 40% from the degree of methylation for the same locus in a normal control.

In some embodiments, the method may include measuring the degree of methylation at loci that have been previously correlated with clinical history including a traumatic experience. In some embodiments, the method may include measuring the degree of methylation at loci that have been correlated with other biomarkers known to be altered in response to a traumatic experiment. Correlating the degree of methylation with the clinical history and/or other biomarkers may improve accuracy of the diagnosis of a biological response to a traumatic experience.

In some embodiments, including, for example, when the type of traumatic experience an individual may have been subjected to is unknown, it may be desirable to measure the degree of methylation at an increased number of loci to prevent an erroneous conclusion that the individual has not experienced trauma.

In some embodiments, including for example, when more than one biological sample has been obtained from an individual, the degree of methylation of the extracted DNA of the samples may be compared.

For example, when the method includes obtaining an additional (e.g., second) biological sample from the individual at an additional (e.g., second) time point, the degree of methylation of the extracted DNA of the biological sample at one or more loci listed in Table 1 may be compared with the degree of methylation of the extracted DNA of an additional biological sample at the same one or more loci listed in Table 1. In some embodiments, an additional biological sample can be obtained after an individual has been subjected to or is suspected of having been subjected to a traumatic experience. In some embodiments, the method may further include determining if the degree of methylation is altered in the additional biological sample compared to the degree of methylation at the same locus in a previous biological sample. In some embodiments, the absolute values of the degree of methylation one or more loci may be compared. In some embodiments, if the degree of methylation for one or more loci listed in Table 1 is altered in the additional biological sample, the individual may be identified as having been subjected to a traumatic experience between collections of the biological samples.

In some embodiments, the method may further include treating the individual with a therapy effective to reverse the methylation of the DNA at one or more loci. In some embodiments, the therapy may be effective to reverse the methylation at one or more loci where the degree of methylation is outside of a normal limit. For example, when the degree of methylation is increased at a locus to a level outside of a normal limit, reversing the methylation may include decreasing the degree of methylation at that locus; when the degree of methylation is decreased at a locus to a level outside of a normal limit, reversing the methylation may include increasing the degree of methylation at that locus.

In some embodiments, the therapy may involve administering an effective amount of a drug that may reverse the abnormal DNA methylation patterns that are associated with exposure to a traumatic experience.

In some embodiments, the therapy may alter the degree of methylation at one or more loci listed in Table 1 by at least 1%, at least 5%, at least 10%, at least 20%, at least 30%, at least 40%, at least 50%, at least 60%, at least 70%, at least 80%, or at least 90%.

In some embodiments, methylation status may be measured before, during, and/or after a therapy is administered to determine if the therapy is effective. Measuring methylation status at such different time points may be an effective method to follow efficacy of a therapy.

The methods disclosed herein may include obtaining a biological sample from an individual before a therapy and an additional (e.g., a second) biological sample from the same individual at a time point after treating the individual with the therapy (e.g., a second time point). The method may further include comparing the degree of methylation of the extracted DNA of a biological sample taken before the therapy with the degree of methylation of the extracted DNA of the biological sample taken after the therapy at the same one or more loci and determining if the degree of methylation for one or more loci (including, for example, the loci listed in Table 1) is altered.

In the preceding description and following claims, the term “and/or” means one or all of the listed elements or a combination of any two or more of the listed elements; the terms “comprises,” “comprising,” and variations thereof are to be construed as open ended—that is, additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (for example, 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments may include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Methods:

Protocol 1: Analyzing Methylation and Cortisol Changes with Trauma-Focused Cognitive Behavioral Therapy (TF-CBT).

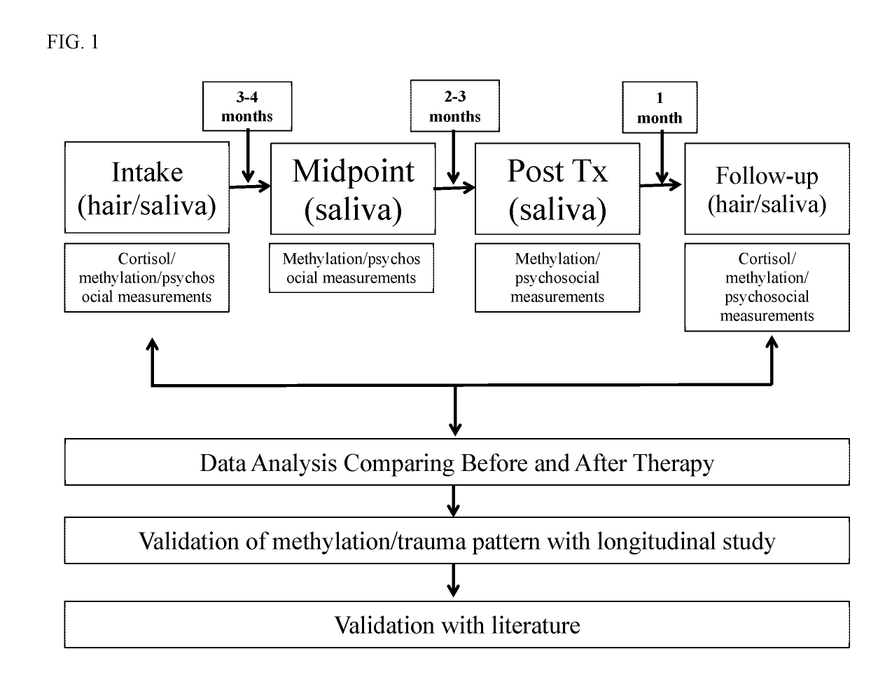

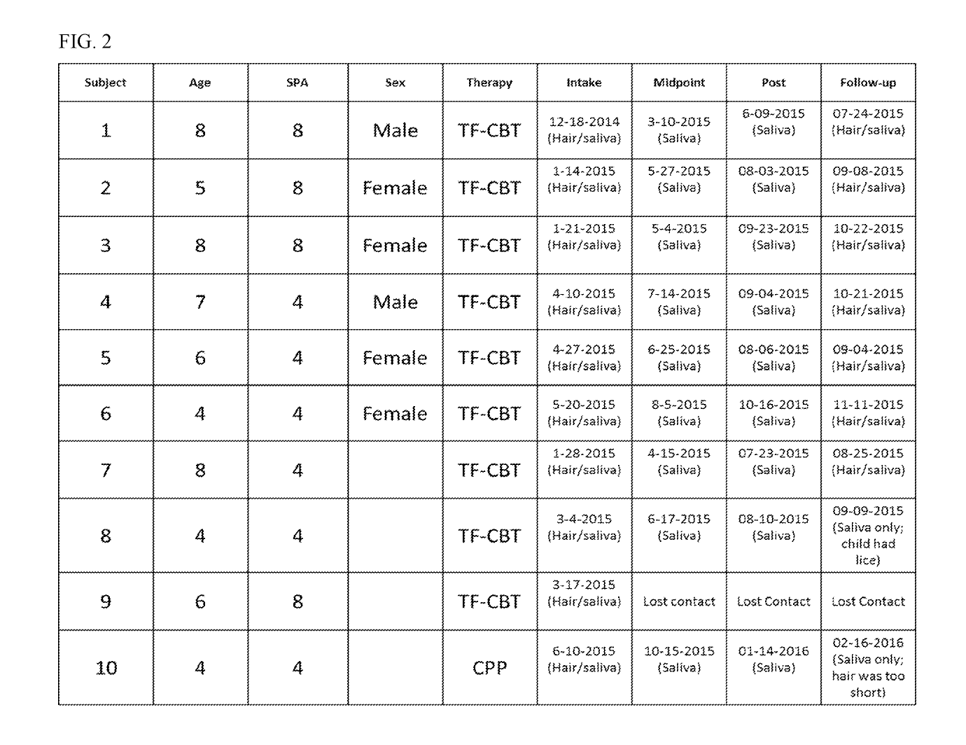

Saliva was collected for DNA methylation analysis, and hair was collected for cortisol analysis from children referred by Los Angeles County Child Protective Services. These children were referred to Children's institute Inc. for recruitment into a Trauma-Focused Cognitive Behavioral Therapy program (TF-CBT), and they were followed for 6-8 months throughout the therapy as shown in FIG. 1. Saliva was sampled at four time points (intake, midpoint, post-treatment, and 1-month follow-up) (FIG. 1). Hair was sampled at two time points (intake and 1-month follow-up) to allow growth in between. Saliva was collected using DNA Genotek OGR-250 collection cups. DNA was extracted and purified from the saliva samples using the PrepIT-L2P protocol and followed with RNase double-digestion to ensure purified DNA absent of RNA contamination. Three centimeters of hair were ground in a ball-grinder and cortisol was extracted using. Six children had reliable cortisol measurements at both intake and 1-month follow-up, thus, DNA methylation analysis was carried out for only these six children at both intake and 1-month follow-up. Psychosocial data was collected for these children at all four time points. Behavioral measures included Eyeberg Child Behavior Inventory (ECBI), Trauma Symptom Checklist for Young Children (TSCYC), Youth Outcome Questionnaire (YOQ), and the UCLA PTSD Reaction Index (UCLA PTSD RI).

### Protocol 2: Changes in Methylation Patterns in Children Exposed to Severe Traumatic Experiences.

In the first study, children (n=10) referred by LA County for trauma-focused behavioral therapy because of reported trauma were enrolled. Six children completed the therapy and donated sufficient biospecimens for analysis. Whole saliva was obtained by spitting into a collection vesicle (ORAGENE saliva collection), stored at room temperature, and DNA extracted with a DNeasy kit (Qiagen, USA).

In addition, a longitudinal study was performed to assess the effects of trauma from childhood into early adulthood. Saliva was collected at two time points, from entry (average age=11 years) to conclusion of the study (average 18 years). Whole saliva was collected by passive drool into 2-mL cryovials (Salimetrics, Inc., Carlsbad, Calif.). Samples were stored at −80° C. after collection. DNA was purified from saliva using an adapted protocol from QIAmp DNA Mini Kit (Qiagen, USA).

Genomic DNA from both types of collection was concentrated and amounts assessed by Qubit 2.0 Fluorometer using the Qubit dsDNA High Sensitivity Assay.

Behavioral data was collected throughout this study by survey instruments from care-givers. Only behavioral data similar to the pilot study was used for comparing two individuals at two time points.

### Microarray Analysis:

Purified DNA was sent to Keck Medical School Epigenome Center for analysis with the Illumina MethylationEPIC BeadChip (Illumina, San Diego, Calif.). DNA from every sample was bisulfite converted and run in duplicate. Genomic DNA (500-1000 ng) was bisulfite converted using the Zymo EZ DNA methylation kit (Zymo Research, Irvine, Calif.) according to the manufacturer's recommendations. After recovery, 8 μl of each bisulfite converted DNA sample further processed using the Illumina FFPE Restoration Solution (Illumina, San Diego, Calif.) as specified by the manufacturer. The Restoration Solution repairs degraded DNAs for use in genome-scale genotyping and DNA methylation assay platforms. The entire restored sample was then used as a substrate for the Illumina HM450 BeadArrays, as recommended by the manufacturer and previously described (Bibikova et al., 2012. Genomics 98(4):288-295).

Specifically, each sample was whole genome amplified (WGA) and then enzymatically fragmented. Samples were then hybridized overnight to an eight sample BeadArray, in which the WGA-DNA molecules anneal to locus-specific DNA oligomers linked to individual bead types. The oligomer probe designs follow the Infinium I and II chemistries, in which base extension follows hybridization to a locus-specific oligomer. With respect to the Infinium I probes, there are two different bead types for each locus, one with an oligomer that anneals specifically to the methylated version of the locus, while the other oligomer anneals to the unmethylated version of the locus. The probes terminate complementary to the interrogated CpG site for methylated loci, or complementary to the TpG for unmethylated alleles. A matched oligomer-template DNA molecule hybrid will allow for the incorporation of a labeled nucleotide immediately upstream (5′) to the interrogated CpG (or TpG) site. However, if the probe and template are mismatched, then primer extension will not occur. Adenine and thymine nucleotides are labeled with cy5 (red), while cytosine nucleotides are labeled with cy3 (green). No insertion of guanine nucleotides occurs in Infinium I assays. Of note, the identity of the dye is representative of the nucleotide adjacent to the CpG dinucleotide. The methylation discrimination is derived from separate measurements from the two different types of beads present for each locus. For some loci, both measurements will be cy3, and for others both will be cy5. The Infinium type II chemistry is a true two-color system. A matched oligomer-template DNA molecule hybrid will allow for the incorporation of a labeled nucleotide at the interrogated C or T of the CpG site. Adenine nucleotides labeled with cy5 (red) are incorporated across from unmethylated (TpG) sites, while guanine nucleotides labeled with cy3 (green) are incorporated across from methylated (CpG) sites.

After the chemistry steps, BeadArrays are scanned and the raw signal intensities are extracted from the *.IDAT files using the R package minfi. The intensities are corrected for background fluorescence and red-green dye-bias using the methods previously described by (Triche et al., 2013. Nucleic Acids Res 41(7):e90). The beta value is calculated as (M/(M+U)), in which M and U refer to the (pre-processed) mean methylated and unmethylated probe signal intensities, respectively. Measurements in which the fluorescent intensity is not statistically significantly above background signal (detection p value>0.05) are removed from the data set. In addition, probes that overlap with known SNPs as well as repetitive elements are masked prior to data analyses. Specifically, all MethylationEPIC probes that overlap with any common SNPs.

### Statistical Analysis

Data preprocessing was done in R using packages adapted for MethylationEPIC data. Data was background corrected using noob (normal-exponential convolution using out-of-band probes) from the R package minfi, which takes the known out-of-band probes present on the array that provide signals in the opposite color channel and uses normal-exponential convolution to subtract the background average intensity (from the out-of-band probes) from the foreground intensity. The data was normalized using dasen in wateRmelon (Illumina, San Diego, Calif.). Quality control was performed using RnBeads (Max-Planck-Institut Informatik, Saarbrucken, Germany), and no samples were removed due to poor quality. All replicates had a pearson correlation greater than 0.99 indicating high reproducibility of the bead chip. Filtering was performed in RnBeads to remove those probes that have a detection p-value greater than 0.05, are located on sex chromosomes, are in non-CG context, or overlapped any SNPs. Differential methylation analysis was carried out in RnBeads to look for differentially methylated sites and regions. Surrogate variable analysis was conducted to account for any underlying variables significantly contributing to the variance seen between time points. No surrogate variables were identified. Principle component analysis identified components that were significantly correlated with traits in our data, primarily sentrix ID and time point. Components relating to minor variance in the data included age and sex thus we carried out a covariate analysis to remove effects due to these traits. Covariates included age and sex. Sentrix ID was also adjusted for to remove batch effects from chip differences. Service planning area was included as a covariate in the pilot study only because we did not have information about this variable for the longitudinal study.

Differential analysis was used to compare time points within subject. Differential analysis was also carried out to compare individuals based on level of abuse. Differential methylation between groups was analyzed in promoter regions, which are assigned as the regions from 1.5 kb upstream to 0.5 kb downstream of the transcription start site according to Ensembl annotations. The statistical tool Limma was used to determine differentially methylated positions (DMPs). False Discovery Rate (FDR) correction was performed at the region level for promoters to adjust p-values.

The complete disclosure of all patents, patent applications, and publications, and electronically available material (including, for instance, nucleotide sequence submissions in, for example, GenBank and RefSeq, and amino acid sequence submissions in, for example, SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

TABLE 1

| Name | UCSC_RefGene_Name | Chromosome | Start | mean.diff |
|---|---|---|---|---|
| cg19377607 | LRRC20; LRRC20; LRRC20 | chr10 | 72138124 | 0.532609284 |
| cg20485607 | | chr1 | 120217696 | 0.459564106 |
| cg01146324 | | chr13 | 30887187 | 0.610803808 |
| cg25826698 | | chr3 | 16101981 | 0.506532186 |
| cg22840887 | | chr10 | 29545279 | −0.337903182 |
| cg01454153 | CYP2F1 | chr19 | 41620211 | 0.452656376 |
| cg23066280 | PTPRN2; PTPRN2; PTPRN2 | chr7 | 158032496 | 0.325749759 |
| cg24648384 | GNA12 | chr7 | 2802942 | 0.55826487 |
| cg10950028 | GSTM1; GSTM1 | chr1 | 110230633 | −0.465856313 |
| cg04850148 | CCL4L2; CCL4L1 | chr17 | 34539744 | −0.489558085 |
| cg19080354 | ATHL1 | chr11 | 288021 | 0.353380836 |
| cg14580085 | | chr2 | 239553406 | 0.384630004 |
| cg02583163 | | chr10 | 132291878 | 0.493446325 |
| cg06210526 | CYP2F1 | chr19 | 41619850 | 0.462579163 |
| cg15498409 | | chr22 | 18832673 | 0.375265263 |
| cg18428180 | KIAA0319; KIAA0319; KIAA0319; KIAA0319; KIAA0319 | chr6 | 24646492 | 0.289515172 |
| cg08692111 | | chr19 | 1737123 | 0.437362248 |
| cg07227024 | ALS2CR12; ALS2CR12 | chr2 | 202163482 | 0.345553278 |
| cg24348479 | DCST1; DCST2; DCST1 | chr1 | 155007323 | 0.453666163 |
| cg11188103 | PTPN14 | chr1 | 214668616 | 0.514252751 |
| cg26785303 | | chr1 | 171096963 | 0.634310754 |
| cg18845598 | OR2A5 | chr7 | 143746594 | 0.275721846 |
| cg02612588 | GPR111 | chr6 | 47623886 | 0.356798079 |
| cg21223983 | | chr12 | 95072169 | 0.312213915 |
| cg13749548 | | chr14 | 75722495 | 0.355527886 |
| cg13136933 | LOC283663 | chr15 | 57592040 | −0.2755441 |
| cg10156055 | MRPL35; MRPL35 | chr2 | 86440464 | 0.412209199 |
| cg13626582 | LOC283663 | chr15 | 57592083 | −0.335115896 |
| cg25593510 | GSTM5 | chr1 | 110254662 | −0.421124042 |
| cg12391372 | SEMA4B; SEMA4B | chr15 | 90755932 | 0.256986489 |
| cg15574437 | MAD1L1; MAD1L1; MAD1L1 | chr7 | 2019874 | 0.48091729 |
| cg02276807 | | chr12 | 133022677 | 0.266166849 |
| cg00009523 | SNTG2 | chr2 | 1165351 | 0.355538723 |
| cg18433519 | KIAA0319; KIAA0319; KIAA0319; KIAA0319; KIAA0319 | chr6 | 24646494 | 0.23581153 |
| cg20141578 | BCL2L14; BCL2L14; BCL2L14 | chr12 | 12225262 | −0.33672506 |
| cg13478757 | EHMT1; EHMT1 | chr9 | 140515874 | 0.362023413 |
| cg20803293 | GSTM5 | chr1 | 110254709 | −0.422195492 |
| cg07102368 | C15orf27 | chr15 | 76455848 | −0.301305215 |
| cg01156747 | | chr7 | 120659 | 0.559152017 |
| cg04035553 | | chr8 | 143757498 | 0.227074793 |
| cg01022117 | PKP1; PKP1 | chr1 | 201258280 | 0.338233668 |
| cg17004290 | CYP2U1 | chr4 | 108853384 | −0.225913347 |
| cg03424554 | WWP2; WWP2 | chr16 | 69830414 | 0.225551048 |
| cg24893378 | PART1 | chr5 | 59837426 | 0.611613177 |
| cg02707854 | SLC27A1 | chr19 | 17600122 | −0.44666273 |
| cg18049365 | KLHL14 | chr18 | 30349476 | −0.110993065 |
| cg24307368 | SLN | chr11 | 107582884 | −0.23656225 |
| cg23208326 | MTHFD2L | chr4 | 75149859 | 0.388822191 |
| cg03077671 | LYL1 | chr19 | 13211225 | −0.364949125 |
| cg07110356 | MPO | chr17 | 56355431 | −0.344832611 |
| cg16528895 | SULT1C4 | chr2 | 108994311 | −0.305192507 |
| cg08328750 | KRT15 | chr17 | 39675355 | 0.380920157 |
| cg13978689 | CD84 | chr1 | 160549261 | −0.31086141 |
| cg08818984 | NR3C1; NR3C1; NR3C1; NR3C1; NR3C1 | chr5 | 142814827 | −0.50971455 |
| cg27638913 | OSBPL1A; OSBPL1A | chr18 | 21849412 | 0.525324971 |
| cg26720913 | NR3C1; NR3C1; NR3C1; NR3C1; NR3C1; NR3C1 | chr5 | 142814934 | −0.489288198 |
| cg22584138 | SLC6A4 | chr17 | 28562220 | 0.307527904 |
| cg21842274 | CRHBP | chr5 | 76248637 | −0.378520564 |
| cg21840153 | OSBPL1A; OSBPL1A | chr18 | 21852436 | 0.199658846 |
| cg02682525 | ANKK1 | chr11 | 113258277 | 0.254898613 |
| cg16545105 | CRHBP; CRHBP | chr5 | 76248749 | −0.295274436 |
| cg25487047 | PCDHA9; PCDHA6; PCDHA6; PCDHA4; PCDHA13; PCDHAC1; PCDHA10; PCDHA8; PCDHA3; | chr5 | 140389945 | −0.201351168 |
| | PCDHA1; PCDHA10; PCDHA5; PCDHAl2; PCDHA1; PCDHAC2; PCDHA2; PCDHA7; PCDHA11 | | | |
| cg05590274 | ANKK1 | chr11 | 113262625 | −0.263462334 |
| cg27047406 | SLC6A2 | chr16 | 55689534 | −0.167616394 |
| cg23365739 | OTX2; OTX2 | chr14 | 57277178 | −0.163255969 |
| cg03492327 | OTX2; OTX2 | chr14 | 57273276 | −0.172554452 |
| cg16405454 | ANKK1 | chr11 | 113258223 | 0.252308453 |
| cg13947929 | CRHR1; CRHR1; CRHR1; CRHR1 | chr17 | 43863356 | 0.158834151 |
| cg19976235 | OTX2; OTX2 | chr14 | 57272858 | −0.156725804 |
| cg17342132 | NR3C1; NR3C1; NR3C1; NR3C1; NR3C1; NR3C1; NR3C1 | chr5 | 142780254 | 0.264534664 |
| cg26998537 | OTX2 | chr14 | 57277366 | −0.174672407 |
| cg04874129 | SLC6A2 | chr16 | 55690873 | −0.132986247 |
| cg25962358 | PCDHAC2; PCDHA7; PCDHAl2; PCDHA6; PCDHA10; PCDHA4; PCDHA11; PCDHA8; PCDHA6, | chr5 | 140347293 | −0.131012321 |
| | PCDHA1; PCDHA2; PCDHA1; PCDHA9; PCDHA13; PCDHA5; PCDHAC1; PCDHA3; PCDHAC2; | | | |
| | PCDHA10 | | | |
| cg02712145 | CRHR2 | chr7 | 30722433 | −0.121070925 |
| cg15607672 | OTX2 | chr14 | 57277227 | −0.12237907 |
| cg05815275 | ANKK1 | chr11 | 113258390 | 0.22029313 |
| cg19547547 | PCDHA1; PCDHA1; PCDHA1 | chr5 | 140166059 | −0.138907076 |
| cg15923943 | PCDHA7; PCDHAl2; PCDHA6; PCDHA10; PCDHA4; PCDHA11; PCDHAC2; PCDHA8; PCDHA6; | chr5 | 140383566 | −0.166229433 |
| | PCDHA1; PCDHA2; PCDHA1; PCDHA9; PCDHA13; PCDHA5; PCDHAC1; PCDHA3; PCDHA10 | | | |
| cg01071966 | CRHBP; CRHBP | chr5 | 76248923 | −0.175544879 |
| cg08845721 | NR3C1; NR3C1; NR3C1; NR3C1; NR3C1; NR3C1; NR3C1 | chr5 | 142780693 | 0.257819518 |
| cg19457823 | NR3C1; NR3C1; NR3C1; NR3C1; NR3C1; NR3C1; NR3C1 | chr5 | 142692961 | 0.17913025 |
| cg24119225 | PCDHA7; PCDHAC2; PCDHAl2; PCDHA6; PCDHA10; PCDHA4; PCDHA11; PCDHA8; PCDHA6; | chr5 | 140344483 | −0.158475717 |
| | PCDHA1; PCDHA2; PCDHA1; PCDHA9; PCDHA13; PCDHA5; PCDHAC1; PCDHA3; PCDHA10 | | | |
| cg26196496 | CRHBP | chr5 | 76247679 | 0.327084551 |
| cg12629325 | PCDHAC1; PCDHA7; PCDHAC1; PCDHAl2; PCDHA6; PCDHA10; PCDHA4; PCDHA11; PCDHA8; | chr5 | 140306458 | 0.087957654 |
| | PCDHAC1; PCDHA6; PCDHA1; PCDHA2; PCDHA9; PCDHA1; PCDHAC1; PCDHA13; | | | |
| | PCDHA5; PCDHA3; PCDHA10 | | | |
| cg15116095 | PCDHA1; PCDHA1; PCDHA1 | chr5 | 140166589 | −0.13094164 |
| cg02693870 | SLC6A2 | chr16 | 55689324 | −0.146610478 |
| cg22158248 | ALDH2 | chr12 | 112204136 | 0.141093196 |
| cg23928118 | PCDHA6; PCDHA2; PCDHA1; PCDHA7; PCDHA1; PCDHA6; PCDHA5; PCDHA3; PCDHA4; | chr5 | 140215201 | −0.097807367 |
| | PCDHA7 | | | |
| | PCDHA6; PCDHA2; PCDHA1; PCDHA9; PCDHA7; PCDHA1; PCDHA6; PCDHA5; PCDHA10; | chr5 | 140240941 | −0.102374718 |
| cg23985374 | PCDHA3; PCDHA4; PCDHA10; PCDHA8 | | | |
| cg03857453 | NR3C1; NR3C1; NR3C1; NR3C1; NR3C1; NR3C1; NR3C1 | chr5 | 142729913 | −0.161627288 |
| cg24076884 | PCDHAC2; PCDHA7; PCDHAl2; PCDHA6; PCDHA10; PCDHA4; PCDHA11; PCDHA8; PCDHA6, | chr5 | 140346921 | −0.078388068 |
| | PCDHA1; PCDHA2; PCDHA1; PCDHA9; PCDHA13; PCDHA5; PCDHAC1; PCDHA3; PCDHAC2; | | | |
| | PCDHA10 | | | |
| cg03391642 | PCDHA6; PCDHA2; PCDHA1; PCDHA9; PCDHA7; PCDHA1; PCDHA6; PCDHA5; PCDHA11; | chr5 | 140250948 | −0.107305925 |
| | PCDHA11; PCDHA10; PCDHA3; PCDHA4; PCDHA10; PCDHA8 | | | |
| cg25704227 | OSBPL1A | chr18 | 21974223 | 0.158403226 |
| cg19928377 | PCDHA6; PCDHA2; PCDHA1; PCDHA9; PCDHA7; PCDHA1; PCDHA6; PCDHA5; PCDHA11; | chr5 | 140249735 | −0.101738139 |
| | PCDHA11; PCDHA10; PCDHA3; PCDHA4; PCDHA10; PCDHA8 | | | |
| cg11976069 | OSBPL1A | chr18 | 21854521 | −0.138253909 |
| cg26282566 | PCDHA2; PCDHA1; PCDHA1; PCDHA4; PCDHA3; PCDHA4 | chr5 | 140186580 | −0.067090942 |
| cg02742676 | PCDHA7; PCDHAl2; PCDHA6; PCDHA10; PCDHA4; PCDHA11; PCDHAC2; PCDHA8; PCDHA6; | chr5 | 140357014 | 0.12572241 |
| | PCDHA1; PCDHA2; PCDHA1; PCDHA9; PCDHA13; PCDHA5; PCDHAC1; PCDHA3; PCDHA10 | | | |
| cg14514813 | PCDHA6; PCDHA2; PCDHA1; PCDHA9; PCDHA7; PCDHA1; PCDHA6; PCDHA5; PCDHA11; | chr5 | 140248772 | −0.062558227 |
| | PCDHA11; PCDHA10; PCDHA3; PCDHA4; PCDHA10; PCDHA8 | | | |
| cg16158779 | ANKK1 | chr11 | 113258455 | 0.094982006 |
| cg13777717 | CRHBP | chr5 | 76250527 | −0.060236215 |
| cg07380705 | CRHBP | chr5 | 76249897 | −0.05925045 |
| cg02047300 | PCDHA6; PCDHA2; PCDHA1; PCDHA1; PCDHA6; PCDHA5; PCDHA3; PCDHA4; PCDHA6 | chr5 | 140208201 | −0.112039348 |
| cg01049782 | CRHR2 | chr7 | 30717954 | 0.099747711 |
| cg12074493 | SLC6A4 | chr17 | 28564117 | −0.057031928 |
| cg21619814 | PCDHA2; PCDHA1; PCDHA1; PCDHA2; PCDHA2; PCDHA2 | chr5 | 140174449 | −0.131135772 |
| cg21963703 | PCDHA7; PCDHAl2; PCDHA6; PCDHA10; PCDHA4; PCDHA11; PCDHAC2; PCDHA8; PCDHA6; | chr5 | 140350766 | 0.146471174 |
| | PCDHA1; PCDHA2; PCDHA1; PCDHA9; PCDHA13; PCDHA5; PCDHAC1; PCDHA3; PCDHA10 | | | |
| cg03758467 | PCDHA2; PCDHA1; PCDHA1; PCDHA6; PCDHA5; PCDHA6; PCDHA3; PCDHA4; PCDHA6 | chr5 | 140207636 | −0.078405055 |
| cg04306063 | CRHBP | chr5 | 76249502 | −0.048427292 |
| cg03667083 | CRHR2 | chr7 | 30721010 | −0.104362797 |
| cg18073390 | PCDHA1; PCDHA1; PCDHA1 | chr5 | 140166568 | −0.122649534 |
| cg05241355 | OTX2 | chr14 | 57278351 | −0.056871409 |
| cg18535456 | ALDH2 | chr12 | 112203801 | 0.093035045 |
| cg09936799 | PCDHA6; PCDHA2; PCDHA1; PCDHA9; PCDHA7; PCDHA1; PCDHA6; PCDHA5; PCDHA10; | chr5 | 140241497 | −0.105693017 |
| | PCDHA3; PCDHA4; PCDHA10; PCDHA8 | | | |
| cg02500479 | PCDHA7; PCDHA13; PCDHAl2; PCDHA6; PCDHA10; PCDHA4; PCDHA11; PCDHA8; PCDHA1; | chr5 | 140263994 | −0.056883386 |
| | PCDHA2; PCDHA6; PCDHA1; PCDHA9; PCDHA13; PCDHA5; PCDHA3; PCDHA10 | | | |
| cg15615793 | CRHR2 | chr7 | 30722643 | −0.106123744 |
| cg00025823 | CRHR1; CRHR1; CRHR1; CRHR1 | chr17 | 43909151 | 0.121632128 |
| cg00682007 | PCDHA7; PCDHAC2; PCDHAl2; PCDHA6; PCDHA10; PCDHA4; PCDHA11; PCDHA8; PCDHA6; | chr5 | 140344745 | −0.076265569 |
| | PCDHA1; PCDHA2; PCDHA1; PCDHA9; PCDHA13; PCDHA5; PCDHAC1; PCDHA3; PCDHA10 | | | |
| cg27122725 | NR3C1; NR3C1; NR3C1; NR3C1; NR3C1; NR3C1; NR3C1 | chr5 | 142781723 | −0.039630362 |
| cg12962191 | PCDHA6; PCDHA2; PCDHA1; PCDHA7; PCDHA1; PCDHA6; PCDHA5; PCDHA7; PCDHA3; | chr5 | 140213924 | −0.097754716 |
| | PCDHA4 | | | |
| cg14579239 | PCDHA6; PCDHA2; PCDHA1; PCDHA8; PCDHA7; PCDHA1; PCDHA6; PCDHA5; PCDHA8; | chr5 | 140220822 | −0.089513536 |
| | PCDHA3; PCDHA4 | | | |
| cg06997114 | OSBPL1A; OSBPL1A | chr18 | 21851455 | 0.03684943 |
| cg22642909 | PCDHA6; PCDHA2; PCDHA1; PCDHA7; PCDHA1; PCDHA8; PCDHA6; PCDHA5; PCDHA8; | chr5 | 140220943 | −0.10643877 |
| | PCDHA3; PCDHA4 | | | |
| cg06841846 | SLC6A4 | chr17 | 28564094 | −0.03650967 |
| cg24748769 | OTX2; OTX2 | chr14 | 57271015 | −0.054095135 |
| cg19186356 | ALDH2 | chr12 | 112204327 | −0.035086838 |
| cg25967418 | PCDHA7; PCDHAl2; PCDHA6; PCDHA10; PCDHA4; PCDHA11; PCDHA8; PCDHA1; PCDHA2; | chr5 | 140261759 | −0.10123284 |
| | PCDHA6; PCDHA1; PCDHA9; PCDHA5; PCDHA13; PCDHA3; PCDHA13; PCDHA10 | | | |
| cg25928477 | OSBPL1A; OSBPL1A | chr18 | 21852313 | −0.032553181 |
| cg27122213 | OTX2 | chr14 | 57274763 | −0.032530078 |
| cg11539296 | PCDHA6; PCDHA2; PCDHA1; PCDHA7; PCDHA1; PCDHA6; PCDHA5; PCDHA3; PCDHA4; | chr5 | 140216145 | −0.068873249 |
| | PCDHA7 | | | |
| cg05212570 | ALDH2 | chr12 | 112247671 | 0.072640391 |
| cg20278291 | OTX2; OTX2; OTX2 | chr14 | 57272340 | −0.031225853 |
| cg03226000 | SLC6A2 | chr16 | 55690378 | 0.031004741 |
| cg27503360 | CRHR1; CRHR1; CRHR1; CRHR1 | chr17 | 43890749 | 0.060134204 |
| cg14995055 | PCDHA6; PCDHA2; PCDHA1; PCDHA9; PCDHA7; PCDHA1; PCDHA6; PCDHA5; PCDHA10; | chr5 | 140240593 | 0.064078756 |
| cg01089834 | PCDHA3; PCDHA4; PCDHA10; PCDHA8 | chr5 | 140180637 | −0.029260197 |
| | PCDHA2; PCDHA1; PCDHA1; PCDHA3; PCDHA3 | | | |
| cg17508591 | PCDHA6; PCDHA2; PCDHA1; PCDHA7; PCDHA1; PCDHA6; PCDHA5; PCDHA3; PCDHA4; | chr5 | 140214138 | −0.051052353 |
| | PCDHA7 | | | |
| cg11445191 | PCDHA6; PCDHA2; PCDHA1; PCDHA7; PCDHA1; PCDHA6; PCDHA5; PCDHA9; PCDHA9; | chr5 | 140227765 | 0.143638164 |
| | PCDHA9; PCDHA3; PCDHA9; PCDHA4; PCDHA8 | | | |
| cg18640030 | CRH | chr8 | 67090752 | −0.082735412 |
| cg13298827 | PCDHA6; PCDHA2; PCDHA1; PCDHA9; PCDHA7; PCDHA1; PCDHA6; PCDHA5; PCDHA10; | chr5 | 140243301 | 0.067018099 |
| | PCDHA3; PCDHA4; PCDHA10; PCDHA8 | | | |
| cg10042106 | OTX2 | chr14 | 57274739 | −0.027855592 |
| cg05884167 | CRHBP | chr5 | 76252686 | 0.167404823 |
| cg18029321 | PCDHA6; PCDHA2; PCDHA1; PCDHA7; PCDHA1; PCDHA6; PCDHA5; PCDHA9; PCDHA9; | chr5 | 140230326 | −0.113346994 |
| | PCDHA3; PCDHA4; PCDHA8 | | | |
| cg08935663 | PCDHAC1; PCDHA7; PCDHAl2; PCDHA6; PCDHA10; PCDHA4; PCDHA11; PCDHA8; PCDHA6, | chr5 | 140309344 | 0.065999446 |
| | PCDHA1; PCDHA2; PCDHA1; PCDHA9; PCDHA13; PCDHAC1; PCDHA5; PCDHAC1; PCDHA3; | | | |
| | PCDHA10 | | | |
| cg19895517 | OTX2; OTX2 | chr14 | 57269996 | −0.065348681 |
| cg00269606 | CRH | chr8 | 67090928 | 0.063608471 |
| cg01972879 | CRHR2 | chr7 | 30720901 | −0.058530196 |
| cg16234335 | PCDHA2; PCDHA1; PCDHA1; PCDHA4; PCDHA3; PCDHA4 | chr5 | 140188119 | −0.060272861 |
| cg11338426 | CRHR1; CRHR1; CRHR1; CRHR1; CRHR1; CRHR1; CRHR1; CRHR1 | chr17 | 43861668 | −0.024392492 |
| cg18998365 | NR3C1; NR3C1; NR3C1; NR3C1; NR3C1; NR3C1; NR3C1 | chr5 | 142781532 | 0.090342395 |
| cg26269677 | CRHBP | chr5 | 76251688 | −0.120035167 |
| cg04736351 | OTX2 | chr14 | 57274550 | −0.0270533 |
| cg16174029 | PCDHAC2; PCDHAC2; PCDHA7; PCDHAl2; PCDHA6; PCDHA10; PCDHA4; PCDHA11; PCDHA8; | chr5 | 140346280 | −0.023334032 |
| | PCDHA6; PCDHA1; PCDHA2; PCDHA1; PCDHA9; PCDHA13; PCDHA5; PCDHAC1; PCDHA3; | | | |
| | PCDHAC2; PCDHA10 | | | |
| cg02326566 | PCDHA6; PCDHA2; PCDHA1; PCDHA7; PCDHA1; PCDHA8; PCDHA6; PCDHA5; PCDHA8; | chr5 | 140220686 | −0.089489203 |
| | PCDHA3; PCDHA4 | | | |
| cg03694936 | PCDHA2; PCDHA1; PCDHA3; PCDHA1; PCDHA3 | chr5 | 140179490 | 0.107038411 |
| cg26882009 | PCDHAC2; PCDHA7; PCDHAl2; PCDHA6; PCDHA10; PCDHA4; PCDHA11; PCDHA8; PCDHA6, | chr5 | 140346106 | 0.025236437 |
| | PCDHA1; PCDHA2; PCDHA1; PCDHA9; PCDHAC2; PCDHA13; PCDHA5; PCDHAC1; PCDHA3; | | | |
| | PCDHAC2; PCDHA10 | | | |
| cg02051771 | PCDHA7; PCDHAl2; PCDHA6; PCDHA10; PCDHA4; PCDHA11; PCDHA8; PCDHA1; PCDHA2; | chr5 | 140261663 | 0.06175743 |
| | PCDHA6; PCDHA1; PCDHA9; PCDHA5; PCDHA13; PCDHA3; PCDHA13; PCDHA10 | | | |
| cg26318513 | PCDHA7; PCDHAl2; PCDHA6; PCDHA13; PCDHA10; PCDHA4; PCDHA11; PCDHA8; PCDHA1; | chr5 | 140260925 | 0.057794224 |
| | PCDHA2; PCDHA6; PCDHA1; PCDHA9; PCDHA13; PCDHA5; PCDHA3; PCDHA10 | | | |
| cg06928315 | PCDHA6; PCDHA2; PCDHA1; PCDHA9; PCDHA7; PCDHA1; PCDHA6; PCDHA5; PCDHA10; | chr5 | 140243526 | 0.056346549 |
| | PCDHA3; PCDHA4; PCDHA10; PCDHA8 | | | |
| cg13521908 | CRHR1; CRHR1; CRHR1; CRHR1; CRHR1; CRHR1; CRHR1; CRHR1 | chr17 | 43861682 | −0.022019942 |
| cg13023623 | OTX2 | chr14 | 57276257 | −0.021771227 |
| cg06952416 | NR3C1; NR3C1; NR3C1; NR3C1; NR3C1; NR3C1; NR3C1 | chr5 | 142781736 | −0.021614641 |
| cg02036261 | OTX2 | chr14 | 57276057 | −0.021313034 |
| cg19777540 | PCDHA2; PCDHA1; PCDHA1; PCDHA3; PCDHA3 | chr5 | 140180691 | −0.096830393 |
| cg16664570 | CRH | chr8 | 67091580 | 0.052993014 |
| cg10649903 | PCDHA2; PCDHA1; PCDHA3; PCDHA3; PCDHA1 | chr5 | 140181058 | 0.084609068 |
| cg02560085 | PCDHA6; PCDHA2; PCDHA1; PCDHA8; PCDHA7; PCDHA1; PCDHA6; PCDHA5; PCDHA8; | chr5 | 140220810 | −0.075125421 |
| | PCDHA3; PCDHA4 | | | |
| cg16642545 | CRHR1; CRHR1; CRHR1; CRHR1 | chr17 | 43878769 | −0.048735574 |
| cg15827318 | PCDHA6; PCDHA2; PCDHA1; PCDHA9; PCDHA7; PCDHA1; PCDHA6; PCDHA5; PCDHA10; | chr5 | 140252671 | 0.154537769 |
| | PCDHA3; PCDHA4; PCDHA11; PCDHA10; PCDHA8 | | | |
| cg10835712 | OTX2; OTX2 | chr14 | 57269102 | 0.049221273 |
| cg23706497 | OTX2; OTX2; OTX2 | chr14 | 57272244 | −0.018557956 |
| cg19106945 | PCDHA2; PCDHA1; PCDHA1; PCDHA5; PCDHA6; PCDHA3; PCDHA6; PCDHA4; PCDHA6 | chr5 | 140206841 | −0.042636007 |
| cg08929103 | CRHR1; CRHR1; CRHR1; CRHR1 | chr17 | 43860355 | 0.042346727 |
| cg20884605 | ALDH2 | chr12 | 112205368 | 0.018288633 |
| cg06282446 | PCDHA1; PCDHA1; PCDHA2; PCDHA2 | chr5 | 140174554 | −0.054430867 |
| cg23356309 | PCDHA7; PCDHAC2; PCDHAl2; PCDHA6; PCDHA10; PCDHA4; PCDHA11; PCDHA8; PCDHA6; | chr5 | 140344620 | −0.041938916 |
| | PCDHA1; PCDHA2; PCDHA1; PCDHA9; PCDHA13; PCDHA5; PCDHAC1; PCDHA3; PCDHA10 | | | |
| cg24869588 | PCDHA7; PCDHAl2; PCDHA6; PCDHA10; PCDHA4; PCDHA11; PCDHA8; PCDHA6; PCDHA1; | chr5 | 140345643 | −0.029390918 |
| | FCDHA2; PCDHA1; PCDHA9; PCDHAC2; PCDHA13; PCDHA5; PCDHAC1; PCDHAC2; PCDHA3; | | | |
| | PCDHA10 | | | |
| cg07555125 | PCDHA1; PCDHA1; PCDHA2; PCDHA2 | chr5 | 140175691 | −0.045541932 |
| cg06966811 | OTX2 | chr14 | 57276789 | −0.06793826 |
| cg26656751 | CRHR1; CRHR1; CRHR1; CRHR1 | chr17 | 43910226 | 0.04508915 |
| cg25225155 | PCDHA6; PCDHA2; PCDHA1; PCDHA7; PCDHA1; PCDHA8; PCDHA6; PCDHA5; PCDHA8; | chr5 | 140222253 | −0.042802765 |
| | PCDHA3; PCDHA4 | | | |
| cg06827234 | PCDHA6; PCDHA2; PCDHA1; PCDHA7; PCDHA1; PCDHA6; PCDHA5; PCDHA7; PCDHA3; | chr5 | 140213795 | −0.053526672 |
| | PCDHA4 | | | |
| cg13826890 | OTX2 | chr14 | 57275282 | −0.016116171 |
| cg20528338 | OSBPL1A | chr18 | 21976748 | −0.044599983 |
| cg13619597 | PCDHA7; PCDHA6; PCDHA10; PCDHA4; PCDHA11; PCDHAl2; PCDHAl2; PCDHA8; PCDHA1; | chr5 | 140256422 | −0.042156155 |
| | PCDHA2; PCDHA6; PCDHA1; PCDHA9; PCDHA5; PCDHA3; PCDHA10 | | | |
| cg13309828 | PCDHA6; PCDHA2; PCDHA1; PCDHA7; PCDHA1; PCDHA8; PCDHA6; PCDHA5; PCDHA8; | chr5 | 140223264 | −0.086645084 |
| | PCDHA3; PCDHA4 | | | |
| cg07733851 | NR3C1; NR3C1; NR3C1; NR3C1; NR3C1; NR3C1; NR3C1 | chr5 | 142781498 | 0.058127341 |
| cg18484679 | NR3C1; NR3C1; NR3C1; NR3C1; NR3C1; NR3C1; NR3C1 | chr5 | 142740314 | 0.109100958 |
| cg27604145 | PCDHA2; PCDHA1; PCDHA1; PCDHA4; PCDHA3; PCDHA4 | chr5 | 140186489 | −0.07671209 |
| cg04634417 | PCDHA6; PCDHA2; PCDHA1; PCDHA8; PCDHA7; PCDHA1; PCDHA6; PCDHA5; PCDHA8; | chr5 | 140220803 | 0.039919673 |
| | PCDHA3; PCDHA4 | | | |
| cg27410679 | CRHR1; CRHR1; CRHR1; CRHR1 | chr17 | 43866278 | −0.036932624 |
| cg17852221 | PCDHA7; PCDHA13; PCDHAl2; PCDHA6; PCDHA10; PCDHA4; PCDHA11; PCDHA8; PCDHA1; | chr5 | 140263655 | −0.039787703 |
| | PCDHA2; PCDHA6; PCDHA1; PCDHA9; PCDHA13; PCDHA5; PCDHA3; PCDHA10 | | | |
| cg17093478 | PCDHA6; PCDHA2; PCDHA1; PCDHA9; PCDHA7; PCDHA1; PCDHA6; PCDHA5; PCDHA10; | chr5 | 140242408 | −0.040433938 |
| | PCDHA3; PCDHA4; PCDHA10; PCDHA8 | | | |
| cg08215831 | CRH | chr8 | 67090776 | −0.047314178 |
| cg11560197 | PCDHA2; PCDHA1; PCDHA3; PCDHA3; PCDHA1 | chr5 | 140182956 | −0.048528918 |
| cg02204262 | PCDHA2; PCDHA1; PCDHA1; PCDHA3; PCDHA4; PCDHA5; PCDHA5 | chr5 | 140201746 | −0.057063215 |
| cg23742217 | OSBPL1A; OSBPL1A | chr18 | 21853511 | 0.038716712 |
| cg23316987 | OTX2; OTX2 | chr14 | 57272594 | −0.013603978 |
| cg24648119 | PCDHA6; PCDHA2; PCDHA1; PCDHA9; PCDHA7; PCDHA1; PCDHA6; PCDHA5; PCDHA10; | chr5 | 140242639 | −0.054996039 |
| | PCDHA3; PCDHA4; PCDHA10; PCDHA8 | | | |
| cg03860054 | SLC6A2 | chr16 | 55691102 | −0.035922293 |
| cg18283342 | PCDHA6; PCDHA2; PCDHA1; PCDHA9; PCDHA7; PCDHA1; PCDHA6; PCDHA10; PCDHA5; | chr5 | 140235435 | −0.038339881 |
| | PCDHA3; PCDHA4; PCDHA10; PCDHA10; PCDHA8 | | | |
| cg26514430 | PCDHA6; PCDHA2; PCDHA1; PCDHA9; PCDHA7; PCDHA1; PCDHA6; PCDHA5; PCDHA11; | chr5 | 140249080 | −0.06973153 |
| | PCDHA11; PCDHA10; PCDHA3; PCDHA4; PCDHA10; PCDHA8 | | | |
| cg07507057 | PCDHA6; PCDHA2; PCDHA1; PCDHA7; PCDHA1; PCDHA8; PCDHA6; PCDHA5; PCDHA8; | chr5 | 140223107 | −0.043636788 |
| | PCDHA3; PCDHA4 | | | |
| cg03405789 | CRH | chr8 | 67088895 | 0.047902753 |
| cg16014000 | PCDHA7; PCDHAC1; PCDHAl2; PCDHA6; PCDHA10; PCDHA4; PCDHA11; PCDHAC1; PCDHA8; | chr5 | 140305713 | −0.012761263 |
| | PCDHA6; PCDHA1; PCDHA2; PCDHA1; PCDHA9; PCDHA13; PCDHA5; PCDHA3; PCDHA10 | | | |
| cg01819552 | CRHR2 | chr7 | 30706917 | 0.030296124 |
| cg17326173 | PCDHA2; PCDHA1; PCDHA1; PCDHA3; PCDHA4; PCDHA5; PCDHA5 | chr5 | 140201539 | −0.038621074 |
| cg21199406 | CRHBP | chr5 | 76250201 | −0.012326887 |
| cg00822495 | OTX2 | chr14 | 57276490 | −0.011993718 |
| cg06968181 | NR3C1; NR3C1; NR3C1; NR3C1; NR3C1; NR3C1; NR3C1 | chr5 | 142784323 | −0.022902515 |
| cg14676529 | PCDHA6; PCDHA2; PCDHA1; PCDHA9; PCDHA7; PCDHA1; PCDHA6; PCDHA10; PCDHA5; | chr5 | 140235607 | −0.054106108 |
| | PCDHA3; PCDHA4; PCDHA10; PCDHA10; PCDHA8 | | | |
| cg25913647 | CRHBP | chr5 | 76248131 | 0.050502806 |
| cg18134262 | PCDHA7; PCDHA13; PCDHAl2; PCDHA6; PCDHA10; PCDHA4; PCDHA11; PCDHA8; PCDHA1; | chr5 | 140263086 | −0.044672531 |
| | PCDHA2; PCDHA6; PCDHA1; PCDHA9; PCDHA13; PCDHA5; PCDHA3; PCDHA10 | | | |
| cg17205316 | OTX2 | chr14 | 57275386 | −0.010806986 |
| cg25500080 | PCDHAC2; PCDHAC2; PCDHA7; PCDHAl2; PCDHA6; PCDHA10; PCDHA4; PCDHA11; PCDHP8; | chr5 | 140346199 | −0.010796095 |
| | PCDHA6; PCDHA1; PCDHA2; PCDHA1; PCDHA9; PCDHA13; PCDHA5; PCDHAC1; PCDHA3; | | | |
| | PCDHAC2; PCDHA10 | | | |
| cg06801483 | ALDH2 | chr12 | 112226133 | −0.073293129 |
| cg19035496 | CRH | chr8 | 67090792 | −0.03532681 |
| cg04922810 | CRHR2 | chr7 | 30721888 | −0.010296013 |
| cg05067406 | SLC6A2 | chr16 | 55705694 | −0.028802626 |
| cg01155497 | PCDHA2; PCDHA1; PCDHA1; PCDHA3; PCDHA4; PCDHA5; PCDHA5 | chr5 | 140203293 | −0.029138295 |
| cg19301632 | OSBPL1A | chr18 | 21978109 | −0.009956014 |
| cg08473090 | CRHR1; CRHR1; CRHR1; CRHR1 | chr17 | 43861104 | −0.009890261 |
| cg09516959 | CRHR2 | chr7 | 30721478 | −0.010206417 |
| cg13648501 | NR3C1; NR3C1; NR3C1; NR3C1 | chr5 | 142785258 | −0.009649595 |
| cg24063856 | CRHR1; CRHR1; CRHR1; CRHR1 | chr17 | 43863303 | 0.042134152 |
| cg03318614 | PCDHA1; PCDHA1; PCDHA2; PCDHA2 | chr5 | 140174612 | −0.048670702 |
| cg24394631 | CRHR1; CRHR1; CRHR1; CRHR1 | chr17 | 43863000 | −0.01423763 |
| cg14557699 | PCDHAl2; PCDHA7; PCDHA6; PCDHA10; PCDHA4; PCDHA11; PCDHA8; PCDHA1; PCDHA2; | chr5 | 140254909 | −0.036624644 |
| | PCDHA6; PCDHA1; PCDHA9; PCDHA5; PCDHA3; PCDHAl2; PCDHA10 | | | |
| cg13784080 | OTX2 | chr14 | 57275413 | −0.009224333 |
| cg25725890 | SLC6A4 | chr17 | 28563054 | 0.009210091 |
| cg07778819 | CRHR1; CRHR1; CRHR1; CRHR1 | chr17 | 43862927 | −0.009194207 |
| cg02829712 | PCDHA6; PCDHA2; PCDHA1; PCDHA10; PCDHA9; PCDHA7; PCDHA1; PCDHA6; PCDHA5; | chr5 | 140236081 | −0.076468682 |
| | PCDHA3; PCDHA4; PCDHA10; PCDHA10; PCDHA8 | | | |
| cg25535999 | NR3C1; NR3C1; NR3C1; NR3C1; NR3C1; NR3C1; NR3C1 | chr5 | 142757312 | 0.026872248 |
| cg11954680 | PCDHAl2; PCDHA7; PCDHA6; PCDHA10; PCDHA4; PCDHA11; PCDHA8; PCDHA1; PCDHA2; | chr5 | 140254793 | −0.05633192 |
| | PCDHA6; PCDHA1; PCDHA9; PCDHA5; PCDHA3; PCDHAl2; PCDHA10 | | | |
| cg05620787 | CRHBP | chr5 | 76264831 | 0.026621044 |
| cg03384992 | OSBPL1A; OSBPL1A | chr18 | 21852325 | −0.017160701 |
| cg16987900 | PCDHA2; PCDHA1; PCDHA1; PCDHA6; PCDHA5; PCDHA6; PCDHA3; PCDHA4; PCDHA6 | chr5 | 140207609 | −0.030853911 |
| cg18780217 | ALDH2; ALDH2 | chr12 | 112204756 | −0.008739782 |
| cg10519368 | PCDHA6; PCDHA2; PCDHA1; PCDHA9; PCDHA7; PCDHA1; PCDHA6; PCDHA10; PCDHA5; | chr5 | 140235586 | −0.030302402 |
| | PCDHA3; PCDHA4; PCDHA10; PCDHA10; PCDHA8 | | | |
| cg21702128 | NR3C1; NR3C1; NR3C1; NR3C1; NR3C1; NR3C1; NR3C1 | chr5 | 142784721 | −0.008496701 |
| cg17482089 | PCDHA6; PCDHA2; PCDHA1; PCDHA9; PCDHA7; PCDHA1; PCDHA6; PCDHA5; PCDHA11; | chr5 | 140250432 | −0.043508532 |
| | PCDHA11; PCDHA10; PCDHA3; PCDHA4; PCDHA10; PCDHA8 | | | |
| cg06593258 | OSBPL1A; OSBPL1A | chr18 | 21852333 | 0.017404369 |
| cg07589972 | NR3C1; NR3C1; NR3C1 | chr5 | 142815463 | 0.02417883 |
| cg04450501 | SLC6A2 | chr16 | 55718994 | −0.024528388 |
| cg14692377 | SLC6A4; SLC6A4 | chr17 | 28562685 | 0.008076521 |
| cg27345592 | NR3C1; NR3C1; NR3C1 | chr5 | 142786405 | 0.024390683 |
| cg23419885 | PCDHA2; PCDHA1; PCDHA4; PCDHA4; PCDHA1; PCDHA3 | chr5 | 140185635 | 0.079164762 |
| cg10205431 | PCDHA6; PCDHA2; PCDHA1; PCDHA7; PCDHA1; PCDHA6; PCDHA5; PCDHA3; PCDHA4; | chr5 | 140215762 | −0.02125484 |
| | PCDHA7 | | | |
| cg23490829 | PCDHA1; PCDHA1 | chr5 | 140171681 | −0.048576608 |
| cg09746736 | SLC6A2 | chr16 | 55690130 | −0.007241839 |
| cg12466613 | NR3C1; NR3C1; NR3C1 | chr5 | 142815469 | 0.021911356 |

| Name | ProbeSeqA | | | | | |
|---|---|---|---|---|---|---|
| cg19377607 | AATCACCTAAATTATTTATAAACTTTTAAATATACACTTATCAACCRAAC | | | | | SEQ ID NO: 1 |
| cg20485607 | ACTTATCATTCTACAATCCACAAAACAACTTTCCAAAAAATTTACTAACC | | | | | SEQ ID NO: 2 |
| cg01146324 | TCAAAATATTTCTTATATATACCAAAATAAACACAAAACCTCTTAACAAC | | | | | SEQ ID NO: 3 |
| cg25826698 | ATTACAACAATAAAAACAAACTCAATATCCATATCAAATAAACAATTATC | | | | | SEQ ID NO: 4 |
| cg22840887 | ACAAAATATTAAAACAACCAAAACTACTTACTAAAATATACAAATAACTC | | | | | SEQ ID NO: 5 |
| cg01454153 | CTCRCTATAAACTTTAAATCCCTCCCATCATCTTACCATACCTTTCCTAC | | | | | SEQ ID NO: 6 |
| cg23066280 | CCAACCAAAATTAACAAAAATTTCATAACCCAATAAACATAAAAAAAACA | | | | | SEQ ID NO: 7 |
| cg24648384 | TTACCTTCCCAAACCCRTAAAAAAAATACTATAAAACTAAAACACAACCC | | | | | SEQ ID NO: 8 |
| cg10950028 | AAACAACTACAAAACAAACTCTAAAAACCATTCCTCTTCAAAACTACCCA | | | | | SEQ ID NO: 9 |
| cg04850148 | TACCTAACCATAATCCATATAAAACAACTCTAATAAATAAAATCCTTCCC | | | | | SEQ ID NO: 10 |
| cg19080354 | AATACAAAACCTACCTTAAATATTATAAAAATTCTATTCTCAACAAAAAC | | | | | SEQ ID NO: 11 |
| cg14580085 | TATTCTCTTTAACTTTCAAAAAACTAAACATATAACTCATTTCTATATAC | | | | | SEQ ID NO: 12 |
| cg02583163 | TACCTCAAAATCTAAAAACAAAACTACATCCTCCCACTCAAAAAAACCCA | | | | | SEQ ID NO: 13 |
| cg06210526 | CTAAAAACCTATTTTTTCATAATTCTCCATACTATTCCCTATCTCTTACC | | | | | SEQ ID NO: 14 |
| cg15498409 | TACAATCTCCTTATCTCCCTCTACTAAAATATTCTTCTCTCACTTCATCC | | | | | SEQ ID NO: 15 |
| cg18428180 | AACTAAACAAAAAAACTAAACAAAAAAACTAAACAAAAACAACTACCACA | | | | | SEQ ID NO: 16 |
| cg08692111 | AATACATCAACCCACAATAAAACAAACAAACATCCAACTTTATACAAACA | | | | | SEQ ID NO: 17 |
| cg07227024 | ATCAAATTTTCAATAATAAAAAAATCACACATCACCTTATAAATTTAAAC | | | | | SEQ ID NO: 18 |
| cg24348479 | AAAAAATCRAAACTTATTTCTTCTATACCCATACATCCCCTATAACTTCC | | | | | SEQ ID NO: 19 |
| cg11188103 | ACRTTAATTCCCTCATCTATAAAATAAAAACAATAACTACATATCACRAC | | | | | SEQ ID NO: 20 |
| cg26785303 | AATACCAAAAAACCTAAAATTCCCTAACAAATAAATTTAATCACTTTTCC | | | | | SEQ ID NO: 21 |
| cg18845598 | ATCTAAAATATACTCTTTAAATATTATTCCTAACRTAATACCTTTCATTC | | | | | SEQ ID NO: 22 |
| cg02612588 | CTTTCTAATTACTAAAATTATAATAATATAATTCCCTAATCATTCCTATC | | | | | SEQ ID NO: 23 |
| cg21223983 | TAATTTAAACTATTATCTACTTAAATATACRATTAATTCACTCCTACTCC | | | | | SEQ ID NO: 24 |
| cg13749548 | AAAAAAATAACAAAATAAATACAACCATCTTATTCAAACTTAACTCTACA | | | | | SEQ ID NO: 25 |
| cg13136933 | TATTTAACATCAAAAACAAAAAATCCAATAAAAAATAAAACCCACAACCA | | | | | SEQ ID NO: 26 |
| cg10156055 | CCTAATTAATCCTAATTCTATTAACATTAAACCAAACTCAATAAATAAAC | | | | | SEQ ID NO: 27 |
| cg13626582 | ACAACCAAACATAAACAACACTAAAAAACAAACTCTCCCAATATACTACA | | | | | SEQ ID NO: 28 |
| cg25593510 | AATCRCTATAAACCTAACAAAAACTAAATAAACTCATAAAACCTCAAAAC | | | | | SEQ ID NO: 29 |
| cg12391372 | TACTATCTAAAAACCCAAATACTTAAATCACCTCCAAAATAACTACAAAC | | | | | SEQ ID NO: 30 |
| cg15574437 | CCCACTAAAAAACACACCTAAACATATCCACTTCCTTCCTAAAAAACACA | | | | | SEQ ID NO: 31 |
| cg02276807 | AATCCTATAATCTTACAATAATAACATCATCTACAAACACAACTAAAACA | | | | | SEQ ID NO: 32 |
| cg00009523 | TACCRACAACAACRCTCATTAAAATCACTATATAACTAACAATAAACCCC | | | | | SEQ ID NO: 33 |
| cg18433519 | AAAAACTAAACAAAAAAACTAAACAAAAAAACTAAACAAAAACRACTACC | | | | | SEQ ID NO: 34 |
| cg20141578 | CAAAAACCATACAACAATACATCTACCAATACCTAAAATATAAATATTCA | | | | | SEQ ID NO: 35 |
| cg13478757 | ATTTACAAAAATAATCAATACATTTTCCTACCAAAAAAAACTAACCAACC | | | | | SEQ ID NO: 36 |
| cg20803293 | ACAAATTAAAAAAACTAACAAAACCAAACCCCACTTTAAACTTCTAAACA | | | | | SEQ ID NO: 37 |
| cg07102368 | TCTCCATCTAAATTACAAAACCCTACATATTCTTAATACTACAAAACCAC | | | | | SEQ ID NO: 38 |
| cg01156747 | ACTTATTCCTAAATAACTCCRACACATAAACTAAAAACACTACACTCACC | | | | | SEQ ID NO: 39 |
| cg04035553 | ACCRAATTCCCTAAAAATCCTATCTTCCACCTAATACAAAACATAAAACC | | | | | SEQ ID NO: 40 |
| cg01022117 | AAATACCCTCAAAAATATTTCAAAATAAACAAAAATAAACTATATCTACA | | | | | SEQ ID NO: 41 |
| cg17004290 | CACCATCACCTATTAATTCAAAATACAAATATAAAACAACCTAAAAACRC | | | | | SEQ ID NO: 42 |
| cg03424554 | ATTATTTACCCCTATAATCACRTAACTAACTAATATTACRACTCACTACC | | | | | SEQ ID NO: 43 |
| cg24893378 | TTCTAAAAATACAAACTCCCTTACTATAAAAAACCTCTCAAAAAAAAACC | | | | | SEQ ID NO: 44 |
| cg02707854 | ACCCCTAAAAACAAAAAAACAAATTACACCAACAAATAAACAAAAAAACA | | | | | SEQ ID NO: 45 |
| cg18049365 | CTTTCAAAAACTCTAAAATCAACTTTCCTCACACACTCTAAATTATTTAC | | | | | SEQ ID NO: 46 |
| cg24307368 | ACTATAATATCACTAATAAAACATCTCAAAAAAACACCCTCCRAAAATTC | | | | | SEQ ID NO: 47 |
| cg23208326 | TATTTATTTATATAAATAAATCATTCCCATCAATATTACCTACTTTTAAC | | | | | SEQ ID NO: 48 |
| cg03077671 | ACTATCATTTTATAACTCCCATACCCTAATATAACATACTCTATTTTACC | | | | | SEQ ID NO: 49 |
| cg07110356 | CAAAAAAAATAAACACATTAATCTAATTACAAATAATAATATTACTCCCA | | | | | SEQ ID NO: 50 |
| cg16528895 | ATAAAAACTATAAACAATATAATACTATATCCTATTAACTCACAAAAAAC | | | | | SEQ ID NO: 51 |
| cg08328750 | TACTCTCTTCTAACATAAAAAAATAAACCTATAATCCAAATATTAAAACC | | | | | SEQ ID NO: 52 |
| cg13978689 | AATAATACTAAACCATCTCTTTCAAAATCTAACCTTCTATAAAAAAACAC | | | | | SEQ ID NO: 53 |
| cg08818984 | AAACCTTAAAAAATCAAAAATCTTTCAAACCCTACAAAACCRTAAAATAC | | | | | SEQ ID NO: 54 |
| cg27638913 | TACTTCTAAATATATTACACCTTCTATAAAAATTTATTTAATCACCTAAC | | | | | SEQ ID NO: 55 |
| cg26720913 | TAATATCAAAAAATCTAAATTTTCTCCTTCTCTCTACCATTTAAAAAAAC | | | | | SEQ ID NO: 56 |
| cg22584138 | ACTTTCRTTTCAACCCATAATTTATATTTAATCTTTACTTTACTATCATC | | | | | SEQ ID NO: 57 |
| cg21842274 | ATATAACATTTAACAAATCAACAAAATACACTCCCTTATTCTTTATCTCC | | | | | SEQ ID NO: 58 |
| cg21840153 | CCTTTACCCTTCAAACCTAACTAAAATAATTTTACCTAAATAAATTTCAC | | | | | SEQ ID NO: 59 |
| cg02682525 | AACCTAAATAAACTAAATCCAACTTCTCAAACTAAACTTCTTCCAACTTC | | | | | SEQ ID NO: 60 |
| cg16545105 | ATACTAAACCACRCTAAAAATTTATAACTAAAAACTAAACCCTCRTCATC | | | | | SEQ ID NO: 61 |
| cg25487047 | AATTAATCTAAACTAACTCRTCTCAACCAATATCTAACACTCRCATACCC | | | | | SEQ ID NO: 62 |
| cg05590274 | ATAATTCAAATCTCTAACTCTTTTTCTTAAAACTTCRTTACTTAAACCAC | | | | | SEQ ID NO: 63 |
| cg27047406 | CCCATTTCRTTAATAAAAAAACTAACCAAATAATTTAAATAACCTACTAC | | | | | SEQ ID NO: 64 |
| cg23365739 | CRTCAAAAAATTACCAAAAAAACRCTTTCTCAACAAATCTCCCTAAAAAC | | | | | SEQ ID NO: 65 |
| cg03492327 | AAACTCTACTACAACTACCRAAAACCCAAATATCTAAAAATTTACCTCCC | | | | | SEQ ID NO: 66 |
| cg16405454 | CATAAAACCTTAAATACACTAAAAAAATACATAAACCRATTTCACTTAAC | | | | | SEQ ID NO: 67 |
| cg13947929 | AATACACACAAACCTTTTAAAAACTATTCCCATTCAATACCAACTACTCA | | | | | SEQ ID NO: 68 |
| cg19976235 | CATAACAAAAATTTTAATTATCTTAACTTTTTACCACTTTCAACTACTAC | | | | | SEQ ID NO: 69 |
| cg17342132 | AAAAAAAAACTACTATAAAAATTTCTACRTCTTCACCCTCACTAACTATC | | | | | SEQ ID NO: 70 |
| cg26998537 | ATATATTTTAAACAAAACCCAATATCTTTAAAAAACATTCTAATAATATC | | | | | SEQ ID NO: 71 |
| cg04874129 | CAATAAACCTAACCAACRTATAACRCTTCCCCTACCTCTACTACAAAAAC | | | | | SEQ ID NO: 72 |
| cg25962358 | CTACACATCAAACCAAAAAAAACAACTCTTCAACATAAATACCAATACCA | | | | | SEQ ID NO: 73 |
| cg02712145 | AAAAAACACCRCAACTCTACTAATCRAAAAACAACCRCAACCCAAAATTC | | | | | SEQ ID NO: 74 |
| cg15607672 | CRCTCTCAAAAAAATTTACTAAAAAAACRCCTCTCTAACAACTTTTTAAC | | | | | SEQ ID NO: 75 |
| cg05815275 | AAATAAAAAAATCCCAAACATAAACCCTAAAACCCCAACAAAAATAAAAC | | | | | SEQ ID NO: 76 |
| cg19547547 | TAACCRCRTTACTCAAAACCTAAAACTAAAACTAACRAAACTAATACCTC | | | | | SEQ ID NO: 77 |
| cg15923943 | AAAAAATATTTCTTCTTTCAATACTACCTCCTTTCCCTTCTAAAATCTCC | | | | | SEQ ID NO: 78 |
| cg01071966 | AAATTAAACRACATACTAACCTTACCTCTACAACTACTATACTCTACTCC | | | | | SEQ ID NO: 79 |
| cg08845721 | ACAACTAAAAACAAAAATATATCRAACTAAACTTAACTATTCATCCTACC | | | | | SEQ ID NO: 80 |
| cg19457823 | ATATAATAAAATCAATTTCTCTAAAATACCTTTCCTAACCTCCTCAAACC | | | | | SEQ ID NO: 81 |
| cg24119225 | ACACTCACTATCAATACTCCACTACCTAACCAATATTATTCCAAAAAAAC | | | | | SEQ ID NO: 82 |
| cg26196496 | AAAATTAAACTATAACACRAAAACCTCCTAATTAAACCAACAAAAAAACC | | | | | SEQ ID NO: 83 |
| cg12629325 | AATCCRAAACATAATCCTAAATCACCRTTAATATAACRTATTAATAAAAC | | | | | SEQ ID NO: 84 |
| cg15116095 | TACAAAATACAATTAAACTACTAATCACCRTCCTCRACRTTAATAATAAC | | | | | SEQ ID NO: 85 |
| cg02693870 | CATCTCTATTATTTATAAACAAAATAACAAATCATTTAAAATTATAACCC | | | | | SEQ ID NO: 86 |
| cg22158248 | AAAAAAAAATAAACAAATCATACAAATCATCCTATTCTAAACTCTAAACC | | | | | SEQ ID NO: 87 |
| cg23928118 | TCAAATTAATATCCACCTTCAAAAATTACTATTCATTAATACTAAACAAC | | | | | SEQ ID NO: 88 |
| cg23985374 | AAACCAATTCCACTACACCATCCTAAAAAAAACCAAAAACRACACATAAC | | | | | SEQ ID NO: 89 |
| cg03857453 | CACATAATTTCAATCATTAAACCTCATAAATTAAACTAAATAAATACCTC | | | | | SEQ ID NO: 90 |
| cg24076884 | AACACTTTACTATTCTCCTACAAAAACTTAAAATCCAACTCRAAATACTC | | | | | SEQ ID NO: 91 |
| cg03391642 | AACCATAAAATCCRTCTTAAATAAACCCTCCTCAAAACACACCCTCTACC | | | | | SEQ ID NO: 92 |
| cg25704227 | CTTCTCTTATTCATATCCATTCCATACCCACACCAACTTAAATACCATAC | | | | | SEQ ID NO: 93 |
| cg19928377 | ATACAATCTAAATAAAAATCTTAAACACCAACRATAACTCTCCTAAAATC | | | | | SEQ ID NO: 94 |
| cg11976069 | ATTTTTTCATCTACCTATATATTAAATATTAATATTACCTAAATTTCTAC | | | | | SEQ ID NO: 95 |
| cg26282566 | ATATAAAAATTTCCACAATTACTACTTACRATTTAAAACCACATAATATC | | | | | SEQ ID NO: 96 |
| cg02742676 | CAAATTTATAAAATAATTTACAAACATTTTCTTTTATTCCCTCTAACACC | | | | | SEQ ID NO: 97 |
| cg14514813 | CAAAAATACCATATTTAACCTCCTCCAAAACAAAATAATAAAACTAACCA | | | | | SEQ ID NO: 98 |
| cg16158779 | AAACCTCAAAACTCATATCTAAAATTTCCCCACCTTTACRAAACAAAAAC | | | | | SEQ ID NO: 99 |
| cg13777717 | CAAAACCATTCCAAAAACACAAAAACTATCTTCAATACACAAATAACCCA | | | | | SEQ ID NO: 100 |
| cg07380705 | CCCCAAAATACCTAAACATACTAAACCTCCAAAACCAATTCACCTTCACC | | | | | SEQ ID NO: 101 |
| cg02047300 | ATACAAATATTAACTCAAATTCCATCTTAACCTATAAACTCAATTCTAAC | | | | | SEQ ID NO: 102 |
| cg01049782 | ACTTCCACATATTTATACTATAATTTAACTCATTTAATCCTTACACCATC | | | | | SEQ ID NO: 103 |
| cg12074493 | ACCAAAACAAAAACCACAAAATAAACAAAAAACTTAAAAACCAAAATACA | | | | | SEQ ID NO: 104 |
| cg21619814 | TTTAAATATTCTAATCTCTATTAAAATCCTACTACTTCTCTTATTCCTCC | | | | | SEQ ID NO: 105 |
| cg21963703 | AAAACATAAACAAATCAAACCAAACTCAACTTTACCACCAACTATAAACC | | | | | SEQ ID NO: 106 |
| cg03758467 | TAAACACCATATCAAATACCTCTAATTTTATTTCCATTAAAAAATCTTCC | | | | | SEQ ID NO: 107 |
| cg04306063 | TTCCTACTCTTCAACRCCAACCTAAAACRAAAACTAACTAAAAAACAACC | | | | | SEQ ID NO: 108 |
| cg03667083 | RAATTAAAAAAAATACAACACCCCRCACCCTCTAACTTAACAAAACCTAC | | | | | SEQ ID NO: 109 |
| cg18073390 | CTAATAAAAACAAACCRAAACTACAAAATACAATTAAACTACTAATCACC | | | | | SEQ ID NO: 110 |
| cg05241355 | TTTTTACCTAACATAAAAAACAAAAACCAAACAAATTTCAAACAAATCCA | | | | | SEQ ID NO: 111 |
| cg18535456 | CAACTTTAATAAAATCTAATATTAAAATCAAAATCCCACCACCAAACACA | | | | | SEQ ID NO: 112 |
| cg09936799 | ACATAATAAACTCCTTTATCAAACACTAAACATCATCATTTACATCCAAC | | | | | SEQ ID NO: 113 |
| cg02500479 | ATTTACTTAATCATTACCATCTACRCRATATCCAACCTATTAATACTCAC | | | | | SEQ ID NO: 114 |
| cg15615793 | ATCTAATAAAATCAAATAACCTAAATAAATTAAAATCCTTAAATCAAACC | | | | | SEQ ID NO: 115 |
| cg00025823 | CTATAATAAAAACCACTTAAACRCCAAATCCRTCAATCAAATAAATTCCC | | | | | SEQ ID NO: 116 |
| cg00682007 | AAAAATTTAAAATCTATTACCTCATATACAAAAAAAACACTCACTAAAAC | | | | | SEQ ID NO: 117 |
| cg27122725 | ACTCCATTCAATACCTCATATTTAAAAATCATTATCACCTCAAATAAACA | | | | | SEQ ID NO: 118 |
| cg12962191 | CACCATTTCAAATTATTAACTTATTTTTCTAATCAACAAAACTTACCTTC | | | | | SEQ ID NO: 119 |
| cg14579239 | TTTTCTCTAACAACATCTATCTCTAAAAACCRAACAACRAAAAATACAAC | | | | | SEQ ID NO: 120 |
| cg06997114 | AACRACCAAACTTAATCCAAAAATATAAATAAATCCAAAAATAATTAAAC | | | | | SEQ ID NO: 121 |
| cg22642909 | CAACTATTTAACATAAATTATCACTAACAAAAAAAACTAAAATCCTAACA | | | | | SEQ ID NO: 122 |
| cg06841846 | CAAAATTCTAATACCACCTAAACACCAAAACAAAAACCACAAAATAAACA | | | | | SEQ ID NO: 123 |
| cg24748769 | AAATACCAAATCTTAACAAACAATACTTCCAACACATCTAACTACACCCA | | | | | SEQ ID NO: 124 |
| cg19186356 | TCAAATCTCACTTATCATTTCCTAACCATAATACTTATAAAAACAATACC | | | | | SEQ ID NO: 125 |
| cg25967418 | TTCCTACAAAAAATATAACAAAAAACRAAATACCTCTTACRAATCCTTCC | | | | | SEQ ID NO: 126 |
| cg25928477 | AACTACCTTTCAATCTTAATTTTAAAACCATAAACACAAACACTTCAACA | | | | | SEQ ID NO: 127 |
| cg27122213 | AACAAATTACACAAATTAACCATTAAATATAAAACTACACAAAAAACACA | | | | | SEQ ID NO: 128 |
| cg11539296 | TAAAACACACCAAAATAAACTTTACCAAACTACATACRCCCTCAAAAAAC | | | | | SEQ ID NO: 129 |
| cg05212570 | AAAATAAATTCAAATATATTATCCTCTCTCTAAAACRCTTCCTATAACTC | | | | | SEQ ID NO: 130 |
| cg20278291 | CCATACTCTTTTTAAATTAATACTAAAACATAAAAAAACTACTACCTCCA | | | | | SEQ ID NO: 131 |
| cg03226000 | TCTTCAACCRCCCCCAAAAAACTATCAAAAATCTCCAACTCTTAAATTCC | | | | | SEQ ID NO: 132 |
| cg27503360 | AACAAAAATACTAACCTTTCACTCAAAAATAAAAAAACTTTATCTCCTCA | | | | | SEQ ID NO: 133 |
| cg14995055 | ATTTTCTTCTATTAAAAAAAATACCTTTCAACCAAATACAATAACACTCA | | | | | SEQ ID NO: 134 |
| cg01089834 | ATAATATCRCTATCCACCACAAAATCTTTCTCCACAAAAAAAATAACAAC | | | | | SEQ ID NO: 135 |
| cg17508591 | ATAACAACTTCATAAACCACATCACACAAAACCTAAAACTAAAACTAACA | | | | | SEQ ID NO: 136 |
| cg11445191 | AAAACTTTTAAAAACAATCAATCTACTAAAAAAACTCCAACAAAAAAAAC | | | | | SEQ ID NO: 137 |
| cg18640030 | ACACRCAATCRAACTATCAAAAAAACRTCAACTTATTAAACAAATACTAC | | | | | SEQ ID NO: 138 |
| cg13298827 | CAACTTCCCAAAATACCAAAATTACAAATAAACATAAACTACCACACCCA | | | | | SEQ ID NO: 139 |
| cg10042106 | AAATATAAAACTACACAAAAAACACAAACAATAAAACAAAACCTCAAACA | | | | | SEQ ID NO: 140 |
| cg05884167 | AAATCAATAATACATTATTCTTTAACAAATAAAAACCCCATCTTTCAACC | | | | | SEQ ID NO: 141 |
| cg18029321 | CACCTAACAAACCAACACTAATATATTCTAACACAATAAAAAATTAATCA | | | | | SEQ ID NO: 142 |
| cg08935663 | CCTTCTTTTAACCCCCATTATAATTTATTTTTACATCTTTTATAATTATC | | | | | SEQ ID NO: 143 |
| cg19895517 | AAAAACAAATCCTTAACTAAACTTTTAAATAAACTCTCTATATAAATACC | | | | | SEQ ID NO: 144 |
| cg00269606 | AAATTTTTATCAATAAACAAATCATAAAAAACCCTTCCATTTTAAAACTC | | | | | SEQ ID NO: 145 |
| cg01972879 | AAACTAACCTACTAAACTAAATCAAAAACTATAAACAACATTATTCCTAC | | | | | SEQ ID NO: 146 |
| cg16234335 | CTTCRCTATAAACCACRACCAATATTTCTATAAAAATAACTAATATAAAC | | | | | SEQ ID NO: 147 |
| cg11338426 | ACACAAACCATAAAAAAAATAACAAAAACAAAACCAACAAAAAACAACCA | | | | | SEQ ID NO: 148 |
| cg18998365 | TACCTTTTATTAAACRAAATTTAACTATAATATCCCAAATACRCTTCTCC | | | | | SEQ ID NO: 149 |
| cg26269677 | ACCAAAAAATAAATTCACATTAACCATAAAAACAAACCCCAACCTCTTTC | | | | | SEQ ID NO: 150 |
| cg04736351 | TCTAAATATAAATTCTCCAAAAACCACCTCRTAAACAATCATAATAAAAC | | | | | SEQ ID NO: 151 |
| cg16174029 | TAAACAATAAAAAAACAAACACAAAATAACACAAACAAAAACAAAACCCA | | | | | SEQ ID NO: 152 |
| cg02326566 | ACAACRACATCATATAACTTCAAAAAATAATAATTACATAAATTACTTCC | | | | | SEQ ID NO: 153 |
| cg03694936 | TCTAAATTTAACCTCTTTCCATAAATTTTCTTTACCCCACTTCCAAATAC | | | | | SEQ ID NO: 154 |
| cg26882009 | TTCCTTTTCCCTCCCCCTAAAACTATAACAACAACAACAACAAAAAACCA | | | | | SEQ ID NO: 155 |
| cg02051771 | ATATAAAAACTAAATCATACTTACACTTACACRTTCATACRCATAATATC | | | | | SEQ ID NO: 156 |
| cg26318513 | TAAAAACTTTAACCTCCTAATTAAATTCTATTTAACCAAAACRCCRATTC | | | | | SEQ ID NO: 157 |
| cg06928315 | TTTCACCCTATCTCCCAAACTATAATACAATAACCTAATCTAAACTCACC | | | | | SEQ ID NO: 158 |
| cg13521908 | AAAAATAACAAAAACAAAACCAACAAAAAACAACCACAAACCAAACTACA | | | | | SEQ ID NO: 159 |
| cg13023623 | AAAACCAAAAAAAACAAACAATAAACACCAAAACAAACAAAAAACCAACA | | | | | SEQ ID NO: 160 |
| cg06952416 | AAAAATAAACTACTTATCTCCCCACCTCTCTACCCCATTTATCTAAAACA | | | | | SEQ ID NO: 161 |
| cg02036261 | CATATTAAAAAAACACTCTTCACATTCACTCAACCTAATAACCAAAATCA | | | | | SEQ ID NO: 162 |
| cg19777540 | ATTACRTATTCAAATACTACTTTACTTCATCCTCTCTAAAATTTAACACC | | | | | SEQ ID NO: 163 |
| cg16664570 | ATTCAATAAAATAAATAAAACTTATTTAACTCATACATCTCTCACTAACC | | | | | SEQ ID NO: 164 |
| cg10649903 | AATAAATCTACAAAATAACATTTTATTTATAAATTCTCAAATAAACCACA | | | | | SEQ ID NO: 165 |
| cg02560085 | ATATTCAAAATCTTTTCTCTAACAACATCTATCTCTAAAAACCRAACAAC | | | | | SEQ ID NO: 166 |
| cg16642545 | AACATTAAATACATCAACTACCCCAAACTTAAAATAAAAACATAATACCC | | | | | SEQ ID NO: 167 |
| cg15827318 | TAATAATATTACAAACTTTCTTAAAAATCATATCTAACTATTTCCCCCCC | | | | | SEQ ID NO: 168 |
| cg10835712 | TAAATTTATAATACRAAAACCATTCTTATCCTTAAAAAACTATCAAAACC | | | | | SEQ ID NO: 169 |
| cg23706497 | AAAATCAACCCAAATCAAAAATACCCAACTAAAAAATCTTAATACACCCA | | | | | SEQ ID NO: 170 |
| cg19106945 | CCCATTATACTTTACTATTTTTAATTCTTCAACTATTCCTAAATAATCTC | | | | | SEQ ID NO: 171 |
| cg08929103 | CTTCTAAAATAACTCCAACTCCAAATAAAAACCAATAAACCCATAAACAC | | | | | SEQ ID NO: 172 |
| cg20884605 | CAACAATAAAAACAACCRAACRTAACCTCRCCCATAATAAACTAAAAAAC | | | | | SEQ ID NO: 173 |
| cg06282446 | ACAAAACATACTCTTCTAATTTAATCAAAACATTAAAAATTAATAATAAC | | | | | SEQ ID NO: 174 |
| cg23356309 | CCTTTAAATTCTAAATATCAAAATCACAACCTTAACTCCTTTAACTATCC | | | | | SEQ ID NO: 175 |
| cg24869588 | AAAAATATAAAAATCAAATAAATAATTCCCATCRCCAATAACCCCAAAAC | | | | | SEQ ID NO: 176 |
| cg07555125 | CCAACTTAAAAAAAACRTAAAACRTCAAAAAACAAATAACATATCCATTC | | | | | SEQ ID NO: 177 |
| cg06966811 | AAAAAAAAATACATCCACCTACAAAACTCCACATTCACAAAAAAACATCA | | | | | SEQ ID NO: 178 |
| cg26656751 | AAACTATAAAAAAAAACCAAAAATACAACCTCATATTAAACCCATACCCA | | | | | SEQ ID NO: 179 |
| cg25225155 | TTCACTATAAACCACCACCAACTTATCTATAAAAATAACCAACATAAACA | | | | | SEQ ID NO: 180 |
| cg06827234 | CTTACTTACATAATAACCACATAATATCRCTCTTTACCRCAAATTCTTTC | | | | | SEQ ID NO: 181 |
| cg13826890 | TACCAAAAAAATTAACCTTAACTATTAATTATCATTAAAAAAAAACCCCA | | | | | SEQ ID NO: 182 |
| cg20528338 | AAAACCTCCTCACCTCTTTACTCAACATCTAAAAAATAATTTACAAATTC | | | | | SEQ ID NO: 183 |
| cg13619597 | TAAAATATCCATAAAAATAACCAACATAAACAACAATACACCTACATTCA | | | | | SEQ ID NO: 184 |
| cg13309828 | TTAAATAAACTTACTTTAAAACCATAATCAACATTTAAATCTTACTCTTC | | | | | SEQ ID NO: 185 |
| cg07733851 | CTATCCCAATTCTCAACCTATTCTCAAAAAAATAAAACTATTAATATATC | | | | | SEQ ID NO: 186 |
| cg18484679 | ATAACACTTAATAAAACATTAAAAACACTATTCCACCTAATAATATTTCC | | | | | SEQ ID NO: 187 |
| cg27604145 | CCCCCACACACTCTACTATAAACTACTTAATTTTATTTATTATTTCAATC | | | | | SEQ ID NO: 188 |
| cg04634417 | TTAATCAATATTCAAAATCTTTTCTCTAACAACATCTATCTCTAAAAACC | | | | | SEQ ID NO: 189 |
| cg27410679 | ACAATTAAAAAAAACCCRTTTCTATTCCCACCAAATACAAATATCAAATC | | | | | SEQ ID NO: 190 |
| cg17852221 | TAAATACAAACCACATAATAACAAAAATACACACAATAAACACCAATTCA | | | | | SEQ ID NO: 191 |
| cg17093478 | CAACCACTAAACTACRAAAAACTAAAACTACTACAATTCTAAATAAAAAC | | | | | SEQ ID NO: 192 |
| cg08215831 | TAATATCRACCCTCTTCAAAAACCACRCAACATTTACCTAATAAACTAAC | | | | | SEQ ID NO: 193 |
| cg11560197 | AATCTATTAATACTCACACTACTACTATATACTACTCTACRATACTCCRC | | | | | SEQ ID NO: 194 |
| cg02204262 | CTACAAATTTTCCATATAAAAATAACAATAAAAAACATCAATAACAATCC | | | | | SEQ ID NO: 195 |
| cg23742217 | AAATTCAAACTTTAAAATCAAACCAATCTAAATCTAAACTCTACTACCTC | | | | | SEQ ID NO: 196 |
| cg23316987 | AATACCCRAAAAAAACAATTACAATCTACCTTCCCCAAAAACAAAAACCC | | | | | SEQ ID NO: 197 |
| cg24648119 | CTATAACRCCTAATTATCRCAACAACTACATCTATCAACTAACAACACCC | | | | | SEQ ID NO: 198 |
| cg03860054 | AAAAATCATTTTCCCAAACTCTTTACAAAATACCTCRAACTCATTCCAAC | | | | | SEQ ID NO: 199 |
| cg18283342 | AACTAATACCTTTATTCTAAAAAACTATTTTATAAACRACAACRACATCC | | | | | SEQ ID NO: 200 |
| cg26514430 | ATTACTTAAATTCCRCAATCAACAACTTTTATTCTCTAAACRAAAACACC | | | | | SEQ ID NO: 201 |
| cg07507057 | ATATAACCAACTCCCCACCACACTAAAACACACCAAAATAAACTTACCCA | | | | | SEQ ID NO: 202 |
| cg03405789 | CACRTACAACAACAACACAATATTATTCRTTTTATCTACRTTTAATTTCC | | | | | SEQ ID NO: 203 |
| cg16014000 | CCTCTTAACAACTCCTAAAATACAAAAAAATACAAAACAAACTTCAACCC | | | | | SEQ ID NO: 204 |
| cg01819552 | CTATAAATAATTAATCTTTAAAACCCACRTCCCATTCTCCAAACATTCTC | | | | | SEQ ID NO: 205 |
| cg17326173 | TTAACCACATCACACAAAACCTAAAACTAAAACTAACAAAACTAATACCA | | | | | SEQ ID NO: 206 |
| cg21199406 | CAAAAACACCAACCCCAACCCCACTAAATATATATAAATCAAAATCTTCA | | | | | SEQ ID NO: 207 |
| cg00822495 | AAACTTCTCCCRCRATCTTTACRTTTTAACCCCCAAATTTAATTAAAATC | | | | | SEQ ID NO: 208 |
| cg06968181 | AAACAACAAACTTAAAAACAAATTACAAACAAAATAATAAATTACTAACA | | | | | SEQ ID NO: 209 |
| cg14676529 | CAAACAACTACATCTAAAAACCATCTTATCCTTCTCTTATTTTACAAATC | | | | | SEQ ID NO: 210 |
| cg25913647 | TTTACATAATATACACACTCCTAAACCAAATAACTATAACCATAACCTTC | | | | | SEQ ID NO: 211 |
| cg18134262 | TCAAACTAATATCCACCTACAAAAACTACTACTCATTAATACTAAACAAC | | | | | SEQ ID NO: 212 |
| cg17205316 | CTCCTACTCCCCTCTATTACTAATAACTTTTAATACCTATAAAAAATCCC | | | | | SEQ ID NO: 213 |
| cg25500080 | TAAAAAATCAACAAAAACAATAAAACCAAAAACCCTTAAAAAAACCTACA | | | | | SEQ ID NO: 214 |
| cg06801483 | CRCCTCCCCRAAAACACTAAACAATATCCAAAAACATTTTTAACAACCTC | | | | | SEQ ID NO: 215 |
| cg19035496 | CAAAAACCACRCAACATTTACCTAATAAACTAACRCTCTCTTAACAACTC | | | | | SEQ ID NO: 216 |
| cg04922810 | CAACCAACRCCAAACTACAATTAACCTCCAACAAACTATAAAACAATACC | | | | | SEQ ID NO: 217 |
| cg05067406 | TCTTAAAACTACCCCCACCCCAAAATCTATAAAAAAATTATCTTCCATAC | | | | | SEQ ID NO: 218 |
| cg01155497 | TATAACTATCAACRAAAACTCTCCATAATCCTTCACCAACACCAAAAAAC | | | | | SEQ ID NO: 219 |
| cg19301632 | AATAACAAACTTCAAACTATTAACCCAAAACTTATATTTTACCTAAAAAC | | | | | SEQ ID NO: 220 |
| cg08473090 | CCTACATTTAAACAATTCCCACCCTACAACTAACTAAAAAAAACTCCTCA | | | | | SEQ ID NO: 221 |
| cg09516959 | TCAACAACTTTATACCACTAAATCCAAAATCCTCTTTACTCCCTAAACCA | | | | | SEQ ID NO: 222 |
| cg13648501 | CAACRCCCTTCATAAAAATTCTTCTAACTAAAAATTCTACRTTAACATAC | | | | | SEQ ID NO: 223 |
| cg24063856 | CCCCATCCRTTACCTAAAAACTAAAAATAAACTCAAATTATTTTCTAAAC | | | | | SEQ ID NO: 224 |
| cg03318614 | CAAAAAAAACCAAAAAACCTAAACACAACTACTCTCACTTCTACTCCTCA | | | | | SEQ ID NO: 225 |
| cg24394631 | CAACTTAACCCRACTCAAAACATTCAAAACRATTACATTCAAAAAACTTC | | | | | SEQ ID NO: 226 |
| cg14557699 | TTAATAAATTTTTTCCAACATCACAATCATTATAAAATCRATCCTATTCC | | | | | SEQ ID NO: 227 |
| cg13784080 | TTTTAATACCTATAAAAAATCCCRAACTAAACAAAAAAAATCCTAACCCC | | | | | SEQ ID NO: 228 |
| cg25725890 | CAAAACAAAACCRAACRAAAAATCCTAACTTTCCTACTCTTTAACTTTAC | | | | | SEQ ID NO: 229 |
| cg07778819 | CTAAATACCCTAAACCRAATCAAACTAATATCTATATTTATAACTAAAAC | | | | | SEQ ID NO: 230 |
| cg02829712 | CRTAACAAAACAAAAACTCTCAATACCTAAATCCAAACTACTTAACTCTC | | | | | SEQ ID NO: 231 |
| cg25535999 | ATTTTTATAACTATCTCTTTATAATCCTCTCATTTCTAAACACTAAACTC | | | | | SEQ ID NO: 232 |
| cg11954680 | CCAAAATCAAAACATTAAAATTAAAAATAACCCAAAAAATAACTAAACCA | | | | | SEQ ID NO: 233 |
| cg05620787 | CACAACTAATTAAAAACCTTTCATACCAATCAATATTCCCAACCTTAAAC | | | | | SEQ ID NO: 234 |
| cg03384992 | TAAATATATAAAAACTACCTTTCAATCTTAATTTTAAAACCATAAACACA | | | | | SEQ ID NO: 235 |
| cg16987900 | TATTTCCATTAAAAAATCTTCCACATAAAAATAAAATACTATATATTCCA | | | | | SEQ ID NO: 236 |
| cg18780217 | ACAAAAAACAAAATCAACTCAATAACCCTAAAACCCTAACTCTACTCTCA | | | | | SEQ ID NO: 237 |
| cg10519368 | CATCTTATCCTTCTCTTATTTTACAAATCRACRAATCAACRATCTAAAAC | | | | | SEQ ID NO: 238 |
| cg21702128 | ACCCACACAACACAACCTTTCCCAAAATCACAAAAATCATAATCTATACC | | | | | SEQ ID NO: 239 |
| cg17482089 | ACAACTCAAACTAACAACACAAAAAACACAATTAACAAACTAATACCACA | | | | | SEQ ID NO: 240 |
| cg06593258 | TATAAACAATAAATATATAAAAACTACCTTTCAATCTTAATTTTAAAACC | | | | | SEQ ID NO: 241 |
| cg07589972 | AAATACTCAACTATCTACAAATCAAATATCTTAAAATAAAATCCRTTCTC | | | | | SEQ ID NO: 242 |
| cg04450501 | CAAATCCTATCTCCATCAACATACATTTACAAAAACTAATCTATAATCAC | | | | | SEQ ID NO: 243 |
| cg14692377 | AACTACACAAAAAAACTAATCCCAAACTAAACAAACAAACTAACCTCACA | | | | | SEQ ID NO: 244 |
| cg27345592 | ATCTCACRTCCTTTCTAACACACTAAAATTCATCTCTTCAATTAAATTCC | | | | | SEQ ID NO: 245 |
| cg23419885 | AACAAATATTTTTTCTTCTTTTACAAACTACAACACCAAAATAAAATCRC | | | | | SEQ ID NO: 246 |
| cg10205431 | ACAATCTATAAACACAAACCATATAATAACAAAAATACATACAATTAACA | | | | | SEQ ID NO: 247 |
| cg23490829 | AAAAAAAATTTCCCCTAAAATCTTCAAAAAAAATACRACCCAAAAAACAC | | | | | SEQ ID NO: 248 |
| cg09746736 | ATAAATACACCCTAAACACAAAACAAAACTAAATCTACCTAAAAAACCCA | | | | | SEQ ID NO: 249 |
| cg12466613 | TACTATAAATACTCAACTATCTACAAATCAAATATCTTAAAATAAAATCC | | | | | SEQ ID NO: 250 |
| Name | ProbeSeqB | | | | | |
| cg19377607 | | | | | | |
| cg20485607 | | | | | | |
| cg01146324 | | | | | | |
| cg25826698 | | | | | | |
| cg22840887 | | | | | | |
| cg01454153 | | | | | | |
| cg23066280 | CCGACCAAAATTAACAAAAATTTCGTAACCCAATAAACGTAAAAAAAACG | | | | | SEQ ID NO: 751 |
| cg24648384 | | | | | | |
| cg10950028 | AAACAACTACGAAACGAACTCTAAAAACCGTTCCTCTTCAAAACTACCCG | | | | | SEQ ID NO: 752 |
| cg04850148 | | | | | | |
| cg19080354 | | | | | | |
| cg14580085 | | | | | | |
| cg02583163 | TACCTCAAAATCTAAAAACAAAACTACGTCCTCCCGCTCAAAAAAACCCG | | | | | SEQ ID NO: 753 |
| cg06210526 | | | | | | |
| cg15498409 | | | | | | |
| cg18428180 | AACTAAACAAAAAAACTAAACAAAAAAACTAAACAAAAACGACTACCGCG | | | | | SEQ ID NO: 754 |
| cg08692111 | AATACATCGACCCACGATAAAACGAACAAACGTCCGACTTTATACAAACG | | | | | SEQ ID NO: 755 |
| cg07227024 | | | | | | |
| cg24348479 | | | | | | |
| cg11188103 | | | | | | |
| cg26785303 | | | | | | |
| cg18845598 | | | | | | |
| cg02612588 | | | | | | |
| cg21223983 | | | | | | |
| cg13749548 | AAAAAAATAACAAAATAAATACGACCATCTTATTCGAACTTAACTCTACG | | | | | SEQ ID NO: 756 |
| cg13136933 | TATTTAACATCAAAAACAAAAAATCCAATAAAAAATAAAACCCACGACCG | | | | | SEQ ID NO: 757 |
| cg10156055 | | | | | | |
| cg13626582 | ACGACCGAACGTAAACAACGCTAAAAAACAAACTCTCCCAATATACTACG | | | | | SEQ ID NO: 758 |
| cg25593510 | | | | | | |
| cg12391372 | | | | | | |
| cg15574437 | CCCGCTAAAAAACACGCCTAAACATATCCACTTCCTTCCTAAAAAACACG | | | | | SEQ ID NO: 759 |
| cg02276807 | AATCCTATAATCTTACGATAATAACGTCATCTACAAACGCGACTAAAACG | | | | | SEQ ID NO: 760 |
| cg00009523 | | | | | | |
| cg18433519 | | | | | | |
| cg20141578 | CGAAAACCGTACGACAATACGTCTACCAATACCTAAAATATAAATATTCG | | | | | SEQ ID NO: 761 |
| cg13478757 | | | | | | |
| cg20803293 | ACGAATTAAAAAAACTAACGAAACCGAACCCCGCTTTAAACTTCTAAACG | | | | | SEQ ID NO: 762 |
| cg07102368 | | | | | | |
| cg01156747 | | | | | | |
| cg04035553 | | | | | | |
| cg01022117 | AAATACCCTCAAAAATATTTCGAAATAAACAAAAATAAACTATATCTACG | | | | | SEQ ID NO: 763 |
| cg17004290 | | | | | | |
| cg03424554 | | | | | | |
| cg24893378 | | | | | | |
| cg02707854 | ACCCCTAAAAACGAAAAAACAAATTACGCCAACAAATAAACAAAAAAACG | | | | | SEQ ID NO: 764 |
| cg18049365 | | | | | | |
| cg24307368 | | | | | | |
| cg23208326 | | | | | | |
| cg03077671 | | | | | | |
| cg07110356 | CGAAAAAAATAAACGCGTTAATCTAATTACGAATAATAATATTACTCCCG | | | | | SEQ ID NO: 765 |
| cg16528895 | | | | | | |
| cg08328750 | | | | | | |
| cg13978689 | | | | | | |
| cg08818984 | | | | | | |
| cg27638913 | | | | | | |
| cg26720913 | | | | | | |
| cg22584138 | | | | | | |
| cg21842274 | | | | | | |
| cg21840153 | | | | | | |
| cg02682525 | | | | | | |
| cg16545105 | | | | | | |
| cg25487047 | | | | | | |
| cg05590274 | | | | | | |
| cg27047406 | | | | | | |
| cg23365739 | | | | | | |
| cg03492327 | | | | | | |
| cg16405454 | | | | | | |
| cg13947929 | GATACACGCGAACCTTTTAAAAACTATTCCCATTCAATACCAACTACTCG | | | | | SEQ ID NO: 766 |
| cg19976235 | | | | | | |
| cg17342132 | | | | | | |
| cg26998537 | | | | | | |
| cg04874129 | | | | | | |
| cg25962358 | CTACACGTCGAACCGAAAAAAACAACTCTTCAACATAAATACCAATACCG | | | | | SEQ ID NO: 767 |
| cg02712145 | | | | | | |
| cg15607672 | | | | | | |
| cg05815275 | | | | | | |
| cg19547547 | | | | | | |
| cg15923943 | | | | | | |
| cg01071966 | | | | | | |
| cg08845721 | | | | | | |
| cg19457823 | | | | | | |
| cg24119225 | | | | | | |
| cg26196496 | | | | | | |
| cg12629325 | | | | | | |
| cg15116095 | | | | | | |
| cg02693870 | | | | | | |
| cg22158248 | | | | | | |
| cg23928118 | | | | | | |
| cg23985374 | | | | | | |
| cg03857453 | | | | | | |
| cg24076884 | | | | | | |
| cg03391642 | | | | | | |
| cg25704227 | | | | | | |
| cg19928377 | | | | | | |
| cg11976069 | | | | | | |
| cg26282566 | | | | | | |
| cg02742676 | | | | | | |
| cg14514813 | CGAAAATACCGTATTTAACCTCCTCCGAAACGAAATAATAAAACTAACCG | | | | | SEQ ID NO: 768 |
| cg16158779 | | | | | | |
| cg13777717 | CAAAACCGTTCCGAAAACACAAAAACTATCTTCAATACGCGAATAACCCG | | | | | SEQ ID NO: 769 |
| cg07380705 | | | | | | |
| cg02047300 | | | | | | |
| cg01049782 | | | | | | |
| cg12074493 | GCCAAAACGAAAACCGCAAAATAAACGAAAAACTTAAAAACCGAAATACG | | | | | SEQ ID NO: 770 |
| cg21619814 | | | | | | |
| cg21963703 | | | | | | |
| cg03758467 | | | | | | |
| cg04306063 | | | | | | |
| cg03667083 | | | | | | |
| cg18073390 | | | | | | |
| cg05241355 | TTTTTACCTAACATAAAAAACAAAAACCGAACAAATTTCAAACGAATCCG | | | | | SEQ ID NO: 771 |
| cg18535456 | CGACTTTAATAAAATCTAATATTAAAATCAAAATCCCACCGCCGAACGCG | | | | | SEQ ID NO: 772 |
| cg09936799 | | | | | | |
| cg02500479 | | | | | | |
| cg15615793 | | | | | | |
| cg00025823 | | | | | | |
| cg00682007 | | | | | | |
| cg27122725 | ACTCCATTCGATACCTCGTATTTAAAAATCGTTATCGCCTCAAATAAACG | | | | | SEQ ID NO: 773 |
| cg12962191 | | | | | | |
| cg14579239 | | | | | | |
| cg06997114 | | | | | | |
| cg22642909 | CGACTATTTAACATAAATTATCACTAACGAAAAAAACTAAAATCCTAACG | | | | | SEQ ID NO: 774 |
| cg06841846 | CGAAATTCTAATACCACCTAAACGCCAAAACGAAAACCGCAAAATAAACG | | | | | SEQ ID NO: 775 |
| cg24748769 | AAATACCGAATCTTAACAAACAATACTTCCAACACATCTAACTACGCCCG | | | | | SEQ ID NO: 776 |
| cg19186356 | | | | | | |
| cg25967418 | | | | | | |
| cg25928477 | AACTACCTTTCAATCTTAATTTTAAAACCGTAAACGCGAACGCTTCAACG | | | | | SEQ ID NO: 777 |
| cg27122213 | GACAAATTACACGAATTAACCGTTAAATATAAAACTACGCGAAAAACGCG | | | | | SEQ ID NO: 778 |
| cg11539296 | | | | | | |
| cg05212570 | | | | | | |
| cg20278291 | CCGTACTCTTTTTAAATTAATACTAAAACGTAAAAAAACTACTACCTCCG | | | | | SEQ ID NO: 779 |
| cg03226000 | | | | | | |
| cg27503360 | AACAAAAATACTAACCTTTCACTCGAAAATAAAAAAACTTTATCTCCTCG | | | | | SEQ ID NO: 780 |
| cg14995055 | ATTTTCTTCTATTAAAAAAAATACCTTTCGACCGAATACGATAACACTCG | | | | | SEQ ID NO: 781 |
| cg01089834 | | | | | | |
| cg17508591 | ATAACAACTTCGTAAACCGCATCGCGCAAAACCTAAAACTAAAACTAACG | | | | | SEQ ID NO: 782 |
| cg11445191 | | | | | | |
| cg18640030 | | | | | | |
| cg13298827 | CGACTTCCCAAAATACCGAAATTACAAATAAACATAAACTACCGCACCCG | | | | | SEQ ID NO: 783 |
| cg10042106 | AAATATAAAACTACGCGAAAAACGCGAACAATAAAACGAAACCTCGAACG | | | | | SEQ ID NO: 784 |
| cg05884167 | | | | | | |
| cg18029321 | CGCCTAACAAACCGACGCTAATATATTCTAACGCGATAAAAAATTAATCG | | | | | SEQ ID NO: 785 |
| cg08935663 | | | | | | |
| cg19895517 | | | | | | |
| cg00269606 | | | | | | |
| cg01972879 | | | | | | |
| cg16234335 | | | | | | |
| cg11338426 | ACGCGAACCGTAAAAAAAATAACGAAAACGAAACCGACAAAAAACGACCG | | | | | SEQ ID NO: 786 |
| cg18998365 | | | | | | |
| cg26269677 | | | | | | |
| cg04736351 | | | | | | |
| cg16174029 | TAAACGATAAAAAAACAAACGCAAAATAACACGAACGAAAACGAAACCCG | | | | | SEQ ID NO: 787 |
| cg02326566 | | | | | | |
| cg03694936 | | | | | | |
| cg26882009 | TTCCTTTTCCCTCCCCCTAAAACTATAACGACAACAACAACAAAAAACCG | | | | | SEQ ID NO: 788 |
| cg02051771 | | | | | | |
| cg26318513 | | | | | | |
| cg06928315 | | | | | | |
| cg13521908 | AAAAATAACGAAAACGAAACCGACAAAAAACGACCGCGAACCGAACTACG | | | | | SEQ ID NO: 789 |
| cg13023623 | AAAACCAAAAAAAACGAACAATAAACGCCGAAACGAACGAAAAACCAACG | | | | | SEQ ID NO: 790 |
| cg06952416 | AAAAATAAACTACTTATCTCCCCACCTCTCTACCCCGTTTATCTAAAACG | | | | | SEQ ID NO: 791 |
| cg02036261 | CATATTAAAAAAACACTCTTCACGTTCGCTCGACCTAATAACCAAAATCG | | | | | SEQ ID NO: 792 |
| cg19777540 | | | | | | |
| cg16664570 | | | | | | |
| cg10649903 | AATAAATCTACAAAATAACATTTTATTTATAAATTCTCGAATAAACCGCG | | | | | SEQ ID NO: 793 |
| cg02560085 | | | | | | |
| cg16642545 | | | | | | |
| cg15827318 | | | | | | |
| cg10835712 | | | | | | |
| cg23706497 | AAAATCGACCCAAATCGAAAATACCCAACTAAAAAATCTTAATACGCCCG | | | | | SEQ ID NO: 794 |
| cg19106945 | | | | | | |
| cg08929103 | | | | | | |
| cg20884605 | | | | | | |
| cg06282446 | | | | | | |
| cg23356309 | | | | | | |
| cg24869588 | | | | | | |
| cg07555125 | | | | | | |
| cg06966811 | AAAAAAAAATACATCCGCCTACAAAACTCCACGTTCGCAAAAAAACATCG | | | | | SEQ ID NO: 795 |
| cg26656751 | AAACTATAAAAAAAAACCGAAAATACAACCTCGTATTAAACCCATACCCG | | | | | SEQ ID NO: 796 |
| cg25225155 | TTCGCTATAAACCACCGCCAACTTATCTATAAAAATAACCGACGTAAACG | | | | | SEQ ID NO: 797 |
| cg06827234 | | | | | | |
| cg13826890 | TACCAAAAAAATTAACCTTAACTATTAATTATCGTTAAAAAAAAACCCCG | | | | | SEQ ID NO: 798 |
| cg20528338 | | | | | | |
| cg13619597 | TAAAATATCCGTAAAAATAACCGACGTAAACGACAATACGCCTACGTTCG | | | | | SEQ ID NO: 799 |
| cg13309828 | | | | | | |
| cg07733851 | | | | | | |
| cg18484679 | | | | | | |
| cg27604145 | | | | | | |
| cg04634417 | | | | | | |
| cg27410679 | | | | | | |
| cg17852221 | TAAATACAAACCACGTAATAACGAAAATACGCGCGATAAACGCCGATTCG | | | | | SEQ ID NO: 800 |
| cg17093478 | | | | | | |
| cg08215831 | | | | | | |
| cg11560197 | | | | | | |
| cg02204262 | | | | | | |
| cg23742217 | | | | | | |
| cg23316987 | | | | | | |
| cg24648119 | | | | | | |
| cg03860054 | | | | | | |
| cg18283342 | | | | | | |
| cg26514430 | | | | | | |
| cg07507057 | ATATAACCAACTCCCCACCGCACTAAAACACACCAAAATAAACTTACCCG | | | | | SEQ ID NO: 801 |
| cg03405789 | | | | | | |
| cg16014000 | | | | | | |
| cg01819552 | | | | | | |
| cg17326173 | TTAACCGCATCGCGCAAAACCTAAAACTAAAACTAACGAAACTAATACCG | | | | | SEQ ID NO: 802 |
| cg21199406 | CAAAAACACCGACCCCAACCCCACTAAATATATATAAATCGAAATCTTCG | | | | | SEQ ID NO: 803 |
| cg00822495 | | | | | | |
| cg06968181 | AAACGACAAACTTAAAAACAAATTACAAACGAAATAATAAATTACTAACG | | | | | SEQ ID NO: 804 |
| cg14676529 | | | | | | |
| cg25913647 | | | | | | |
| cg18134262 | | | | | | |
| cg17205316 | | | | | | |
| cg25500080 | TAAAAAATCAACGAAAACGATAAAACCAAAAACCCTTAAAAAAACCTACG | | | | | SEQ ID NO: 805 |
| cg06801483 | | | | | | |
| cg19035496 | | | | | | |
| cg04922810 | | | | | | |
| cg05067406 | | | | | | |
| cg01155497 | | | | | | |
| cg19301632 | | | | | | |
| cg08473090 | CCTACGTTTAAACAATTCCCGCCCTACGACTAACTAAAAAAAACTCCTCG | | | | | SEQ ID NO: 806 |
| cg09516959 | TCAACAACTTTATACCGCTAAATCCGAAATCCTCTTTACTCCCTAAACCG | | | | | SEQ ID NO: 807 |
| cg13648501 | | | | | | |
| cg24063856 | | | | | | |
| cg03318614 | CAAAAAAAACCGAAAAACCTAAACACGACTACTCTCGCTTCTACTCCTCG | | | | | SEQ ID NO: 808 |
| cg24394631 | | | | | | |
| cg14557699 | | | | | | |
| cg13784080 | | | | | | |
| cg25725890 | | | | | | |
| cg07778819 | | | | | | |
| cg02829712 | | | | | | |
| cg25535999 | | | | | | |
| cg11954680 | CCAAAATCGAAACATTAAAATTAAAAATAACCCAAAAAATAACTAAACCG | | | | | SEQ ID NO: 809 |
| cg05620787 | | | | | | |
| cg03384992 | TAAATATATAAAAACTACCTTTCAATCTTAATTTTAAAACCGTAAACGCG | | | | | SEQ ID NO: 810 |
| cg16987900 | TATTTCCATTAAAAAATCTTCCGCATAAAAATAAAATACTATATATTCCG | | | | | SEQ ID NO: 811 |
| cg18780217 | ACGAAAAACGAAATCGACTCAATAACCCTAAAACCCTAACTCTACTCTCG | | | | | SEQ ID NO: 812 |
| cg10519368 | | | | | | |
| cg21702128 | | | | | | |
| cg17482089 | ACGACTCAAACTAACAACGCGAAAAACGCAATTAACAAACTAATACCGCG | | | | | SEQ ID NO: 813 |
| cg06593258 | | | | | | |
| cg07589972 | | | | | | |
| cg04450501 | | | | | | |
| cg14692377 | AACTACGCGAAAAAACTAATCCCGAACTAAACAAACGAACTAACCTCGCG | | | | | SEQ ID NO: 814 |
| cg27345592 | | | | | | |
| cg23419885 | | | | | | |
| cg10205431 | GCGATCTATAAACGCGAACCATATAATAACGAAAATACGTACAATTAACG | | | | | SEQ ID NO: 815 |
| cg23490829 | | | | | | |
| cg09746736 | GTAAATACGCCCTAAACGCGAAACAAAACTAAATCTACCTAAAAAACCCG | | | | | SEQ ID NO: 817 |
| cg12466613 | | | | | | |
| Name | NextBase | Color | Probe_rs | Probe_maf | CpG_rs | CpG_maf |
| cg19377607 | | | NA | NA | rs10823532 | 0.44768 |
| cg20485607 | | | rs640195 | 0.255909 | NA | NA |
| cg01146324 | | | NA | NA | rs17633138 | 0.124826 |
| cg25826698 | | | NA | NA | rs17041841 | 0.159884 |
| cg22840887 | | | NA | NA | rs75594963 | 0.044271 |
| cg01454153 | | | NA | NA | rs77890115 | 0.05599 |
| cg23066280 | T | Red | rs7792381 | 0.200306 | NA | NA |
| cg24648384 | | | NA | NA | rs798487 | 0.240365 |
| cg10950028 | C | Grn | rs145941576 | 0.0215 | NA | NA |
| cg04850148 | | | NA | NA | NA | NA |
| cg19080354 | | | NA | NA | rs10751646 | 0.49249 |
| cg14580085 | | | NA | NA | NA | NA |
| cg02583163 | A | Red | NA | NA | rs56870189 | 0.23193 |
| cg06210526 | | | NA | NA | rs62117444 | 0.059469 |
| cg15498409 | | | NA | NA | NA | NA |
| cg18428180 | C | Grn | NA | NA | NA | NA |
| cg08692111 | A | Red | NA | NA | rs12611251 | 0.180201 |
| cg07227024 | | | NA | NA | rs9288316 | 0.00067 |
| cg24348479 | | | rs4845405 | 0.440212 | NA | NA |
| cg11188103 | | | NA | NA | rs12089869 | 0.257741 |
| cg26785303 | | | NA | NA | rs66880604 | 0.101602 |
| cg18845598 | | | NA | NA | rs55979718 | 0.366088 |
| cg02612588 | | | rs16876142 | 0.000228 | NA | NA |
| cg21223983 | | | NA | NA | NA | NA |
| cg13749548 | T | Red | NA | NA | rs741847 | 0.381776 |
| cg13136933 | A | Red | NA | NA | rs112427003 | 0.013724 |
| cg10156055 | | | NA | NA | rs73946347 | 0.010064 |
| cg13626582 | A | Red | rs112427003 | 0.013724 | NA | NA |
| cg25593510 | | | NA | NA | NA | NA |
| cg12391372 | | | NA | NA | rs78141882 | 0.254803 |
| cg15574437 | C | Grn | NA | NA | rs12668156 | 0.325908 |
| cg02276807 | A | Red | rs116613177 | 0.030408 | NA | NA |
| cg00009523 | | | NA | NA | rs5024308 | 0.372029 |
| cg18433519 | | | NA | NA | NA | NA |
| cg20141578 | T | Red | NA | NA | NA | NA |
| cg13478757 | | | NA | NA | rs10867031 | 0.125075 |
| cg20803293 | C | Grn | rs1758104 | 0.14444 | NA | NA |
| cg07102368 | | | NA | NA | NA | NA |
| cg01156747 | | | NA | NA | NA | NA |
| cg04035553 | | | NA | NA | NA | NA |
| cg01022117 | A | Red | rs12025174 | 0.457534 | NA | NA |
| cg17004290 | | | NA | NA | rs6849089 | 0.448449 |
| cg03424554 | | | NA | NA | rs60231938 | 0.322536 |
| cg24893378 | | | NA | NA | rs79435314 | 0.037969 |
| cg02707854 | C | Grn | rs7251644 | 0.320763 | NA | NA |
| cg18049365 | | | NA | NA | rs76613127 | 0.222781 |
| cg24307368 | | | NA | NA | rs7948284 | 0.332841 |
| cg23208326 | | | NA | NA | NA | NA |
| cg03077671 | | | NA | NA | NA | NA |
| cg07110356 | A | Red | NA | NA | NA | NA |
| cg16528895 | | | rs6739225 | 0.010952 | NA | NA |
| cg08328750 | | | NA | NA | NA | NA |
| cg13978689 | | | NA | NA | NA | NA |
| cg08818984 | | | NA | NA | NA | NA |
| cg27638913 | | | NA | NA | NA | NA |
| cg26720913 | | | NA | NA | NA | NA |
| cg22584138 | | | NA | NA | NA | NA |
| cg21842274 | | | NA | NA | NA | NA |
| cg21840153 | | | NA | NA | NA | NA |
| cg02682525 | | | NA | NA | NA | NA |
| cg16545105 | | | NA | NA | NA | NA |
| cg25487047 | | | NA | NA | NA | NA |
| cg05590274 | | | NA | NA | NA | NA |
| cg27047406 | | | NA | NA | NA | NA |
| cg23365739 | | | NA | NA | NA | NA |
| cg03492327 | | | NA | NA | NA | NA |
| cg16405454 | | | NA | NA | NA | NA |
| cg13947929 | A | Red | NA | NA | NA | NA |
| cg19976235 | | | NA | NA | NA | NA |
| cg17342132 | | | NA | NA | NA | NA |
| cg26998537 | | | NA | NA | NA | NA |
| cg04874129 | | | NA | NA | NA | NA |
| cg25962358 | A | Red | NA | NA | NA | NA |
| cg02712145 | | | NA | NA | NA | NA |
| cg15607672 | | | NA | NA | NA | NA |
| cg05815275 | | | NA | NA | NA | NA |
| cg19547547 | | | NA | NA | NA | NA |
| cg15923943 | | | NA | NA | NA | NA |
| cg01071966 | | | NA | NA | NA | NA |
| cg08845721 | | | NA | NA | NA | NA |
| cg19457823 | | | NA | NA | NA | NA |
| cg24119225 | | | NA | NA | NA | NA |
| cg26196496 | | | NA | NA | NA | NA |
| cg12629325 | | | NA | NA | NA | NA |
| cg15116095 | | | NA | NA | NA | NA |
| cg02693870 | | | NA | NA | NA | NA |
| cg22158248 | | | NA | NA | NA | NA |
| cg23928118 | | | NA | NA | NA | NA |
| cg23985374 | | | NA | NA | NA | NA |
| cg03857453 | | | NA | NA | NA | NA |
| cg24076884 | | | NA | NA | NA | NA |
| cg03391642 | | | NA | NA | NA | NA |
| cg25704227 | | | NA | NA | NA | NA |
| cg19928377 | | | NA | NA | NA | NA |
| cg11976069 | | | NA | NA | NA | NA |
| cg26282566 | | | NA | NA | NA | NA |
| cg02742676 | | | NA | NA | NA | NA |
| cg14514813 | C | Grn | NA | NA | NA | NA |
| cg16158779 | | | NA | NA | NA | NA |
| cg13777717 | A | Red | NA | NA | NA | NA |
| cg07380705 | | | NA | NA | NA | NA |
| cg02047300 | | | NA | NA | NA | NA |
| cg01049782 | | | NA | NA | NA | NA |
| cg12074493 | A | Red | NA | NA | NA | NA |
| cg21619814 | | | NA | NA | NA | NA |
| cg21963703 | | | NA | NA | NA | NA |
| cg03758467 | | | NA | NA | NA | NA |
| cg04306063 | | | NA | NA | NA | NA |
| cg03667083 | | | NA | NA | NA | NA |
| cg18073390 | | | NA | NA | NA | NA |
| cg05241355 | A | Red | NA | NA | NA | NA |
| cg18535456 | A | Red | NA | NA | rs116422312 | 0.020851 |
| cg09936799 | | | NA | NA | NA | NA |
| cg02500479 | | | NA | NA | NA | NA |
| cg15615793 | | | NA | NA | NA | NA |
| cg00025823 | | | NA | NA | NA | NA |
| cg00682007 | | | NA | NA | NA | NA |
| cg27122725 | A | Red | NA | NA | NA | NA |
| cg12962191 | | | NA | NA | NA | NA |
| cg14579239 | | | NA | NA | NA | NA |
| cg06997114 | | | NA | NA | NA | NA |
| cg22642909 | A | Red | NA | NA | NA | NA |
| cg06841846 | A | Red | NA | NA | NA | NA |
| cg24748769 | A | Red | NA | NA | NA | NA |
| cg19186356 | | | NA | NA | NA | NA |
| cg25967418 | | | NA | NA | NA | NA |
| cg25928477 | T | Red | NA | NA | rs73392253 | 0.178914 |
| cg27122213 | A | Red | rs12878110 | 0.058097 | NA | NA |
| cg11539296 | | | NA | NA | NA | NA |
| cg05212570 | | | NA | NA | NA | NA |
| cg20278291 | A | Red | NA | NA | NA | NA |
| cg03226000 | | | NA | NA | NA | NA |
| cg27503360 | C | Grn | NA | NA | NA | NA |
| cg14995055 | A | Red | rs78394916 | 0.011285 | NA | NA |
| cg01089834 | | | NA | NA | NA | NA |
| cg17508591 | A | Red | NA | NA | NA | NA |
| cg11445191 | | | NA | NA | NA | NA |
| cg18640030 | | | NA | NA | NA | NA |
| cg13298827 | A | Red | NA | NA | NA | NA |
| cg10042106 | C | Grn | rs12878110 | 0.058097 | NA | NA |
| cg05884167 | | | NA | NA | rs75342827 | 0.05033 |
| cg18029321 | T | Red | NA | NA | NA | NA |
| cg08935663 | | | NA | NA | NA | NA |
| cg19895517 | | | NA | NA | NA | NA |
| cg00269606 | | | NA | NA | NA | NA |
| cg01972879 | | | NA | NA | NA | NA |
| cg16234335 | | | NA | NA | NA | NA |
| cg11338426 | C | Grn | NA | NA | NA | NA |
| cg18998365 | | | rs10482616 | 0.161024 | NA | NA |
| cg26269677 | | | NA | NA | NA | NA |
| cg04736351 | | | NA | NA | NA | NA |
| cg16174029 | A | Red | NA | NA | NA | NA |
| cg02326566 | | | NA | NA | NA | NA |
| cg03694936 | | | NA | NA | NA | NA |
| cg26882009 | A | Red | NA | NA | NA | NA |
| cg02051771 | | | NA | NA | NA | NA |
| cg26318513 | | | NA | NA | NA | NA |
| cg06928315 | | | NA | NA | NA | NA |
| cg13521908 | T | Red | NA | NA | NA | NA |
| cg13023623 | C | Grn | NA | NA | NA | NA |
| cg06952416 | A | Red | NA | NA | NA | NA |
| cg02036261 | A | Red | NA | NA | NA | NA |
| cg19777540 | | | NA | NA | NA | NA |
| cg16664570 | | | NA | NA | NA | NA |
| cg10649903 | A | Red | NA | NA | NA | NA |
| cg02560085 | | | NA | NA | NA | NA |
| cg16642545 | | | NA | NA | NA | NA |
| cg15827318 | | | NA | NA | NA | NA |
| cg10835712 | | | NA | NA | NA | NA |
| cg23706497 | A | Red | NA | NA | NA | NA |
| cg19106945 | | | NA | NA | NA | NA |
| cg08929103 | | | NA | NA | NA | NA |
| cg20884605 | | | NA | NA | NA | NA |
| cg06282446 | | | NA | NA | NA | NA |
| cg23356309 | | | NA | NA | NA | NA |
| cg24869588 | | | NA | NA | NA | NA |
| cg07555125 | | | NA | NA | NA | NA |
| cg06966811 | A | Red | NA | NA | NA | NA |
| cg26656751 | T | Red | rs17763199 | 0.116279 | NA | NA |
| cg25225155 | A | Red | NA | NA | NA | NA |
| cg06827234 | | | NA | NA | rs3756334 | 0.41123 |
| cg13826890 | A | Red | NA | NA | NA | NA |
| cg20528338 | | | NA | NA | NA | NA |
| cg13619597 | C | Grn | NA | NA | NA | NA |
| cg13309828 | | | NA | NA | NA | NA |
| cg07733851 | | | NA | NA | NA | NA |
| cg18484679 | | | NA | NA | NA | NA |
| cg27604145 | | | NA | NA | NA | NA |
| cg04634417 | | | NA | NA | NA | NA |
| cg27410679 | | | NA | NA | NA | NA |
| cg17852221 | A | Red | NA | NA | NA | NA |
| cg17093478 | | | NA | NA | NA | NA |
| cg08215831 | | | NA | NA | NA | NA |
| cg11560197 | | | NA | NA | NA | NA |
| cg02204262 | | | NA | NA | NA | NA |
| cg23742217 | | | NA | NA | NA | NA |
| cg23316987 | | | NA | NA | NA | NA |
| cg24648119 | | | NA | NA | NA | NA |
| cg03860054 | | | NA | NA | NA | NA |
| cg18283342 | | | NA | NA | NA | NA |
| cg26514430 | | | NA | NA | NA | NA |
| cg07507057 | C | Grn | NA | NA | NA | NA |
| cg03405789 | | | NA | NA | NA | NA |
| cg16014000 | | | NA | NA | NA | NA |
| cg01819552 | | | NA | NA | NA | NA |
| cg17326173 | C | Grn | NA | NA | NA | NA |
| cg21199406 | A | Red | NA | NA | NA | NA |
| cg00822495 | | | NA | NA | NA | NA |
| cg06968181 | A | Red | NA | NA | NA | NA |
| cg14676529 | | | NA | NA | NA | NA |
| cg25913647 | | | NA | NA | NA | NA |
| cg18134262 | | | NA | NA | NA | NA |
| cg17205316 | | | NA | NA | NA | NA |
| cg25500080 | A | Red | NA | NA | NA | NA |
| cg06801483 | | | NA | NA | rs78991894 | 0.036056 |
| cg19035496 | | | NA | NA | NA | NA |
| cg04922810 | | | NA | NA | NA | NA |
| cg05067406 | | | NA | NA | NA | NA |
| cg01155497 | | | NA | NA | NA | NA |
| cg19301632 | | | NA | NA | NA | NA |
| cg08473090 | C | Grn | rs62057073 | 0.126484 | NA | NA |
| cg09516959 | C | Grn | NA | NA | NA | NA |
| cg13648501 | | | NA | NA | NA | NA |
| cg24063856 | | | NA | NA | NA | NA |
| cg03318614 | C | Grn | NA | NA | NA | NA |
| cg24394631 | | | NA | NA | NA | NA |
| cg14557699 | | | NA | NA | NA | NA |
| cg13784080 | | | NA | NA | NA | NA |
| cg25725890 | | | NA | NA | NA | NA |
| cg07778819 | | | NA | NA | NA | NA |
| cg02829712 | | | NA | NA | NA | NA |
| cg25535999 | | | NA | NA | NA | NA |
| cg11954680 | A | Red | rs60784460 | 0.022831 | NA | NA |
| cg05620787 | | | NA | NA | NA | NA |
| cg03384992 | A | Red | NA | NA | NA | NA |
| cg16987900 | A | Red | NA | NA | NA | NA |
| cg18780217 | A | Red | NA | NA | NA | NA |
| cg10519368 | | | NA | NA | NA | NA |
| cg21702128 | | | NA | NA | NA | NA |
| cg17482089 | A | Red | NA | NA | NA | NA |
| cg06593258 | | | NA | NA | NA | NA |
| cg07589972 | | | NA | NA | NA | NA |
| cg04450501 | | | NA | NA | NA | NA |
| cg14692377 | C | Grn | NA | NA | NA | NA |
| cg27345592 | | | NA | NA | NA | NA |
| cg23419885 | | | NA | NA | NA | NA |
| cg10205431 | C | Grn | NA | NA | NA | NA |
| cg23490829 | | | NA | NA | NA | NA |
| cg09746736 | A | Red | rs148624899 | 0.0147 | NA | NA |
| cg12466613 | | | NA | NA | NA | NA |

| Name | SBE_rs | SBE_maf | Islands_Name | Relation_to_Island |
|---|---|---|---|---|
| cg19377607 | rs10823532 | 0.44768 | chr10: 72141560-72142637 | N_Shelf |
| cg20485607 | NA | NA | | OpenSea |
| cg01146324 | rs17633138 | 0.124826 | | OpenSea |
| cg25826698 | rs17041841 | 0.159884 | | OpenSea |
| cg22840887 | rs75594963 | 0.044271 | | OpenSea |
| cg01454153 | rs77890115 | 0.05599 | | OpenSea |
| cg23066280 | NA | NA | | OpenSea |
| cg24648384 | rs798487 | 0.240365 | chr7: 2802440-2802698 | S_Shore |
| cg10950028 | NA | NA | chr1: 110230238-110230614 | S_Shore |
| cg04850148 | NA | NA | | OpenSea |
| cg19080354 | rs10751646 | 0.49249 | chr11: 288847-289239 | N_Shore |
| cg14580085 | NA | NA | | OpenSea |
| cg02583163 | NA | NA | | OpenSea |
| cg06210526 | rs62117444 | 0.059469 | | OpenSea |
| cg15498409 | NA | NA | chr22: 18833786-18834072 | N_Shore |
| cg18428180 | NA | NA | chr6: 24645931-24646591 | Island |
| cg08692111 | NA | NA | chr19: 1734221-1734627 | S_Shelf |
| cg07227024 | rs9288316 | 0.00067 | | OpenSea |
| cg24348479 | NA | NA | | OpenSea |
| cg11188103 | rs12089869 | 0.257741 | | OpenSea |
| cg26785303 | rs66880604 | 0.101602 | | OpenSea |
| cg18845598 | rs55979718 | 0.366088 | | OpenSea |
| cg02612588 | NA | NA | | OpenSea |
| cg21223983 | NA | NA | | OpenSea |
| cg13749548 | NA | NA | chr14: 75725748-75726029 | N_Shelf |
| cg13136933 | NA | NA | | OpenSea |
| cg10156055 | rs73946347 | 0.010064 | | OpenSea |
| cg13626582 | rs28454221 | 0.047989 | | OpenSea |
| cg25593510 | NA | NA | | OpenSea |
| cg12391372 | rs78141882 | 0.254803 | | OpenSea |
| cg15574437 | NA | NA | chr7: 2019763-2020100 | Island |
| cg02276807 | NA | NA | chr12: 133022649-133022863 | Island |
| cg00009523 | rs5024308 | 0.372029 | chr2: 1163016-1163820 | S_Shore |
| cg18433519 | NA | NA | chr6: 24645931-24646591 | Island |
| cg20141578 | NA | NA | | OpenSea |
| cg13478757 | rs10867031 | 0.125075 | chr9: 140512892-140513420 | S_Shelf |
| cg20803293 | NA | NA | | OpenSea |
| cg07102368 | NA | NA | | OpenSea |
| cg01156747 | NA | NA | chr7: 120519-120758 | Island |
| cg04035553 | NA | NA | | OpenSea |
| cg01022117 | NA | NA | | OpenSea |
| cg17004290 | rs6849089 | 0.448449 | chr4: 108852495-108853387 | Island |
| cg03424554 | rs60231938 | 0.322536 | | OpenSea |
| cg24893378 | rs79435314 | 0.037969 | | OpenSea |
| cg02707854 | NA | NA | chr19: 17597344-17598000 | S_Shelf |
| cg18049365 | rs76613127 | 0.222781 | chr18: 30349690-30352302 | N_Shore |
| cg24307368 | rs7948284 | 0.332841 | | OpenSea |
| cg23208326 | NA | NA | | OpenSea |
| cg03077671 | NA | NA | chr19: 13208343-13210525 | S_Shore |
| cg07110356 | NA | NA | chr17: 56355207-56355502 | Island |
| cg16528895 | NA | NA | | OpenSea |
| cg08328750 | NA | NA | | OpenSea |
| cg13978689 | NA | NA | | OpenSea |
| cg08818984 | NA | NA | | OpenSea |
| cg27638913 | NA | NA | chr18: 21851399-21852169 | N_Shore |
| cg26720913 | NA | NA | | OpenSea |
| cg22584138 | NA | NA | chr17: 28562387-28563186 | N_Shore |
| cg21842274 | NA | NA | chr5: 76249435-76250528 | N_Shore |
| cg21840153 | NA | NA | chr18: 21851399-21852169 | S_Shore |
| cg02682525 | NA | NA | chr11: 113258443-113258821 | N_Shore |
| cg16545105 | NA | NA | chr5: 76249435-76250528 | N_Shore |
| cg25487047 | NA | NA | | OpenSea |
| cg05590274 | NA | NA | chr11: 113258443-113258821 | S_Shelf |
| cg27047406 | NA | NA | chr16: 55689598-55690895 | N_Shore |
| cg23365739 | NA | NA | chr14: 57278068-57278287 | N_Shore |
| cg03492327 | NA | NA | chr14: 57274607-57276840 | N_Shore |
| cg16405454 | NA | NA | chr11: 113258443-113258821 | N_Shore |
| cg13947929 | NA | NA | chr17: 43860684-43862928 | S_Shore |
| cg19976235 | NA | NA | chr14: 57274607-57276840 | N_Shore |
| cg17342132 | NA | NA | chr5: 142782071-142785071 | N_Shore |
| cg26998537 | NA | NA | chr14: 57278068-57278287 | N_Shore |
| cg04874129 | NA | NA | chr16: 55689598-55690895 | Island |
| cg25962358 | NA | NA | chr5: 140346105-140346931 | S_Shore |
| cg02712145 | NA | NA | chr7: 30721372-30722445 | Island |
| cg15607672 | NA | NA | chr14: 57278068-57278287 | N_Shore |
| cg05815275 | NA | NA | chr11: 113258443-113258821 | N_Shore |
| cg19547547 | NA | NA | chr5: 140165958-140166214 | Island |
| cg15923943 | NA | NA | | OpenSea |
| cg01071966 | NA | NA | chr5: 76249435-76250528 | N_Shore |
| cg08845721 | NA | NA | chr5: 142782071-142785071 | N_Shore |
| cg19457823 | NA | NA | | OpenSea |
| cg24119225 | NA | NA | chr5: 140346105-140346931 | N_Shore |
| cg26196496 | NA | NA | chr5: 76249435-76250528 | N_Shore |
| cg12629325 | NA | NA | chr5: 140305712-140307193 | Island |
| cg15116095 | NA | NA | chr5: 140167091-140168139 | N_Shore |
| cg02693870 | NA | NA | chr16: 55689598-55690895 | N_Shore |
| cg22158248 | NA | NA | chr12: 112204498-112204979 | N_Shore |
| cg23928118 | NA | NA | chr5: 140215200-140216159 | Island |
| cg23985374 | NA | NA | chr5: 140242073-140243007 | N_Shore |
| cg03857453 | NA | NA | | OpenSea |
| cg24076884 | NA | NA | chr5: 140346105-140346931 | Island |
| cg03391642 | NA | NA | chr5: 140249734-140250949 | Island |
| cg25704227 | NA | NA | chr18: 21977275-21978110 | N_Shelf |
| cg19928377 | NA | NA | chr5: 140249734-140250949 | Island |
| cg11976069 | NA | NA | chr18: 21851399-21852169 | S_Shelf |
| cg26282566 | NA | NA | chr5: 140186792-140187268 | N_Shore |
| cg02742676 | NA | NA | | OpenSea |
| cg14514813 | NA | NA | chr5: 140248789-140249140 | N_Shore |
| cg16158779 | NA | NA | chr11: 113258443-113258821 | Island |
| cg13777717 | NA | NA | chr5: 76249435-76250528 | Island |
| cg07380705 | NA | NA | chr5: 76249435-76250528 | Island |
| cg02047300 | NA | NA | chr5: 140208753-140209957 | N_Shore |
| cg01049782 | NA | NA | chr7: 30721372-30722445 | N_Shelf |
| cg12074493 | NA | NA | chr17: 28562387-28563186 | S_Shore |
| cg21619814 | NA | NA | chr5: 140174573-140174888 | N_Shore |
| cg21963703 | NA | NA | chr5: 140346105-140346931 | S_Shelf |
| cg03758467 | NA | NA | chr5: 140207725-140208078 | N_Shore |
| cg04306063 | NA | NA | chr5: 76249435-76250528 | Island |
| cg03667083 | NA | NA | chr7: 30721372-30722445 | N_Shore |
| cg18073390 | NA | NA | chr5: 140167091-140168139 | N_Shore |
| cg05241355 | NA | NA | chr14: 57278709-57279116 | N_Shore |
| cg18535456 | NA | NA | chr12: 112204498-112204979 | N_Shore |
| cg09936799 | NA | NA | chr5: 140242073-140243007 | N_Shore |
| cg02500479 | NA | NA | chr5: 140263085-140264154 | Island |
| cg15615793 | NA | NA | chr7: 30721372-30722445 | S_Shore |
| cg00025823 | NA | NA | | OpenSea |
| cg00682007 | NA | NA | chr5: 140346105-140346931 | N_Shore |
| cg27122725 | NA | NA | chr5: 142782071-142785071 | N_Shore |
| cg12962191 | NA | NA | chr5: 140214068-140214464 | N_Shore |
| cg14579239 | NA | NA | chr5: 140221007-140221381 | N_Shore |
| cg06997114 | NA | NA | chr18: 21851399-21852169 | Island |
| cg22642909 | NA | NA | chr5: 140221007-140221381 | N_Shore |
| cg06841846 | NA | NA | chr17: 28562387-28563186 | S_Shore |
| cg24748769 | NA | NA | chr14: 57274607-57276840 | N_Shelf |
| cg19186356 | NA | NA | chr12: 112204498-112204979 | N_Shore |
| cg25967418 | NA | NA | chr5: 140261884-140262153 | N_Shore |
| cg25928477 | NA | NA | chr18: 21851399-21852169 | S_Shore |
| cg27122213 | NA | NA | chr14: 57274607-57276840 | Island |
| cg11539296 | NA | NA | chr5: 140215200-140216159 | Island |
| cg05212570 | NA | NA | | OpenSea |
| cg20278291 | NA | NA | chr14: 57274607-57276840 | N_Shelf |
| cg03226000 | NA | NA | chr16: 55689598-55690895 | Island |
| cg27503360 | NA | NA | | OpenSea |
| cg14995055 | NA | NA | chr5: 140242073-140243007 | N_Shore |
| cg01089834 | NA | NA | chr5: 140180844-140181082 | N_Shore |
| cg17508591 | NA | NA | chr5: 140214068-140214464 | Island |
| cg11445191 | NA | NA | chr5: 140228129-140228576 | N_Shore |
| cg18640030 | NA | NA | chr8: 67089249-67089962 | S_Shore |
| cg13298827 | NA | NA | chr5: 140242073-140243007 | S_Shore |
| cg10042106 | NA | NA | chr14: 57274607-57276840 | Island |
| cg05884167 | rs75342827 | 0.05033 | chr5: 76249435-76250528 | S_Shelf |
| cg18029321 | NA | NA | chr5: 140229322-140230333 | Island |
| cg08935663 | NA | NA | chr5: 140305712-140307193 | S_Shelf |
| cg19895517 | NA | NA | | OpenSea |
| cg00269606 | NA | NA | chr8: 67089249-67089962 | S_Shore |
| cg01972879 | NA | NA | chr7: 30721372-30722445 | N_Shore |
| cg16234335 | NA | NA | chr5: 140188014-140188986 | Island |
| cg11338426 | NA | NA | chr17: 43860684-43862928 | Island |
| cg18998365 | NA | NA | chr5: 142782071-142785071 | N_Shore |
| cg26269677 | NA | NA | chr5: 76249435-76250528 | S_Shore |
| cg04736351 | NA | NA | chr14: 57274607-57276840 | N_Shore |
| cg16174029 | NA | NA | chr5: 140346105-140346931 | Island |
| cg02326566 | NA | NA | chr5: 140221007-140221381 | N_Shore |
| cg03694936 | NA | NA | chr5: 140180844-140181082 | N_Shore |
| cg26882009 | NA | NA | chr5: 140346105-140346931 | Island |
| cg02051771 | NA | NA | chr5: 140261884-140262153 | N_Shore |
| cg26318513 | NA | NA | chr5: 140261884-140262153 | N_Shore |
| cg06928315 | NA | NA | chr5: 140242073-140243007 | S_Shore |
| cg13521908 | NA | NA | chr17: 43860684-43862928 | Island |
| cg13023623 | NA | NA | chr14: 57274607-57276840 | Island |
| cg06952416 | NA | NA | chr5: 142782071-142785071 | N_Shore |
| cg02036261 | NA | NA | chr14: 57274607-57276840 | Island |
| cg19777540 | NA | NA | chr5: 140180844-140181082 | N_Shore |
| cg16664570 | NA | NA | chr8: 67089249-67089962 | S_Shore |
| cg10649903 | NA | NA | chr5: 140180844-140181082 | Island |
| cg02560085 | NA | NA | chr5: 140221007-140221381 | N_Shore |
| cg16642545 | NA | NA | | OpenSea |
| cg15827318 | NA | NA | chr5: 140249734-140250949 | S_Shore |
| cg10835712 | NA | NA | chr14: 57264638-57265561 | S_Shelf |
| cg23706497 | NA | NA | chr14: 57274607-57276840 | N_Shelf |
| cg19106945 | NA | NA | chr5: 140207725-140208078 | N_Shore |
| cg08929103 | NA | NA | chr17: 43860684-43862928 | N_Shore |
| cg20884605 | NA | NA | chr12: 112204498-112204979 | S_Shore |
| cg06282446 | NA | NA | chr5: 140174573-140174888 | N_Shore |
| cg23356309 | NA | NA | chr5: 140346105-140346931 | N_Shore |
| cg24869588 | NA | NA | chr5: 140346105-140346931 | N_Shore |
| cg07555125 | NA | NA | chr5: 140175666-140176813 | Island |
| cg06966811 | NA | NA | chr14: 57274607-57276840 | Island |
| cg26656751 | NA | NA | | OpenSea |
| cg25225155 | NA | NA | chr5: 140222122-140223108 | Island |
| cg06827234 | rs3756334 | 0.41123 | chr5: 140214068-140214464 | N_Shore |
| cg13826890 | NA | NA | chr14: 57274607-57276840 | Island |
| cg20528338 | NA | NA | chr18: 21977275-21978110 | N_Shore |
| cg13619597 | NA | NA | chr5: 140256273-140257290 | Island |
| cg13309828 | NA | NA | chr5: 140221007-140221381 | S_Shore |
| cg07733851 | NA | NA | chr5: 142782071-142785071 | N_Shore |
| cg18484679 | NA | NA | | OpenSea |
| cg27604145 | NA | NA | chr5: 140186792-140187268 | N_Shore |
| cg04634417 | NA | NA | chr5: 140221007-140221381 | N_Shore |
| cg27410679 | NA | NA | chr17: 43860684-43862928 | S_Shelf |
| cg17852221 | NA | NA | chr5: 140263085-140264154 | Island |
| cg17093478 | NA | NA | chr5: 140242073-140243007 | Island |
| cg08215831 | NA | NA | chr8: 67089249-67089962 | S_Shore |
| cg11560197 | NA | NA | chr5: 140181888-140183014 | Island |
| cg02204262 | NA | NA | chr5: 140202573-140203610 | N_Shore |
| cg23742217 | NA | NA | chr18: 21851399-21852169 | S_Shore |
| cg23316987 | NA | NA | chr14: 57274607-57276840 | N_Shelf |
| cg24648119 | NA | NA | chr5: 140242073-140243007 | Island |
| cg03860054 | NA | NA | chr16: 55689598-55690895 | S_Shore |
| cg18283342 | NA | NA | chr5: 140235737-140236033 | N_Shore |
| cg26514430 | NA | NA | chr5: 140248789-140249140 | Island |
| cg07507057 | NA | NA | chr5: 140222122-140223108 | Island |
| cg03405789 | NA | NA | chr8: 67089249-67089962 | N_Shore |
| cg16014000 | NA | NA | chr5: 140305712-140307193 | Island |
| cg01819552 | NA | NA | | OpenSea |
| cg17326173 | NA | NA | chr5: 140201440-140201657 | Island |
| cg21199406 | NA | NA | chr5: 76249435-76250528 | Island |
| cg00822495 | NA | NA | chr14: 57274607-57276840 | Island |
| cg06968181 | NA | NA | chr5: 142782071-142785071 | Island |
| cg14676529 | NA | NA | chr5: 140235737-140236033 | N_Shore |
| cg25913647 | NA | NA | chr5: 76249435-76250528 | N_Shore |
| cg18134262 | NA | NA | chr5: 140263085-140264154 | Island |
| cg17205316 | NA | NA | chr14: 57274607-57276840 | Island |
| cg25500080 | NA | NA | chr5: 140346105-140346931 | Island |
| cg06801483 | rs78991894 | 0.036056 | | OpenSea |
| cg19035496 | NA | NA | chr8: 67089249-67089962 | S_Shore |
| cg04922810 | NA | NA | chr7: 30721372-30722445 | Island |
| cg05067406 | NA | NA | | OpenSea |
| cg01155497 | NA | NA | chr5: 140202573-140203610 | Island |
| cg19301632 | NA | NA | chr18: 21977275-21978110 | Island |
| cg08473090 | NA | NA | chr17: 43860684-43862928 | Island |
| cg09516959 | NA | NA | chr7: 30721372-30722445 | Island |
| cg13648501 | NA | NA | chr5: 142782071-142785071 | S_Shore |
| cg24063856 | NA | NA | chr17: 43860684-43862928 | S_Shore |
| cg03318614 | NA | NA | chr5: 140174573-140174888 | Island |
| cg24394631 | NA | NA | chr17: 43860684-43862928 | S_Shore |
| cg14557699 | NA | NA | chr5: 140255158-140255450 | N_Shore |
| cg13784080 | NA | NA | chr14: 57274607-57276840 | Island |
| cg25725890 | NA | NA | chr17: 28562387-28563186 | Island |
| cg07778819 | NA | NA | chr17: 43860684-43862928 | Island |
| cg02829712 | NA | NA | chr5: 140236862-140237891 | N_Shore |
| cg25535999 | NA | NA | | OpenSea |
| cg11954680 | NA | NA | chr5: 140255158-140255450 | N_Shore |
| cg05620787 | NA | NA | | OpenSea |
| cg03384992 | NA | NA | chr18: 21851399-21852169 | S_Shore |
| cg16987900 | NA | NA | chr5: 140207725-140208078 | N_Shore |
| cg18780217 | NA | NA | chr12: 112204498-112204979 | Island |
| cg10519368 | NA | NA | chr5: 140235737-140236033 | N_Shore |
| cg21702128 | NA | NA | chr5: 142782071-142785071 | Island |
| cg17482089 | NA | NA | chr5: 140249734-140250949 | Island |
| cg06593258 | NA | NA | chr18: 21851399-21852169 | S_Shore |
| cg07589972 | NA | NA | | OpenSea |
| cg04450501 | NA | NA | | OpenSea |
| cg14692377 | NA | NA | chr17: 28562387-28563186 | Island |
| cg27345592 | NA | NA | chr5: 142782071-142785071 | S_Shore |
| cg23419885 | NA | NA | chr5: 140186792-140187268 | N_Shore |
| cg10205431 | NA | NA | chr5: 140215200-140216159 | Island |
| cg23490829 | NA | NA | chr5: 140174573-140174888 | N_Shelf |
| cg09746736 | NA | NA | chr16: 55689598-55690895 | Island |
| cg12466613 | NA | NA | | OpenSea |

| Name | Forward_Sequence | |
|---|---|---|
| cg19377607 | GACTTAGCACAAGTCACCTGGATTATTTATGAGCTTTTAAATATACACTTGTCAGCCGGG[CG]CAGTG | SEQ ID NO: 251 |
| | ACTCATGCCTGTAATCCCAGCACTTTAGGAGGCCTAGGCGGGTGGATCACTTGAG | |
| cg20485607 | CCAGGGTTGCTGCTTATCATTCTACAATCCACAGGGCAGCTTTCCAAAGAATTTGCTGGC[CG]CAAAT | SEQ ID NO: 252 |
| | ACCAACAGGTCTGAGATTAAGAAACCCTGTTCTAGATGAATATACACCAAACTTT | |
| cg01146324 | ATTTCAGGAACTCAAAATATTTCTTATGTATGCCAAGATAAACACAAAACCTCTTGGCAG[CG]ATCTA | SEQ ID NO: 253 |
| | TGAGGATTCTCCTTGCTTGTGATTTTGCATTTCTTTATGCTAATTCAGAAATATG | |
| cg25826698 | TTTGAGATGGTATTGCAGCAATGAAAACAAGCTCAATGTCCATGTCAGATAAACAATTGT[CG]TCAC | SEQ ID NO: 254 |
| | AGCTCTGGAAGTTTGGGAAGTGACTCATCTTCAGGAATGGGCAGTGAGTGAAGACA | |
| cg22840887 | GAAGATGTTAGGCAAAATATTAAGACAGCCAAAGCTGCTTACTGAGATGTGCAAGTGACT[CG]CAG | SEQ ID NO: 255 |
| | GGGGACAGACATGCCACTTCAGCACCAGAAGGTTTTAATTTAAACAATATGATTCCA | |
| cg01454153 | GGGCCCCCAGGCGCAGCCCCCTCCCACGGCACCTTTCCAGCTGGCTGTGAGCCCTAGGGC[CG]CAGG | SEQ ID NO: 256 |
| | AAAGGTATGGCAAGATGATGGGAGGGATTTAAAGTTCACAGCGAGAACAAGAAAAG | |
| cg23066280 | CATCTGGATGCACGCCAAGACAAGACGTCAGTCATTTCCTGGTGAAATGAAAGAAAGCCA[CG]CTTC | SEQ ID NO: 257 |
| | CTCCACGCCCATTGGGTCACGAAATCCTTGCTAATCCTGGCCGGGGCACTGGAGGA | |
| cg24648384 | AGAAAACACATTTGCCTTCCCAGGCCCGTGGGAGGAGTGCTGTGAGGCTGAGACACAGCC[CG]GCT | SEQ ID NO: 258 |
| | TGCTGACGGCACAAATGCCGGTGCGCCCTCGGCACAGTTTGTTTGCTTCATCGGGCG | |
| cg10950028 | GAGGGAAGTGCGAAGCAGCTGCGGGACGGACTCTAGGGACCGTTCCTCTTCAGGGCTGCC[CG]CCT | SEQ ID NO: 259 |
| | CAGAAGGGCCTGTGCATGCCGCTGTGTGTGTGTTGGGGGTGTGGGCGGGTAGAGGAG | |
| cg04850148 | GGGGCCCACAGCTAAATCCAGTGAGTGGAAGTTACAGGGAGTCTGCTTCCAGTGCTGCTC[CG]GGA | SEQ ID NO: 260 |
| | AGGATCCCATCCACCAGAGCTGCCCCACATGGACCATGGTCAGGCAGAGGAAGATGC | |
| cg19080354 | CTTCTGGGCAGAGTGCAGGGCCTGCCTTAAATGTTGTGGGGGTTCTGTTCTCAACAAAGG[CG]TAAG | SEQ ID NO: 261 |
| | TTGGGAGCGACTGAGCCACTCCAGGAGGAGGTGTGGGCCCACGGCGTGTTGGAGGT | |
| cg14580085 | TAATTATTCCCGACTTCACTTCCCTTTTACTCTGCGTTGCTTCACAAAGACGCACACACA[CG]CACACA | SEQ ID NO: 262 |
| | GAAATGAGTCATATGTTCAGCTTTCTGAAAGTCAAAGAGAATAAACAGATGCCA | |
| cg02583163 | CTGAGTCCAAGGTGCCTCAGGGTCTGAAGACAAAGCTGCGTCCTCCCGCTCAGGAGGGCC[CG]AGG | SEQ ID NO: 263 |
| | AGCGGACGGCAGAATCTTGGGGCCCAGCCCGTCTGTCCCTCATGGGTCTGTGACCTT | |
| cg06210526 | TCAGAGGCAGAGACTGTAGAGGCAGAAGCAAAGCAAGACACAGAGCATTTCAGAGACCCA[CG]GC | SEQ ID NO: 264 |
| | AAGAGACAGGGAACAGCATGGAGAACTATGAAAAAATAGGCTTCCAGAGAAAGAGAAA | |
| cg15498409 | ACAAGGGACATGAAAAGGGCAAGGAAATAAGAAAATGAGCTGAGTTTTGAAGGCAATGAT[CG]GA | SEQ ID NO: 265 |
| | TGAAGTGAGAGAAGAATATTCCAGCAGAGGGAGACAAGGAGACTGCAGAGATTTCAAG | |
| cg18428180 | CCGCCGCGGTTACCACGGCGACGCGCGCGCACGCGCACACACGCCCTCAGGGATGTGGGG[CG]CGG | SEQ ID NO: 266 |
| | CAGCCGCCCCTGCCCAGCTCCCCTGCTCAGCTCCCCTGCTCAGCTCCCCGGTTGCGC | |
| cg08692111 | TTTGATCGCCACCGAGCGGGTTGGTGCAGCCCCCCTCCCCATCTCGAGCCCTCCTACCTC[CG]CCTGC | SEQ ID NO: 267 |
| | ACAAAGCCGGACGCCTGCCCGCCCCACCGTGGGTCGATGCACCCTGCCAGGGGAG | |
| cg07227024 | TCTGGATCCAAGTCAAATTTTCAGTGATGGAAGAATCACACATCACCTTGTGGATTTGAA[CG]GCTCC | SEQ ID NO: 268 |
| | TCTTCAGTTGTCTCCCACAGACTGCCATAATTTGCCCCAGAATAGAGTCCCTGAG | |
| cg24348479 | GCATGCCCAGGAAGGAGTCGGGACTTGTTTCTTCTGTACCCATACATCCCCTGTGGCTTC[CG]CCTCC | SEQ ID NO: 269 |
| | TCCTGGGCTTTACGGGGAGGTCCAGAGGGAAGAGGCTGCCAATTTAAGAAGGAAG | |
| cg11188103 | AAACTCTCTGAGCGTTGGTTCCCTCATCTGTAAGATGAAAACAGTAACTACATGTCACGG[CG]TCAGT | SEQ ID NO: 270 |
| | GAAAAGATTAAATAAGATAATGTATATAAAGCACTATCAGAGTCCCTGGCCCAGA | |
| cg26785303 | CCAAATCCCAAGGTACCAGAGAGCCTAAAATTCCCTGGCAGATGAATTTAATCACTTTTC[CG]TGTTG | SEQ ID NO: 271 |
| | TTAATAAGAAAACACTACAGAATGTCAGCTGGTGGAATCGGAATTTTCAGTGCTC | |
| cg18845598 | ACTTGATTGAAATCTAGGATATGCTCTTTGAATGTTATTCCTAACGTAATGCCTTTCATT[CG]TCCATC | SEQ ID NO: 272 |
| | CATCCATCCATCCATCAATTCATCCAACAAGTGTAGTGGGCATTGTGCCAGCAA | |
| cg02612588 | CCCCACTGACCACAAGGAGGTGAGGAATCACAATGCTACCTTGTGTTTGCAACTGGAGAC[CG]ATAG | SEQ ID NO: 273 |
| | GAATGATTAGGGAACCATACCACCATAATCCTAGTAACCAGAAAGCCAGCCAGCTA | |
| cg21223983 | CCACAGAGACCGAATGCATCAATTGTTTCACTTATGGAACATTCTGGGCTCTGAGAACCT[CG]GAGT | SEQ ID NO: 274 |
| | AGGAGTGAATCAATCGTACATCCAAGCAGATAACAGCTCAAATCACCAGCTCAGCA | |
| cg13749548 | GAGACCTCAAGAGAGAGAATGGCAAAGTAAATACGACCATCTTATTCGAACTTAACTCTG[CG]TTTC | SEQ ID NO: 275 |
| | AAGTAGGGAGATAAGAAGGACTGCTGTTCCACTTCTTTTCTAGATGACAAGATGGG | |
| cg13136933 | ATGAATTGGGTATATTTGACATCAAAGACAGGAGATCCAGTGGGGAATGAGACCCACGGC[CG]GGC | SEQ ID NO: 276 |
| | GTGGGCAGCGCTGGAGGACAGGCTCTCCCAGTGTGCTGCGGGGGAGGGCAGCCCCTG | |
| cg10156055 | CCAACTCTTAACCTAGTTAATCCTAGTTCTGTTGACATTGGACCAGGCTCAGTAAATAAA[CG]AATGG | SEQ ID NO: 277 |
| | ATTTCCAGCCTTTTTTTCCCATCTGTTCCTGCTTTTAGTCCTCTGAATCTGCTTC | |
| cg13626582 | GGAATGAGACCCACGGCCGGGCGTGGGCAGCGCTGGAGGACAGGCTCTCCCAGTGTGCTG[CG]GG | SEQ ID NO: 278 |
| | GGAGGGCAGCCCCTGCAGTGTGCGTGTTGAGTGCTGGGAAACCCCCAAGTAACACTTC | |
| cg25593510 | GCGCACAGCCAAGTCGCTGTGGACCTAGCAAGGGCTGGATGGACTCATGGAGCCTCAGGG[CG]GGT | SEQ ID NO: 279 |
| | TGGGGAAGCTGGCGAGGCCGAGCCCCGCTTTGGGCTTCTGGGCGCCCTGACCTCGCT | |
| cg12391372 | GTGTACACACACACACACACACACACACCACAGGCAAGCTCAGGCAGGGTCCTTCACAGA[CG]TCTG | SEQ ID NO: 280 |
| | CAGTCACTCTGGAGGTGACCCAAGCACCTGGGCCCTTAGACAGTAACACCACTCAC | |
| cg15574437 | CGTGCGCACCTCCCCGCTGGGGGACACGCCTGAGCATGTCCACTTCCTTCCTGGGGGACA[CG]CATG | SEQ ID NO: 281 |
| | AGCGTGCCCACCTCCCCCCACCCCACAGGACACACCTGAGCACGTCCGCCTCTTCC | |
| cg02276807 | ACAGGACTCCTCCCCAGCCGCTCCTGTATATGACGTCACCGCCGTAAGACCACAGGACAC[CG]CCCC | SEQ ID NO: 282 |
| | AGCCGCGCCTGTAGATGACGTCACCATCGTAAGACCACAGGACCCTTCCCAGCCGC | |
| cg00009523 | TCTCTGTTGAGATTCACATCCTGACTTCTGTCCGTGGGCTCCACTAAAACCTGCAGGCTC[CG]GGGTC | SEQ ID NO: 283 |
| | CATTGCCAGTCACATAGTGACTTCAATGAGCGTTGCTGCCGGCATCCTGAGCGGG | |
| cg18433519 | GCCGCGGTTACCACGGCGACGCGCGCGCACGCGCACACACGCCCTCAGGGATGTGGGGCG[CG]GC | SEQ ID NO: 284 |
| | AGCCGCCCCTGCCCAGCTCCCCTGCTCAGCTCCCCTGCTCAGCTCCCCGGTTGCGCTC | |
| cg20141578 | GAGTCCGCAGAGCGAGAACCGTGCGACAGTGCGTCTGCCAATACCTGGAATGTAGGTGTT[CG]TTG | SEQ ID NO: 285 |
| | CAGTGTGGCCTTCCCAGCCGGCCTCTCAGCACACCCGGCCAGGCAGCCAGTGTTCCC | |
| cg13478757 | TCTTTTCCTTGATTTACAAAGATAGTCAATACATTTTCCTACCAAAAAGAACTGGCCAGC[CG]TGGTG | SEQ ID NO: 286 |
| | GCTCATGCCTGTAATCCTAGCAGTTTAGGAAGCCGAGGTGGGCGGATCACTTGAG | |
| cg20803293 | TGGAGCCTCAGGGCGGGTTGGGGAAGCTGGCGAGGCCGAGCCCCGCTTTGGGCTTCTGGG[CG]CCC | SEQ ID NO: 287 |
| | TGACCTCGCTCCCGGAACCCTGGGGACTGGGAGGCGGGAGGGGGCTTATTGATTCCA | |
| cg07102368 | GATGCCCATGAGACTTCCTGGGCCCCAGTCTTCCAGGTCACGCAGGGTCACTGCGCTTCA[CG]TGGC | SEQ ID NO: 288 |
| | CCTGCAGCACCAAGAATATGCAGGGCCTTGCAATCCAGATGGAGAATTGTGCAAAC | |
| cg01156747 | CAAGTTCAGGAGCTTGTTCCTAGGTGACTCCGGCACATAGGCTGGAAACACTACACTCAC[CG]GGGG | SEQ ID NO: 289 |
| | CTCAGCCCCGCACACCTGTGTGGGGAGGGAGCGGGGAGCGTCGGGGAAGAAGGAGG | |
| cg04035553 | CTCCCTGCAAGGTGAGTCAGCTCCCAGAAGCCCCAGGGCCTGGTCCGGCTGTCCTGGCCC[CG]GCCT | SEQ ID NO: 290 |
| | CATGCCCTGCACTAGGTGGAAGACAGGATCTCCAGGGAACCCGGCTGCTTTGGGCC | |
| cg01022117 | CCAGCAGAGGGGGCAGGAGGCTGGCCAGCCCAGTCCTCCCTCCAGGAGCTGGAACCATCC[CG]CAG | SEQ ID NO: 291 |
| | ACACAGTCCACTCCTGCCCACTCCGAAACACCTCTGAGGGTATTCTCTCAGCAGTAG | |
| cg17004290 | TGCCGCGGATGAGTCTCCAGGTGCGTGGGGGCTGCAGTTCCTGTGCCCCTTCCGGCCGCC[CG]CGCC | SEQ ID NO: 292 |
| | CCCAGGCTGCCTCACACCTGCATCCTGAACTAACAGGTGATGGTGGTGGCGGCGCT | |
| cg03424554 | GCAGAGTATCAATTGTTTGCCCCTGTGATCACGTGGCTGACTGGTGTTGCGGCTCACTGC[CG]CTGCC | SEQ ID NO: 293 |
| | CAGTAGTACCGCATATGACTATTCTGCGAAAAGACCAAAATTCAAAATTTAAAAT | |
| cg24893378 | GAACATGCTAATTCTAAAAATACAGACTCCCTTGCTGTGAAAGACCTCTCAGGGGAGAAC[CG]ACAT | SEQ ID NO: 294 |
| | TGAAGCAAAACAATGATTTCATCCCTCCCGCCCACCATCCCCCAGCCTGTGAACCA | |
| cg02707854 | TCCTCTCCCAGCCAGGCCCCGCCCCCCGACCAGGCTCTATTCCCTTAACTCCCTTATGTG[CG]CCCTCC | SEQ ID NO: 295 |
| | TGTCCACCTGCTGGCGCAACCTGCTCCCCCGTCTCTAGGGGTGGGGTGGGGAGC | |
| cg18049365 | TCCCTATTGACCTTTCAGGGACTCTGAAATCAACTTTCCTCACACACTCTGAATTGTTTG[CG]TTTGTTT | SEQ ID NO: 296 |
| | TCATCCTGCCAGAGCCACCCGCCCAGGCCACCCATCTCTTCCCTCCTCCCTGC | |
| cg24307368 | AGCTGGCAATCACTGTGATGTCACTGATGAAACATCTCAAAGGAACACCCTCCGAAGGTT[CG]AGGC | SEQ ID NO: 297 |
| | TATTTGTGTTCGGGGGACATGGAAATATGTTGACTAAGACTTTGTTCCTTTGGCTG | |
| cg23208326 | TTGAAGTAAATCGTCCTTGCAGAAATCTTTGTTTCATTAGTAAAGAAAACTGGATGACCC[CG]TTAAA | SEQ ID NO: 298 |
| | AGTAGGTAACATTGATGGGAATGATTTATCCACATAAATAAACAAAATGGCTGTG | |
| cg03077671 | ATTCCAGTGGAGCTGTCATTTTGTGGCTCCCATGCCCTGGTGTGGCATGCTCTGTTTTGC[CG]TGACT | SEQ ID NO: 299 |
| | GCGGCTACATGGCTTGGCCTGTTGGGTGGCGGTGGTTGTGTGGGGCTGGCTATGG | |
| cg07110356 | TGCTGGCGTCCACGAAGGAAGTGAGCGCGTTGATCTGGTTGCGGATGGTGATGTTGCTCC[CG]GGG | SEQ ID NO: 300 |
| | CAAGCCGGGCAGGAGCGGAAGAACGGGATGCAGTCGGCTTGGTTCTTGATGCGGGGG | |
| cg16528895 | TGGACACAGACATGAGAGCTATAAACAATGTGATACTATGTCCTGTTAACTCACAGAGAA[CG]GAAG | SEQ ID NO: 301 |
| | GGCTGGAACGGGACCCAGCCAGCAGGGTTTGTGACCGCCTCTGCTGTGACGTCACA | |
| cg08328750 | ATTTTTTTTTAATGCAACAAAAGAGATGATTCAGAATTAATAGTGTTTTTCCCCAGGGCA[CG]GTTTTA | SEQ ID NO: 302 |
| | ACACTTGGATTACAGGTTCATCTCTCCATGCCAGAAGAGAGCAGCAGGGAGCGA | |
| cg13978689 | CAAGATCCATAGGTGGTGCTGAGCCATCTCTTTCAGGGTCTAACCTTCTGTGGAAAAGCA[CG]GCAC | SEQ ID NO: 303 |
| | TGTTCTAGCAGAGTCAGTTTCACTTGAGTTTTCTTCCTCCTTTTTATAACGACATT | |
| cg08818984 | ACCCTCTTTCTGTTTCTATTCCTTCCCCACTCATGCCCCAGTGCTTCCGTTGGACACATG[CG]CATTTTA | SEQ ID NO: 304 |
| | CGGTCCTGCAGGGCTTGAAAGATTTCTGACCTTCTAAGGTCCAGTGATTTGGT | |
| cg27638913 | CCCTCTTCTGTTGCTTCTGGGTATGTTGCACCTTCTGTAAGAATTTATTTAGTCACCTGA[CG]CCGGTA | SEQ ID NO: 305 |
| | TAAGTCAGATGACTACATAGATTTTAGTCATCTAATTTTATGAAGACAGACTGG | |
| cg26720913 | GTCCAGTGATTTGGTATCAGAAGATCTAAGTTTTCTCCTTCTCTCTGCCATTTAAAGAAG[CG]TGTTGC | SEQ ID NO: 306 |
| | AATTTCCCCAGCAGAAGTGAAAGTGATTCGCCTCTACTCAAATGTCTGCATAGG | |
| cg22584138 | CTAGCTGTCTGGGCATTTGTGTCAACCAGAACTCTCCCTTTGCATAAAGCCACCTGCACG[CG]ATGAC | SEQ ID NO: 307 |
| | AGCAAAGTAAAGATCAAACATAAACCATGGGTTGAAACGAAAGCAAGCAACTCTT | |
| cg21842274 | AGAAAAGTATAATGTAGCATTTAGCAGGTCAACAAAATGCACTCCCTTATTCTTTGTCTC[CG]CCTCAT | SEQ ID NO: 308 |
| | TCTCCAACACTTTAGTGCACTAAGTTGTAGTGACTGAAATCATTCTTGGTGAAT | |
| cg21840153 | TGTTTATACCCTAGTTGGGACACTGAGCAACAAGGAAAACAACGGTATTAATCCTACTAC[CG]TGAA | SEQ ID NO: 309 |
| | ACCTACTTAGGTAAAATTATTCCAGCCAGGCTTGAAGGGCAAAGGTTCAGAGGACA | |
| cg02682525 | ACTTAGCGCTGGGCCTAAATAAACTGGATCCAACTTCTCAGGCTGGACTTCTTCCAGCTT[CG]GGACA | SEQ ID NO: 310 |
| | CCTTCTCCCAGCATCCCTCCCTAGGGGGAACTGGGGAAAATCAAAGGCTGAGACA | |
| cg16545105 | CTTGGTGAATAATGCTGGGCCACGCTGAAAATTTGTGGCTGAGAGCTGGACCCTCGTCAT[CG]CCAC | SEQ ID NO: 311 |
| | GTACTCTGTCAATGAGACAGGGTAACCCATGGTTACTGAGCTCCGATCAGTTAAGG | |
| cg25487047 | GAAAAGTTTTGAGATTGAGCAGCTTGGGAGTTTGTGGCCACTGGGTATGTGTGGCTACCG[CG]GGT | SEQ ID NO: 312 |
| | ATGCGAGTGCCAGATATTGGCTGAGACGAGCCAGCTTAGACTAATTGGTACAAGGAA | |
| cg05590274 | GCAAAGAAAACAGAGAGGAAGTGACCAGACAAGTAGGAGAAAAGCAGGAGAATGTTGACA[CG]TG | SEQ ID NO: 313 |
| | GCTCAAGTAACGAAGTTTCAAGAAAAAGAGTCAGAGATCTGAACTATACACTGAGAAC | |
| cg27047406 | AGCCGCACATTCCCATTTCGTTGATAAAGAAACTGACCAGATAGTTTAAGTGGCCTGCTG[CG]GAAG | SEQ ID NO: 314 |
| | ACAGAGCTGGTGCTGCACCGGTCGCTGCTTCCCCAGTCCTTTTTTGGCCTCCTTTC | |
| cg23365739 | TGCTCTAGCAATTTTTATTTTTAATCACAGTGGAGGCTGTGTTGGAGCTGAGGCCGGTCC[CG]CTCTC | SEQ ID NO: 315 |
| | AGGGAGATTTGCTGAGAAAGCGCCTCTCTGGCAACTTTTTGACGCAAACTCTCCA | |
| cg03492327 | CACATTTGTAGTGCAAGCTGCTTCCATGTTGATTTTGATGTGTGGAGGCTGCAGATCCTG[CG]GGAG | SEQ ID NO: 316 |
| | GCAAATTTTCAGACATTTGGGTCCTCGGCAGCTGCAGCAGAGTTTCCTGTTAACTC | |
| cg16405454 | CCTTCATAAACCATAAAACCTTGAATGCACTGAAGGAATGCATAAACCGATTTCACTTAG[CG]CTGGG | SEQ ID NO: 317 |
| | CCTAAATAAACTGGATCCAACTTCTCAGGCTGGACTTCTTCCAGCTTCGGGACAC | |
| cg13947929 | TTCTGGGCGTTCGAGGAGACAGACAGGTGAGGAAGGTAGAAGAAAAACATCCAGAGAGGT[CG]AG | SEQ ID NO: 318 |
| | TAGTTGGCACTGAATGGGAATAGTTTCCAAAAGGCTCGCGTGTACCGAAGCCTGGTCT | |
| cg19976235 | TGTTGCCTTTCCATGGCAGAGATTTTAATTATCTTAGCTTTTTACCACTTTCAACTGCTG[CG]AACCTA | SEQ ID NO: 319 |
| | AAATGTCAGTGCGTTTAACATCTAAAATACCAACTCACTAAACCACAAACTTGG | |
| cg17342132 | TTACTGAGCCTTTTGGAAAATCAACCAAAAGTCTTCGCTGCTTGGAGTCTGATTGAGAAG[CG]ACAG | SEQ ID NO: 320 |
| | CCAGTGAGGGTGAAGACGCAGAAACCTTCACAGTAGCTCCTCCTCTTAGGGTTTTA | |
| cg26998537 | GGGGGAGGAAAACAGGCCGCTGCTGCACGGGGGGACGAGCAGGCAACAAATCCAGGCCTC[CG]AC | SEQ ID NO: 321 |
| | ATTACCAGAATGTTCTTTAAAGACACTGGGCTTTGCTTAAAATATACATTATGGGCCG | |
| cg04874129 | AGTCGGCTTCGCAGTGGACCTGGCCAACGTGTGGCGCTTCCCCTACCTCTGCTACAAGAA[CG]GCGG | SEQ ID NO: 322 |
| | CGGTGAGCGTGGGGTCGGGCTGGGAATTTGAATCTGGGAGGTCCACTGTCTGCAGC | |
| cg25962358 | CTCCTTGAGCAGCTACACGTCGGACCGGGAGAGGCAGCTCTTCAGCATAGATGCCAGTAC[CG]GGG | SEQ ID NO: 323 |
| | AAGTGCGAGTAATTGGGGGGCTGGATTATGAGGAAGCCTCCTCCTACCAGATCTATG | |
| cg02712145 | CGCCTGCCCGAGGAGAACACCGCAGCTCTGCTAATCGAGGGACAGCCGCAACCCAAAGTT[CG]GCA | SEQ ID NO: 324 |
| | GCTTTCCGCCTGAGCTTCCCCTCCCTGAGCGAGGACACTGGAGGGAGGCAGAGACGG | |
| cg15607672 | GAGGCCGGTCCCGCTCTCAGGGAGATTTGCTGAGAAAGCGCCTCTCTGGCAACTTTTTGA[CG]CAAA | SEQ ID NO: 325 |
| | CTCTCCAACCAATGGCAGGGAGAGAATGATTGACACATCTAAGCCAGAGGGAAAAT | |
| cg05815275 | GCTGAGACAGGGGAAATGCGAGGGCTTCGGAGGGACATACCCTCTTCCCCAGGCCCAGGT[CG]CTC | SEQ ID NO: 326 |
| | CATCCCTGCTGGGGCCTCAGGGCTCATGTCTGGGATTTCCCCACCTTTGCGGGGCAG | |
| cg19547547 | GGCACCTTCGTTGGCCGCGTTGCTCAGGACCTGGGACTGGAGCTGGCGGAGCTGGTGCCT[CG]CCT | SEQ ID NO: 327 |
| | GTTCCGGGTGGCGTCCAAAACACACAGGGACCTTCTGGAGGTAAATCTGCAGAATGG | |
| cg15923943 | CAGTCTTGTCTTTGCCTGAGGGTGAGCTAGGACCCTGGGAGCATAAGGGAGGGGACTTGC[CG]GAG | SEQ ID NO: 328 |
| | ATCCTAGAAGGGAAAGGAGGCAGCACTGAAAGAAGAAACATTTCCTTGATTGATCAT | |
| cg01071966 | CCCTACCAGCTTCTAGCTCTCAGTCTGCGCGAGGGTGTAGGAAGGAAAGCCCAGGACCTC[CG]GAG | SEQ ID NO: 329 |
| | CAGAGCACAGCAGCTGCAGAGGCAAGGCCAGCATGTCGCCCAACTTCAAACTTCAGT | |
| cg08845721 | AATCCATTTGCACAGCTGAGGGCAAAAGTGTATCGAACTAAGCTTGGCTATTCATCCTGC[CG]CTCAC | SEQ ID NO: 330 |
| | TGAACGTGTAGCTTTGTTAATCACAGACATTATAATTCATTACATCTGATTATTC | |
| cg19457823 | ATACAACATTTATATAGTGAAATCAGTTTCTCTAAAATACCTTTCCTGACCTCCTCAGGC[CG]AGTTTC | SEQ ID NO: 331 |
| | TCTCTCCCTCATCTATATTATAATCTCTGTACCTTGTCATTGTTAACTCATCTT | |
| cg24119225 | CTCTGTACCCTTGAGGAAACAAAGAGGACAAATAAGAAGCAGCAGTGATAGGAGGCTGGG[CG]CTT | SEQ ID NO: 332 |
| | CTCTGGAATAACACTGGCTAGGTAGTGGAGCATTGATAGTGAGTGTGCTTGGTTGGT | |
| cg26196496 | GGACTGCTTACAAAGTTGGACTGTGGCACGAAGGCCTCCTGATTAAACCAACAAAGAGGC[CG]CCTC | SEQ ID NO: 333 |
| | TCCAGAGCTCAGAATTGTCCTGACATGAAAATAATCAAGTTCTGACACTGCCAGGA | |
| cg12629325 | TGCTCTCCAGGAGTCCGGAGCATGGTCCTGGGTCACCGTTGGTGTAGCGTGTTGGTGGAA[CG]TGG | SEQ ID NO: 334 |
| | ACGCCTAGAGGGAGGATGGTGGGCTGTGGGGTGGCAGTTTTATGTTTGTGGGTTTCC | |
| cg15116095 | CAAACCGGAGCTGCAAGGTACAGTTGAGCTGCTGATCACCGTCCTCGACGTTAATGATAA[CG]CCCC | SEQ ID NO: 335 |
| | ACTGTTTGACCAGGCCGTATACAGAGTCCACTTGTTAGAGACTACAGCAAATGGAA | |
| cg02693870 | GAAGGTGTGAATGAATGAATAATTGAAAGAATAAATAGTAGTTCTCAGCCTCACAGAACA[CG]GGT | SEQ ID NO: 336 |
| | CACAACCTCAAATGACCTGCTACCCTGCCCATAAATAACAGAGATGCAGGAGTAAGT | |
| cg22158248 | AAAAAAAAAAAAAAAAAGAATAAGCAAATCATGCAAATCATCCTGTTCTAAACTCTGAAC[CG]CTGC | SEQ ID NO: 337 |
| | TTCCAAGACCGTCCCTGCCCTTGCCCTGGCCGCCTCTCTCCTTGTTAACTGGGCTC | |
| cg23928118 | CCGCGTTCCCTTCAAGTTGGTGTCCACCTTCAAGAATTACTATTCATTGGTGCTGGACAG[CG]CTCTG | SEQ ID NO: 338 |
| | GACCGCGAGAGTGTGTCCGCCTATGAGCTGGTGGTTACCGCGCGGGACGGGGGCT | |
| cg23985374 | GAAGCGGGAAAGGACCAGTTCCACTACACCATCCTGGAAGAGGCCAAGGACGGCACATAG[CG]TCT | SEQ ID NO: 339 |
| | TCGCGGTCCCTATTGCGCAGGACCTGGGTCTGGAGCTTGTGGAGCTGATACCGCGCC | |
| cg03857453 | CAAAATTATGGTAGCCTGGCTTAAGGTAGGAGCAGTTAAGAATATAAGTAGAAGGGTTTG[CG]AGG | SEQ ID NO: 340 |
| | TATTTATTTAGTCCAATCTATGAGGCCTAATGACTGAAATTATGTGCACAGAGAGGG | |
| cg24076884 | GAGTGCGCAGGACCCCGACGTGGGCGCCAACTCAGTACAGACCTACGAGCTCAGCCCCAG[CG]AGC | SEQ ID NO: 341 |
| | ACTTCGAGCTGGACCTTAAGCCCCTGCAGGAGAACAGTAAAGTGCTTGAGCTGGTGC | |
| cg03391642 | GGGAAGCCCACGCTGGTGTGCTCCCGCGCGGTGGGGAGCTGGTCATACTCGCAGCAGAGG[CG]GC | SEQ ID NO: 342 |
| | AGAGGGTGTGCTCTGAGGAGGGCCCACCTAAGACGGACCTCATGGCCTTCAGCCCCAG | |
| cg25704227 | ATTTACATTACCTTCTCTTATTCATATCCATTCCATACCCACACCAACTTGGATACCATA[CG]GACATAA | SEQ ID NO: 343 |
| | TAAAAAACCAACCATATCACCAGGTATTTATCTTACCCATATTCGGTTGGAAA | |
| cg19928377 | GGCAGGTCACTGTACAGTCTGGGTGGAAATCTTGGACACCAACGATAACTCTCCTGAAGT[CG]CCGT | SEQ ID NO: 344 |
| | GACTTCCTTGTCCCTCCCAGTACGAGAGGACGCTCAGCCCAGCACGGTCATTGCCC | |
| cg11976069 | TTCTAGTTAAAAGAATGGGTGACATCATCCAGGGAGAATATGAGGAGTGAGAAGGTCACT[CG]CAG | SEQ ID NO: 345 |
| | AAACCTAGGTAATACTAATATTTAATACATAGGCAGATGAAAAAATGCTTTAGAAGG | |
| cg26282566 | TACAGTAGAGTGTGTGGGGGTTTCCACAATTGCTACTTACGGTTTGGAGCCACATGATGT[CG]CTCTT | SEQ ID NO: 346 |
| | TACCACAAAATACATGAGAGAAGGAGGAAGAAGGGAAAATTCCTTCTATTCTTAC | |
| cg02742676 | AGTCATATTTCCAGGTTTATAAAATGATTTGCAAACATTTTCTTTTGTTCCCTCTAACAC[CG]CTAGTA | SEQ ID NO: 347 |
| | GATAATGGAAACTCCTTTGATTTGGTCTGTAGATAATTTTATGCTATGTTATAT | |
| cg14514813 | ATTGGGCACCCCACGACTACAGCTCTGGCTTCTCCTCCTCGAATTCTGGGAGGTGGGGAG[CG]GCCA | SEQ ID NO: 348 |
| | GCTCCACTACTCCGTCTCGGAGGAGGCCAAACACGGCACCTTCGTGGGCCGCATCG | |
| cg16158779 | CATCCCTGCTGGGGCCTCAGGGCTCATGTCTGGGATTTCCCCACCTTTGCGGGGCAGGAG[CG]GCTC | SEQ ID NO: 349 |
| | CTCTTGGGCGGGGAAGGAGGCAGGGCCGGCTCGTCTCCCCATTCCCCTCTCCCGGA | |
| cg13777717 | GTCCACGCTCGGCAGAGCCGTTCCGAGAGCACAGAGGCTGTCTTCAGTGCGCGGGTGACC[CG]ATTT | SEQ ID NO: 350 |
| | TGGAGCAGTAGGGGACTGGCTGCCGAGGGGGCATCTAGATTGAGATAGGTGGGAGG | |
| cg07380705 | ATGCCCTCCTCCCCCAGGGTGCCTGGACATGCTGAGCCTCCAGGGCCAGTTCACCTTCAC[CG]CCGAC | SEQ ID NO: 351 |
| | CGGCCGCAGCTGCACTGCGCAGCCTTCTTCATCAGCGAGCCCGAGGAGTTCATTA | |
| cg02047300 | GGGCGCGTCCGATGCAGATGTTGGCTCAAATTCCATCTTAACCTATAAACTCAGTTCTAG[CG]AATAC | SEQ ID NO: 352 |
| | TTCGGGCTAGATGTGAAAATAAACAGTGATGACAATAAACAAATTGGGCTCTTAT | |
| cg01049782 | TTGCTCCACACCTCAGTTTCCCCATGTGTAAAATGAGGATAATGCCATGTCTCTGTCACT[CG]ATGGT | SEQ ID NO: 353 |
| | GCAAGGATTAAATGAGTTAAACCACAGTACAAACATGTGGAAGCTCAGCCACTGA | |
| cg12074493 | TGCCACCTAGACGCCAGGGCGGGGACCGCAAGGTGGGCGGGAGGCTTGGAGGCCGGGATG[CG]G | SEQ ID NO: 354 |
| | GGGAATACTGGTAGGGTGCAAGGAGAATGCTGGAGGGGTGCAGGGGGGATGCCGGGGGT | |
| cg21619814 | CACCATAGAGTGTTCTCTAGGAAGGAAAATACCCAGAGCCCCTTTGTTACTTCAGAGAAG[CG]GAGG | SEQ ID NO: 355 |
| | AATAAGAGAAGCAGCAGGACTTTAACAGAGACTAGAATATTTAAATTTTTGCAAAA | |
| cg21963703 | CAGGTGGTGAAGAGGCATGAACAAGTCAGGCCAGGCTCAGCTTTGCCACCAGCTGTAAGC[CG]GAA | SEQ ID NO: 356 |
| | GCTCAGAGGAAAGACACTGTTACCCTCCCTCTCCTGACTCAAAGCTTGGGGCAATTT | |
| cg03758467 | TGGCGGACGCAGAAGAAATGGATTAATAAATTCCGGAACATACAGTATTTTATCTTTATG[CG]GAAG | SEQ ID NO: 357 |
| | ATCTTCTAATGGAAATAAAACCAGAGGTATTTGACATGGTGTTTACCCCGGAGGAT | |
| cg04306063 | ACTACGATCCTTTCCTGCTCTTCAGCGCCAACCTGAAGCGGGAGCTGGCTGGGGAGCAGC[CG]TACC | SEQ ID NO: 358 |
| | GCCGCGCTCTGCGTGAGTCGAGGCTGCCCGGCTCGCGGGCGCCCGGGACGCGGGGA | |
| cg03667083 | CCTGGGACCTCGGATTAGGGAAGATGCAACACCCCGCACCCTCTGGCTTAGCAGAACCTA[CG]CCTG | SEQ ID NO: 359 |
| | CATGTCCTCCCCACGAAACATGCGCTCGCCCCAGCCCTCCTGCCTCCTACCCTCCT | |
| cg18073390 | ACTGACTGCCACTGATGGGGGCAAACCGGAGCTGCAAGGTACAGTTGAGCTGCTGATCAC[CG]TCCT | SEQ ID NO: 360 |
| | CGACGTTAATGATAACGCCCCACTGTTTGACCAGGCCGTATACAGAGTCCACTTGT | |
| cg05241355 | CCCAGCCCAACTTTTTTGCCTGGCATAGAAAGCAGGGACCGGACAGGTTTCAAACGGATC[CG]AGTC | SEQ ID NO: 361 |
| | CCTAGAAAAGCAAAAACAGACTGGGCGGAGTGTGGGGTGGGGGGTGGCTAGTGGAA | |
| cg18535456 | GAGCCCCCAGGGCGACTTTGGTGGAATCTGGTGTTGAAATCAGAGTCCCACCGCCGGGCG[CG]GTG | SEQ ID NO: 362 |
| | GCTCACGCCTGTAATCCCAGCACTTTGGGAGGCCGAGGCAGGCGGATCATGAGGTGA | |
| cg09936799 | TCAAAGCAACTGATGGGGGCGAAGCTGAGCTGACAGGCACCAAAGCATTATATATCACAG[CG]CTG | SEQ ID NO: 363 |
| | GATGTAAATGATGATGCCCAGTGTTTGACAAAGGAGTCTATCATGTGAAATTACTGG | |
| cg02500479 | TGGATGTCAATGTTTACTTGATCATTGCCATCTGCGCGGTGTCCAGCCTGTTGGTGCTCA[CG]TTGCT | SEQ ID NO: 364 |
| | GCTGTATACTGCGCTGCGGTGCTCGGCACCGCCCACCGAGGGCGCGTGCGCGCCG | |
| cg15615793 | GACACCCATGAGTCTAATGAGATCAAGTGGCCTGAGTGGGTTGAAGTCCTTGGATCAGAC[CG]TGG | SEQ ID NO: 365 |
| | AAGCTGGAATGGAGAGAGATTTCGGAGGGGCAGCCAGACCTGGTGAGGAGGAACTGG | |
| cg00025823 | GAAAAGGTGGACATGGATGGCAGGGGAGCCCCAGGACCTTCTTTGCCAAACAAGTGTTCC[CG]GGA | SEQ ID NO: 366 |
| | ACCTATCTGACTGACGGACCTGGCGTCCAAGTGGCCTCCACCACAGTGACAAGCCCT | |
| cg00682007 | AAAATGACCCATAGAGGAAGATCTACAGATGACAGAATACTGGGAGATGAAGAGAAAGGA[CG]TTT | SEQ ID NO: 367 |
| | TAGTGAGTGCTTTTTTTGCACATGAGGCAATAGATTTCAAATTCCTGTGGAAGAAAT | |
| cg27122725 | GTGTGGACTTGCCACTACAAATCTTGAGGGTAAACTGCTTATCTCCCCACCTCTCTGCCC[CG]TTTATC | SEQ ID NO: 368 |
| | TGAGGCGATAACGATCTCTAAACACGAGGTATCGAATGGAGCCCAATGAATTTC | |
| cg12962191 | TGCACAACAATGGCCACACATCGAGATTGAAATGAAGGGATAAAAACATTCCTATAAATT[CG]AAGG | SEQ ID NO: 369 |
| | CAAGTTTTGCTGACTAGAAAAACAAGCCAATAATTTGAAATGGTGTGCCCGAATGG | |
| cg14579239 | TGTTCAAAGTCTTTTCTCTGACAGCATCTGTCTCTAAAGGCCGAACAACGGGAGATGCAG[CG]GAAT | SEQ ID NO: 370 |
| | TGGATTAAAAGACTCTGAAAGTACAGTCGTTCATCTTTATATTAAGATAATATTTT | |
| cg06997114 | TCTCCCGGCTGGTGCGCCCCTCCCCACGCGCCCGCGGCTGCACCCCACTCTGCACACACA[CG]TCCAA | SEQ ID NO: 371 |
| | CTATTCTTGGATCTACTCACATCCCTGGATCAAGTCTGGCCGCCCTCCCCGCTCC | |
| cg22642909 | TCTGATAGGAAACGACTATTTAACATGGATTATCACTGGCGAGGAGAGCTGGGATCCTGG[CG]ACTA | SEQ ID NO: 372 |
| | CTACTCTTGCTTCTGCTCCTCGCAGCCTGGAAGGTGGGGAGCGGCCAGCTCCACTA | |
| cg06841846 | GTGGAGTCCGCGCGGGATTCTGGTGCCACCTAGACGCCAGGGCGGGGACCGCAAGGTGGG[CG]GG | SEQ ID NO: 373 |
| | AGGCTTGGAGGCCGGGATGCGGGGGAATACTGGTAGGGTGCAAGGAGAATGCTGGAGG | |
| cg24748769 | ATGAAGATGTCTGGGTACCGGGTCTTGGCAAACAGTGCTTCCAGCACATCTAGCTGCGCC[CG]AGTG | SEQ ID NO: 374 |
| | AACGTCGTCCTCTCCCGGCGCTGTTTCCGGGGGGTGGCTGCGGGACAAGAAGCCCA | |
| cg19186356 | CCTGTCATCATTCAGGTCTCACTTGTCATTTCCTGACCATGGTACTTATAAAAGCAGTGC[CG]TCTGCC | SEQ ID NO: 375 |
| | CCATCCATGTCACCTCGTTCATCTCCTTCACCTCCGAAATGATCTCGCTTTTGG | |
| cg25967418 | CCGTTTCTTTCTTCCTGCAGAAAATATAGCAGAAAGCGGAATACCTCTTGCGAATCCTTC[CG]CACTA | SEQ ID NO: 376 |
| | GGAAGCCATAAAAATTGGGCCTTGAGAGACAGTTTGAGGTAAGGCGTTGTATATA | |
| cg25928477 | GAAACTCTGGAAAGCAAAGGAATCTTCTCTCCAGTCCTGCCTGTCACTTACTCCGGCTCA[CG]CTGAA | SEQ ID NO: 377 |
| | GCGTTCGCGTTTACGGTTCCAAAACCAAGACTGAAAGGCAGTCTTCACATATTTA | |
| cg27122213 | CCGCAGCCCCGCAGCCCCGCAGCCCCGCAGGCCTGGCGCCCGAGGTCCCGCTCCACTGCC[CG]CGCC | SEQ ID NO: 378 |
| | CCCCGCGCAGCCTTATATCTAACGGTCAATTCGTGCAATCTGTCGCTTCTCCCTCC | |
| cg11539296 | CGGTGTCCAGTCTGTTGGTGCTTACCCTGCTGCTGTACACGGCGTTGCGGTGCTCAGCGC[CG]TCCTC | SEQ ID NO: 379 |
| | TGAGGGCGCATGTAGTTTGGTAAAGCCCACTCTGGTGTGCTCCAGCGCGGTGGGG | |
| cg05212570 | ATGATTTTTAAAAAATAGATTCAAATGTGTTATCCTCTCTCTGAAACGCTTCCTATAACT[CG]AGTTTA | SEQ ID NO: 380 |
| | TAGGGGAAGAAAAAGCTATTGTTTACAATTATATCACCATTAAGGCAACTGCTA | |
| cg20278291 | TTGGAGCAGTGGAACTAAGGGCAAAGCAAACAAACAAACAGAGGGGCTGGTTTACTGCTT[CG]GAG | SEQ ID NO: 381 |
| | GCAGCAGCTCTTCCACGTTCCAGCACTAACTTAAAAAGAGCACGGACTAGGCGAGGC | |
| cg03226000 | GTGCTGCAGGGTCTTCAGCCGCCCCCAGAGGGCTGTCAGAAGTCTCCAACTCTTGAGTTC[CG]GCGT | SEQ ID NO: 382 |
| | GCCCCAACCTCTGTTTCCAAATTTTTCCAGCGGACGCGCGCCCTTTTCTGGGAACC | |
| cg27503360 | AGCCCCTGACCCAGCAGGAGTGCTGACCTTTCACTCGGGGATGGGGGAACTTTGTCTCCT[CG]CCCC | SEQ ID NO: 383 |
| | TTTTCATTTGGCTTCTCCTCCCCGTTCTTGCAGCCTCTCTCTCCCTTTCTTCCCGT | |
| cg14995055 | AGGATGGTCTAGATCTCCTGACCTCGTGATCCACCGGCCTCGGCTTCCCAAAGTGCTGGC[CG]AGTG | SEQ ID NO: 384 |
| | CCACCGCACCCGGCCGAAAGGTATTTTTTTTAACAGAAGAAAATCAGATTATTCCT | |
| cg01089834 | CGTTTAGCCACATGATGTCGCTGTCCACCACAAGGTCTTTCTCCACAAAAGAAATAACAG[CG]TGCAT | SEQ ID NO: 385 |
| | TACGTATTCAGATACTGCTTTGCTTCATCCTCTCTAAAATTTAACACCGAGGAGT | |
| cg17508591 | AGGAGGCTAAACATGGCAACTTCGTGGGCCGCATCGCGCAGGACCTGGGGCTGGAGCTGG[CG]GA | SEQ ID NO: 386 |
| | GCTGGTGCCGCGCCTGTTCCGGGCGGTGTGCAAATTCCGTGGGGATCTTCTGGAGGTA | |
| cg11445191 | CAAGGTCATACAAAACTTTTAGAGGCAGTCAGTCTGCTAAGAAGGCTCCAGCAAGAGAAA[CG]GGA | SEQ ID NO: 387 |
| | TCTTCTGTTTCAACAATCATTACTTAAGAAAAAATTAAGAAAATGAAATAAGTTTTG | |
| cg18640030 | AGTTTCTCCACTCCAGAGCCTGGAGTGGGATTTTATAGTGTCGACCCTCTTCAAAAACCA[CG]CAGCA | SEQ ID NO: 388 |
| | TTTGCCTAATAAGCTGACGCTCTCTTGACAGCTCGATTGCGTGCTGCCTCTGCTC | |
| cg13298827 | CTTTATTTATTTTGCTTTTGCTTTTTCTTTTGAGATGCTTTATTTAAAGTACACTGGGGC[CG]GGTGCG | SEQ ID NO: 389 |
| | GTAGCTCATGCCTATTTGTAATCCCGGCACTTTGGGAAGCCGAGGCGGGCGGAT | |
| cg10042106 | TTCCTGCCCGGCAGCCCCGCAGCCCCGCAGCCCCGCAGCCCCGCAGCCCCGCAGGCCTGG[CG]CCCG | SEQ ID NO: 390 |
| | AGGTCCCGCTCCACTGCCCGCGCCCCCCGCGCAGCCTTATATCTAACGGTCAATTC | |
| cg05884167 | AGAAAAGGAACACATATCACTCATTTTTGCATACAGTAGAATTAATGGTAACACTTCATG[CG]GCTGA | SEQ ID NO: 391 |
| | AAGATGGGGCCTTTATCTGCCAAAGAATAATGCACTACTGATCTTTTGATTTCTA | |
| cg18029321 | AGGGCGAGTGCGCGCCTGGCAAGCCGACGCTGGTGTGTTCTAGCGCGGTGGGGAGTTGGT[CG]TAC | SEQ ID NO: 392 |
| | TCGCAGCAGAGGAGGCAGAGGGTGTGCTCTGGCGAGGGTAAGCAGAAGACCGACCTC | |
| cg08935663 | AATATAATATGGAAGGTGAAACATTTACTTAAACTATTGCAATGCATTATTAAAAGATGA[CG]ATAAC | SEQ ID NO: 393 |
| | CACAAAAGATGTAAAAATAAATTACAATGGGGGTTAAAAGAAGGGAGAAAGGGAA | |
| cg19895517 | GGCATTTTCTGGAGGACAAATCCTTAGCTGAACTTTTGAATGAGCTCTCTGTATGGGTGC[CG]TAACT | SEQ ID NO: 394 |
| | GTGGTAAAAGTGAGGCCACCAAGGCCAAGCTCAAGACCACCAACCATTTGGAGCC | |
| cg00269606 | AACACTGAATCTCACATCCAATTATATCAACAGATATTTATCGCCTCTTGGTGACGTCAA[CG]AGCCCT | SEQ ID NO: 395 |
| | AAAATGGAAGGGCTTCTTATGACTTGTCCATTGACAAAAATTCTTGAATGAGAT | |
| cg01972879 | GGTTGTTCTCCTTGAGAGATGGTGTTGGGGTGCTGATTCAGACACCACTCATTATGGGGG[CG]CAGG | SEQ ID NO: 396 |
| | AATAATGCTGCCCACAGCCCCTGACTCAGCCCAGCAGGTCAGTCCTGGGACCTCGG | |
| cg16234335 | CGGGGGCTCGCCTTCGCTGTGGGCCACGGCCAGTGTTTCTGTGGAGGTGGCTGATGTGAA[CG]ACA | SEQ ID NO: 397 |
| | ACGCTCCGGCGTTCGCGCAGCCCGAGTACACAGTGTTCGTGAAGGAGAACAACCCGC | |
| cg11338426 | AGGCGAGGAGCGGCGCGGACCGTGGGGGAGGTGGCGGGGGCGAGGCCGGCAAGAGGCGGC[CG] | SEQ ID NO: 398 |
| | CGGGCCGGGCTGCGTCGGGAAACGGCGGCCAGACTTCCCCGGGAAGGGGCGAGCGAGAGC | |
| cg18998365 | TCAAGAGAGTAAGACTGTTAGTATATCGTTAATCAGGGTGTTTTAAGAGATGGGCTAAGA[CG]GAG | SEQ ID NO: 399 |
| | AAGCGCATTTGGGACACTATAGTCAAATCCCGTCCAATAAAAGGTAACTTTTCCTAT | |
| cg26269677 | CGAGTCCATGAACCAGGAAATGGATTCACATTAACCATAAAGACAGACCCCAACCTCTTT[CG]TAAG | SEQ ID NO: 400 |
| | TGTTCTCAGTCAAAAGGCAGAACTTCGGATATAGACCCGCTTTTTGAAAGTAGTAT | |
| cg04736351 | CTCTTTTCCTCTCGAAGCAGGGTCCAGAACGGGGAAAAGAAATAACTTTTGAAAAATCCA[CG]CCTC | SEQ ID NO: 401 |
| | ACTATGACTGCTCACGAGGTGGCCTCTGGAGAACTCATACCTAGAAGCTGACCGCG | |
| cg16174029 | CTTTCTAGAGCGTGAGCGGTGGGGGAGCAGGCGCAGGGTGGCACGAGCGGAGGCGGGGCC[CG]G | SEQ ID NO: 402 |
| | GCGTGGGGCACGGCTGGGGAAGCTGCCGCCTCCGGCCCTGCCCGGCTGCCTCCGCCGCG | |
| cg02326566 | AGGATTTACTATTAAATTATGATAAAGAAATAGAAAGTGCATAGAAGAACCCAGATATTG[CG]GAAG | SEQ ID NO: 403 |
| | TAATTCATGTAATCATTACCTTTTGAAGCCACATGATGTCGCTGTCTACCAAGAAG | |
| cg03694936 | TATTTTATTTCTCTAAATTTGACCTCTTTCCATAAATTTTCTTTGCCCCACTTCCAAATG[CG]GCGAGAA | SEQ ID NO: 404 |
| | TTCATGCTTCTCATTGAATTCACAGATATCTTTAGAATGTTTTATTGCTTAGT | |
| cg26882009 | CTTCCCTTCTGTTTCCTTTTCCCTCCCCCTGGAGCTGTAGCGGCAGCAGCAGCAGGAAGC[CG]AGCCG | SEQ ID NO: 405 |
| | GGTTGAGCGACTCGGAGGCGAGCGGAGGAGCTGGAATATGGGGAGTCAGCGAGGA | |
| cg02051771 | ATCAATGGAAAATATGAAGACTGAGTCATACTTACACTTACACGTTCATGCGCATGGTGT[CG]CTCTT | SEQ ID NO: 406 |
| | CACTGAGAACGTTTCCGCGAAGAAAGCCGCCGTTTCTTTCTTCCTGCAGAAAATA | |
| cg26318513 | GAGAGAGAAAATGGAAACTTTAACCTCCTGGTTAAATTCTATTTAACCAGGACGCCGATT[CG]GGCT | SEQ ID NO: 407 |
| | ACAATGCGTGGCTTTCGTATGAATTGCAGCCGGCGGCGGTAAGTTAAATTCTATTA | |
| cg06928315 | ATCCCAGCTACTCAGGAGGCTGAGGCACAAGAATTGCTTGAACCTAGGAGGCAGAGGTTG[CG]GTG | SEQ ID NO: 408 |
| | AGCCTAGATTAGGCCACTGCACTACAGCCTGGGAGACAGGGTGAAACTCTGTCTTAA | |
| cg13521908 | GCGGACCGTGGGGGAGGTGGCGGGGGCGAGGCCGGCAAGAGGCGGCCGCGGGCCGGGCTG[CG]T | SEQ ID NO: 409 |
| | CGGGAAACGGCGGCCAGACTTCCCCGGGAAGGGGCGAGCGAGAGCCGGGCCGGGCCGGG | |
| cg13023623 | GCTGAGGGAGAGGAGGCCAAAGAGAGCGAGCAGTGAGCGCCGGGGCGGACGAGAAGCCAG[CG]C | SEQ ID NO: 410 |
| | TCCCAGCCTCCTCGGTTATCCGCTCCGGTTTCCGCTCACGTTCAACAGGGGCCGACAGG | |
| cg06952416 | ACTACAAATCTTGAGGGTAAACTGCTTATCTCCCCACCTCTCTGCCCCGTTTATCTGAGG[CG]ATAAC | SEQ ID NO: 411 |
| | GATCTCTAAACACGAGGTATCGAATGGAGCCCAATGAATTTCCATGCCGCTTTTT | |
| cg02036261 | ACCCGGCAAAGTCATGTTGAAAAAACACTCTTCACGTTCGCTCGGCCTGGTGACCAGGGT[CG]GGGA | SEQ ID NO: 412 |
| | CCACGACAACCGGGGGTTGGGAGGCTGCGTAATTACAACCCAGGGTGGTTTGGATT | |
| cg19777540 | TAACAGCGTGCATTACGTATTCAGATACTGCTTTGCTTCATCCTCTCTAAAATTTAACAC[CG]AGGAGT | SEQ ID NO: 413 |
| | TTAAGAAATGAAGATAAGGAACTCGAATTATTTTTAAACTTTGGATCAATGTAA | |
| cg16664570 | TTCTCACAATCTAAAATTAATTGTGGACTCCCTTTTCTATTCCCAAATTAAAGAGAGAGG[CG]GCCAG | SEQ ID NO: 414 |
| | TGAGAGATGCATGAGTTAAACAAGCTTTACTTATTCTACTGAATGCAAACTCAAT | |
| cg10649903 | GGACCTTCTGGAGGTAAATCTGCAGAATGGCATTTTGTTTGTGAATTCTCGGATAGACCG[CG]AGGA | SEQ ID NO: 415 |
| | ACTGTGCGGGCGGAGCGCGGAGTGCAGCATCCACCTGGAGGTGATCGTGGACAGGC | |
| cg02560085 | CTGGTTGGTCAATGTTCAAAGTCTTTTCTCTGACAGCATCTGTCTCTAAAGGCCGAACAA[CG]GGAGA | SEQ ID NO: 416 |
| | TGCAGCGGAATTGGATTAAAAGACTCTGAAAGTACAGTCGTTCATCTTTATATTA | |
| cg16642545 | AACAGTTATGTGACATTGGGTACATCAACTACCCCAGACTTAAAATGGGAGCATGATACC[CG]CCCA | SEQ ID NO: 417 |
| | GTCTGCTTTCCCTTCCATCCCAGGGACCCACTAGATCTTTGGAAAATCCTTTTACG | |
| cg15827318 | TACACGTAGAGTGGTGGTATTACAGACTTTCTTGAGAATCATATCTAGCTATTTCCCCCC[CG]TATAG | SEQ ID NO: 418 |
| | AGGCATAACATACCATTTGGCACAATATTCAAAGAGTCTATGCTGAAGCCTATTC | |
| cg10835712 | CGGCACTTAGCTCTTCGATTCTTAAACCATACCTTGGAAGGGAAAGAAAATTCTTTAACT[CG]GTTTT | SEQ ID NO: 419 |
| | GATAGTTCCTTAAGGACAAGAATGGCTCCCGTATTATAAATCTATCCTACATGGG | |
| cg23706497 | GTTTGGAGGTGCAAAGTCGGCCCAAATCGGGGGTACCCAGCTGGAAGATCTTGATGCGCC[CG]GGG | SEQ ID NO: 420 |
| | TGGACAGGTTCAGAGTCCTTGGTGGGTGGGTTTGGAGCAGTGGAACTAAGGGCAAAG | |
| cg19106945 | AGTGAGTGAGAACAATTAAAGGAAAGGATATGGGACAGGGTTGAAGTTGAGAGAGAAAAA[CG]AG | SEQ ID NO: 421 |
| | ATCATTTAGGAACAGCTGAAGAATCAAAAACAGCAAAGCACAATGGGTAGAAATTCCA | |
| cg08929103 | GATCTTACGATCTTCTAAAATAACTCCAGCTCCAAATGAAGGCCAGTGGACCCATGAACA[CG]CTGG | SEQ ID NO: 422 |
| | CTCTTGCCCAGTGTGCTGAGAAGAGGGCAGGAGGGAGGAGTAGGGTGGGCACGGCA | |
| cg20884605 | CTCATGCCCTCATCCAAGGAGTTCAAAAGGAAAAACCAGGAAAGAGGTGATTTGCAGCCA[CG]TTCT | SEQ ID NO: 423 |
| | CCAGTTCACCATGGGCGAGGCCACGCTCGGCTGCTTCCATTGCTGGGAGTAGGGAA | |
| cg06282446 | TTTAAATTTTTGCAAAACATGCTCTTCTAATTTGATCAAAACATTGAGGATTGGTAATGG[CG]TCTTCT | SEQ ID NO: 424 |
| | ATCAGAAGGGGCCGAGGGGCCTGGACACGGCTGCTCTCGCTTCTGCTCCTCGCA | |
| cg23356309 | GCTAGTTCAGGCCTTTGAATTCTGGGTGTCAGGATCACAACCTTGGCTCCTTTAACTGTC[CG]CTCTA | SEQ ID NO: 425 |
| | GGAGGGAAGCATGCCAAATGTCAGGTCTCACTCTGACTTTGTTCCAGTCCATAGC | |
| cg24869588 | GAGTGTCCAGGGAAGATGTGGGAGTCAGGTGAATAGTTCCCATCGCCAGTAACCCCAGAG[CG]CCT | SEQ ID NO: 426 |
| | CCCCCTGCGTTGTTTCCTTTTAATGTTAGATCCGCCGCCTGGGTGCCCATACTTAGC | |
| cg07555125 | AGAACGCTTCCCTGGGCACTGTCATTGCTCTCATCACGGTGTCGGATCGCGACTCTGGTA[CG]AATG | SEQ ID NO: 427 |
| | GACATGTCACCTGCTCCCTGACGCCCCACGTCCCTTTCAAGCTGGTGTCCACCTTC | |
| cg06966811 | CAAAATTTGTAAGAGAAAAAATACATCCGCCTACAGAACTCCACGTTCGCAGAGGGGCAT[CG]GTCG | SEQ ID NO: 428 |
| | GTCTATTGGAGGTACTGGGGTGATGGCCGACAGACACGGGGACACGTAGATATGGA | |
| cg26656751 | GCCACTGCCAGGGGACTGTGAGGAGGAGCCGAGGATGCAGCCTCGTGTTGGGCCCATGCC[CG]TTC | SEQ ID NO: 429 |
| | CCAGCCACTCAGTCTTTCCCACTGTCTACCTCCTGCCCCGGTGCTTGCTTCCAACTT | |
| cg25225155 | CGGGGGCTCGCCTTCGCTGTGGGCCACCGCCAGCTTGTCTGTGGAGGTGGCCGACGTGAA[CG]ACA | SEQ ID NO: 430 |
| | ATGCTCCGGCGTTCGCGCAGCCCGAGTACACGGTGTTCGTGAAGGAGAACAACCCGC | |
| cg06827234 | GGTTTGTAATTCTTACTTACATGATGGCCACATGATGTCGCTCTTTACCGCAAATTCTTT[CG]TAATCA | SEQ ID NO: 431 |
| | GCAAAAGGAAGTCATTCCTTTTCGCACTGGAAAGACGCTCCACTCTCTTTCACT | |
| cg13826890 | GAGGAAGAAACGTGCCAAAAGGGTTGGCCTTGACTATTAATTATCGTTGGGAGAAAGCCC[CG]GTG | SEQ ID NO: 432 |
| | TTAGCGCTGAGGTCTGGGTGTCTACCTTACTCTGGGGGGAGGAGTTCCCTCTCCTAC | |
| cg20528338 | AACTGCTGCAAAAAATTCATTGTATCGGGGTTAGACTGCAAATACTTCTACCCCTCAAAA[CG]AATCT | SEQ ID NO: 433 |
| | GCAAATTATTTCCCAGATGCTGAGTAAAGAGGTGAGGAGGTTCCAAACTCCAAAT | |
| cg13619597 | GTGGGCCACGGCTAGAGTGTCCGTGGAGGTGGCCGACGTGAACGACAATGCGCCTGCGTT[CG]CGC | SEQ ID NO: 434 |
| | AGCCCGAGTACACAGTGTTCGTGAAGGAGAACAACCCGCCGGGCTGCCACATCTTCA | |
| cg13309828 | GACGGACCTCATGGCCTTCAGCCCCTGCCTTCCTCCTGATCTGGGATCAGTTGATGTAGG[CG]AAGA | SEQ ID NO: 435 |
| | GCAAGATTTAAATGTTGATCATGGCCTCAAAGTAAGTCCATTTAAATTTAGAACTC | |
| cg07733851 | CAAACCTTCACCTATCCCAATTCTCAGCCTATTCTCAAGAGAGTAAGACTGTTAGTATAT[CG]TTAATC | SEQ ID NO: 436 |
| | AGGGTGTTTTAAGAGATGGGCTAAGACGGAGAAGCGCATTTGGGACACTATAGT | |
| cg18484679 | TTGTCTTACCTATAACACTTAATAAAGCATTAAGGACACTGTTCCACCTAATGATGTTTC[CG]GGGTTT | SEQ ID NO: 437 |
| | CTGGTATCTCATTAGGGACTCATCTAATATGACTGATATATCAACAGTAAGCAG | |
| cg27604145 | AATCAATCAGTGGTACAATGTGTACCACAGTAGTACAAAATGTATTCCTATTCTCCGAAA[CG]ACTGA | SEQ ID NO: 438 |
| | AATAATAAATAAAACCAAGTAGTTTACAGTAGAGTGTGTGGGGGTTTCCACAATT | |
| cg04634417 | AGAAGTTCTGGTTGGTCAATGTTCAAAGTCTTTTCTCTGACAGCATCTGTCTCTAAAGGC[CG]AACAA | SEQ ID NO: 439 |
| | CGGGAGATGCAGCGGAATTGGATTAAAAGACTCTGAAAGTACAGTCGTTCATCTT | |
| cg27410679 | TTTGGTATTGTACAATTGGAAAGAACCCGTTTCTGTTCCCACCAGATGCAGATGTCAGGT[CG]AAACA | SEQ ID NO: 440 |
| | GAAGGCTGAGAGATGCTTAAACCACCCTAGCAGAAAAGCCATCTCTGATGCAGAA | |
| cg17852221 | TGCCGCGGTCGGTGGGTGCAGGCCACGTGGTGGCGAAGGTGCGCGCGGTGGACGCCGATT[CG]GG | SEQ ID NO: 441 |
| | CTACAATGCGTGGCTTTCGTATGAATTGCAGCTGGCGGCGGTCGGCGCGCGCATCCCG | |
| cg17093478 | GGTACGCGTTGCAGCCACTGGACTACGAGGAGCTGGAGCTGCTGCAGTTCTAGGTGAGAG[CG]CGC | SEQ ID NO: 442 |
| | GACGCGGGCATGCCGCCTCTGAGCAGCAATGTGACGCTGCCAGGTATTAGTGCTGGA | |
| cg08215831 | GTGGGATTTTATAGTGTCGACCCTCTTCAAAAACCACGCAGCATTTGCCTAATAAGCTGA[CG]CTCTC | SEQ ID NO: 443 |
| | TTGACAGCTCGATTGCGTGCTGCCTCTGCTCCTGCATAAATCATAGGGCCCTGCC | |
| cg11560197 | GCGCGGTGTCCAGTCTGTTGGTGCTCACACTGCTGCTATATACTGCTCTGCGGTGCTCCG[CG]CCGCC | SEQ ID NO: 444 |
| | AACCGAAGGCGACTGTGGGCCGGGCAAGCCCACGCTGGTGTGCTCCAGCGCGGTG | |
| cg02204262 | TGGACAGGCCGCTGCAGGTTTTCCATGTGGAGGTGGCAGTGAAGGACATCAATGACAATC[CG]CCC | SEQ ID NO: 445 |
| | AGGTTCTCCAGACAAGAACAAAGATTATTCATTTTAGAGTCAAGAATGCCAGATTCG | |
| cg23742217 | ATGCGCTCAATTTCAGATGTGGAAACAGAATTCCTAAACATATATTCATTTGTTCAAGGT[CG]AGGTA | SEQ ID NO: 446 |
| | GCAGAGTTTAGATCCAGATTGGTCTGATCCCAAAGCCTGAATTTTTTCCTTTGTA | |
| cg23316987 | AAAAAAAAAAAAGTACCCGAAGAAAGCAATTACAATCTGCCTTCCCCAAGAACAAAAACC[CG]TGCC | SEQ ID NO: 447 |
| | TTCAAATGCACACATTGCATTCCTATCCCTACATTTGCATAGGACTCATTGGCTGG | |
| cg24648119 | GTGGACTCCAGCTATGACGCCTGGTTGTCGCAACAGCTGCATCTGTCAGCTGGCAGCACC[CG]TTCC | SEQ ID NO: 448 |
| | ACGTGGGGCTCTGCACGGGCGAGATCAGCACGACGAGTACCCTGGACGAGGCGAAA | |
| cg03860054 | GGAGTCCTCTGGAAGGTCATTTTCCCAGGCTCTTTGCAGAGTACCTCGAGCTCATTCCAG[CG]GAAGT | SEQ ID NO: 449 |
| | GTCAGGATTGGGCACCCTGGAAGCAAAACAGCAGAAGAGTGAAATCGAGTCATGA | |
| cg18283342 | TAACATATGAATGAAAGAAAAGTTGTGAACTCATGGAATCGTACTTACACCTAAAGCCAC[CG]GATG | SEQ ID NO: 450 |
| | TCGCTGTCGTCCACAAAATAGCTTTTTAGAACAAAGGCATCAGCCAGTTTCTCAAG | |
| cg26514430 | ACAGGCCGCTGCAGGTTTTCCATGTGAACGTGGAGGTGAAGGACATTAACGACAACCCGC[CG]GTG | SEQ ID NO: 451 |
| | TTCTCGCTCAGAGAACAAAAGCTGCTGATTGCGGAATCTAAGCAATCGGACTCGCGT | |
| cg07507057 | CGCTGCTGCTGTACACTGCGCTGCGGTGCTCAGCACTGCCCACTGAGGGCGGGTGCCGGG[CG]GGC | SEQ ID NO: 452 |
| | AAGCCCACTCTGGTGTGCTCCAGTGCGGTGGGGAGCTGGTCATACTCGCAACAACAG | |
| cg03405789 | ACAAAACGTTTTCTCACAGGTCTACATTCTCTTTAAAACACCACTATAAACACCTGGAAA[CG]GAAAC | SEQ ID NO: 453 |
| | TAAACGTAGACAAAACGAATAACATTGTGTTGCTGCTGCACGTGAATACACTTTG | |
| cg16014000 | GTCTCTATTGCCCTCTTGGCAGCTCCTGGGATACAAGAGGGTGCAGGACAGACTTCAACC[CG]CAGC | SEQ ID NO: 454 |
| | AGGATCCAGCGCGGAAAGCTCTGCAGCAGGATCCAGCGCGGAAAGCCCCCCGCAGC | |
| cg01819552 | ATGGGCTCACACTGTGAGTAGTTGATCTTTGAGGCCCACGTCCCATTCTCCAAGCATTCT[CG]ATAGG | SEQ ID NO: 455 |
| | CATTCCCTACAAAAAATGCCAACTGCCAAGAGTCAGGTCACTCCCCTCCTCAAGA | |
| cg17326173 | ACGGAACCTTCGTTGGCCGCATCGCGCAGGACCTAGGGCTGGAGCTGGCGGAGCTGGTGC[CG]CGC | SEQ ID NO: 456 |
| | CTGTTCCGGGTGGCGTCCAAGGGCCGCGGGGACCTTCTGGAGGTAAATCTGCAGAAT | |
| cg21199406 | GTGCCCGAGTCGGAGAGGCGCGTTCGAGGACCCCCACACGGCGAGAATCTCGCCCTTACC[CG]AAG | SEQ ID NO: 457 |
| | ACCTCGACCCACACACATCCAGTGGGGCTGGGGCCGGTGCCTCTGGACGCTGCCATG | |
| cg00822495 | CTAGTTACATCTGCATCTTTTTGTCTCGGACTCTAAAAAGGTCCCTGGGATCCATCCAAA[CG]ACCCCA | SEQ ID NO: 458 |
| | ACCAAATCTGGGGGCCAAAACGCAAAGATCGCGGGAGAAGCCCAGAACGGCGTT | |
| cg06968181 | AAGGAAAGCAGGGGGCGGCAGGCTTAAAAGCAAGTTGCAGGCGAAATAGTAAGTTGCTGG[CG]AA | SEQ ID NO: 459 |
| | ATATAGCCCCCCTTCCGAAACTCCTGACCTCTTCTTCCACCCCGGCCAGGGCCCGCGT | |
| cg14676529 | GATATAAGAAATAGGACAGAAAATGTCAGATCGTATGTGCGTTCTAGACCGCTGATTCGT[CG]ATTT | SEQ ID NO: 460 |
| | GTAAAACAAGAGAAGGATAAGATGGTTTCCAGATGTAGCTGCCTGGGGGTCCAGTG | |
| cg25913647 | GGTATTTGAAGGCACGAAATGGAATGACTTAATCTCATCTCTCAGTGGTTCAGATGGATG[CG]AAGG | SEQ ID NO: 461 |
| | TCATGGCTACAGCTACCTGGTCTAGGAGTGTGTACATCATGTAAACATCCAGGTCA | |
| cg18134262 | GCATGTCCCCTTCAAGCTGGTGTCCACCTACAAGAACTACTACTCATTAGTGCTGGACAG[CG]CCCTG | SEQ ID NO: 462 |
| | GACCGCGAGAGCGTATCAGCCTATGAACTGGTGGTGACCGCGCGGGACGGGGGCT | |
| cg17205316 | AGGAGTTCCCTCTCCTACTCCCCTCTGTTGCTAATAACTTTTGGTGCCTGTAAAAAGTCC[CG]AGCTGA | SEQ ID NO: 463 |
| | GCAGGAGAAATCCTGACCCCGAAGCTCTAGGATGGAGGGGAGAATTTCTAGGAG | |
| cg25500080 | GGAGCTGGAATATGGGGAGTCAGCGAGGACGGTGGGGCCAGGAGCCCTTGGGAGGGCCTA[CG]G | SEQ ID NO: 464 |
| | AGGGAGCGGCCCCAGGCGCTTTCTAGAGCGTGAGCGGTGGGGGAGCAGGCGCAGGGTGG | |
| cg06801483 | CAGGGCAATCACGCCTCCCCGGGGACACTGGGCAATGTCCAGAGACATTTTTGGCAGCCT[CG]ACTG | SEQ ID NO: 465 |
| | CGAGGGGAAGGGTACTACTGGCATCTAGTGGATAGAGGTCAGAGATGCTGCTCAAC | |
| cg19035496 | TCGACCCTCTTCAAAAACCACGCAGCATTTGCCTAATAAGCTGACGCTCTCTTGACAGCT[CG]ATTGC | SEQ ID NO: 466 |
| | GTGCTGCCTCTGCTCCTGCATAAATCATAGGGCCCTGCCAAGGAACGAGGGGCAG | |
| cg04922810 | GAGCAGCTCTTCAGCCAGCGCCAGGCTGCAGTTGGCCTCCAGCAGGCTGTGGAGCAGTGC[CG]CGT | SEQ ID NO: 467 |
| | CCATCGCGTCCCGCAGCCGCGTGCGGAGAGGGAGTGGGAGTGCGCGCCCGGCGTGAC | |
| cg05067406 | GAACGGTTTCATCTTGAAACTACCCCCACCCCAAAATCTGTGGAAAAATTGTCTTCCATG[CG]ACAGG | SEQ ID NO: 468 |
| | TCACTGGTGCTGAAAAGGTTGGGGACCGCTGTTCTGCAGGAAACGAGAGACAGAG | |
| cg01155497 | CTGTACACAGGCGAGATCAGCACAACACGCTCTCTGGATGAGACCGAAGCACCGCGCCAC[CG]CCTT | SEQ ID NO: 469 |
| | CTGGTGCTGGTGAAGGACCATGGAGAGCCCCCGCTGACAGCCACAGCCACAGTGCT | |
| cg19301632 | AGTGGCAGGCCTTACCCGGAGACTACAAGGAACCGCCCGTGTGAGCCAGTGGAAGACTCG[CG]TCC | SEQ ID NO: 470 |
| | CCAGGCAAAATATAAGTCCTGGGTTAATAGTCTGAAGTCTGTCATTAAGCAATAGGT | |
| cg08473090 | ATCAGTGCCAAAGGTGCTGGGGGTCGGGCCCAGCGCCCGGCGCGGCTGGAATGGGGGCGG[CG]AG | SEQ ID NO: 471 |
| | GAGCTCCTCCTAGCCAGCCGCAGGGCGGGAACTGCCCAAACGCAGGTCGCAGCCTCAC | |
| cg09516959 | GTACCGCGGCCGTCAGCAGCTTTGTACCGCTGGGTCCGGAATCCTCTTTACTCCCTAAAC[CG]CCTTC | SEQ ID NO: 472 |
| | TCAGGAGGCCGGTGTAGAGCAGGCAGCGAGGGCCGGGGGCACTCACGGGTCGTGT | |
| cg13648501 | AGCCGCCATCTTGGCAAAGGAAGAGATACTCGCCACCTCGGAAAAGAAGAGGGAACCAAC[CG]TAT | SEQ ID NO: 473 |
| | GCCAACGTAGAACCCTCAGCCAGAAGAATTTTTATGAAGGGCGCTGAGCAACTCCAT | |
| cg24063856 | CACATCCCCTCCCCCATCCGTTACCTGAGGGCTAGGGATGAACTCAGATTATTTTCTGGG[CG]TTCGA | SEQ ID NO: 474 |
| | GGAGACAGACAGGTGAGGAAGGTAGAAGAAAAACATCCAGAGAGGTCGAGTAGTT | |
| cg03318614 | GGCGTCTTCTATCAGAAGGGGCCGAGGGGCCTGGACACGGCTGCTCTCGCTTCTGCTCCT[CG]CAGC | SEQ ID NO: 475 |
| | CTGGGAGGTGGGGAGCGGCCAGCTCCGCTACTCCGTCCCCGAGGAGGCCAAACACG | |
| cg24394631 | AACATAGACACCAGCTTGACCCGGCTCAGGGCATTCAGAGCGATTACATTCAAGAGACTT[CG]GTGG | SEQ ID NO: 476 |
| | ATTTGGGGGAGGGGTGCGGATTGGCGGAATTTCATTTTTCCATCAGTTATTAAACC | |
| cg14557699 | CTACACTCAGAAGGTGAAACAGGAAAATTTTTTTCTCCGCACCCACATTCCAATCATTCA[CG]GAATA | SEQ ID NO: 477 |
| | GGATCGACTCCATAATGACTGTGATGCTGGAAAAAATTTACTAAATATGTACTTA | |
| cg13784080 | TTGCTAATAACTTTTGGTGCCTGTAAAAAGTCCCGAGCTGAGCAGGAGAAATCCTGACCC[CG]AAGC | SEQ ID NO: 478 |
| | TCTAGGATGGAGGGGAGAATTTCTAGGAGCGACTTCCCCGTCCCCTCCCCAAGCAA | |
| cg25725890 | GGAGCAGCCGGCAGGGCAGGGCCGAGCGAGGAATCCTGACTTTCCTGCTCTTTAACTTTG[CG]GGA | SEQ ID NO: 479 |
| | GGGGGGAGGCTGCACAAAGGAGCAGGTGTGCGCCTCCCCTGCCGCCCCGCGCCCACA | |
| cg07778819 | CACCCCGCTTCGCGCCAGTCTGGGCATCGCCTCACCCGGGGCTCGGGACTCCGCAGGGTC[CG]CTCC | SEQ ID NO: 480 |
| | AGCCACAAACATAGACACCAGCTTGACCCGGCTCAGGGCATTCAGAGCGATTACAT | |
| cg02829712 | CCCAGGTTCTCCGTAACAGAACAAAAGCTCTCAATACCTGAATCCAGACTGCTTGACTCT[CG]ATTTC | SEQ ID NO: 481 |
| | CACTAGAAGGCGCATCTGATGCGGATGTTGGAGAGAACGCATTGCTTACTTACAA | |
| cg25535999 | TAGGGAGAAACATTTTTATGGCTATCTCTTTATGATCCTCTCATTTCTGAACACTGAACT[CG]ACCTGC | SEQ ID NO: 482 |
| | CTCTTAAGTTTCTCTTCTCTCCAAGTAACTGTCTTTCCCAAATTAAGAGTCTTT | |
| cg11954680 | GTGAAAAATAACCCAAAGTCGAAACATTAGAATTAAGAGTGACCCAGGAAGTGGCTAAAC[CG]AAA | SEQ ID NO: 483 |
| | AGAACCTCAGGATCTTTCTTGTACTTACATAATCAGTCACATGATGTCGCTCTACAC | |
| cg05620787 | TGATTTTGATGCACAACTAGTTAAAAGCCTTTCATACCAGTCAGTATTCCCAGCCTTGAG[CG]CACGC | SEQ ID NO: 484 |
| | GCGCACACACACACACACATACACACACGCATTAATTTTTGTACTTTGCTTCTTT | |
| cg03384992 | AGCAAAGGAATCTTCTCTCCAGTCCTGCCTGTCACTTACTCCGGCTCACGCTGAAGCGTT[CG]CGTTT | SEQ ID NO: 485 |
| | ACGGTTCCAAAACCAAGACTGAAAGGCAGTCTTCACATATTTATTGTTTATACCC | |
| cg16987900 | CTGCATCATTCCCTGACTGTTGAATGATGGCGGACGCAGAAGAAATGGATTAATAAATTC[CG]GAAC | SEQ ID NO: 486 |
| | ATACAGTATTTTATCTTTATGCGGAAGATCTTCTAATGGAAATAAAACCAGAGGTA | |
| cg18780217 | CTGCCGCGCGGGGCGGGGAGCGGGGTCGGCTCAGTGGCCCTGAGACCCTAGCTCTGCTCT[CG]GTC | SEQ ID NO: 487 |
| | CGCTCGCTGTCCGCTAGCCCGCTGCGATGTTGCGCGCTGCCGCCCGCTTCGGGCCCC | |
| cg10519368 | CAGCCATTTCGATAAAAAATAGATATAAGAAATAGGACAGAAAATGTCAGATCGTATGTG[CG]TTCT | SEQ ID NO: 488 |
| | AGACCGCTGATTCGTCGATTTGTAAAACAAGAGAAGGATAAGATGGTTTCCAGATG | |
| cg21702128 | CCCCAGCTCGCCGCGTCGGGAAGGCTTGGACGATGCCGGGACCGAGCCTCCTACCTTGTG[CG]GCA | SEQ ID NO: 489 |
| | CAGATTATGATTTTTGTGACTCTGGGAAAGGTTGTGCTGTGTGGGTTTAGGGTTTGG | |
| cg17482089 | CCGGCACTGCTGGCGACTCAGGCTGGCAGCGCGGGAGGCGCAGTTAACAAGCTAGTACCG[CG]GTC | SEQ ID NO: 490 |
| | GGTGGGTGCGGGCCACGTGGTGGCGAAGGTGCGCGCAGTGGATGCGGACTCAGGCTA | |
| cg06593258 | AATCTTCTCTCCAGTCCTGCCTGTCACTTACTCCGGCTCACGCTGAAGCGTTCGCGTTTA[CG]GTTCCA | SEQ ID NO: 491 |
| | AAACCAAGACTGAAAGGCAGTCTTCACATATTTATTGTTTATACCCTAGTTGGG | |
| cg07589972 | AACAGTGGAACAAGTGAGGCTTGTATTACATGGCCAAATTGATATTAATGATGGTGGCCT[CG]AGAA | SEQ ID NO: 492 |
| | CGGATTCCATCCCAAGACATCTGATCTGCAGACAGTTGAGCATTCACAGCAGTTTT | |
| cg04450501 | GGGAGAGTGGCCAGGTCCTGTCTCCATCAGCATGCATTTGCAGGGACTGGTCTGTGGTCA[CG]GCCT | SEQ ID NO: 493 |
| | CTGTCGTCCTCCCTGACGACATTTACCCTGGTCCCCTCCCCTCTCCTCTGGGCAGG | |
| cg14692377 | ACCCACCTGCCAGGCTGCGCGGGGAGGCTGGTCCCGGGCTGGGCAGGCGGGCTGGCCTCG[CG]CCC | SEQ ID NO: 494 |
| | TCGAGGCACCCGGCGGCGCTGGCTGTGCGGAGGGGCGCCGGCGCGGCCGTATTTGTA | |
| cg27345592 | CTCAATCATTAACATGAAGTAGTATATCAATTCTGATAGACTACTTACAATATGTGCTTA[CG]GAATTT | SEQ ID NO: 495 |
| | AATTGAAGAGATGAACTCCAGTGTGCCAGAAAGGACGTGAGATTTAGCATTATA | |
| cg23419885 | AAAAAAGGACACACATACTCAAATGCTTATAAGAGATAAATTAATGTAATAAATGGCCCA[CG]CGAT | SEQ ID NO: 496 |
| | TCCACCCTGGTGCTGCAGTTTGTAAAAGAAGAAAAAATACCTGTTCAATTGGTTTG | |
| cg10205431 | AGAGCTTGTGCCGCGGTCTGTGGGCGCGGGCCATGTGGTGGCGAAGGTACGTGCAGTTGA[CG]CTG | SEQ ID NO: 497 |
| | ACTCAGGCTACAACGCGTGGCTTTCGTATGAGTTGCAACCGGTGGCGGCCGGTGCGA | |
| cg23490829 | ACAGAGTCTCTGTAAGAATAAATTTCTTCTCTCTTATGGAGGCCAGAAGTCCAAAATCAG[CG]TGTTT | SEQ ID NO: 498 |
| | CCTGGGCCGCATTTCTTCTGAAGACTTTAGGGGAAATTTTTTTCTTGGATCTTCC | |
| cg09746736 | GCCGCCGGACACGTGAGTGCGCCCTGAGCGCGGGACAGGGCTAGGTCTGCCTGGGAGGCC[CG]GG | SEQ ID NO: 499 |
| | CCGAGACGCGCCAGCAGAGGGCTAGCGAGTTTGTAGTGCAGTGACGTTAAGTGTCCGA | |
| cg12466613 | GGAACAAGTGAGGCTTGTATTACATGGCCAAATTGATATTAATGATGGTGGCCTCGAGAA[CG]GATT | SEQ ID NO: 500 |
| | CCATCCCAAGACATCTGATCTGCAGACAGTTGAGCATTCACAGCAGTTTTGACTTT | |
| Name | SourceSeq | |
| cg19377607 | CGCCCGGCTGACAAGTGTATATTTAAAAGCTCATAAATAATCCAGGTGAC | SEQ ID NO: 501 |
| cg20485607 | CGGCCAGCAAATTCTTTGGAAAGCTGCCCTGTGGATTGTAGAATGATAAG | SEQ ID NO: 502 |
| cg01146324 | CGCTGCCAAGAGGTTTTGTGTTTATCTTGGCATACATAAGAAATATTTTG | SEQ ID NO: 503 |
| cg25826698 | TTGCAGCAATGAAAACAAGCTCAATGTCCATGTCAGATAAACAATTGTCG | SEQ ID NO: 504 |
| cg22840887 | CAAAATATTAAGACAGCCAAAGCTGCTTACTGAGATGTGCAAGTGACTCG | SEQ ID NO: 505 |
| cg01454153 | TCGCTGTGAACTTTAAATCCCTCCCATCATCTTGCCATACCTTTCCTGCG | SEQ ID NO: 506 |
| cg23066280 | CGCTTCCTCCACGCCCATTGGGTCACGAAATCCTTGCTAATCCTGGCCGG | SEQ ID NO: 507 |
| cg24648384 | CGGGCTGTGTCTCAGCCTCACAGCACTCCTCCCACGGGCCTGGGAAGGCA | SEQ ID NO: 508 |
| cg10950028 | CGGGCAGCCCTGAAGAGGAACGGTCCCTAGAGTCCGTCCCGCAGCTGCTT | SEQ ID NO: 509 |
| cg04850148 | CGGGAAGGATCCCATCCACCAGAGCTGCCCCACATGGACCATGGTCAGGC | SEQ ID NO: 510 |
| cg19080354 | CGCCTTTGTTGAGAACAGAACCCCCACAACATTTAAGGCAGGCCCTGCAC | SEQ ID NO: 511 |
| cg14580085 | CGCACACAGAAATGAGTCATATGTTCAGCTTTCTGAAAGTCAAAGAGAAT | SEQ ID NO: 512 |
| cg02583163 | CGGGCCCTCCTGAGCGGGAGGACGCAGCTTTGTCTTCAGACCCTGAGGCA | SEQ ID NO: 513 |
| cg06210526 | CGGCAAGAGACAGGGAACAGCATGGAGAACTATGAAAAAATAGGCTTCCA | SEQ ID NO: 514 |
| cg15498409 | CGGATGAAGTGAGAGAAGAATATTCCAGCAGAGGGAGACAAGGAGACTGC | SEQ ID NO: 515 |
| cg18428180 | CGCGGCAGCCGCCCCTGCCCAGCTCCCCTGCTCAGCTCCCCTGCTCAGCT | SEQ ID NO: 516 |
| cg08692111 | CGCCTGCACAAAGCCGGACGCCTGCCCGCCCCACCGTGGGTCGATGCACC | SEQ ID NO: 517 |
| cg07227024 | TCAAATTTTCAGTGATGGAAGAATCACACATCACCTTGTGGATTTGAACG | SEQ ID NO: 518 |
| cg24348479 | AGGAGTCGGGACTTGTTTCTTCTGTACCCATACATCCCCTGTGGCTTCCG | SEQ ID NO: 519 |
| cg11188103 | CGCCGTGACATGTAGTTACTGTTTTCATCTTACAGATGAGGGAACCAACG | SEQ ID NO: 520 |
| cg26785303 | CGGAAAAGTGATTAAATTCATCTGCCAGGGAATTTTAGGCTCTCTGGTAC | SEQ ID NO: 521 |
| cg18845598 | TCTAGGATATGCTCTTTGAATGTTATTCCTAACGTAATGCCTTTCATTCG | SEQ ID NO: 522 |
| cg02612588 | TTTCTGGTTACTAGGATTATGGTGGTATGGTTCCCTAATCATTCCTATCG | SEQ ID NO: 523 |
| cg21223983 | CGGAGTAGGAGTGAATCAATCGTACATCCAAGCAGATAACAGCTCAAATC | SEQ ID NO: 524 |
| cg13749548 | CGCAGAGTTAAGTTCGAATAAGATGGTCGTATTTACTTTGCCATTCTCTC | SEQ ID NO: 525 |
| cg13136933 | TATTTGACATCAAAGACAGGAGATCCAGTGGGGAATGAGACCCACGGCCG | SEQ ID NO: 526 |
| cg10156055 | CTAGTTAATCCTAGTTCTGTTGACATTGGACCAGGCTCAGTAAATAAACG | SEQ ID NO: 527 |
| cg13626582 | ACGGCCGGGCGTGGGCAGCGCTGGAGGACAGGCTCTCCCAGTGTGCTGCG | SEQ ID NO: 528 |
| cg25593510 | CGCCCTGAGGCTCCATGAGTCCATCCAGCCCTTGCTAGGTCCACAGCGAC | SEQ ID NO: 529 |
| cg12391372 | CGTCTGCAGTCACTCTGGAGGTGACCCAAGCACCTGGGCCCTTAGACAGT | SEQ ID NO: 530 |
| cg15574437 | CCCGCTGGGGGACACGCCTGAGCATGTCCACTTCCTTCCTGGGGGACACG | SEQ ID NO: 531 |
| cg02276807 | CGCCCCAGCCGCGCCTGTAGATGACGTCACCATCGTAAGACCACAGGACC | SEQ ID NO: 532 |
| cg00009523 | CGGGGTCCATTGCCAGTCACATAGTGACTTCAATGAGCGTTGCTGCCGGC | SEQ ID NO: 533 |
| cg18433519 | GGAGCTGAGCAGGGGAGCTGAGCAGGGGAGCTGGGCAGGGGCGGCTGCCG | SEQ ID NO: 534 |
| cg20141578 | CGAGAACCGTGCGACAGTGCGTCTGCCAATACCTGGAATGTAGGTGTTCG | SEQ ID NO: 535 |
| cg13478757 | CGGCTGGCCAGTTCTTTTTGGTAGGAAAATGTATTGACTATCTTTGTAAA | SEQ ID NO: 536 |
| cg20803293 | GCGGGTTGGGGAAGCTGGCGAGGCCGAGCCCCGCTTTGGGCTTCTGGGCG | SEQ ID NO: 537 |
| cg07102368 | CGTGGCCCTGCAGCACCAAGAATATGCAGGGCCTTGCAATCCAGATGGAG | SEQ ID NO: 538 |
| cg01156747 | CTTGTTCCTAGGTGACTCCGGCACATAGGCTGGAAACACTACACTCACCG | SEQ ID NO: 539 |
| cg04035553 | CCGGGTTCCCTGGAGATCCTGTCTTCCACCTAGTGCAGGGCATGAGGCCG | SEQ ID NO: 540 |
| cg01022117 | GAATACCCTCAGAGGTGTTTCGGAGTGGGCAGGAGTGGACTGTGTCTGCG | SEQ ID NO: 541 |
| cg17004290 | ACCATCACCTGTTAGTTCAGGATGCAGGTGTGAGGCAGCCTGGGGGCGCG | SEQ ID NO: 542 |
| cg03424554 | CGGCAGTGAGCCGCAACACCAGTCAGCCACGTGATCACAGGGGCAAACAA | SEQ ID NO: 543 |
| cg24893378 | CGGTTCTCCCCTGAGAGGTCTTTCACAGCAAGGGAGTCTGTATTTTTAGA | SEQ ID NO: 544 |
| cg02707854 | CGCCCTCCTGTCCACCTGCTGGCGCAACCTGCTCCCCCGTCTCTAGGGGT | SEQ ID NO: 545 |
| cg18049365 | CGCAAACAATTCAGAGTGTGTGAGGAAAGTTGATTTCAGAGTCCCTGAAA | SEQ ID NO: 546 |
| cg24307368 | CTGTGATGTCACTGATGAAACATCTCAAAGGAACACCCTCCGAAGGTTCG | SEQ ID NO: 547 |
| cg23208326 | GTTTATTTATGTGGATAAATCATTCCCATCAATGTTACCTACTTTTAACG | SEQ ID NO: 548 |
| cg03077671 | CGGCAAAACAGAGCATGCCACACCAGGGCATGGGAGCCACAAAATGACAG | SEQ ID NO: 549 |
| cg07110356 | CGAAGGAAGTGAGCGCGTTGATCTGGTTGCGGATGGTGATGTTGCTCCCG | SEQ ID NO: 550 |
| cg16528895 | TGAGAGCTATAAACAATGTGATACTATGTCCTGTTAACTCACAGAGAACG | SEQ ID NO: 551 |
| cg08328750 | CGGTTTTAACACTTGGATTACAGGTTCATCTCTCCATGCCAGAAGAGAGC | SEQ ID NO: 552 |
| cg13978689 | GTGGTGCTGAGCCATCTCTTTCAGGGTCTAACCTTCTGTGGAAAAGCACG | SEQ ID NO: 553 |
| cg08818984 | GACCTTAGAAGGTCAGAAATCTTTCAAGCCCTGCAGGACCGTAAAATGCG | SEQ ID NO: 554 |
| cg27638913 | GCTTCTGGGTATGTTGCACCTTCTGTAAGAATTTATTTAGTCACCTGACG | SEQ ID NO: 555 |
| cg26720913 | CGCTTCTTTAAATGGCAGAGAGAAGGAGAAAACTTAGATCTTCTGATACC | SEQ ID NO: 556 |
| cg22584138 | CTTTCGTTTCAACCCATGGTTTATGTTTGATCTTTACTTTGCTGTCATCG | SEQ ID NO: 557 |
| cg21842274 | TGTAGCATTTAGCAGGTCAACAAAATGCACTCCCTTATTCTTTGTCTCCG | SEQ ID NO: 558 |
| cg21840153 | CTTTGCCCTTCAAGCCTGGCTGGAATAATTTTACCTAAGTAGGTTTCACG | SEQ ID NO: 559 |
| cg02682525 | GCCTAAATAAACTGGATCCAACTTCTCAGGCTGGACTTCTTCCAGCTTCG | SEQ ID NO: 560 |
| cg16545105 | TGCTGGGCCACGCTGAAAATTTGTGGCTGAGAGCTGGACCCTCGTCATCG | SEQ ID NO: 561 |
| cg25487047 | ATTAGTCTAAGCTGGCTCGTCTCAGCCAATATCTGGCACTCGCATACCCG | SEQ ID NO: 562 |
| cg05590274 | CGTGGCTCAAGTAACGAAGTTTCAAGAAAAAGAGTCAGAGATCTGAACTA | SEQ ID NO: 563 |
| cg27047406 | CGCAGCAGGCCACTTAAACTATCTGGTCAGTTTCTTTATCAACGAAATGG | SEQ ID NO: 564 |
| cg23365739 | GTCAAAAAGTTGCCAGAGAGGCGCTTTCTCAGCAAATCTCCCTGAGAGCG | SEQ ID NO: 565 |
| cg03492327 | CGGGAGGCAAATTTTCAGACATTTGGGTCCTCGGCAGCTGCAGCAGAGTT | SEQ ID NO: 566 |
| cg16405454 | ATAAAACCTTGAATGCACTGAAGGAATGCATAAACCGATTTCACTTAGCG | SEQ ID NO: 567 |
| cg13947929 | GGTACACGCGAGCCTTTTGGAAACTATTCCCATTCAGTGCCAACTACTCG | SEQ ID NO: 568 |
| cg19976235 | CGCAGCAGTTGAAAGTGGTAAAAAGCTAAGATAATTAAAATCTCTGCCAT | SEQ ID NO: 569 |
| cg17342132 | AGGAGGAGCTACTGTGAAGGTTTCTGCGTCTTCACCCTCACTGGCTGTCG | SEQ ID NO: 570 |
| cg26998537 | TATATTTTAAGCAAAGCCCAGTGTCTTTAAAGAACATTCTGGTAATGTCG | SEQ ID NO: 571 |
| cg04874129 | AGTGGACCTGGCCAACGTGTGGCGCTTCCCCTACCTCTGCTACAAGAACG | SEQ ID NO: 572 |
| cg25962358 | CTACACGTCGGACCGGGAGAGGCAGCTCTTCAGCATAGATGCCAGTACCG | SEQ ID NO: 573 |
| cg02712145 | GAGAACACCGCAGCTCTGCTAATCGAGGGACAGCCGCAACCCAAAGTTCG | SEQ ID NO: 574 |
| cg15607672 | GCTCTCAGGGAGATTTGCTGAGAAAGCGCCTCTCTGGCAACTTTTTGACG | SEQ ID NO: 575 |
| cg05815275 | CGCTCCATCCCTGCTGGGGCCTCAGGGCTCATGTCTGGGATTTCCCCACC | SEQ ID NO: 576 |
| cg19547547 | GGCCGCGTTGCTCAGGACCTGGGACTGGAGCTGGCGGAGCTGGTGCCTCG | SEQ ID NO: 577 |
| cg15923943 | GGAAATGTTTCTTCTTTCAGTGCTGCCTCCTTTCCCTTCTAGGATCTCCG | SEQ ID NO: 578 |
| cg01071966 | AGTTGGGCGACATGCTGGCCTTGCCTCTGCAGCTGCTGTGCTCTGCTCCG | SEQ ID NO: 579 |
| cg08845721 | CGGCAGGATGAATAGCCAAGCTTAGTTCGATACACTTTTGCCCTCAGCTG | SEQ ID NO: 580 |
| cg19457823 | CGGCCTGAGGAGGTCAGGAAAGGTATTTTAGAGAAACTGATTTCACTATA | SEQ ID NO: 581 |
| cg24119225 | CACTCACTATCAATGCTCCACTACCTAGCCAGTGTTATTCCAGAGAAGCG | SEQ ID NO: 582 |
| cg26196496 | CGGCCTCTTTGTTGGTTTAATCAGGAGGCCTTCGTGCCACAGTCCAACTT | SEQ ID NO: 583 |
| cg12629325 | GTCCGGAGCATGGTCCTGGGTCACCGTTGGTGTAGCGTGTTGGTGGAACG | SEQ ID NO: 584 |
| cg15116095 | GCAAGGTACAGTTGAGCTGCTGATCACCGTCCTCGACGTTAATGATAACG | SEQ ID NO: 585 |
| cg02693870 | CGGGTCACAACCTCAAATGACCTGCTACCCTGCCCATAAATAACAGAGAT | SEQ ID NO: 586 |
| cg22158248 | AAAAAGAATAAGCAAATCATGCAAATCATCCTGTTCTAAACTCTGAACCG | SEQ ID NO: 587 |
| cg23928118 | CAAGTTGGTGTCCACCTTCAAGAATTACTATTCATTGGTGCTGGACAGCG | SEQ ID NO: 588 |
| cg23985374 | CGCTATGTGCCGTCCTTGGCCTCTTCCAGGATGGTGTAGTGGAACTGGTC | SEQ ID NO: 589 |
| cg03857453 | ACATAATTTCAGTCATTAGGCCTCATAGATTGGACTAAATAAATACCTCG | SEQ ID NO: 590 |
| cg24076884 | GCACTTTACTGTTCTCCTGCAGGGGCTTAAGGTCCAGCTCGAAGTGCTCG | SEQ ID NO: 591 |
| cg03391642 | CGGCAGAGGGTGTGCTCTGAGGAGGGCCCACCTAAGACGGACCTCATGGC | SEQ ID NO: 592 |
| cg25704227 | TTCTCTTATTCATATCCATTCCATACCCACACCAACTTGGATACCATACG | SEQ ID NO: 593 |
| cg19928377 | TACAGTCTGGGTGGAAATCTTGGACACCAACGATAACTCTCCTGAAGTCG | SEQ ID NO: 594 |
| cg11976069 | CGCAGAAACCTAGGTAATACTAATATTTAATACATAGGCAGATGAAAAAA | SEQ ID NO: 595 |
| cg26282566 | TGTGGGGGTTTCCACAATTGCTACTTACGGTTTGGAGCCACATGATGTCG | SEQ ID NO: 596 |
| cg02742676 | CGGTGTTAGAGGGAACAAAAGAAAATGTTTGCAAATCATTTTATAAACCT | SEQ ID NO: 597 |
| cg14514813 | CGGCCAGCTCCACTACTCCGTCTCGGAGGAGGCCAAACACGGCACCTTCG | SEQ ID NO: 598 |
| cg16158779 | GGCCTCAGGGCTCATGTCTGGGATTTCCCCACCTTTGCGGGGCAGGAGCG | SEQ ID NO: 599 |
| cg13777717 | CGGGTCACCCGCGCACTGAAGACAGCCTCTGTGCTCTCGGAACGGCTCTG | SEQ ID NO: 600 |
| cg07380705 | CCCAGGGTGCCTGGACATGCTGAGCCTCCAGGGCCAGTTCACCTTCACCG | SEQ ID NO: 601 |
| cg02047300 | CGCTAGAACTGAGTTTATAGGTTAAGATGGAATTTGAGCCAACATCTGCA | SEQ ID NO: 602 |
| cg01049782 | CTTCCACATGTTTGTACTGTGGTTTAACTCATTTAATCCTTGCACCATCG | SEQ ID NO: 603 |
| cg12074493 | GCCAGGGCGGGGACCGCAAGGTGGGCGGGAGGCTTGGAGGCCGGGATGCG | SEQ ID NO: 604 |
| cg21619814 | CGGAGGAATAAGAGAAGCAGCAGGACTTTAACAGAGACTAGAATATTTAA | SEQ ID NO: 605 |
| cg21963703 | CGGCTTACAGCTGGTGGCAAAGCTGAGCCTGGCCTGACTTGTTCATGCCT | SEQ ID NO: 606 |
| cg03758467 | CGGAAGATCTTCTAATGGAAATAAAACCAGAGGTATTTGACATGGTGTTT | SEQ ID NO: 607 |
| cg04306063 | CGGCTGCTCCCCAGCCAGCTCCCGCTTCAGGTTGGCGCTGAAGAGCAGGA | SEQ ID NO: 608 |
| cg03667083 | GATTAGGGAAGATGCAACACCCCGCACCCTCTGGCTTAGCAGAACCTACG | SEQ ID NO: 609 |
| cg18073390 | CGGTGATCAGCAGCTCAACTGTACCTTGCAGCTCCGGTTTGCCCCCATCA | SEQ ID NO: 610 |
| cg05241355 | CGGATCCGTTTGAAACCTGTCCGGTCCCTGCTTTCTATGCCAGGCAAAAA | SEQ ID NO: 611 |
| cg18535456 | CGCGCCCGGCGGTGGGACTCTGATTTCAACACCAGATTCCACCAAAGTCG | SEQ ID NO: 612 |
| cg09936799 | CGCTGGATGTAAATGATGATGCCCAGTGTTTGACAAAGGAGTCTATCATG | SEQ ID NO: 613 |
| cg02500479 | CGTGAGCACCAACAGGCTGGACACCGCGCAGATGGCAATGATCAAGTAAA | SEQ ID NO: 614 |
| cg15615793 | CGGTCTGATCCAAGGACTTCAACCCACTCAGGCCACTTGATCTCATTAGA | SEQ ID NO: 615 |
| cg00025823 | CGGGAACCTATCTGACTGACGGACCTGGCGTCCAAGTGGCCTCCACCACA | SEQ ID NO: 616 |
| cg00682007 | GGAATTTGAAATCTATTGCCTCATGTGCAAAAAAAGCACTCACTAAAACG | SEQ ID NO: 617 |
| cg27122725 | GCTCCATTCGATACCTCGTGTTTAGAGATCGTTATCGCCTCAGATAAACG | SEQ ID NO: 618 |
| cg12962191 | CGAAGGCAAGTTTTGCTGACTAGAAAAACAAGCCAATAATTTGAAATGGT | SEQ ID NO: 619 |
| cg14579239 | CGCTGCATCTCCCGTTGTTCGGCCTTTAGAGACAGATGCTGTCAGAGAAA | SEQ ID NO: 620 |
| cg06997114 | CGTCCAACTATTCTTGGATCTACTCACATCCCTGGATCAAGTCTGGCCGC | SEQ ID NO: 621 |
| cg22642909 | CGCCAGGATCCCAGCTCTCCTCGCCAGTGATAATCCATGTTAAATAGTCG | SEQ ID NO: 622 |
| cg06841846 | CGCCCACCTTGCGGTCCCCGCCCTGGCGTCTAGGTGGCACCAGAATCCCG | SEQ ID NO: 623 |
| cg24748769 | CGGGCGCAGCTAGATGTGCTGGAAGCACTGTTTGCCAAGACCCGGTACCC | SEQ ID NO: 624 |
| cg19186356 | CGGCACTGCTTTTATAAGTACCATGGTCAGGAAATGACAAGTGAGACCTG | SEQ ID NO: 625 |
| cg25967418 | TCCTGCAGAAAATATAGCAGAAAGCGGAATACCTCTTGCGAATCCTTCCG | SEQ ID NO: 626 |
| cg25928477 | CGCTGAAGCGTTCGCGTTTACGGTTCCAAAACCAAGACTGAAAGGCAGTC | SEQ ID NO: 627 |
| cg27122213 | CGCGCCCCCCGCGCAGCCTTATATCTAACGGTCAATTCGTGCAATCTGTC | SEQ ID NO: 628 |
| cg11539296 | GGAGCACACCAGAGTGGGCTTTACCAAACTACATGCGCCCTCAGAGGACG | SEQ ID NO: 629 |
| cg05212570 | CGAGTTATAGGAAGCGTTTCAGAGAGAGGATAACACATTTGAATCTATTT | SEQ ID NO: 630 |
| cg20278291 | CGGAGGCAGCAGCTCTTCCACGTTCCAGCACTAACTTAAAAAGAGCACGG | SEQ ID NO: 631 |
| cg03226000 | CTTCAGCCGCCCCCAGAGGGCTGTCAGAAGTCTCCAACTCTTGAGTTCCG | SEQ ID NO: 632 |
| cg27503360 | AGCAGGAGTGCTGACCTTTCACTCGGGGATGGGGGAACTTTGTCTCCTCG | SEQ ID NO: 633 |
| cg14995055 | ATTTTCTTCTGTTAAAAAAAATACCTTTCGGCCGGGTGCGGTGGCACTCG | SEQ ID NO: 634 |
| cg01089834 | CGCTGTTATTTCTTTTGTGGAGAAAGACCTTGTGGTGGACAGCGACATCA | SEQ ID NO: 635 |
| cg17508591 | CGCCAGCTCCAGCCCCAGGTCCTGCGCGATGCGGCCCACGAAGTTGCCAT | SEQ ID NO: 636 |
| cg11445191 | AAACTTTTAGAGGCAGTCAGTCTGCTAAGAAGGCTCCAGCAAGAGAAACG | SEQ ID NO: 637 |
| cg18640030 | CGCAGCATTTGCCTAATAAGCTGACGCTCTCTTGACAGCTCGATTGCGTG | SEQ ID NO: 638 |
| cg13298827 | CGGCTTCCCAAAGTGCCGGGATTACAAATAGGCATGAGCTACCGCACCCG | SEQ ID NO: 639 |
| cg10042106 | CGCCCGAGGTCCCGCTCCACTGCCCGCGCCCCCCGCGCAGCCTTATATCT | SEQ ID NO: 640 |
| cg05884167 | CGGCTGAAAGATGGGGCCTTTATCTGCCAAAGAATAATGCACTACTGATC | SEQ ID NO: 641 |
| cg18029321 | CGACCAACTCCCCACCGCGCTAGAACACACCAGCGTCGGCTTGCCAGGCG | SEQ ID NO: 642 |
| cg08935663 | CTTCTTTTAACCCCCATTGTAATTTATTTTTACATCTTTTGTGGTTATCG | SEQ ID NO: 643 |
| cg19895517 | CGGCACCCATACAGAGAGCTCATTCAAAAGTTCAGCTAAGGATTTGTCCT | SEQ ID NO: 644 |
| cg00269606 | CGAGCCCTAAAATGGAAGGGCTTCTTATGACTTGTCCATTGACAAAAATT | SEQ ID NO: 645 |
| cg01972879 | GACTGACCTGCTGGGCTGAGTCAGGGGCTGTGGGCAGCATTATTCCTGCG | SEQ ID NO: 646 |
| cg16234335 | TTCGCTGTGGGCCACGGCCAGTGTTTCTGTGGAGGTGGCTGATGTGAACG | SEQ ID NO: 647 |
| cg11338426 | GCGCGGACCGTGGGGGAGGTGGCGGGGGCGAGGCCGGCAAGAGGCGGCCG | SEQ ID NO: 648 |
| cg18998365 | CGGAGAAGCGCATTTGGGACACTATAGTCAAATCCCGTCCAATAAAAGGT | SEQ ID NO: 649 |
| cg26269677 | CGAAAGAGGTTGGGGTCTGTCTTTATGGTTAATGTGAATCCATTTCCTGG | SEQ ID NO: 650 |
| cg04736351 | CGCCTCACTATGACTGCTCACGAGGTGGCCTCTGGAGAACTCATACCTAG | SEQ ID NO: 651 |
| cg16174029 | CGGGCCCCGCCTCCGCTCGTGCCACCCTGCGCCTGCTCCCCCACCGCTCA | SEQ ID NO: 652 |
| cg02326566 | CGGAAGTAATTCATGTAATCATTACCTTTTGAAGCCACATGATGTCGCTG | SEQ ID NO: 653 |
| cg03694936 | CTAAATTTGACCTCTTTCCATAAATTTTCTTTGCCCCACTTCCAAATGCG | SEQ ID NO: 654 |
| cg26882009 | CGGCTTCCTGCTGCTGCTGCCGCTACAGCTCCAGGGGGAGGGAAAAGGAA | SEQ ID NO: 655 |
| cg02051771 | TATGAAGACTGAGTCATACTTACACTTACACGTTCATGCGCATGGTGTCG | SEQ ID NO: 656 |
| cg26318513 | GGAAACTTTAACCTCCTGGTTAAATTCTATTTAACCAGGACGCCGATTCG | SEQ ID NO: 657 |
| cg06928315 | CGGTGAGCCTAGATTAGGCCACTGCACTACAGCCTGGGAGACAGGGTGAA | SEQ ID NO: 658 |
| cg13521908 | CGCAGCCCGGCCCGCGGCCGCCTCTTGCCGGCCTCGCCCCCGCCACCTCC | SEQ ID NO: 659 |
| cg13023623 | GAGGCCAAAGAGAGCGAGCAGTGAGCGCCGGGGCGGACGAGAAGCCAGCG | SEQ ID NO: 660 |
| cg06952416 | CGCCTCAGATAAACGGGGCAGAGAGGTGGGGAGATAAGCAGTTTACCCTC | SEQ ID NO: 661 |
| cg02036261 | CATGTTGAAAAAACACTCTTCACGTTCGCTCGGCCTGGTGACCAGGGTCG | SEQ ID NO: 662 |
| cg19777540 | CGGTGTTAAATTTTAGAGAGGATGAAGCAAAGCAGTATCTGAATACGTAA | SEQ ID NO: 663 |
| cg16664570 | CGGCCAGTGAGAGATGCATGAGTTAAACAAGCTTTACTTATTCTACTGAA | SEQ ID NO: 664 |
| cg10649903 | CGCGGTCTATCCGAGAATTCACAAACAAAATGCCATTCTGCAGATTTACC | SEQ ID NO: 665 |
| cg02560085 | TGTTCAAAGTCTTTTCTCTGACAGCATCTGTCTCTAAAGGCCGAACAACG | SEQ ID NO: 666 |
| cg16642545 | ACATTGGGTACATCAACTACCCCAGACTTAAAATGGGAGCATGATACCCG | SEQ ID NO: 667 |
| cg15827318 | CGGGGGGGAAATAGCTAGATATGATTCTCAAGAAAGTCTGTAATACCACC | SEQ ID NO: 668 |
| cg10835712 | CGGTTTTGATAGTTCCTTAAGGACAAGAATGGCTCCCGTATTATAAATCT | SEQ ID NO: 669 |
| cg23706497 | CGGGCGCATCAAGATCTTCCAGCTGGGTACCCCCGATTTGGGCCGACTTT | SEQ ID NO: 670 |
| cg19106945 | CGAGATCATTTAGGAACAGCTGAAGAATCAAAAACAGCAAAGCACAATGG | SEQ ID NO: 671 |
| cg08929103 | TTCTAAAATAACTCCAGCTCCAAATGAAGGCCAGTGGACCCATGAACACG | SEQ ID NO: 672 |
| cg20884605 | AGCAATGGAAGCAGCCGAGCGTGGCCTCGCCCATGGTGAACTGGAGAACG | SEQ ID NO: 673 |
| cg06282446 | CGCCATTACCAATCCTCAATGTTTTGATCAAATTAGAAGAGCATGTTTTG | SEQ ID NO: 674 |
| cg23356309 | CGGACAGTTAAAGGAGCCAAGGTTGTGATCCTGACACCCAGAATTCAAAG | SEQ ID NO: 675 |
| cg24869588 | AAGATGTGGGAGTCAGGTGAATAGTTCCCATCGCCAGTAACCCCAGAGCG | SEQ ID NO: 676 |
| cg07555125 | CAGCTTGAAAGGGACGTGGGGCGTCAGGGAGCAGGTGACATGTCCATTCG | SEQ ID NO: 677 |
| cg06966811 | GAGAAAAAATACATCCGCCTACAGAACTCCACGTTCGCAGAGGGGCATCG | SEQ ID NO: 678 |
| cg26656751 | CGGGCATGGGCCCAACACGAGGCTGCATCCTCGGCTCCTCCTCACAGTCC | SEQ ID NO: 679 |
| cg25225155 | TTCGCTGTGGGCCACCGCCAGCTTGTCTGTGGAGGTGGCCGACGTGAACG | SEQ ID NO: 680 |
| cg06827234 | CGAAAGAATTTGCGGTAAAGAGCGACATCATGTGGCCATCATGTAAGTAA | SEQ ID NO: 681 |
| cg13826890 | CGGGGCTTTCTCCCAACGATAATTAATAGTCAAGGCCAACCCTTTTGGCA | SEQ ID NO: 682 |
| cg20528338 | CGAATCTGCAAATTATTTCCCAGATGCTGAGTAAAGAGGTGAGGAGGTTC | SEQ ID NO: 683 |
| cg13619597 | TAGAGTGTCCGTGGAGGTGGCCGACGTGAACGACAATGCGCCTGCGTTCG | SEQ ID NO: 684 |
| cg13309828 | TAAATGGACTTACTTTGAGGCCATGATCAACATTTAAATCTTGCTCTTCG | SEQ ID NO: 685 |
| cg07733851 | TATCCCAATTCTCAGCCTATTCTCAAGAGAGTAAGACTGTTAGTATATCG | SEQ ID NO: 686 |
| cg18484679 | TAACACTTAATAAAGCATTAAGGACACTGTTCCACCTAATGATGTTTCCG | SEQ ID NO: 687 |
| cg27604145 | CGACTGAAATAATAAATAAAACCAAGTAGTTTACAGTAGAGTGTGTGGGG | SEQ ID NO: 688 |
| cg04634417 | CGGCCTTTAGAGACAGATGCTGTCAGAGAAAAGACTTTGAACATTGACCA | SEQ ID NO: 689 |
| cg27410679 | CGACCTGACATCTGCATCTGGTGGGAACAGAAACGGGTTCTTTCCAATTG | SEQ ID NO: 690 |
| cg17852221 | TGGGTGCAGGCCACGTGGTGGCGAAGGTGCGCGCGGTGGACGCCGATTCG | SEQ ID NO: 691 |
| cg17093478 | AGCCACTGGACTACGAGGAGCTGGAGCTGCTGCAGTTCTAGGTGAGAGCG | SEQ ID NO: 692 |
| cg08215831 | AGTGTCGACCCTCTTCAAAAACCACGCAGCATTTGCCTAATAAGCTGACG | SEQ ID NO: 693 |
| cg11560197 | GTCTGTTGGTGCTCACACTGCTGCTATATACTGCTCTGCGGTGCTCCGCG | SEQ ID NO: 694 |
| cg02204262 | TGCAGGTTTTCCATGTGGAGGTGGCAGTGAAGGACATCAATGACAATCCG | SEQ ID NO: 695 |
| cg23742217 | CGAGGTAGCAGAGTTTAGATCCAGATTGGTCTGATCCCAAAGCCTGAATT | SEQ ID NO: 696 |
| cg23316987 | CGGGTTTTTGTTCTTGGGGAAGGCAGATTGTAATTGCTTTCTTCGGGTAC | SEQ ID NO: 697 |
| cg24648119 | CGGGTGCTGCCAGCTGACAGATGCAGCTGTTGCGACAACCAGGCGTCATA | SEQ ID NO: 698 |
| cg03860054 | CGCTGGAATGAGCTCGAGGTACTCTGCAAAGAGCCTGGGAAAATGACCTT | SEQ ID NO: 699 |
| cg18283342 | GCTGATGCCTTTGTTCTAAAAAGCTATTTTGTGGACGACAGCGACATCCG | SEQ ID NO: 700 |
| cg26514430 | TTGCTTAGATTCCGCAATCAGCAGCTTTTGTTCTCTGAGCGAGAACACCG | SEQ ID NO: 701 |
| cg07507057 | GTATGACCAGCTCCCCACCGCACTGGAGCACACCAGAGTGGGCTTGCCCG | SEQ ID NO: 702 |
| cg03405789 | CGGAAACTAAACGTAGACAAAACGAATAACATTGTGTTGCTGCTGCACGT | SEQ ID NO: 703 |
| cg16014000 | CGGGTTGAAGTCTGTCCTGCACCCTCTTGTATCCCAGGAGCTGCCAAGAG | SEQ ID NO: 704 |
| cg01819552 | CGAGAATGCTTGGAGAATGGGACGTGGGCCTCAAAGATCAACTACTCACA | SEQ ID NO: 705 |
| cg17326173 | TTGGCCGCATCGCGCAGGACCTAGGGCTGGAGCTGGCGGAGCTGGTGCCG | SEQ ID NO: 706 |
| cg21199406 | CAGAGGCACCGGCCCCAGCCCCACTGGATGTGTGTGGGTCGAGGTCTTCG | SEQ ID NO: 707 |
| cg00822495 | CGACCCCAACCAAATCTGGGGGCCAAAACGCAAAGATCGCGGGAGAAGCC | SEQ ID NO: 708 |
| cg06968181 | CGCCAGCAACTTACTATTTCGCCTGCAACTTGCTTTTAAGCCTGCCGCCC | SEQ ID NO: 709 |
| cg14676529 | AGGCAGCTACATCTGGAAACCATCTTATCCTTCTCTTGTTTTACAAATCG | SEQ ID NO: 710 |
| cg25913647 | TTACATGATGTACACACTCCTAGACCAGGTAGCTGTAGCCATGACCTTCG | SEQ ID NO: 711 |
| cg18134262 | CAAGCTGGTGTCCACCTACAAGAACTACTACTCATTAGTGCTGGACAGCG | SEQ ID NO: 712 |
| cg17205316 | CGGGACTTTTTACAGGCACCAAAAGTTATTAGCAACAGAGGGGAGTAGGA | SEQ ID NO: 713 |
| cg25500080 | TGGGGAGTCAGCGAGGACGGTGGGGCCAGGAGCCCTTGGGAGGGCCTACG | SEQ ID NO: 714 |
| cg06801483 | CGAGGCTGCCAAAAATGTCTCTGGACATTGCCCAGTGTCCCCGGGGAGGC | SEQ ID NO: 715 |
| cg19035496 | CGAGCTGTCAAGAGAGCGTCAGCTTATTAGGCAAATGCTGCGTGGTTTTT | SEQ ID NO: 716 |
| cg04922810 | AGCCAGCGCCAGGCTGCAGTTGGCCTCCAGCAGGCTGTGGAGCAGTGCCG | SEQ ID NO: 717 |
| cg05067406 | CGCATGGAAGACAATTTTTCCACAGATTTTGGGGTGGGGGTAGTTTCAAG | SEQ ID NO: 718 |
| cg01155497 | CGCCTTCTGGTGCTGGTGAAGGACCATGGAGAGCCCCCGCTGACAGCCAC | SEQ ID NO: 719 |
| cg19301632 | ATGACAGACTTCAGACTATTAACCCAGGACTTATATTTTGCCTGGGGACG | SEQ ID NO: 720 |
| cg08473090 | CCTGCGTTTGGGCAGTTCCCGCCCTGCGGCTGGCTAGGAGGAGCTCCTCG | SEQ ID NO: 721 |
| cg09516959 | TCAGCAGCTTTGTACCGCTGGGTCCGGAATCCTCTTTACTCCCTAAACCG | SEQ ID NO: 722 |
| cg13648501 | AGCGCCCTTCATAAAAATTCTTCTGGCTGAGGGTTCTACGTTGGCATACG | SEQ ID NO: 723 |
| cg24063856 | CGCCCAGAAAATAATCTGAGTTCATCCCTAGCCCTCAGGTAACGGATGGG | SEQ ID NO: 724 |
| cg03318614 | CAGAAGGGGCCGAGGGGCCTGGACACGGCTGCTCTCGCTTCTGCTCCTCG | SEQ ID NO: 725 |
| cg24394631 | AGCTTGACCCGGCTCAGGGCATTCAGAGCGATTACATTCAAGAGACTTCG | SEQ ID NO: 726 |
| cg14557699 | CGGAATAGGATCGACTCCATAATGACTGTGATGCTGGAAAAAATTTACTA | SEQ ID NO: 727 |
| cg13784080 | CGGGGTCAGGATTTCTCCTGCTCAGCTCGGGACTTTTTACAGGCACCAAA | SEQ ID NO: 728 |
| cg25725890 | AGGGCAGGGCCGAGCGAGGAATCCTGACTTTCCTGCTCTTTAACTTTGCG | SEQ ID NO: 729 |
| cg07778819 | TGAATGCCCTGAGCCGGGTCAAGCTGGTGTCTATGTTTGTGGCTGGAGCG | SEQ ID NO: 730 |
| cg02829712 | CGAGAGTCAAGCAGTCTGGATTCAGGTATTGAGAGCTTTTGTTCTGTTAC | SEQ ID NO: 731 |
| cg25535999 | TTTTTATGGCTATCTCTTTATGATCCTCTCATTTCTGAACACTGAACTCG | SEQ ID NO: 732 |
| cg11954680 | CGGTTTAGCCACTTCCTGGGTCACTCTTAATTCTAATGTTTCGACTTTGG | SEQ ID NO: 733 |
| cg05620787 | ACAACTAGTTAAAAGCCTTTCATACCAGTCAGTATTCCCAGCCTTGAGCG | SEQ ID NO: 734 |
| cg03384992 | CGCGTTTACGGTTCCAAAACCAAGACTGAAAGGCAGTCTTCACATATTTA | SEQ ID NO: 735 |
| cg16987900 | CGGAACATACAGTATTTTATCTTTATGCGGAAGATCTTCTAATGGAAATA | SEQ ID NO: 736 |
| cg18780217 | GCGGGGAGCGGGGTCGGCTCAGTGGCCCTGAGACCCTAGCTCTGCTCTCG | SEQ ID NO: 737 |
| cg10519368 | ATCTTATCCTTCTCTTGTTTTACAAATCGACGAATCAGCGGTCTAGAACG | SEQ ID NO: 738 |
| cg21702128 | CGGCACAGATTATGATTTTTGTGACTCTGGGAAAGGTTGTGCTGTGTGGG | SEQ ID NO: 739 |
| cg17482089 | CGCGGTACTAGCTTGTTAACTGCGCCTCCCGCGCTGCCAGCCTGAGTCGC | SEQ ID NO: 740 |
| cg06593258 | CGGTTCCAAAACCAAGACTGAAAGGCAGTCTTCACATATTTATTGTTTAT | SEQ ID NO: 741 |
| cg07589972 | AATGCTCAACTGTCTGCAGATCAGATGTCTTGGGATGGAATCCGTTCTCG | SEQ ID NO: 742 |
| cg04450501 | AGGTCCTGTCTCCATCAGCATGCATTTGCAGGGACTGGTCTGTGGTCACG | SEQ ID NO: 743 |
| cg14692377 | GGCTGCGCGGGGAGGCTGGTCCCGGGCTGGGCAGGCGGGCTGGCCTCGCG | SEQ ID NO: 744 |
| cg27345592 | CGGAATTTAATTGAAGAGATGAACTCCAGTGTGCCAGAAAGGACGTGAGA | SEQ ID NO: 745 |
| cg23419885 | CGCGATTCCACCCTGGTGCTGCAGTTTGTAAAAGAAGAAAAAATACCTGT | SEQ ID NO: 746 |
| cg10205431 | GCGGTCTGTGGGCGCGGGCCATGTGGTGGCGAAGGTACGTGCAGTTGACG | SEQ ID NO: 747 |
| cg23490829 | AAAAAAATTTCCCCTAAAGTCTTCAGAAGAAATGCGGCCCAGGAAACACG | SEQ ID NO: 748 |
| cg09746736 | GTGAGTGCGCCCTGAGCGCGGGACAGGGCTAGGTCTGCCTGGGAGGCCCG | SEQ ID NO: 749 |
| cg12466613 | CGGATTCCATCCCAAGACATCTGATCTGCAGACAGTTGAGCATTCACAGC | SEQ ID NO: 750 |

1. A method comprising:
obtaining a biological sample from an individual, the sample comprising cells;
extracting DNA from at least a portion of the cells, the extracted DNA comprising a degree of methylation at one or more loci listed in Table 1;
measuring the degree of methylation of the extracted DNA at one or more loci listed in Table 1; and
identifying the individual as having been subjected to a traumatic experience if the degree of methylation at one or more loci listed in Table 1 is outside of a normal limit.

2. The method of claim 1, wherein exhibiting a degree of methylation at one or more loci listed in Table 1 that is outside a normal limit comprises exhibiting a degree of methylation at a locus, wherein the absolute value of the degree of methylation at the locus is at least as great as the absolute value of the mean difference for the same locus listed in Table 1.

3. The method of claim 1, wherein exhibiting a degree of methylation at one or more loci listed in Table 1 that is outside a normal limit comprises exhibiting a degree of methylation at a locus, wherein the degree of methylation at the locus is altered from the degree of methylation for the same locus in a normal control.

4. The method of claim 1, the method further comprising:
obtaining a second biological sample from the individual at a second time point;
extracting DNA from cells of the second biological sample, the extracted DNA comprising a degree of methylation at one or more loci listed in Table 1;
measuring the degree of methylation of the extracted DNA of the second biological sample at one or more loci listed in Table 1;
comparing the degree of methylation of the extracted DNA of the biological sample at one or more loci listed in Table 1 with the degree of methylation of the extracted DNA of the second biological sample at the same one or more loci listed in Table 1; and
identifying the individual as having been subjected to a traumatic experience between collection of the biological sample and collection of the second biological sample if the degree of methylation for one or more loci listed in Table 1 is altered in the second biological sample.

5. The method of claim 1, further comprising treating the individual with a therapy effective to reverse the methylation at one or more loci listed in Table 1.

6. The method of claim 1, wherein the degree of methylation is measured at a plurality of loci.

7. The method of claim 1, wherein the biological sample comprises saliva.

8. The method of claim 1, wherein the biological sample comprises at least one of a keratinocyte and a white blood cell.

9. The method of claim 8, wherein the keratinocyte is a buccal keratinocyte.

10. The method of claim 1, wherein a traumatic experience comprises physical abuse, emotional abuse, sexual abuse, or neglect.

11. The method of claim 1, wherein the one or more loci comprises at least ten loci listed in Table 1.

12. The method of claim 11, wherein the one or more loci comprises at least 50 loci listed in Table 1.

13. The method of claim 12, wherein the one or more loci comprises at least 100 loci listed in Table 1.

14. The method of claim 13, wherein the one or more loci comprises at least 150 loci listed in Table 1.

15. The method of claim 14, wherein the one or more loci comprises at least 200 loci listed in Table 1.

16. The method of claim 15, wherein the one or more loci comprises each of the loci listed in Table 1.

17. The method of claim 1, wherein the one or more loci comprises loci from at least ten genes.

18. The method of claim 17, wherein the one or more loci comprises loci from at least 30 genes.

19. The method of claim 18, wherein the one or more loci comprises loci from at least 50 genes.

20. The method of claim 1, wherein the one or more loci comprises loci having an absolute value of the mean difference as listed in Table 1 of at least 0.1.

21. The method of claim 20, wherein the one or more loci comprises loci having an absolute value of the mean difference as listed in Table 1 of at least 0.3.

22. The method of claim 1, wherein the one or more loci comprises loci having an absolute value of the mean difference as listed in Table 1 of at least 0.4.

23. A method comprising:
obtaining or having obtained a biological sample from an individual, the sample comprising cells;
extracting or having extracted DNA from at least a portion of the cells, the extracted DNA comprising a degree of methylation at one or more loci listed in Table 1;
measuring or having measured the degree of methylation of the extracted DNA at one or more loci listed in Table 1; and
determining whether the degree of methylation at one or more loci listed in Table 1 is outside of a normal limit.

24. The method of claim 23, wherein exhibiting a degree of methylation at one or more loci listed in Table 1 that is outside a normal limit comprises exhibiting a degree of methylation at a locus, wherein the absolute value of the degree of methylation at the locus is at least as great as the absolute value of the mean difference for the same locus listed in Table 1.

25. The method of claim 23, wherein exhibiting a degree of methylation at one or more loci listed in Table 1 that is outside a normal limit comprises exhibiting a degree of methylation at a locus, wherein the degree of methylation at the locus is altered from the degree of methylation for the same locus in a normal control.

26. The method of claim 23, the method further comprising:
obtaining or having obtained a second biological sample from the individual at a second time point;
extracting or having extracted DNA from cells of the second biological sample;
measuring or having measured the degree of methylation of the extracted DNA of the second biological sample at one or more loci listed in Table 1; and
comparing the degree of methylation for one or more loci of the biological sample with the degree of methylation for the same one or more loci of the second biological sample;
determining if the degree of methylation for one or more loci listed in Table 1 is altered in the second biological sample.

27. The method of claim 26, wherein the method further comprises treating the individual with a therapy after obtaining the first biological sample and before obtaining the second biological sample.

28. The method of claim 26, wherein the individual has been subjected to or is suspected of having been subjected to a traumatic experience between collection of the biological sample and collection of the second biological sample.

29. A method comprising:
obtaining or having obtained a first biological sample from an individual at a first time point and obtaining or having obtained a second biological sample from the same individual at a second time point, the first and second biological samples comprising cells;
extracting or having extracted DNA from at least a portion of the cells of the first biological sample, the extracted DNA comprising a degree of methylation at one or more loci listed in Table 1;
extracting or having extracted DNA from at least a portion of the cells of the second biological sample, the extracted DNA comprising a degree of methylation at one or more loci listed in Table 1;
measuring or having measured the degree of methylation of the extracted DNA at one or more loci listed in Table 1; and
comparing the degree of methylation of the extracted DNA of the first biological sample at one or more loci listed in Table 1 with the degree of methylation of the extracted DNA of the second biological sample at the same one or more loci listed in Table 1; and
determining if the degree of methylation for one or more loci listed in Table 1 is altered in the second biological sample.

30. The method of claim 29, wherein the method further comprises treating the individual with a therapy between obtaining the first biological sample and the second biological sample.

31. The method of claim 29, wherein the individual has been subjected to or is suspected of having been subjected to a traumatic experience between collection of the biological sample and collection of the second biological sample.

32. The method of claim 1, wherein the one or more loci comprise one or more CpG sites.


SEQUENCE LISTING (816 sequences)

SEQ ID NO: 1 - DNA, 50 bp, artificial
  Probe Sequence
aatcacctaaattatttataaacttttaaatatacacttatcaaccraac

SEQ ID NO: 2 - DNA, 50 bp, artificial
  Probe Sequence
acttatcattctacaatccacaaaacaactttccaaaaaatttactaacc

SEQ ID NO: 3 - DNA, 50 bp, artificial
  Probe Sequence
tcaaaatatttcttatatataccaaaataaacacaaaacctcttaacaac

SEQ ID NO: 4 - DNA, 50 bp, artificial
  Probe Sequence
attacaacaataaaaacaaactcaatatccatatcaaataaacaattatc

SEQ ID NO: 5 - DNA, 50 bp, artificial
  Probe Sequence
acaaaatattaaaacaaccaaaactacttactaaaatatacaaataactc

SEQ ID NO: 6 - DNA, 50 bp, artificial
  Probe Sequence
ctcrctataaactttaaatccctcccatcatcttaccatacctttcctac

SEQ ID NO: 7 - DNA, 50 bp, artificial
  Probe Sequence
ccaaccaaaattaacaaaaatttcataacccaataaacataaaaaaaaca

SEQ ID NO: 8 - DNA, 50 bp, artificial
  Probe Sequence
ttaccttcccaaacccrtaaaaaaaatactataaaactaaaacacaaccc

SEQ ID NO: 9 - DNA, 50 bp, artificial
  Probe Sequence
aaacaactacaaaacaaactctaaaaaccattcctcttcaaaactaccca

SEQ ID NO: 10 - DNA, 50 bp, artificial
  Probe Sequence
tacctaaccataatccatataaaacaactctaataaataaaatccttccc

SEQ ID NO: 11 - DNA, 50 bp, artificial
  Probe Sequence
aatacaaaacctaccttaaatattataaaaattctattctcaacaaaaac

SEQ ID NO: 12 - DNA, 50 bp, artificial
  Probe Sequence
tattctctttaactttcaaaaaactaaacatataactcatttctatatac

SEQ ID NO: 13 - DNA, 50 bp, artificial
  Probe Sequence
tacctcaaaatctaaaaacaaaactacatcctcccactcaaaaaaaccca

SEQ ID NO: 14 - DNA, 50 bp, artificial
  Probe Sequence
ctaaaaacctattttttcataattctccatactattccctatctcttacc

SEQ ID NO: 15 - DNA, 50 bp, artificial
  Probe Sequence
tacaatctccttatctccctctactaaaatattcttctctcacttcatcc

SEQ ID NO: 16 - DNA, 50 bp, artificial
  Probe Sequence
aactaaacaaaaaaactaaacaaaaaaactaaacaaaaacaactaccaca

SEQ ID NO: 17 - DNA, 50 bp, artificial
  Probe Sequence
aatacatcaacccacaataaaacaaacaaacatccaactttatacaaaca

SEQ ID NO: 18 - DNA, 50 bp, artificial
  Probe Sequence
atcaaattttcaataataaaaaaatcacacatcaccttataaatttaaac

SEQ ID NO: 19 - DNA, 50 bp, artificial
  Probe Sequence
aaaaaatcraaacttatttcttctatacccatacatcccctataacttcc

SEQ ID NO: 20 - DNA, 50 bp, artificial
  Probe Sequence
acrttaattccctcatctataaaataaaaacaataactacatatcacrac

SEQ ID NO: 21 - DNA, 50 bp, artificial
  Probe Sequence
aataccaaaaaacctaaaattccctaacaaataaatttaatcacttttcc

SEQ ID NO: 22 - DNA, 50 bp, artificial
  Probe Sequence
atctaaaatatactctttaaatattattcctaacrtaatacctttcattc

SEQ ID NO: 23 - DNA, 50 bp, artificial
  Probe Sequence
ctttctaattactaaaattataataatataattccctaatcattcctatc

SEQ ID NO: 24 - DNA, 50 bp, artificial
  Probe Sequence
taatttaaactattatctacttaaatatacrattaattcactcctactcc

SEQ ID NO: 25 - DNA, 50 bp, artificial
  Probe Sequence
aaaaaaataacaaaataaatacaaccatcttattcaaacttaactctaca

SEQ ID NO: 26 - DNA, 50 bp, artificial
  Probe Sequence
tatttaacatcaaaaacaaaaaatccaataaaaaataaaacccacaacca

SEQ ID NO: 27 - DNA, 50 bp, artificial
  Probe Sequence
cctaattaatcctaattctattaacattaaaccaaactcaataaataaac

SEQ ID NO: 28 - DNA, 50 bp, artificial
  Probe Sequence
acaaccaaacataaacaacactaaaaaacaaactctcccaatatactaca

SEQ ID NO: 29 - DNA, 50 bp, artificial
  Probe Sequence
aatcrctataaacctaacaaaaactaaataaactcataaaacctcaaaac

SEQ ID NO: 30 - DNA, 50 bp, artificial
  Probe Sequence
tactatctaaaaacccaaatacttaaatcacctccaaaataactacaaac

SEQ ID NO: 31 - DNA, 50 bp, artificial
  Probe Sequence
cccactaaaaaacacacctaaacatatccacttccttcctaaaaaacaca

SEQ ID NO: 32 - DNA, 50 bp, artificial
  Probe Sequence
aatcctataatcttacaataataacatcatctacaaacacaactaaaaca

SEQ ID NO: 33 - DNA, 50 bp, artificial
  Probe Sequence
taccracaacaacrctcattaaaatcactatataactaacaataaacccc

SEQ ID NO: 34 - DNA, 50 bp, artificial
  Probe Sequence
aaaaactaaacaaaaaaactaaacaaaaaaactaaacaaaaacractacc

SEQ ID NO: 35 - DNA, 50 bp, artificial
  Probe Sequence
caaaaaccatacaacaatacatctaccaatacctaaaatataaatattca

SEQ ID NO: 36 - DNA, 50 bp, artificial
  Probe Sequence
atttacaaaaataatcaatacattttcctaccaaaaaaaactaaccaacc

SEQ ID NO: 37 - DNA, 50 bp, artificial
  Probe Sequence
acaaattaaaaaaactaacaaaaccaaaccccactttaaacttctaaaca

SEQ ID NO: 38 - DNA, 50 bp, artificial
  Probe Sequence
tctccatctaaattacaaaaccctacatattcttaatactacaaaaccac

SEQ ID NO: 39 - DNA, 50 bp, artificial
  Probe Sequence
acttattcctaaataactccracacataaactaaaaacactacactcacc

SEQ ID NO: 40 - DNA, 50 bp, artificial
  Probe Sequence
accraattccctaaaaatcctatcttccacctaatacaaaacataaaacc

SEQ ID NO: 41 - DNA, 50 bp, artificial
  Probe Sequence
aaataccctcaaaaatatttcaaaataaacaaaaataaactatatctaca

SEQ ID NO: 42 - DNA, 50 bp, artificial
  Probe Sequence
caccatcacctattaattcaaaatacaaatataaaacaacctaaaaacrc

SEQ ID NO: 43 - DNA, 50 bp, artificial
  Probe Sequence
attatttacccctataatcacrtaactaactaatattacractcactacc

SEQ ID NO: 44 - DNA, 50 bp, artificial
  Probe Sequence
ttctaaaaatacaaactcccttactataaaaaacctctcaaaaaaaaacc

SEQ ID NO: 45 - DNA, 50 bp, artificial
  Probe Sequence
acccctaaaaacaaaaaaacaaattacaccaacaaataaacaaaaaaaca

SEQ ID NO: 46 - DNA, 50 bp, artificial
  Probe Sequence
ctttcaaaaactctaaaatcaactttcctcacacactctaaattatttac

SEQ ID NO: 47 - DNA, 50 bp, artificial
  Probe Sequence
actataatatcactaataaaacatctcaaaaaaacaccctccraaaattc

SEQ ID NO: 48 - DNA, 50 bp, artificial
  Probe Sequence
tatttatttatataaataaatcattcccatcaatattacctacttttaac

SEQ ID NO: 49 - DNA, 50 bp, artificial
  Probe Sequence
actatcattttataactcccataccctaatataacatactctattttacc

SEQ ID NO: 50 - DNA, 50 bp, artificial
  Probe Sequence
caaaaaaaataaacacattaatctaattacaaataataatattactccca

SEQ ID NO: 51 - DNA, 50 bp, artificial
  Probe Sequence
ataaaaactataaacaatataatactatatcctattaactcacaaaaaac

SEQ ID NO: 52 - DNA, 50 bp, artificial
  Probe Sequence
tactctcttctaacataaaaaaataaacctataatccaaatattaaaacc

SEQ ID NO: 53 - DNA, 50 bp, artificial
  Probe Sequence
aataatactaaaccatctctttcaaaatctaaccttctataaaaaaacac

SEQ ID NO: 54 - DNA, 50 bp, artificial
  Probe Sequence
aaaccttaaaaaatcaaaaatctttcaaaccctacaaaaccrtaaaatac

SEQ ID NO: 55 - DNA, 50 bp, artificial
  Probe Sequence
tacttctaaatatattacaccttctataaaaatttatttaatcacctaac

SEQ ID NO: 56 - DNA, 50 bp, artificial
  Probe Sequence
taatatcaaaaaatctaaattttctccttctctctaccatttaaaaaaac

SEQ ID NO: 57 - DNA, 50 bp, artificial
  Probe Sequence
actttcrtttcaacccataatttatatttaatctttactttactatcatc

SEQ ID NO: 58 - DNA, 50 bp, artificial
  Probe Sequence
atataacatttaacaaatcaacaaaatacactcccttattctttatctcc

SEQ ID NO: 59 - DNA, 50 bp, artificial
  Probe Sequence
cctttacccttcaaacctaactaaaataattttacctaaataaatttcac

SEQ ID NO: 60 - DNA, 50 bp, artificial
  Probe Sequence
aacctaaataaactaaatccaacttctcaaactaaacttcttccaacttc

SEQ ID NO: 61 - DNA, 50 bp, artificial
  Probe Sequence
atactaaaccacrctaaaaatttataactaaaaactaaaccctcrtcatc

SEQ ID NO: 62 - DNA, 50 bp, artificial
  Probe Sequence
aattaatctaaactaactcrtctcaaccaatatctaacactcrcataccc

SEQ ID NO: 63 - DNA, 50 bp, artificial
  Probe Sequence
ataattcaaatctctaactctttttcttaaaacttcrttacttaaaccac

SEQ ID NO: 64 - DNA, 50 bp, artificial
  Probe Sequence
cccatttcrttaataaaaaaactaaccaaataatttaaataacctactac

SEQ ID NO: 65 - DNA, 50 bp, artificial
  Probe Sequence
crtcaaaaaattaccaaaaaaacrctttctcaacaaatctccctaaaaac

SEQ ID NO: 66 - DNA, 50 bp, artificial
  Probe Sequence
aaactctactacaactaccraaaacccaaatatctaaaaatttacctccc

SEQ ID NO: 67 - DNA, 50 bp, artificial
  Probe Sequence
cataaaaccttaaatacactaaaaaaatacataaaccratttcacttaac

SEQ ID NO: 68 - DNA, 50 bp, artificial
  Probe Sequence
aatacacacaaaccttttaaaaactattcccattcaataccaactactca

SEQ ID NO: 69 - DNA, 50 bp, artificial
  Probe Sequence
cataacaaaaattttaattatcttaactttttaccactttcaactactac

SEQ ID NO: 70 - DNA, 50 bp, artificial
  Probe Sequence
aaaaaaaaactactataaaaatttctacrtcttcaccctcactaactatc

SEQ ID NO: 71 - DNA, 50 bp, artificial
  Probe Sequence
atatattttaaacaaaacccaatatctttaaaaaacattctaataatatc

SEQ ID NO: 72 - DNA, 50 bp, artificial
  Probe Sequence
caataaacctaaccaacrtataacrcttcccctacctctactacaaaaac

SEQ ID NO: 73 - DNA, 50 bp, artificial
  Probe Sequence
ctacacatcaaaccaaaaaaaacaactcttcaacataaataccaatacca

SEQ ID NO: 74 - DNA, 50 bp, artificial
  Probe Sequence
aaaaaacaccrcaactctactaatcraaaaacaaccrcaacccaaaattc

SEQ ID NO: 75 - DNA, 50 bp, artificial
  Probe Sequence
crctctcaaaaaaatttactaaaaaaacrcctctctaacaactttttaac

SEQ ID NO: 76 - DNA, 50 bp, artificial
  Probe Sequence
aaataaaaaaatcccaaacataaaccctaaaaccccaacaaaaataaaac

SEQ ID NO: 77 - DNA, 50 bp, artificial
  Probe Sequence
taaccrcrttactcaaaacctaaaactaaaactaacraaactaatacctc

SEQ ID NO: 78 - DNA, 50 bp, artificial
  Probe Sequence
aaaaaatatttcttctttcaatactacctcctttcccttctaaaatctcc

SEQ ID NO: 79 - DNA, 50 bp, artificial
  Probe Sequence
aaattaaacracatactaaccttacctctacaactactatactctactcc

SEQ ID NO: 80 - DNA, 50 bp, artificial
  Probe Sequence
acaactaaaaacaaaaatatatcraactaaacttaactattcatcctacc

SEQ ID NO: 81 - DNA, 50 bp, artificial
  Probe Sequence
atataataaaatcaatttctctaaaatacctttcctaacctcctcaaacc

SEQ ID NO: 82 - DNA, 50 bp, artificial
  Probe Sequence
acactcactatcaatactccactacctaaccaatattattccaaaaaaac

SEQ ID NO: 83 - DNA, 50 bp, artificial
  Probe Sequence
aaaattaaactataacacraaaacctcctaattaaaccaacaaaaaaacc

SEQ ID NO: 84 - DNA, 50 bp, artificial
  Probe Sequence
aatccraaacataatcctaaatcaccrttaatataacrtattaataaaac

SEQ ID NO: 85 - DNA, 50 bp, artificial
  Probe Sequence
tacaaaatacaattaaactactaatcaccrtcctcracrttaataataac

SEQ ID NO: 86 - DNA, 50 bp, artificial
  Probe Sequence
catctctattatttataaacaaaataacaaatcatttaaaattataaccc

SEQ ID NO: 87 - DNA, 50 bp, artificial
  Probe Sequence
aaaaaaaaataaacaaatcatacaaatcatcctattctaaactctaaacc

SEQ ID NO: 88 - DNA, 50 bp, artificial
  Probe Sequence
tcaaattaatatccaccttcaaaaattactattcattaatactaaacaac

SEQ ID NO: 89 - DNA, 50 bp, artificial
  Probe Sequence
aaaccaattccactacaccatcctaaaaaaaaccaaaaacracacataac

SEQ ID NO: 90 - DNA, 50 bp, artificial
  Probe Sequence
cacataatttcaatcattaaacctcataaattaaactaaataaatacctc

SEQ ID NO: 91 - DNA, 50 bp, artificial
  Probe Sequence
aacactttactattctcctacaaaaacttaaaatccaactcraaatactc

SEQ ID NO: 92 - DNA, 50 bp, artificial
  Probe Sequence
aaccataaaatccrtcttaaataaaccctcctcaaaacacaccctctacc

SEQ ID NO: 93 - DNA, 50 bp, artificial
  Probe Sequence
cttctcttattcatatccattccatacccacaccaacttaaataccatac

SEQ ID NO: 94 - DNA, 50 bp, artificial
  Probe Sequence
atacaatctaaataaaaatcttaaacaccaacrataactctcctaaaatc

SEQ ID NO: 95 - DNA, 50 bp, artificial
  Probe Sequence
attttttcatctacctatatattaaatattaatattacctaaatttctac

SEQ ID NO: 96 - DNA, 50 bp, artificial
  Probe Sequence
atataaaaatttccacaattactacttacratttaaaaccacataatatc

SEQ ID NO: 97 - DNA, 50 bp, artificial
  Probe Sequence
caaatttataaaataatttacaaacattttcttttattccctctaacacc

SEQ ID NO: 98 - DNA, 50 bp, artificial
  Probe Sequence
caaaaataccatatttaacctcctccaaaacaaaataataaaactaacca

SEQ ID NO: 99 - DNA, 50 bp, artificial
  Probe Sequence
aaacctcaaaactcatatctaaaatttccccacctttacraaacaaaaac

SEQ ID NO: 100 - DNA, 50 bp, artificial
  Probe Sequence
caaaaccattccaaaaacacaaaaactatcttcaatacacaaataaccca

SEQ ID NO: 101 - DNA, 50 bp, artificial
  Probe Sequence
ccccaaaatacctaaacatactaaacctccaaaaccaattcaccttcacc

SEQ ID NO: 102 - DNA, 50 bp, artificial
  Probe Sequence
atacaaatattaactcaaattccatcttaacctataaactcaattctaac

SEQ ID NO: 103 - DNA, 50 bp, artificial
  Probe Sequence
acttccacatatttatactataatttaactcatttaatccttacaccatc

SEQ ID NO: 104 - DNA, 50 bp, artificial
  Probe Sequence
accaaaacaaaaaccacaaaataaacaaaaaacttaaaaaccaaaataca

SEQ ID NO: 105 - DNA, 50 bp, artificial
  Probe Sequence
tttaaatattctaatctctattaaaatcctactacttctcttattcctcc

SEQ ID NO: 106 - DNA, 50 bp, artificial
  Probe Sequence
aaaacataaacaaatcaaaccaaactcaactttaccaccaactataaacc

SEQ ID NO: 107 - DNA, 50 bp, artificial
  Probe Sequence
taaacaccatatcaaatacctctaattttatttccattaaaaaatcttcc

SEQ ID NO: 108 - DNA, 50 bp, artificial
  Probe Sequence
ttcctactcttcaacrccaacctaaaacraaaactaactaaaaaacaacc

SEQ ID NO: 109 - DNA, 50 bp, artificial
  Probe Sequence
raattaaaaaaaatacaacaccccrcaccctctaacttaacaaaacctac

SEQ ID NO: 110 - DNA, 50 bp, artificial
  Probe Sequence
ctaataaaaacaaaccraaactacaaaatacaattaaactactaatcacc

SEQ ID NO: 111 - DNA, 50 bp, artificial
  Probe Sequence
tttttacctaacataaaaaacaaaaaccaaacaaatttcaaacaaatcca

SEQ ID NO: 112 - DNA, 50 bp, artificial
  Probe Sequence
caactttaataaaatctaatattaaaatcaaaatcccaccaccaaacaca

SEQ ID NO: 113 - DNA, 50 bp, artificial
  Probe Sequence
acataataaactcctttatcaaacactaaacatcatcatttacatccaac

SEQ ID NO: 114 - DNA, 50 bp, artificial
  Probe Sequence
atttacttaatcattaccatctacrcratatccaacctattaatactcac

SEQ ID NO: 115 - DNA, 50 bp, artificial
  Probe Sequence
atctaataaaatcaaataacctaaataaattaaaatccttaaatcaaacc

SEQ ID NO: 116 - DNA, 50 bp, artificial
  Probe Sequence
ctataataaaaaccacttaaacrccaaatccrtcaatcaaataaattccc

SEQ ID NO: 117 - DNA, 50 bp, artificial
  Probe Sequence
aaaaatttaaaatctattacctcatatacaaaaaaaacactcactaaaac

SEQ ID NO: 118 - DNA, 50 bp, artificial
  Probe Sequence
actccattcaatacctcatatttaaaaatcattatcacctcaaataaaca

SEQ ID NO: 119 - DNA, 50 bp, artificial
  Probe Sequence
caccatttcaaattattaacttatttttctaatcaacaaaacttaccttc

SEQ ID NO: 120 - DNA, 50 bp, artificial
  Probe Sequence
ttttctctaacaacatctatctctaaaaaccraacaacraaaaatacaac

SEQ ID NO: 121 - DNA, 50 bp, artificial
  Probe Sequence
aacraccaaacttaatccaaaaatataaataaatccaaaaataattaaac

SEQ ID NO: 122 - DNA, 50 bp, artificial
  Probe Sequence
caactatttaacataaattatcactaacaaaaaaaactaaaatcctaaca

SEQ ID NO: 123 - DNA, 50 bp, artificial
  Probe Sequence
caaaattctaataccacctaaacaccaaaacaaaaaccacaaaataaaca

SEQ ID NO: 124 - DNA, 50 bp, artificial
  Probe Sequence
aaataccaaatcttaacaaacaatacttccaacacatctaactacaccca

SEQ ID NO: 125 - DNA, 50 bp, artificial
  Probe Sequence
tcaaatctcacttatcatttcctaaccataatacttataaaaacaatacc

SEQ ID NO: 126 - DNA, 50 bp, artificial
  Probe Sequence
ttcctacaaaaaatataacaaaaaacraaatacctcttacraatccttcc

SEQ ID NO: 127 - DNA, 50 bp, artificial
  Probe Sequence
aactacctttcaatcttaattttaaaaccataaacacaaacacttcaaca

SEQ ID NO: 128 - DNA, 50 bp, artificial
  Probe Sequence
aacaaattacacaaattaaccattaaatataaaactacacaaaaaacaca

SEQ ID NO: 129 - DNA, 50 bp, artificial
  Probe Sequence
taaaacacaccaaaataaactttaccaaactacatacrccctcaaaaaac

SEQ ID NO: 130 - DNA, 50 bp, artificial
  Probe Sequence
aaaataaattcaaatatattatcctctctctaaaacrcttcctataactc

SEQ ID NO: 131 - DNA, 50 bp, artificial
  Probe Sequence
ccatactctttttaaattaatactaaaacataaaaaaactactacctcca

SEQ ID NO: 132 - DNA, 50 bp, artificial
  Probe Sequence
tcttcaaccrcccccaaaaaactatcaaaaatctccaactcttaaattcc

SEQ ID NO: 133 - DNA, 50 bp, artificial
  Probe Sequence
aacaaaaatactaacctttcactcaaaaataaaaaaactttatctcctca

SEQ ID NO: 134 - DNA, 50 bp, artificial
  Probe Sequence
attttcttctattaaaaaaaatacctttcaaccaaatacaataacactca

SEQ ID NO: 135 - DNA, 50 bp, artificial
  Probe Sequence
ataatatcrctatccaccacaaaatctttctccacaaaaaaaataacaac

SEQ ID NO: 136 - DNA, 50 bp, artificial
  Probe Sequence
ataacaacttcataaaccacatcacacaaaacctaaaactaaaactaaca

SEQ ID NO: 137 - DNA, 50 bp, artificial
  Probe Sequence
aaaacttttaaaaacaatcaatctactaaaaaaactccaacaaaaaaaac

SEQ ID NO: 138 - DNA, 50 bp, artificial
  Probe Sequence
acacrcaatcraactatcaaaaaaacrtcaacttattaaacaaatactac

SEQ ID NO: 139 - DNA, 50 bp, artificial
  Probe Sequence
caacttcccaaaataccaaaattacaaataaacataaactaccacaccca

SEQ ID NO: 140 - DNA, 50 bp, artificial
  Probe Sequence
aaatataaaactacacaaaaaacacaaacaataaaacaaaacctcaaaca

SEQ ID NO: 141 - DNA, 50 bp, artificial
  Probe Sequence
aaatcaataatacattattctttaacaaataaaaaccccatctttcaacc

SEQ ID NO: 142 - DNA, 50 bp, artificial
  Probe Sequence
cacctaacaaaccaacactaatatattctaacacaataaaaaattaatca

SEQ ID NO: 143 - DNA, 50 bp, artificial
  Probe Sequence
ccttcttttaacccccattataatttatttttacatcttttataattatc

SEQ ID NO: 144 - DNA, 50 bp, artificial
  Probe Sequence
aaaaacaaatccttaactaaacttttaaataaactctctatataaatacc

SEQ ID NO: 145 - DNA, 50 bp, artificial
  Probe Sequence
aaatttttatcaataaacaaatcataaaaaacccttccattttaaaactc

SEQ ID NO: 146 - DNA, 50 bp, artificial
  Probe Sequence
aaactaacctactaaactaaatcaaaaactataaacaacattattcctac

SEQ ID NO: 147 - DNA, 50 bp, artificial
  Probe Sequence
cttcrctataaaccacraccaatatttctataaaaataactaatataaac

SEQ ID NO: 148 - DNA, 50 bp, artificial
  Probe Sequence
acacaaaccataaaaaaaataacaaaaacaaaaccaacaaaaaacaacca

SEQ ID NO: 149 - DNA, 50 bp, artificial
  Probe Sequence
taccttttattaaacraaatttaactataatatcccaaatacrcttctcc

SEQ ID NO: 150 - DNA, 50 bp, artificial
  Probe Sequence
accaaaaaataaattcacattaaccataaaaacaaaccccaacctctttc

SEQ ID NO: 151 - DNA, 50 bp, artificial
  Probe Sequence
tctaaatataaattctccaaaaaccacctcrtaaacaatcataataaaac

SEQ ID NO: 152 - DNA, 50 bp, artificial
  Probe Sequence
taaacaataaaaaaacaaacacaaaataacacaaacaaaaacaaaaccca

SEQ ID NO: 153 - DNA, 50 bp, artificial
  Probe Sequence
acaacracatcatataacttcaaaaaataataattacataaattacttcc

SEQ ID NO: 154 - DNA, 50 bp, artificial
  Probe Sequence
tctaaatttaacctctttccataaattttctttaccccacttccaaatac

SEQ ID NO: 155 - DNA, 50 bp, artificial
  Probe Sequence
ttccttttccctccccctaaaactataacaacaacaacaacaaaaaacca

SEQ ID NO: 156 - DNA, 50 bp, artificial
  Probe Sequence
atataaaaactaaatcatacttacacttacacrttcatacrcataatatc

SEQ ID NO: 157 - DNA, 50 bp, artificial
  Probe Sequence
taaaaactttaacctcctaattaaattctatttaaccaaaacrccrattc

SEQ ID NO: 158 - DNA, 50 bp, artificial
  Probe Sequence
tttcaccctatctcccaaactataatacaataacctaatctaaactcacc

SEQ ID NO: 159 - DNA, 50 bp, artificial
  Probe Sequence
aaaaataacaaaaacaaaaccaacaaaaaacaaccacaaaccaaactaca

SEQ ID NO: 160 - DNA, 50 bp, artificial
  Probe Sequence
aaaaccaaaaaaaacaaacaataaacaccaaaacaaacaaaaaaccaaca

SEQ ID NO: 161 - DNA, 50 bp, artificial
  Probe Sequence
aaaaataaactacttatctccccacctctctaccccatttatctaaaaca

SEQ ID NO: 162 - DNA, 50 bp, artificial
  Probe Sequence
catattaaaaaaacactcttcacattcactcaacctaataaccaaaatca

SEQ ID NO: 163 - DNA, 50 bp, artificial
  Probe Sequence
attacrtattcaaatactactttacttcatcctctctaaaatttaacacc

SEQ ID NO: 164 - DNA, 50 bp, artificial
  Probe Sequence
attcaataaaataaataaaacttatttaactcatacatctctcactaacc

SEQ ID NO: 165 - DNA, 50 bp, artificial
  Probe Sequence
aataaatctacaaaataacattttatttataaattctcaaataaaccaca

SEQ ID NO: 166 - DNA, 50 bp, artificial
  Probe Sequence
atattcaaaatcttttctctaacaacatctatctctaaaaaccraacaac

SEQ ID NO: 167 - DNA, 50 bp, artificial
  Probe Sequence
aacattaaatacatcaactaccccaaacttaaaataaaaacataataccc

SEQ ID NO: 168 - DNA, 50 bp, artificial
  Probe Sequence
taataatattacaaactttcttaaaaatcatatctaactatttccccccc

SEQ ID NO: 169 - DNA, 50 bp, artificial
  Probe Sequence
taaatttataatacraaaaccattcttatccttaaaaaactatcaaaacc

SEQ ID NO: 170 - DNA, 50 bp, artificial
  Probe Sequence
aaaatcaacccaaatcaaaaatacccaactaaaaaatcttaatacaccca

SEQ ID NO: 171 - DNA, 50 bp, artificial
  Probe Sequence
cccattatactttactatttttaattcttcaactattcctaaataatctc

SEQ ID NO: 172 - DNA, 50 bp, artificial
  Probe Sequence
cttctaaaataactccaactccaaataaaaaccaataaacccataaacac

SEQ ID NO: 173 - DNA, 50 bp, artificial
  Probe Sequence
caacaataaaaacaaccraacrtaacctcrcccataataaactaaaaaac

SEQ ID NO: 174 - DNA, 50 bp, artificial
  Probe Sequence
acaaaacatactcttctaatttaatcaaaacattaaaaattaataataac

SEQ ID NO: 175 - DNA, 50 bp, artificial
  Probe Sequence
cctttaaattctaaatatcaaaatcacaaccttaactcctttaactatcc

SEQ ID NO: 176 - DNA, 50 bp, artificial
  Probe Sequence
aaaaatataaaaatcaaataaataattcccatcrccaataaccccaaaac

SEQ ID NO: 177 - DNA, 50 bp, artificial
  Probe Sequence
ccaacttaaaaaaaacrtaaaacrtcaaaaaacaaataacatatccattc

SEQ ID NO: 178 - DNA, 50 bp, artificial
  Probe Sequence
aaaaaaaaatacatccacctacaaaactccacattcacaaaaaaacatca

SEQ ID NO: 179 - DNA, 50 bp, artificial
  Probe Sequence
aaactataaaaaaaaaccaaaaatacaacctcatattaaacccataccca

SEQ ID NO: 180 - DNA, 50 bp, artificial
  Probe Sequence
ttcactataaaccaccaccaacttatctataaaaataaccaacataaaca

SEQ ID NO: 181 - DNA, 50 bp, artificial
  Probe Sequence
cttacttacataataaccacataatatcrctctttaccrcaaattctttc

SEQ ID NO: 182 - DNA, 50 bp, artificial
  Probe Sequence
taccaaaaaaattaaccttaactattaattatcattaaaaaaaaacccca

SEQ ID NO: 183 - DNA, 50 bp, artificial
  Probe Sequence
aaaacctcctcacctctttactcaacatctaaaaaataatttacaaattc

SEQ ID NO: 184 - DNA, 50 bp, artificial
  Probe Sequence
taaaatatccataaaaataaccaacataaacaacaatacacctacattca

SEQ ID NO: 185 - DNA, 50 bp, artificial
  Probe Sequence
ttaaataaacttactttaaaaccataatcaacatttaaatcttactcttc

SEQ ID NO: 186 - DNA, 50 bp, artificial
  Probe Sequence
ctatcccaattctcaacctattctcaaaaaaataaaactattaatatatc

SEQ ID NO: 187 - DNA, 50 bp, artificial
  Probe Sequence
ataacacttaataaaacattaaaaacactattccacctaataatatttcc

SEQ ID NO: 188 - DNA, 50 bp, artificial
  Probe Sequence
cccccacacactctactataaactacttaattttatttattatttcaatc

SEQ ID NO: 189 - DNA, 50 bp, artificial
  Probe Sequence
ttaatcaatattcaaaatcttttctctaacaacatctatctctaaaaacc

SEQ ID NO: 190 - DNA, 50 bp, artificial
  Probe Sequence
acaattaaaaaaaacccrtttctattcccaccaaatacaaatatcaaatc

SEQ ID NO: 191 - DNA, 50 bp, artificial
  Probe Sequence
taaatacaaaccacataataacaaaaatacacacaataaacaccaattca

SEQ ID NO: 192 - DNA, 50 bp, artificial
  Probe Sequence
caaccactaaactacraaaaactaaaactactacaattctaaataaaaac

SEQ ID NO: 193 - DNA, 50 bp, artificial
  Probe Sequence
taatatcraccctcttcaaaaaccacrcaacatttacctaataaactaac

SEQ ID NO: 194 - DNA, 50 bp, artificial
  Probe Sequence
aatctattaatactcacactactactatatactactctacratactccrc

SEQ ID NO: 195 - DNA, 50 bp, artificial
  Probe Sequence
ctacaaattttccatataaaaataacaataaaaaacatcaataacaatcc

SEQ ID NO: 196 - DNA, 50 bp, artificial
  Probe Sequence
aaattcaaactttaaaatcaaaccaatctaaatctaaactctactacctc

SEQ ID NO: 197 - DNA, 50 bp, artificial
  Probe Sequence
aatacccraaaaaaacaattacaatctaccttccccaaaaacaaaaaccc

SEQ ID NO: 198 - DNA, 50 bp, artificial
  Probe Sequence
ctataacrcctaattatcrcaacaactacatctatcaactaacaacaccc

SEQ ID NO: 199 - DNA, 50 bp, artificial
  Probe Sequence
aaaaatcattttcccaaactctttacaaaatacctcraactcattccaac

SEQ ID NO: 200 - DNA, 50 bp, artificial
  Probe Sequence
aactaatacctttattctaaaaaactattttataaacracaacracatcc

SEQ ID NO: 201 - DNA, 50 bp, artificial
  Probe Sequence
attacttaaattccrcaatcaacaacttttattctctaaacraaaacacc

SEQ ID NO: 202 - DNA, 50 bp, artificial
  Probe Sequence
atataaccaactccccaccacactaaaacacaccaaaataaacttaccca

SEQ ID NO: 203 - DNA, 50 bp, artificial
  Probe Sequence
cacrtacaacaacaacacaatattattcrttttatctacrtttaatttcc

SEQ ID NO: 204 - DNA, 50 bp, artificial
  Probe Sequence
cctcttaacaactcctaaaatacaaaaaaatacaaaacaaacttcaaccc

SEQ ID NO: 205 - DNA, 50 bp, artificial
  Probe Sequence
ctataaataattaatctttaaaacccacrtcccattctccaaacattctc

SEQ ID NO: 206 - DNA, 50 bp, artificial
  Probe Sequence
ttaaccacatcacacaaaacctaaaactaaaactaacaaaactaatacca

SEQ ID NO: 207 - DNA, 50 bp, artificial
  Probe Sequence
caaaaacaccaaccccaaccccactaaatatatataaatcaaaatcttca

SEQ ID NO: 208 - DNA, 50 bp, artificial
  Probe Sequence
aaacttctcccrcratctttacrttttaacccccaaatttaattaaaatc

SEQ ID NO: 209 - DNA, 50 bp, artificial
  Probe Sequence
aaacaacaaacttaaaaacaaattacaaacaaaataataaattactaaca

SEQ ID NO: 210 - DNA, 50 bp, artificial
  Probe Sequence
caaacaactacatctaaaaaccatcttatccttctcttattttacaaatc

SEQ ID NO: 211 - DNA, 50 bp, artificial
  Probe Sequence
tttacataatatacacactcctaaaccaaataactataaccataaccttc

SEQ ID NO: 212 - DNA, 50 bp, artificial
  Probe Sequence
tcaaactaatatccacctacaaaaactactactcattaatactaaacaac

SEQ ID NO: 213 - DNA, 50 bp, artificial
  Probe Sequence
ctcctactcccctctattactaataacttttaatacctataaaaaatccc

SEQ ID NO: 214 - DNA, 50 bp, artificial
  Probe Sequence
taaaaaatcaacaaaaacaataaaaccaaaaacccttaaaaaaacctaca

SEQ ID NO: 215 - DNA, 50 bp, artificial
  Probe Sequence
crcctccccraaaacactaaacaatatccaaaaacatttttaacaacctc

SEQ ID NO: 216 - DNA, 50 bp, artificial
  Probe Sequence
caaaaaccacrcaacatttacctaataaactaacrctctcttaacaactc

SEQ ID NO: 217 - DNA, 50 bp, artificial
  Probe Sequence
caaccaacrccaaactacaattaacctccaacaaactataaaacaatacc

SEQ ID NO: 218 - DNA, 50 bp, artificial
  Probe Sequence
tcttaaaactacccccaccccaaaatctataaaaaaattatcttccatac

SEQ ID NO: 219 - DNA, 50 bp, artificial
  Probe Sequence
tataactatcaacraaaactctccataatccttcaccaacaccaaaaaac

SEQ ID NO: 220 - DNA, 50 bp, artificial
  Probe Sequence
aataacaaacttcaaactattaacccaaaacttatattttacctaaaaac

SEQ ID NO: 221 - DNA, 50 bp, artificial
  Probe Sequence
cctacatttaaacaattcccaccctacaactaactaaaaaaaactcctca

SEQ ID NO: 222 - DNA, 50 bp, artificial
  Probe Sequence
tcaacaactttataccactaaatccaaaatcctctttactccctaaacca

SEQ ID NO: 223 - DNA, 50 bp, artificial
  Probe Sequence
caacrcccttcataaaaattcttctaactaaaaattctacrttaacatac

SEQ ID NO: 224 - DNA, 50 bp, artificial
  Probe Sequence
ccccatccrttacctaaaaactaaaaataaactcaaattattttctaaac

SEQ ID NO: 225 - DNA, 50 bp, artificial
  Probe Sequence
caaaaaaaaccaaaaaacctaaacacaactactctcacttctactcctca

SEQ ID NO: 226 - DNA, 50 bp, artificial
  Probe Sequence
caacttaacccractcaaaacattcaaaacrattacattcaaaaaacttc

SEQ ID NO: 227 - DNA, 50 bp, artificial
  Probe Sequence
ttaataaattttttccaacatcacaatcattataaaatcratcctattcc

SEQ ID NO: 228 - DNA, 50 bp, artificial
  Probe Sequence
ttttaatacctataaaaaatcccraactaaacaaaaaaaatcctaacccc

SEQ ID NO: 229 - DNA, 50 bp, artificial
  Probe Sequence
caaaacaaaaccraacraaaaatcctaactttcctactctttaactttac

SEQ ID NO: 230 - DNA, 50 bp, artificial
  Probe Sequence
ctaaataccctaaaccraatcaaactaatatctatatttataactaaaac

SEQ ID NO: 231 - DNA, 50 bp, artificial
  Probe Sequence
crtaacaaaacaaaaactctcaatacctaaatccaaactacttaactctc

SEQ ID NO: 232 - DNA, 50 bp, artificial
  Probe Sequence
atttttataactatctctttataatcctctcatttctaaacactaaactc

SEQ ID NO: 233 - DNA, 50 bp, artificial
  Probe Sequence
ccaaaatcaaaacattaaaattaaaaataacccaaaaaataactaaacca

SEQ ID NO: 234 - DNA, 50 bp, artificial
  Probe Sequence
cacaactaattaaaaacctttcataccaatcaatattcccaaccttaaac

SEQ ID NO: 235 - DNA, 50 bp, artificial
  Probe Sequence
taaatatataaaaactacctttcaatcttaattttaaaaccataaacaca

SEQ ID NO: 236 - DNA, 50 bp, artificial
  Probe Sequence
tatttccattaaaaaatcttccacataaaaataaaatactatatattcca

SEQ ID NO: 237 - DNA, 50 bp, artificial
  Probe Sequence
acaaaaaacaaaatcaactcaataaccctaaaaccctaactctactctca

SEQ ID NO: 238 - DNA, 50 bp, artificial
  Probe Sequence
catcttatccttctcttattttacaaatcracraatcaacratctaaaac

SEQ ID NO: 239 - DNA, 50 bp, artificial
  Probe Sequence
acccacacaacacaacctttcccaaaatcacaaaaatcataatctatacc

SEQ ID NO: 240 - DNA, 50 bp, artificial
  Probe Sequence
acaactcaaactaacaacacaaaaaacacaattaacaaactaataccaca

SEQ ID NO: 241 - DNA, 50 bp, artificial
  Probe Sequence
tataaacaataaatatataaaaactacctttcaatcttaattttaaaacc

SEQ ID NO: 242 - DNA, 50 bp, artificial
  Probe Sequence
aaatactcaactatctacaaatcaaatatcttaaaataaaatccrttctc

SEQ ID NO: 243 - DNA, 50 bp, artificial
  Probe Sequence
caaatcctatctccatcaacatacatttacaaaaactaatctataatcac

SEQ ID NO: 244 - DNA, 50 bp, artificial
  Probe Sequence
aactacacaaaaaaactaatcccaaactaaacaaacaaactaacctcaca

SEQ ID NO: 245 - DNA, 50 bp, artificial
  Probe Sequence
atctcacrtcctttctaacacactaaaattcatctcttcaattaaattcc

SEQ ID NO: 246 - DNA, 50 bp, artificial
  Probe Sequence
aacaaatattttttcttcttttacaaactacaacaccaaaataaaatcrc

SEQ ID NO: 247 - DNA, 50 bp, artificial
  Probe Sequence
acaatctataaacacaaaccatataataacaaaaatacatacaattaaca

SEQ ID NO: 248 - DNA, 50 bp, artificial
  Probe Sequence
aaaaaaaatttcccctaaaatcttcaaaaaaaatacracccaaaaaacac

SEQ ID NO: 249 - DNA, 50 bp, artificial
  Probe Sequence
ataaatacaccctaaacacaaaacaaaactaaatctacctaaaaaaccca

SEQ ID NO: 250 - DNA, 50 bp, artificial
  Probe Sequence
tactataaatactcaactatctacaaatcaaatatcttaaaataaaatcc

SEQ ID NO: 251 - DNA, 122 bp, homo sapiens
gacttagcacaagtcacctggattatttatgagcttttaaatatacacttgtcagccgggcgcagtgactcatgcctgtaatcccagcactttaggaggcctaggcgggtggatcacttgag

SEQ ID NO: 252 - DNA, 122 bp, homo sapiens
ccagggttgctgcttatcattctacaatccacagggcagctttccaaagaatttgctggccgcaaataccaacaggtctgagattaagaaaccctgttctagatgaatatacaccaaacttt

SEQ ID NO: 253 - DNA, 122 bp, homo sapiens
atttcaggaactcaaaatatttcttatgtatgccaagataaacacaaaacctcttggcagcgatctatgaggattctccttgcttgtgattttgcatttctttatgctaattcagaaatatg

SEQ ID NO: 254 - DNA, 122 bp, homo sapiens
tttgagatggtattgcagcaatgaaaacaagctcaatgtccatgtcagataaacaattgtcgtcacagctctggaagtttgggaagtgactcatcttcaggaatgggcagtgagtgaagaca

SEQ ID NO: 255 - DNA, 122 bp, homo sapiens
gaagatgttaggcaaaatattaagacagccaaagctgcttactgagatgtgcaagtgactcgcagggggacagacatgccacttcagcaccagaaggttttaatttaaacaatatgattcca

SEQ ID NO: 256 - DNA, 122 bp, homo sapiens
gggcccccaggcgcagccccctcccacggcacctttccagctggctgtgagccctagggccgcaggaaaggtatggcaagatgatgggagggatttaaagttcacagcgagaacaagaaaag

SEQ ID NO: 257 - DNA, 122 bp, homo sapiens
catctggatgcacgccaagacaagacgtcagtcatttcctggtgaaatgaaagaaagccacgcttcctccacgcccattgggtcacgaaatccttgctaatcctggccggggcactggagga

SEQ ID NO: 258 - DNA, 122 bp, homo sapiens
agaaaacacatttgccttcccaggcccgtgggaggagtgctgtgaggctgagacacagcccggcttgctgacggcacaaatgccggtgcgccctcggcacagtttgtttgcttcatcgggcg

SEQ ID NO: 259 - DNA, 122 bp, homo sapiens
gagggaagtgcgaagcagctgcgggacggactctagggaccgttcctcttcagggctgcccgcctcagaagggcctgtgcatgccgctgtgtgtgtgttgggggtgtgggcgggtagaggag

SEQ ID NO: 260 - DNA, 122 bp, homo sapiens
ggggcccacagctaaatccagtgagtggaagttacagggagtctgcttccagtgctgctccgggaaggatcccatccaccagagctgccccacatggaccatggtcaggcagaggaagatgc

SEQ ID NO: 261 - DNA, 122 bp, homo sapiens
cttctgggcagagtgcagggcctgccttaaatgttgtgggggttctgttctcaacaaaggcgtaagttgggagcgactgagccactccaggaggaggtgtgggcccacggcgtgttggaggt

SEQ ID NO: 262 - DNA, 122 bp, homo sapiens
taattattcccgacttcacttcccttttactctgcgttgcttcacaaagacgcacacacacgcacacagaaatgagtcatatgttcagctttctgaaagtcaaagagaataaacagatgcca

SEQ ID NO: 263 - DNA, 122 bp, homo sapiens
ctgagtccaaggtgcctcagggtctgaagacaaagctgcgtcctcccgctcaggagggcccgaggagcggacggcagaatcttggggcccagcccgtctgtccctcatgggtctgtgacctt

SEQ ID NO: 264 - DNA, 122 bp, homo sapiens
tcagaggcagagactgtagaggcagaagcaaagcaagacacagagcatttcagagacccacggcaagagacagggaacagcatggagaactatgaaaaaataggcttccagagaaagagaaa

SEQ ID NO: 265 - DNA, 122 bp, homo sapiens
acaagggacatgaaaagggcaaggaaataagaaaatgagctgagttttgaaggcaatgatcggatgaagtgagagaagaatattccagcagagggagacaaggagactgcagagatttcaag

SEQ ID NO: 266 - DNA, 122 bp, homo sapiens
ccgccgcggttaccacggcgacgcgcgcgcacgcgcacacacgccctcagggatgtggggcgcggcagccgcccctgcccagctcccctgctcagctcccctgctcagctccccggttgcgc

SEQ ID NO: 267 - DNA, 122 bp, homo sapiens
tttgatcgccaccgagcgggttggtgcagcccccctccccatctcgagccctcctacctccgcctgcacaaagccggacgcctgcccgccccaccgtgggtcgatgcaccctgccaggggag

SEQ ID NO: 268 - DNA, 122 bp, homo sapiens
tctggatccaagtcaaattttcagtgatggaagaatcacacatcaccttgtggatttgaacggctcctcttcagttgtctcccacagactgccataatttgccccagaatagagtccctgag

SEQ ID NO: 269 - DNA, 122 bp, homo sapiens
gcatgcccaggaaggagtcgggacttgtttcttctgtacccatacatcccctgtggcttccgcctcctcctgggctttacggggaggtccagagggaagaggctgccaatttaagaaggaag

SEQ ID NO: 270 - DNA, 122 bp, homo sapiens
aaactctctgagcgttggttccctcatctgtaagatgaaaacagtaactacatgtcacggcgtcagtgaaaagattaaataagataatgtatataaagcactatcagagtccctggcccaga

SEQ ID NO: 271 - DNA, 122 bp, homo sapiens
ccaaatcccaaggtaccagagagcctaaaattccctggcagatgaatttaatcacttttccgtgttgttaataagaaaacactacagaatgtcagctggtggaatcggaattttcagtgctc

SEQ ID NO: 272 - DNA, 122 bp, homo sapiens
acttgattgaaatctaggatatgctctttgaatgttattcctaacgtaatgcctttcattcgtccatccatccatccatccatcaattcatccaacaagtgtagtgggcattgtgccagcaa

SEQ ID NO: 273 - DNA, 122 bp, homo sapiens
ccccactgaccacaaggaggtgaggaatcacaatgctaccttgtgtttgcaactggagaccgataggaatgattagggaaccataccaccataatcctagtaaccagaaagccagccagcta

SEQ ID NO: 274 - DNA, 122 bp, homo sapiens
ccacagagaccgaatgcatcaattgtttcacttatggaacattctgggctctgagaacctcggagtaggagtgaatcaatcgtacatccaagcagataacagctcaaatcaccagctcagca

SEQ ID NO: 275 - DNA, 122 bp, homo sapiens
gagacctcaagagagagaatggcaaagtaaatacgaccatcttattcgaacttaactctgcgtttcaagtagggagataagaaggactgctgttccacttcttttctagatgacaagatggg

SEQ ID NO: 276 - DNA, 122 bp, homo sapiens
atgaattgggtatatttgacatcaaagacaggagatccagtggggaatgagacccacggccgggcgtgggcagcgctggaggacaggctctcccagtgtgctgcgggggagggcagcccctg

SEQ ID NO: 277 - DNA, 122 bp, homo sapiens
ccaactcttaacctagttaatcctagttctgttgacattggaccaggctcagtaaataaacgaatggatttccagcctttttttcccatctgttcctgcttttagtcctctgaatctgcttc

SEQ ID NO: 278 - DNA, 122 bp, homo sapiens
ggaatgagacccacggccgggcgtgggcagcgctggaggacaggctctcccagtgtgctgcgggggagggcagcccctgcagtgtgcgtgttgagtgctgggaaacccccaagtaacacttc

SEQ ID NO: 279 - DNA, 122 bp, homo sapiens
gcgcacagccaagtcgctgtggacctagcaagggctggatggactcatggagcctcagggcgggttggggaagctggcgaggccgagccccgctttgggcttctgggcgccctgacctcgct

SEQ ID NO: 280 - DNA, 122 bp, homo sapiens
gtgtacacacacacacacacacacacaccacaggcaagctcaggcagggtccttcacagacgtctgcagtcactctggaggtgacccaagcacctgggcccttagacagtaacaccactcac

SEQ ID NO: 281 - DNA, 122 bp, homo sapiens
cgtgcgcacctccccgctgggggacacgcctgagcatgtccacttccttcctgggggacacgcatgagcgtgcccacctccccccaccccacaggacacacctgagcacgtccgcctcttcc

SEQ ID NO: 282 - DNA, 122 bp, homo sapiens
acaggactcctccccagccgctcctgtatatgacgtcaccgccgtaagaccacaggacaccgccccagccgcgcctgtagatgacgtcaccatcgtaagaccacaggacccttcccagccgc

SEQ ID NO: 283 - DNA, 122 bp, homo sapiens
tctctgttgagattcacatcctgacttctgtccgtgggctccactaaaacctgcaggctccggggtccattgccagtcacatagtgacttcaatgagcgttgctgccggcatcctgagcggg

SEQ ID NO: 284 - DNA, 122 bp, homo sapiens
gccgcggttaccacggcgacgcgcgcgcacgcgcacacacgccctcagggatgtggggcgcggcagccgcccctgcccagctcccctgctcagctcccctgctcagctccccggttgcgctc

SEQ ID NO: 285 - DNA, 122 bp, homo sapiens
gagtccgcagagcgagaaccgtgcgacagtgcgtctgccaatacctggaatgtaggtgttcgttgcagtgtggccttcccagccggcctctcagcacacccggccaggcagccagtgttccc

SEQ ID NO: 286 - DNA, 122 bp, homo sapiens
tcttttccttgatttacaaagatagtcaatacattttcctaccaaaaagaactggccagccgtggtggctcatgcctgtaatcctagcagtttaggaagccgaggtgggcggatcacttgag

SEQ ID NO: 287 - DNA, 122 bp, homo sapiens
tggagcctcagggcgggttggggaagctggcgaggccgagccccgctttgggcttctgggcgccctgacctcgctcccggaaccctggggactgggaggcgggagggggcttattgattcca

SEQ ID NO: 288 - DNA, 122 bp, homo sapiens
gatgcccatgagacttcctgggccccagtcttccaggtcacgcagggtcactgcgcttcacgtggccctgcagcaccaagaatatgcagggccttgcaatccagatggagaattgtgcaaac

SEQ ID NO: 289 - DNA, 122 bp, homo sapiens
caagttcaggagcttgttcctaggtgactccggcacataggctggaaacactacactcaccgggggctcagccccgcacacctgtgtggggagggagcggggagcgtcggggaagaaggagg

SEQ ID NO: 290 - DNA, 122 bp, homo sapiens
ctccctgcaaggtgagtcagctcccagaagccccagggcctggtccggctgtcctggccccggcctcatgccctgcactaggtggaagacaggatctccagggaacccggctgctttgggcc

SEQ ID NO: 291 - DNA, 122 bp, homo sapiens
ccagcagagggggcaggaggctggccagcccagtcctccctccaggagctggaaccatcccgcagacacagtccactcctgcccactccgaaacacctctgagggtattctctcagcagtag

SEQ ID NO: 292 - DNA, 122 bp, homo sapiens
tgccgcggatgagtctccaggtgcgtgggggctgcagttcctgtgccccttccggccgcccgcgcccccaggctgcctcacacctgcatcctgaactaacaggtgatggtggtggcggcgct

SEQ ID NO: 293 - DNA, 122 bp, homo sapiens
gcagagtatcaattgtttgcccctgtgatcacgtggctgactggtgttgcggctcactgccgctgcccagtagtaccgcatatgactattctgcgaaaagaccaaaattcaaaatttaaaat

SEQ ID NO: 294 - DNA, 122 bp, homo sapiens
gaacatgctaattctaaaaatacagactcccttgctgtgaaagacctctcaggggagaaccgacattgaagcaaaacaatgatttcatccctcccgcccaccatcccccagcctgtgaacca

SEQ ID NO: 295 - DNA, 122 bp, homo sapiens
tcctctcccagccaggccccgccccccgaccaggctctattcccttaactcccttatgtgcgccctcctgtccacctgctggcgcaacctgctcccccgtctctaggggtggggtggggagc

SEQ ID NO: 296 - DNA, 122 bp, homo sapiens
tccctattgacctttcagggactctgaaatcaactttcctcacacactctgaattgtttgcgtttgttttcatcctgccagagccacccgcccaggccacccatctcttccctcctccctgc

SEQ ID NO: 297 - DNA, 122 bp, homo sapiens
agctggcaatcactgtgatgtcactgatgaaacatctcaaaggaacaccctccgaaggttcgaggctatttgtgttcgggggacatggaaatatgttgactaagactttgttcctttggctg

SEQ ID NO: 298 - DNA, 122 bp, homo sapiens
ttgaagtaaatcgtccttgcagaaatctttgtttcattagtaaagaaaactggatgaccccgttaaaagtaggtaacattgatgggaatgatttatccacataaataaacaaaatggctgtg

SEQ ID NO: 299 - DNA, 122 bp, homo sapiens
attccagtggagctgtcattttgtggctcccatgccctggtgtggcatgctctgttttgccgtgactgcggctacatggcttggcctgttgggtggcggtggttgtgtggggctggctatgg

SEQ ID NO: 300 - DNA, 122 bp, homo sapiens
tgctggcgtccacgaaggaagtgagcgcgttgatctggttgcggatggtgatgttgctcccggggcaagccgggcaggagcggaagaacgggatgcagtcggcttggttcttgatgcggggg

SEQ ID NO: 301 - DNA, 122 bp, homo sapiens
tggacacagacatgagagctataaacaatgtgatactatgtcctgttaactcacagagaacggaagggctggaacgggacccagccagcagggtttgtgaccgcctctgctgtgacgtcaca

SEQ ID NO: 302 - DNA, 122 bp, homo sapiens
atttttttttaatgcaacaaaagagatgattcagaattaatagtgtttttccccagggcacggttttaacacttggattacaggttcatctctccatgccagaagagagcagcagggagcga

SEQ ID NO: 303 - DNA, 122 bp, homo sapiens
caagatccataggtggtgctgagccatctctttcagggtctaaccttctgtggaaaagcacggcactgttctagcagagtcagtttcacttgagttttcttcctcctttttataacgacatt

SEQ ID NO: 304 - DNA, 122 bp, homo sapiens
accctctttctgtttctattccttccccactcatgccccagtgcttccgttggacacatgcgcattttacggtcctgcagggcttgaaagatttctgaccttctaaggtccagtgatttggt

SEQ ID NO: 305 - DNA, 122 bp, homo sapiens
ccctcttctgttgcttctgggtatgttgcaccttctgtaagaatttatttagtcacctgacgccggtataagtcagatgactacatagattttagtcatctaattttatgaagacagactgg

SEQ ID NO: 306 - DNA, 122 bp, homo sapiens
gtccagtgatttggtatcagaagatctaagttttctccttctctctgccatttaaagaagcgtgttgcaatttccccagcagaagtgaaagtgattcgcctctactcaaatgtctgcatagg

SEQ ID NO: 307 - DNA, 122 bp, homo sapiens
ctagctgtctgggcatttgtgtcaaccagaactctccctttgcataaagccacctgcacgcgatgacagcaaagtaaagatcaaacataaaccatgggttgaaacgaaagcaagcaactctt

SEQ ID NO: 308 - DNA, 122 bp, homo sapiens
agaaaagtataatgtagcatttagcaggtcaacaaaatgcactcccttattctttgtctccgcctcattctccaacactttagtgcactaagttgtagtgactgaaatcattcttggtgaat

SEQ ID NO: 309 - DNA, 122 bp, homo sapiens
tgtttataccctagttgggacactgagcaacaaggaaaacaacggtattaatcctactaccgtgaaacctacttaggtaaaattattccagccaggcttgaagggcaaaggttcagaggaca

SEQ ID NO: 310 - DNA, 122 bp, homo sapiens
acttagcgctgggcctaaataaactggatccaacttctcaggctggacttcttccagcttcgggacaccttctcccagcatccctccctagggggaactggggaaaatcaaaggctgagaca

SEQ ID NO: 311 - DNA, 122 bp, homo sapiens
cttggtgaataatgctgggccacgctgaaaatttgtggctgagagctggaccctcgtcatcgccacgtactctgtcaatgagacagggtaacccatggttactgagctccgatcagttaagg

SEQ ID NO: 312 - DNA, 122 bp, homo sapiens
gaaaagttttgagattgagcagcttgggagtttgtggccactgggtatgtgtggctaccgcgggtatgcgagtgccagatattggctgagacgagccagcttagactaattggtacaaggaa

SEQ ID NO: 313 - DNA, 122 bp, homo sapiens
gcaaagaaaacagagaggaagtgaccagacaagtaggagaaaagcaggagaatgttgacacgtggctcaagtaacgaagtttcaagaaaaagagtcagagatctgaactatacactgagaac

SEQ ID NO: 314 - DNA, 122 bp, homo sapiens
agccgcacattcccatttcgttgataaagaaactgaccagatagtttaagtggcctgctgcggaagacagagctggtgctgcaccggtcgctgcttccccagtccttttttggcctcctttc

SEQ ID NO: 315 - DNA, 122 bp, homo sapiens
tgctctagcaatttttatttttaatcacagtggaggctgtgttggagctgaggccggtcccgctctcagggagatttgctgagaaagcgcctctctggcaactttttgacgcaaactctcca

SEQ ID NO: 316 - DNA, 122 bp, homo sapiens
cacatttgtagtgcaagctgcttccatgttgattttgatgtgtggaggctgcagatcctgcgggaggcaaattttcagacatttgggtcctcggcagctgcagcagagtttcctgttaactc

SEQ ID NO: 317 - DNA, 122 bp, homo sapiens
ccttcataaaccataaaaccttgaatgcactgaaggaatgcataaaccgatttcacttagcgctgggcctaaataaactggatccaacttctcaggctggacttcttccagcttcgggacac

SEQ ID NO: 318 - DNA, 122 bp, homo sapiens
ttctgggcgttcgaggagacagacaggtgaggaaggtagaagaaaaacatccagagaggtcgagtagttggcactgaatgggaatagtttccaaaaggctcgcgtgtaccgaagcctggtct

SEQ ID NO: 319 - DNA, 122 bp, homo sapiens
tgttgcctttccatggcagagattttaattatcttagctttttaccactttcaactgctgcgaacctaaaatgtcagtgcgtttaacatctaaaataccaactcactaaaccacaaacttgg

SEQ ID NO: 320 - DNA, 122 bp, homo sapiens
ttactgagccttttggaaaatcaaccaaaagtcttcgctgcttggagtctgattgagaagcgacagccagtgagggtgaagacgcagaaaccttcacagtagctcctcctcttagggtttta

SEQ ID NO: 321 - DNA, 122 bp, homo sapiens
gggggaggaaaacaggccgctgctgcacggggggacgagcaggcaacaaatccaggcctccgacattaccagaatgttctttaaagacactgggctttgcttaaaatatacattatgggccg

SEQ ID NO: 322 - DNA, 122 bp, homo sapiens
agtcggcttcgcagtggacctggccaacgtgtggcgcttcccctacctctgctacaagaacggcggcggtgagcgtggggtcgggctgggaatttgaatctgggaggtccactgtctgcagc

SEQ ID NO: 323 - DNA, 122 bp, homo sapiens
ctccttgagcagctacacgtcggaccgggagaggcagctcttcagcatagatgccagtaccggggaagtgcgagtaattggggggctggattatgaggaagcctcctcctaccagatctatg

SEQ ID NO: 324 - DNA, 122 bp, homo sapiens
cgcctgcccgaggagaacaccgcagctctgctaatcgagggacagccgcaacccaaagttcggcagctttccgcctgagcttcccctccctgagcgaggacactggagggaggcagagacgg

SEQ ID NO: 325 - DNA, 122 bp, homo sapiens
gaggccggtcccgctctcagggagatttgctgagaaagcgcctctctggcaactttttgacgcaaactctccaaccaatggcagggagagaatgattgacacatctaagccagagggaaaat

SEQ ID NO: 326 - DNA, 122 bp, homo sapiens
gctgagacaggggaaatgcgagggcttcggagggacataccctcttccccaggcccaggtcgctccatccctgctggggcctcagggctcatgtctgggatttccccacctttgcggggcag

SEQ ID NO: 327 - DNA, 122 bp, homo sapiens
ggcaccttcgttggccgcgttgctcaggacctgggactggagctggcggagctggtgcctcgcctgttccgggtggcgtccaaaacacacagggaccttctggaggtaaatctgcagaatgg

SEQ ID NO: 328 - DNA, 122 bp, homo sapiens
cagtcttgtctttgcctgagggtgagctaggaccctgggagcataagggaggggacttgccggagatcctagaagggaaaggaggcagcactgaaagaagaaacatttccttgattgatcat

SEQ ID NO: 329 - DNA, 122 bp, homo sapiens
ccctaccagcttctagctctcagtctgcgcgagggtgtaggaaggaaagcccaggacctccggagcagagcacagcagctgcagaggcaaggccagcatgtcgcccaacttcaaacttcagt

SEQ ID NO: 330 - DNA, 122 bp, homo sapiens
aatccatttgcacagctgagggcaaaagtgtatcgaactaagcttggctattcatcctgccgctcactgaacgtgtagctttgttaatcacagacattataattcattacatctgattattc

SEQ ID NO: 331 - DNA, 122 bp, homo sapiens
atacaacatttatatagtgaaatcagtttctctaaaatacctttcctgacctcctcaggccgagtttctctctccctcatctatattataatctctgtaccttgtcattgttaactcatctt

SEQ ID NO: 332 - DNA, 122 bp, homo sapiens
ctctgtacccttgaggaaacaaagaggacaaataagaagcagcagtgataggaggctgggcgcttctctggaataacactggctaggtagtggagcattgatagtgagtgtgcttggttggt

SEQ ID NO: 333 - DNA, 122 bp, homo sapiens
ggactgcttacaaagttggactgtggcacgaaggcctcctgattaaaccaacaaagaggccgcctctccagagctcagaattgtcctgacatgaaaataatcaagttctgacactgccagga

SEQ ID NO: 334 - DNA, 122 bp, homo sapiens
tgctctccaggagtccggagcatggtcctgggtcaccgttggtgtagcgtgttggtggaacgtggacgcctagagggaggatggtgggctgtggggtggcagttttatgtttgtgggtttcc

SEQ ID NO: 335 - DNA, 122 bp, homo sapiens
caaaccggagctgcaaggtacagttgagctgctgatcaccgtcctcgacgttaatgataacgccccactgtttgaccaggccgtatacagagtccacttgttagagactacagcaaatggaa

SEQ ID NO: 336 - DNA, 122 bp, homo sapiens
gaaggtgtgaatgaatgaataattgaaagaataaatagtagttctcagcctcacagaacacgggtcacaacctcaaatgacctgctaccctgcccataaataacagagatgcaggagtaagt

SEQ ID NO: 337 - DNA, 122 bp, homo sapiens
aaaaaaaaaaaaaaaaagaataagcaaatcatgcaaatcatcctgttctaaactctgaaccgctgcttccaagaccgtccctgcccttgccctggccgcctctctccttgttaactgggctc

SEQ ID NO: 338 - DNA, 122 bp, homo sapiens
ccgcgttcccttcaagttggtgtccaccttcaagaattactattcattggtgctggacagcgctctggaccgcgagagtgtgtccgcctatgagctggtggttaccgcgcgggacgggggct

SEQ ID NO: 339 - DNA, 122 bp, homo sapiens
gaagcgggaaaggaccagttccactacaccatcctggaagaggccaaggacggcacatagcgtcttcgcggtccctattgcgcaggacctgggtctggagcttgtggagctgataccgcgcc

SEQ ID NO: 340 - DNA, 122 bp, homo sapiens
caaaattatggtagcctggcttaaggtaggagcagttaagaatataagtagaagggtttgcgaggtatttatttagtccaatctatgaggcctaatgactgaaattatgtgcacagagaggg

SEQ ID NO: 341 - DNA, 122 bp, homo sapiens
gagtgcgcaggaccccgacgtgggcgccaactcagtacagacctacgagctcagccccagcgagcacttcgagctggaccttaagcccctgcaggagaacagtaaagtgcttgagctggtgc

SEQ ID NO: 342 - DNA, 122 bp, homo sapiens
gggaagcccacgctggtgtgctcccgcgcggtggggagctggtcatactcgcagcagaggcggcagagggtgtgctctgaggagggcccacctaagacggacctcatggccttcagccccag

SEQ ID NO: 343 - DNA, 122 bp, homo sapiens
atttacattaccttctcttattcatatccattccatacccacaccaacttggataccatacggacataataaaaaaccaaccatatcaccaggtatttatcttacccatattcggttggaaa

SEQ ID NO: 344 - DNA, 122 bp, homo sapiens
ggcaggtcactgtacagtctgggtggaaatcttggacaccaacgataactctcctgaagtcgccgtgacttccttgtccctcccagtacgagaggacgctcagcccagcacggtcattgccc

SEQ ID NO: 345 - DNA, 122 bp, homo sapiens
ttctagttaaaagaatgggtgacatcatccagggagaatatgaggagtgagaaggtcactcgcagaaacctaggtaatactaatatttaatacataggcagatgaaaaaatgctttagaagg

SEQ ID NO: 346 - DNA, 122 bp, homo sapiens
tacagtagagtgtgtgggggtttccacaattgctacttacggtttggagccacatgatgtcgctctttaccacaaaatacatgagagaaggaggaagaagggaaaattccttctattcttac

SEQ ID NO: 347 - DNA, 122 bp, homo sapiens
agtcatatttccaggtttataaaatgatttgcaaacattttcttttgttccctctaacaccgctagtagataatggaaactcctttgatttggtctgtagataattttatgctatgttatat

SEQ ID NO: 348 - DNA, 122 bp, homo sapiens
attgggcaccccacgactacagctctggcttctcctcctcgaattctgggaggtggggagcggccagctccactactccgtctcggaggaggccaaacacggcaccttcgtgggccgcatcg

SEQ ID NO: 349 - DNA, 122 bp, homo sapiens
catccctgctggggcctcagggctcatgtctgggatttccccacctttgcggggcaggagcggctcctcttgggcggggaaggaggcagggccggctcgtctccccattcccctctcccgga

SEQ ID NO: 350 - DNA, 122 bp, homo sapiens
gtccacgctcggcagagccgttccgagagcacagaggctgtcttcagtgcgcgggtgacccgattttggagcagtaggggactggctgccgagggggcatctagattgagataggtgggagg

SEQ ID NO: 351 - DNA, 122 bp, homo sapiens
atgccctcctcccccagggtgcctggacatgctgagcctccagggccagttcaccttcaccgccgaccggccgcagctgcactgcgcagccttcttcatcagcgagcccgaggagttcatta

SEQ ID NO: 352 - DNA, 122 bp, homo sapiens
gggcgcgtccgatgcagatgttggctcaaattccatcttaacctataaactcagttctagcgaatacttcgggctagatgtgaaaataaacagtgatgacaataaacaaattgggctcttat

SEQ ID NO: 353 - DNA, 122 bp, homo sapiens
ttgctccacacctcagtttccccatgtgtaaaatgaggataatgccatgtctctgtcactcgatggtgcaaggattaaatgagttaaaccacagtacaaacatgtggaagctcagccactga

SEQ ID NO: 354 - DNA, 122 bp, homo sapiens
tgccacctagacgccagggcggggaccgcaaggtgggcgggaggcttggaggccgggatgcgggggaatactggtagggtgcaaggagaatgctggaggggtgcaggggggatgccgggggt

SEQ ID NO: 355 - DNA, 122 bp, homo sapiens
caccatagagtgttctctaggaaggaaaatacccagagcccctttgttacttcagagaagcggaggaataagagaagcagcaggactttaacagagactagaatatttaaatttttgcaaaa

SEQ ID NO: 356 - DNA, 122 bp, homo sapiens
caggtggtgaagaggcatgaacaagtcaggccaggctcagctttgccaccagctgtaagccggaagctcagaggaaagacactgttaccctccctctcctgactcaaagcttggggcaattt

SEQ ID NO: 357 - DNA, 122 bp, homo sapiens
tggcggacgcagaagaaatggattaataaattccggaacatacagtattttatctttatgcggaagatcttctaatggaaataaaaccagaggtatttgacatggtgtttaccccggaggat

SEQ ID NO: 358 - DNA, 122 bp, homo sapiens
actacgatcctttcctgctcttcagcgccaacctgaagcgggagctggctggggagcagccgtaccgccgcgctctgcgtgagtcgaggctgcccggctcgcgggcgcccgggacgcgggga

SEQ ID NO: 359 - DNA, 122 bp, homo sapiens
cctgggacctcggattagggaagatgcaacaccccgcaccctctggcttagcagaacctacgcctgcatgtcctccccacgaaacatgcgctcgccccagccctcctgcctcctaccctcct

SEQ ID NO: 360 - DNA, 122 bp, homo sapiens
actgactgccactgatgggggcaaaccggagctgcaaggtacagttgagctgctgatcaccgtcctcgacgttaatgataacgccccactgtttgaccaggccgtatacagagtccacttgt

SEQ ID NO: 361 - DNA, 122 bp, homo sapiens
cccagcccaacttttttgcctggcatagaaagcagggaccggacaggtttcaaacggatccgagtccctagaaaagcaaaaacagactgggcggagtgtggggtggggggtggctagtggaa

SEQ ID NO: 362 - DNA, 122 bp, homo sapiens
gagcccccagggcgactttggtggaatctggtgttgaaatcagagtcccaccgccgggcgcggtggctcacgcctgtaatcccagcactttgggaggccgaggcaggcggatcatgaggtga

SEQ ID NO: 363 - DNA, 122 bp, homo sapiens
tcaaagcaactgatgggggcgaagctgagctgacaggcaccaaagcattatatatcacagcgctggatgtaaatgatgatgcccagtgtttgacaaaggagtctatcatgtgaaattactgg

SEQ ID NO: 364 - DNA, 122 bp, homo sapiens
tggatgtcaatgtttacttgatcattgccatctgcgcggtgtccagcctgttggtgctcacgttgctgctgtatactgcgctgcggtgctcggcaccgcccaccgagggcgcgtgcgcgccg

SEQ ID NO: 365 - DNA, 122 bp, homo sapiens
gacacccatgagtctaatgagatcaagtggcctgagtgggttgaagtccttggatcagaccgtggaagctggaatggagagagatttcggaggggcagccagacctggtgaggaggaactgg

SEQ ID NO: 366 - DNA, 122 bp, homo sapiens
gaaaaggtggacatggatggcaggggagccccaggaccttctttgccaaacaagtgttcccgggaacctatctgactgacggacctggcgtccaagtggcctccaccacagtgacaagccct

SEQ ID NO: 367 - DNA, 122 bp, homo sapiens
aaaatgacccatagaggaagatctacagatgacagaatactgggagatgaagagaaaggacgttttagtgagtgctttttttgcacatgaggcaatagatttcaaattcctgtggaagaaat

SEQ ID NO: 368 - DNA, 122 bp, homo sapiens
gtgtggacttgccactacaaatcttgagggtaaactgcttatctccccacctctctgccccgtttatctgaggcgataacgatctctaaacacgaggtatcgaatggagcccaatgaatttc

SEQ ID NO: 369 - DNA, 122 bp, homo sapiens
tgcacaacaatggccacacatcgagattgaaatgaagggataaaaacattcctataaattcgaaggcaagttttgctgactagaaaaacaagccaataatttgaaatggtgtgcccgaatgg

SEQ ID NO: 370 - DNA, 122 bp, homo sapiens
tgttcaaagtcttttctctgacagcatctgtctctaaaggccgaacaacgggagatgcagcggaattggattaaaagactctgaaagtacagtcgttcatctttatattaagataatatttt

SEQ ID NO: 371 - DNA, 122 bp, homo sapiens
tctcccggctggtgcgcccctccccacgcgcccgcggctgcaccccactctgcacacacacgtccaactattcttggatctactcacatccctggatcaagtctggccgccctccccgctcc

SEQ ID NO: 372 - DNA, 122 bp, homo sapiens
tctgataggaaacgactatttaacatggattatcactggcgaggagagctgggatcctggcgactactactcttgcttctgctcctcgcagcctggaaggtggggagcggccagctccacta

SEQ ID NO: 373 - DNA, 122 bp, homo sapiens
gtggagtccgcgcgggattctggtgccacctagacgccagggcggggaccgcaaggtgggcgggaggcttggaggccgggatgcgggggaatactggtagggtgcaaggagaatgctggagg

SEQ ID NO: 374 - DNA, 122 bp, homo sapiens
atgaagatgtctgggtaccgggtcttggcaaacagtgcttccagcacatctagctgcgcccgagtgaacgtcgtcctctcccggcgctgtttccggggggtggctgcgggacaagaagccca

SEQ ID NO: 375 - DNA, 122 bp, homo sapiens
cctgtcatcattcaggtctcacttgtcatttcctgaccatggtacttataaaagcagtgccgtctgccccatccatgtcacctcgttcatctccttcacctccgaaatgatctcgcttttgg

SEQ ID NO: 376 - DNA, 122 bp, homo sapiens
ccgtttctttcttcctgcagaaaatatagcagaaagcggaatacctcttgcgaatccttccgcactaggaagccataaaaattgggccttgagagacagtttgaggtaaggcgttgtatata

SEQ ID NO: 377 - DNA, 122 bp, homo sapiens
gaaactctggaaagcaaaggaatcttctctccagtcctgcctgtcacttactccggctcacgctgaagcgttcgcgtttacggttccaaaaccaagactgaaaggcagtcttcacatattta

SEQ ID NO: 378 - DNA, 122 bp, homo sapiens
ccgcagccccgcagccccgcagccccgcaggcctggcgcccgaggtcccgctccactgcccgcgccccccgcgcagccttatatctaacggtcaattcgtgcaatctgtcgcttctccctcc

SEQ ID NO: 379 - DNA, 122 bp, homo sapiens
cggtgtccagtctgttggtgcttaccctgctgctgtacacggcgttgcggtgctcagcgccgtcctctgagggcgcatgtagtttggtaaagcccactctggtgtgctccagcgcggtgggg

SEQ ID NO: 380 - DNA, 122 bp, homo sapiens
atgatttttaaaaaatagattcaaatgtgttatcctctctctgaaacgcttcctataactcgagtttataggggaagaaaaagctattgtttacaattatatcaccattaaggcaactgcta

SEQ ID NO: 381 - DNA, 122 bp, homo sapiens
ttggagcagtggaactaagggcaaagcaaacaaacaaacagaggggctggtttactgcttcggaggcagcagctcttccacgttccagcactaacttaaaaagagcacggactaggcgaggc

SEQ ID NO: 382 - DNA, 122 bp, homo sapiens
gtgctgcagggtcttcagccgcccccagagggctgtcagaagtctccaactcttgagttccggcgtgccccaacctctgtttccaaatttttccagcggacgcgcgcccttttctgggaacc

SEQ ID NO: 383 - DNA, 122 bp, homo sapiens
agcccctgacccagcaggagtgctgacctttcactcggggatgggggaactttgtctcctcgccccttttcatttggcttctcctccccgttcttgcagcctctctctccctttcttcccgt

SEQ ID NO: 384 - DNA, 122 bp, homo sapiens
aggatggtctagatctcctgacctcgtgatccaccggcctcggcttcccaaagtgctggccgagtgccaccgcacccggccgaaaggtattttttttaacagaagaaaatcagattattcct

SEQ ID NO: 385 - DNA, 122 bp, homo sapiens
cgtttagccacatgatgtcgctgtccaccacaaggtctttctccacaaaagaaataacagcgtgcattacgtattcagatactgctttgcttcatcctctctaaaatttaacaccgaggagt

SEQ ID NO: 386 - DNA, 122 bp, homo sapiens
aggaggctaaacatggcaacttcgtgggccgcatcgcgcaggacctggggctggagctggcggagctggtgccgcgcctgttccgggcggtgtgcaaattccgtggggatcttctggaggta

SEQ ID NO: 387 - DNA, 122 bp, homo sapiens
caaggtcatacaaaacttttagaggcagtcagtctgctaagaaggctccagcaagagaaacgggatcttctgtttcaacaatcattacttaagaaaaaattaagaaaatgaaataagttttg

SEQ ID NO: 388 - DNA, 122 bp, homo sapiens
agtttctccactccagagcctggagtgggattttatagtgtcgaccctcttcaaaaaccacgcagcatttgcctaataagctgacgctctcttgacagctcgattgcgtgctgcctctgctc

SEQ ID NO: 389 - DNA, 122 bp, homo sapiens
ctttatttattttgcttttgctttttcttttgagatgctttatttaaagtacactggggccgggtgcggtagctcatgcctatttgtaatcccggcactttgggaagccgaggcgggcggat

SEQ ID NO: 390 - DNA, 122 bp, homo sapiens
ttcctgcccggcagccccgcagccccgcagccccgcagccccgcagccccgcaggcctggcgcccgaggtcccgctccactgcccgcgccccccgcgcagccttatatctaacggtcaattc

SEQ ID NO: 391 - DNA, 122 bp, homo sapiens
agaaaaggaacacatatcactcatttttgcatacagtagaattaatggtaacacttcatgcggctgaaagatggggcctttatctgccaaagaataatgcactactgatcttttgatttcta

SEQ ID NO: 392 - DNA, 122 bp, homo sapiens
agggcgagtgcgcgcctggcaagccgacgctggtgtgttctagcgcggtggggagttggtcgtactcgcagcagaggaggcagagggtgtgctctggcgagggtaagcagaagaccgacctc

SEQ ID NO: 393 - DNA, 122 bp, homo sapiens
aatataatatggaaggtgaaacatttacttaaactattgcaatgcattattaaaagatgacgataaccacaaaagatgtaaaaataaattacaatgggggttaaaagaagggagaaagggaa

SEQ ID NO: 394 - DNA, 122 bp, homo sapiens
ggcattttctggaggacaaatccttagctgaacttttgaatgagctctctgtatgggtgccgtaactgtggtaaaagtgaggccaccaaggccaagctcaagaccaccaaccatttggagcc

SEQ ID NO: 395 - DNA, 122 bp, homo sapiens
aacactgaatctcacatccaattatatcaacagatatttatcgcctcttggtgacgtcaacgagccctaaaatggaagggcttcttatgacttgtccattgacaaaaattcttgaatgagat

SEQ ID NO: 396 - DNA, 122 bp, homo sapiens
ggttgttctccttgagagatggtgttggggtgctgattcagacaccactcattatgggggcgcaggaataatgctgcccacagcccctgactcagcccagcaggtcagtcctgggacctcgg

SEQ ID NO: 397 - DNA, 122 bp, homo sapiens
cgggggctcgccttcgctgtgggccacggccagtgtttctgtggaggtggctgatgtgaacgacaacgctccggcgttcgcgcagcccgagtacacagtgttcgtgaaggagaacaacccgc

SEQ ID NO: 398 - DNA, 122 bp, homo sapiens
aggcgaggagcggcgcggaccgtgggggaggtggcgggggcgaggccggcaagaggcggccgcgggccgggctgcgtcgggaaacggcggccagacttccccgggaaggggcgagcgagagc

SEQ ID NO: 399 - DNA, 122 bp, homo sapiens
tcaagagagtaagactgttagtatatcgttaatcagggtgttttaagagatgggctaagacggagaagcgcatttgggacactatagtcaaatcccgtccaataaaaggtaacttttcctat

SEQ ID NO: 400 - DNA, 122 bp, homo sapiens
cgagtccatgaaccaggaaatggattcacattaaccataaagacagaccccaacctctttcgtaagtgttctcagtcaaaaggcagaacttcggatatagacccgctttttgaaagtagtat

SEQ ID NO: 401 - DNA, 122 bp, homo sapiens
ctcttttcctctcgaagcagggtccagaacggggaaaagaaataacttttgaaaaatccacgcctcactatgactgctcacgaggtggcctctggagaactcatacctagaagctgaccgcg

SEQ ID NO: 402 - DNA, 122 bp, homo sapiens
ctttctagagcgtgagcggtgggggagcaggcgcagggtggcacgagcggaggcggggcccgggcgtggggcacggctggggaagctgccgcctccggccctgcccggctgcctccgccgcg

SEQ ID NO: 403 - DNA, 122 bp, homo sapiens
aggatttactattaaattatgataaagaaatagaaagtgcatagaagaacccagatattgcggaagtaattcatgtaatcattaccttttgaagccacatgatgtcgctgtctaccaagaag

SEQ ID NO: 404 - DNA, 122 bp, homo sapiens
tattttatttctctaaatttgacctctttccataaattttctttgccccacttccaaatgcggcgagaattcatgcttctcattgaattcacagatatctttagaatgttttattgcttagt

SEQ ID NO: 405 - DNA, 122 bp, homo sapiens
cttcccttctgtttccttttccctccccctggagctgtagcggcagcagcagcaggaagccgagccgggttgagcgactcggaggcgagcggaggagctggaatatggggagtcagcgagga

SEQ ID NO: 406 - DNA, 122 bp, homo sapiens
atcaatggaaaatatgaagactgagtcatacttacacttacacgttcatgcgcatggtgtcgctcttcactgagaacgtttccgcgaagaaagccgccgtttctttcttcctgcagaaaata

SEQ ID NO: 407 - DNA, 122 bp, homo sapiens
gagagagaaaatggaaactttaacctcctggttaaattctatttaaccaggacgccgattcgggctacaatgcgtggctttcgtatgaattgcagccggcggcggtaagttaaattctatta

SEQ ID NO: 408 - DNA, 122 bp, homo sapiens
atcccagctactcaggaggctgaggcacaagaattgcttgaacctaggaggcagaggttgcggtgagcctagattaggccactgcactacagcctgggagacagggtgaaactctgtcttaa

SEQ ID NO: 409 - DNA, 122 bp, homo sapiens
gcggaccgtgggggaggtggcgggggcgaggccggcaagaggcggccgcgggccgggctgcgtcgggaaacggcggccagacttccccgggaaggggcgagcgagagccgggccgggccggg

SEQ ID NO: 410 - DNA, 122 bp, homo sapiens
gctgagggagaggaggccaaagagagcgagcagtgagcgccggggcggacgagaagccagcgctcccagcctcctcggttatccgctccggtttccgctcacgttcaacaggggccgacagg

SEQ ID NO: 411 - DNA, 122 bp, homo sapiens
actacaaatcttgagggtaaactgcttatctccccacctctctgccccgtttatctgaggcgataacgatctctaaacacgaggtatcgaatggagcccaatgaatttccatgccgcttttt

SEQ ID NO: 412 - DNA, 122 bp, homo sapiens
acccggcaaagtcatgttgaaaaaacactcttcacgttcgctcggcctggtgaccagggtcggggaccacgacaaccgggggttgggaggctgcgtaattacaacccagggtggtttggatt

SEQ ID NO: 413 - DNA, 122 bp, homo sapiens
taacagcgtgcattacgtattcagatactgctttgcttcatcctctctaaaatttaacaccgaggagtttaagaaatgaagataaggaactcgaattatttttaaactttggatcaatgtaa

SEQ ID NO: 414 - DNA, 122 bp, homo sapiens
ttctcacaatctaaaattaattgtggactcccttttctattcccaaattaaagagagaggcggccagtgagagatgcatgagttaaacaagctttacttattctactgaatgcaaactcaat

SEQ ID NO: 415 - DNA, 122 bp, homo sapiens
ggaccttctggaggtaaatctgcagaatggcattttgtttgtgaattctcggatagaccgcgaggaactgtgcgggcggagcgcggagtgcagcatccacctggaggtgatcgtggacaggc

SEQ ID NO: 416 - DNA, 122 bp, homo sapiens
ctggttggtcaatgttcaaagtcttttctctgacagcatctgtctctaaaggccgaacaacgggagatgcagcggaattggattaaaagactctgaaagtacagtcgttcatctttatatta

SEQ ID NO: 417 - DNA, 122 bp, homo sapiens
aacagttatgtgacattgggtacatcaactaccccagacttaaaatgggagcatgatacccgcccagtctgctttcccttccatcccagggacccactagatctttggaaaatccttttacg

SEQ ID NO: 418 - DNA, 122 bp, homo sapiens
tacacgtagagtggtggtattacagactttcttgagaatcatatctagctatttcccccccgtatagaggcataacataccatttggcacaatattcaaagagtctatgctgaagcctattc

SEQ ID NO: 419 - DNA, 122 bp, homo sapiens
cggcacttagctcttcgattcttaaaccataccttggaagggaaagaaaattctttaactcggttttgatagttccttaaggacaagaatggctcccgtattataaatctatcctacatggg

SEQ ID NO: 420 - DNA, 122 bp, homo sapiens
gtttggaggtgcaaagtcggcccaaatcgggggtacccagctggaagatcttgatgcgcccggggtggacaggttcagagtccttggtgggtgggtttggagcagtggaactaagggcaaag

SEQ ID NO: 421 - DNA, 122 bp, homo sapiens
agtgagtgagaacaattaaaggaaaggatatgggacagggttgaagttgagagagaaaaacgagatcatttaggaacagctgaagaatcaaaaacagcaaagcacaatgggtagaaattcca

SEQ ID NO: 422 - DNA, 122 bp, homo sapiens
gatcttacgatcttctaaaataactccagctccaaatgaaggccagtggacccatgaacacgctggctcttgcccagtgtgctgagaagagggcaggagggaggagtagggtgggcacggca

SEQ ID NO: 423 - DNA, 122 bp, homo sapiens
ctcatgccctcatccaaggagttcaaaaggaaaaaccaggaaagaggtgatttgcagccacgttctccagttcaccatgggcgaggccacgctcggctgcttccattgctgggagtagggaa

SEQ ID NO: 424 - DNA, 122 bp, homo sapiens
tttaaatttttgcaaaacatgctcttctaatttgatcaaaacattgaggattggtaatggcgtcttctatcagaaggggccgaggggcctggacacggctgctctcgcttctgctcctcgca

SEQ ID NO: 425 - DNA, 122 bp, homo sapiens
gctagttcaggcctttgaattctgggtgtcaggatcacaaccttggctcctttaactgtccgctctaggagggaagcatgccaaatgtcaggtctcactctgactttgttccagtccatagc

SEQ ID NO: 426 - DNA, 122 bp, homo sapiens
gagtgtccagggaagatgtgggagtcaggtgaatagttcccatcgccagtaaccccagagcgcctccccctgcgttgtttccttttaatgttagatccgccgcctgggtgcccatacttagc

SEQ ID NO: 427 - DNA, 122 bp, homo sapiens
agaacgcttccctgggcactgtcattgctctcatcacggtgtcggatcgcgactctggtacgaatggacatgtcacctgctccctgacgccccacgtccctttcaagctggtgtccaccttc

SEQ ID NO: 428 - DNA, 122 bp, homo sapiens
caaaatttgtaagagaaaaaatacatccgcctacagaactccacgttcgcagaggggcatcggtcggtctattggaggtactggggtgatggccgacagacacggggacacgtagatatgga

SEQ ID NO: 429 - DNA, 122 bp, homo sapiens
gccactgccaggggactgtgaggaggagccgaggatgcagcctcgtgttgggcccatgcccgttcccagccactcagtctttcccactgtctacctcctgccccggtgcttgcttccaactt

SEQ ID NO: 430 - DNA, 122 bp, homo sapiens
cgggggctcgccttcgctgtgggccaccgccagcttgtctgtggaggtggccgacgtgaacgacaatgctccggcgttcgcgcagcccgagtacacggtgttcgtgaaggagaacaacccgc

SEQ ID NO: 431 - DNA, 122 bp, homo sapiens
ggtttgtaattcttacttacatgatggccacatgatgtcgctctttaccgcaaattctttcgtaatcagcaaaaggaagtcattccttttcgcactggaaagacgctccactctctttcact

SEQ ID NO: 432 - DNA, 122 bp, homo sapiens
gaggaagaaacgtgccaaaagggttggccttgactattaattatcgttgggagaaagccccggtgttagcgctgaggtctgggtgtctaccttactctggggggaggagttccctctcctac

SEQ ID NO: 433 - DNA, 122 bp, homo sapiens
aactgctgcaaaaaattcattgtatcggggttagactgcaaatacttctacccctcaaaacgaatctgcaaattatttcccagatgctgagtaaagaggtgaggaggttccaaactccaaat

SEQ ID NO: 434 - DNA, 122 bp, homo sapiens
gtgggccacggctagagtgtccgtggaggtggccgacgtgaacgacaatgcgcctgcgttcgcgcagcccgagtacacagtgttcgtgaaggagaacaacccgccgggctgccacatcttca

SEQ ID NO: 435 - DNA, 122 bp, homo sapiens
gacggacctcatggccttcagcccctgccttcctcctgatctgggatcagttgatgtaggcgaagagcaagatttaaatgttgatcatggcctcaaagtaagtccatttaaatttagaactc

SEQ ID NO: 436 - DNA, 122 bp, homo sapiens
caaaccttcacctatcccaattctcagcctattctcaagagagtaagactgttagtatatcgttaatcagggtgttttaagagatgggctaagacggagaagcgcatttgggacactatagt

SEQ ID NO: 437 - DNA, 122 bp, homo sapiens
ttgtcttacctataacacttaataaagcattaaggacactgttccacctaatgatgtttccggggtttctggtatctcattagggactcatctaatatgactgatatatcaacagtaagcag

SEQ ID NO: 438 - DNA, 122 bp, homo sapiens
aatcaatcagtggtacaatgtgtaccacagtagtacaaaatgtattcctattctccgaaacgactgaaataataaataaaaccaagtagtttacagtagagtgtgtgggggtttccacaatt

SEQ ID NO: 439 - DNA, 122 bp, homo sapiens
agaagttctggttggtcaatgttcaaagtcttttctctgacagcatctgtctctaaaggccgaacaacgggagatgcagcggaattggattaaaagactctgaaagtacagtcgttcatctt

SEQ ID NO: 440 - DNA, 122 bp, homo sapiens
tttggtattgtacaattggaaagaacccgtttctgttcccaccagatgcagatgtcaggtcgaaacagaaggctgagagatgcttaaaccaccctagcagaaaagccatctctgatgcagaa

SEQ ID NO: 441 - DNA, 122 bp, homo sapiens
tgccgcggtcggtgggtgcaggccacgtggtggcgaaggtgcgcgcggtggacgccgattcgggctacaatgcgtggctttcgtatgaattgcagctggcggcggtcggcgcgcgcatcccg

SEQ ID NO: 442 - DNA, 122 bp, homo sapiens
ggtacgcgttgcagccactggactacgaggagctggagctgctgcagttctaggtgagagcgcgcgacgcgggcatgccgcctctgagcagcaatgtgacgctgccaggtattagtgctgga

SEQ ID NO: 443 - DNA, 122 bp, homo sapiens
gtgggattttatagtgtcgaccctcttcaaaaaccacgcagcatttgcctaataagctgacgctctcttgacagctcgattgcgtgctgcctctgctcctgcataaatcatagggccctgcc

SEQ ID NO: 444 - DNA, 122 bp, homo sapiens
gcgcggtgtccagtctgttggtgctcacactgctgctatatactgctctgcggtgctccgcgccgccaaccgaaggcgactgtgggccgggcaagcccacgctggtgtgctccagcgcggtg

SEQ ID NO: 445 - DNA, 122 bp, homo sapiens
tggacaggccgctgcaggttttccatgtggaggtggcagtgaaggacatcaatgacaatccgcccaggttctccagacaagaacaaagattattcattttagagtcaagaatgccagattcg

SEQ ID NO: 446 - DNA, 122 bp, homo sapiens
atgcgctcaatttcagatgtggaaacagaattcctaaacatatattcatttgttcaaggtcgaggtagcagagtttagatccagattggtctgatcccaaagcctgaattttttcctttgta

SEQ ID NO: 447 - DNA, 122 bp, homo sapiens
aaaaaaaaaaaagtacccgaagaaagcaattacaatctgccttccccaagaacaaaaacccgtgccttcaaatgcacacattgcattcctatccctacatttgcataggactcattggctgg

SEQ ID NO: 448 - DNA, 122 bp, homo sapiens
gtggactccagctatgacgcctggttgtcgcaacagctgcatctgtcagctggcagcacccgttccacgtggggctctgcacgggcgagatcagcacgacgagtaccctggacgaggcgaaa

SEQ ID NO: 449 - DNA, 122 bp, homo sapiens
ggagtcctctggaaggtcattttcccaggctctttgcagagtacctcgagctcattccagcggaagtgtcaggattgggcaccctggaagcaaaacagcagaagagtgaaatcgagtcatga

SEQ ID NO: 450 - DNA, 122 bp, homo sapiens
taacatatgaatgaaagaaaagttgtgaactcatggaatcgtacttacacctaaagccaccggatgtcgctgtcgtccacaaaatagctttttagaacaaaggcatcagccagtttctcaag

SEQ ID NO: 451 - DNA, 122 bp, homo sapiens
acaggccgctgcaggttttccatgtgaacgtggaggtgaaggacattaacgacaacccgccggtgttctcgctcagagaacaaaagctgctgattgcggaatctaagcaatcggactcgcgt

SEQ ID NO: 452 - DNA, 122 bp, homo sapiens
cgctgctgctgtacactgcgctgcggtgctcagcactgcccactgagggcgggtgccgggcgggcaagcccactctggtgtgctccagtgcggtggggagctggtcatactcgcaacaacag

SEQ ID NO: 453 - DNA, 122 bp, homo sapiens
acaaaacgttttctcacaggtctacattctctttaaaacaccactataaacacctggaaacggaaactaaacgtagacaaaacgaataacattgtgttgctgctgcacgtgaatacactttg

SEQ ID NO: 454 - DNA, 122 bp, homo sapiens
gtctctattgccctcttggcagctcctgggatacaagagggtgcaggacagacttcaacccgcagcaggatccagcgcggaaagctctgcagcaggatccagcgcggaaagccccccgcagc

SEQ ID NO: 455 - DNA, 122 bp, homo sapiens
atgggctcacactgtgagtagttgatctttgaggcccacgtcccattctccaagcattctcgataggcattccctacaaaaaatgccaactgccaagagtcaggtcactcccctcctcaaga

SEQ ID NO: 456 - DNA, 122 bp, homo sapiens
acggaaccttcgttggccgcatcgcgcaggacctagggctggagctggcggagctggtgccgcgcctgttccgggtggcgtccaagggccgcggggaccttctggaggtaaatctgcagaat

SEQ ID NO: 457 - DNA, 122 bp, homo sapiens
gtgcccgagtcggagaggcgcgttcgaggacccccacacggcgagaatctcgcccttacccgaagacctcgacccacacacatccagtggggctggggccggtgcctctggacgctgccatg

SEQ ID NO: 458 - DNA, 122 bp, homo sapiens
ctagttacatctgcatctttttgtctcggactctaaaaaggtccctgggatccatccaaacgaccccaaccaaatctgggggccaaaacgcaaagatcgcgggagaagcccagaacggcgtt

SEQ ID NO: 459 - DNA, 122 bp, homo sapiens
aaggaaagcagggggcggcaggcttaaaagcaagttgcaggcgaaatagtaagttgctggcgaaatatagccccccttccgaaactcctgacctcttcttccaccccggccagggcccgcgt

SEQ ID NO: 460 - DNA, 122 bp, homo sapiens
gatataagaaataggacagaaaatgtcagatcgtatgtgcgttctagaccgctgattcgtcgatttgtaaaacaagagaaggataagatggtttccagatgtagctgcctgggggtccagtg

SEQ ID NO: 461 - DNA, 122 bp, homo sapiens
ggtatttgaaggcacgaaatggaatgacttaatctcatctctcagtggttcagatggatgcgaaggtcatggctacagctacctggtctaggagtgtgtacatcatgtaaacatccaggtca

SEQ ID NO: 462 - DNA, 122 bp, homo sapiens
gcatgtccccttcaagctggtgtccacctacaagaactactactcattagtgctggacagcgccctggaccgcgagagcgtatcagcctatgaactggtggtgaccgcgcgggacgggggct

SEQ ID NO: 463 - DNA, 122 bp, homo sapiens
aggagttccctctcctactcccctctgttgctaataacttttggtgcctgtaaaaagtcccgagctgagcaggagaaatcctgaccccgaagctctaggatggaggggagaatttctaggag

SEQ ID NO: 464 - DNA, 122 bp, homo sapiens
ggagctggaatatggggagtcagcgaggacggtggggccaggagcccttgggagggcctacggagggagcggccccaggcgctttctagagcgtgagcggtgggggagcaggcgcagggtgg

SEQ ID NO: 465 - DNA, 122 bp, homo sapiens
cagggcaatcacgcctccccggggacactgggcaatgtccagagacatttttggcagcctcgactgcgaggggaagggtactactggcatctagtggatagaggtcagagatgctgctcaac

SEQ ID NO: 466 - DNA, 122 bp, homo sapiens
tcgaccctcttcaaaaaccacgcagcatttgcctaataagctgacgctctcttgacagctcgattgcgtgctgcctctgctcctgcataaatcatagggccctgccaaggaacgaggggcag

SEQ ID NO: 467 - DNA, 122 bp, homo sapiens
gagcagctcttcagccagcgccaggctgcagttggcctccagcaggctgtggagcagtgccgcgtccatcgcgtcccgcagccgcgtgcggagagggagtgggagtgcgcgcccggcgtgac

SEQ ID NO: 468 - DNA, 122 bp, homo sapiens
gaacggtttcatcttgaaactacccccaccccaaaatctgtggaaaaattgtcttccatgcgacaggtcactggtgctgaaaaggttggggaccgctgttctgcaggaaacgagagacagag

SEQ ID NO: 469 - DNA, 122 bp, homo sapiens
ctgtacacaggcgagatcagcacaacacgctctctggatgagaccgaagcaccgcgccaccgccttctggtgctggtgaaggaccatggagagcccccgctgacagccacagccacagtgct

SEQ ID NO: 470 - DNA, 122 bp, homo sapiens
agtggcaggccttacccggagactacaaggaaccgcccgtgtgagccagtggaagactcgcgtccccaggcaaaatataagtcctgggttaatagtctgaagtctgtcattaagcaataggt

SEQ ID NO: 471 - DNA, 122 bp, homo sapiens
atcagtgccaaaggtgctgggggtcgggcccagcgcccggcgcggctggaatgggggcggcgaggagctcctcctagccagccgcagggcgggaactgcccaaacgcaggtcgcagcctcac

SEQ ID NO: 472 - DNA, 122 bp, homo sapiens
gtaccgcggccgtcagcagctttgtaccgctgggtccggaatcctctttactccctaaaccgccttctcaggaggccggtgtagagcaggcagcgagggccgggggcactcacgggtcgtgt

SEQ ID NO: 473 - DNA, 122 bp, homo sapiens
agccgccatcttggcaaaggaagagatactcgccacctcggaaaagaagagggaaccaaccgtatgccaacgtagaaccctcagccagaagaatttttatgaagggcgctgagcaactccat

SEQ ID NO: 474 - DNA, 122 bp, homo sapiens
cacatcccctcccccatccgttacctgagggctagggatgaactcagattattttctgggcgttcgaggagacagacaggtgaggaaggtagaagaaaaacatccagagaggtcgagtagtt

SEQ ID NO: 475 - DNA, 122 bp, homo sapiens
ggcgtcttctatcagaaggggccgaggggcctggacacggctgctctcgcttctgctcctcgcagcctgggaggtggggagcggccagctccgctactccgtccccgaggaggccaaacacg

SEQ ID NO: 476 - DNA, 122 bp, homo sapiens
aacatagacaccagcttgacccggctcagggcattcagagcgattacattcaagagacttcggtggatttgggggaggggtgcggattggcggaatttcatttttccatcagttattaaacc

SEQ ID NO: 477 - DNA, 122 bp, homo sapiens
ctacactcagaaggtgaaacaggaaaatttttttctccgcacccacattccaatcattcacggaataggatcgactccataatgactgtgatgctggaaaaaatttactaaatatgtactta

SEQ ID NO: 478 - DNA, 122 bp, homo sapiens
ttgctaataacttttggtgcctgtaaaaagtcccgagctgagcaggagaaatcctgaccccgaagctctaggatggaggggagaatttctaggagcgacttccccgtcccctccccaagcaa

SEQ ID NO: 479 - DNA, 122 bp, homo sapiens
ggagcagccggcagggcagggccgagcgaggaatcctgactttcctgctctttaactttgcgggaggggggaggctgcacaaaggagcaggtgtgcgcctcccctgccgccccgcgcccaca

SEQ ID NO: 480 - DNA, 122 bp, homo sapiens
caccccgcttcgcgccagtctgggcatcgcctcacccggggctcgggactccgcagggtccgctccagccacaaacatagacaccagcttgacccggctcagggcattcagagcgattacat

SEQ ID NO: 481 - DNA, 122 bp, homo sapiens
cccaggttctccgtaacagaacaaaagctctcaatacctgaatccagactgcttgactctcgatttccactagaaggcgcatctgatgcggatgttggagagaacgcattgcttacttacaa

SEQ ID NO: 482 - DNA, 122 bp, homo sapiens
tagggagaaacatttttatggctatctctttatgatcctctcatttctgaacactgaactcgacctgcctcttaagtttctcttctctccaagtaactgtctttcccaaattaagagtcttt

SEQ ID NO: 483 - DNA, 122 bp, homo sapiens
gtgaaaaataacccaaagtcgaaacattagaattaagagtgacccaggaagtggctaaaccgaaaagaacctcaggatctttcttgtacttacataatcagtcacatgatgtcgctctacac

SEQ ID NO: 484 - DNA, 122 bp, homo sapiens
tgattttgatgcacaactagttaaaagcctttcataccagtcagtattcccagccttgagcgcacgcgcgcacacacacacacacatacacacacgcattaatttttgtactttgcttcttt

SEQ ID NO: 485 - DNA, 122 bp, homo sapiens
agcaaaggaatcttctctccagtcctgcctgtcacttactccggctcacgctgaagcgttcgcgtttacggttccaaaaccaagactgaaaggcagtcttcacatatttattgtttataccc

SEQ ID NO: 486 - DNA, 122 bp, homo sapiens
ctgcatcattccctgactgttgaatgatggcggacgcagaagaaatggattaataaattccggaacatacagtattttatctttatgcggaagatcttctaatggaaataaaaccagaggta

SEQ ID NO: 487 - DNA, 122 bp, homo sapiens
ctgccgcgcggggcggggagcggggtcggctcagtggccctgagaccctagctctgctctcggtccgctcgctgtccgctagcccgctgcgatgttgcgcgctgccgcccgcttcgggcccc

SEQ ID NO: 488 - DNA, 122 bp, homo sapiens
cagccatttcgataaaaaatagatataagaaataggacagaaaatgtcagatcgtatgtgcgttctagaccgctgattcgtcgatttgtaaaacaagagaaggataagatggtttccagatg

SEQ ID NO: 489 - DNA, 122 bp, homo sapiens
ccccagctcgccgcgtcgggaaggcttggacgatgccgggaccgagcctcctaccttgtgcggcacagattatgatttttgtgactctgggaaaggttgtgctgtgtgggtttagggtttgg

SEQ ID NO: 490 - DNA, 122 bp, homo sapiens
ccggcactgctggcgactcaggctggcagcgcgggaggcgcagttaacaagctagtaccgcggtcggtgggtgcgggccacgtggtggcgaaggtgcgcgcagtggatgcggactcaggcta

SEQ ID NO: 491 - DNA, 122 bp, homo sapiens
aatcttctctccagtcctgcctgtcacttactccggctcacgctgaagcgttcgcgtttacggttccaaaaccaagactgaaaggcagtcttcacatatttattgtttataccctagttggg

SEQ ID NO: 492 - DNA, 122 bp, homo sapiens
aacagtggaacaagtgaggcttgtattacatggccaaattgatattaatgatggtggcctcgagaacggattccatcccaagacatctgatctgcagacagttgagcattcacagcagtttt

SEQ ID NO: 493 - DNA, 122 bp, homo sapiens
gggagagtggccaggtcctgtctccatcagcatgcatttgcagggactggtctgtggtcacggcctctgtcgtcctccctgacgacatttaccctggtcccctcccctctcctctgggcagg

SEQ ID NO: 494 - DNA, 122 bp, homo sapiens
acccacctgccaggctgcgcggggaggctggtcccgggctgggcaggcgggctggcctcgcgccctcgaggcacccggcggcgctggctgtgcggaggggcgccggcgcggccgtatttgta

SEQ ID NO: 495 - DNA, 122 bp, homo sapiens
ctcaatcattaacatgaagtagtatatcaattctgatagactacttacaatatgtgcttacggaatttaattgaagagatgaactccagtgtgccagaaaggacgtgagatttagcattata

SEQ ID NO: 496 - DNA, 122 bp, homo sapiens
aaaaaaggacacacatactcaaatgcttataagagataaattaatgtaataaatggcccacgcgattccaccctggtgctgcagtttgtaaaagaagaaaaaatacctgttcaattggtttg

SEQ ID NO: 497 - DNA, 122 bp, homo sapiens
agagcttgtgccgcggtctgtgggcgcgggccatgtggtggcgaaggtacgtgcagttgacgctgactcaggctacaacgcgtggctttcgtatgagttgcaaccggtggcggccggtgcga

SEQ ID NO: 498 - DNA, 122 bp, homo sapiens
acagagtctctgtaagaataaatttcttctctcttatggaggccagaagtccaaaatcagcgtgtttcctgggccgcatttcttctgaagactttaggggaaatttttttcttggatcttcc

SEQ ID NO: 499 - DNA, 122 bp, homo sapiens
gccgccggacacgtgagtgcgccctgagcgcgggacagggctaggtctgcctgggaggcccgggccgagacgcgccagcagagggctagcgagtttgtagtgcagtgacgttaagtgtccga

SEQ ID NO: 500 - DNA, 122 bp, homo sapiens
ggaacaagtgaggcttgtattacatggccaaattgatattaatgatggtggcctcgagaacggattccatcccaagacatctgatctgcagacagttgagcattcacagcagttttgacttt

SEQ ID NO: 501 - DNA, 50 bp, homo sapiens
cgcccggctgacaagtgtatatttaaaagctcataaataatccaggtgac

SEQ ID NO: 502 - DNA, 50 bp, homo sapiens
cggccagcaaattctttggaaagctgccctgtggattgtagaatgataag

SEQ ID NO: 503 - DNA, 50 bp, homo sapiens
cgctgccaagaggttttgtgtttatcttggcatacataagaaatattttg

SEQ ID NO: 504 - DNA, 50 bp, homo sapiens
ttgcagcaatgaaaacaagctcaatgtccatgtcagataaacaattgtcg

SEQ ID NO: 505 - DNA, 50 bp, homo sapiens
caaaatattaagacagccaaagctgcttactgagatgtgcaagtgactcg

SEQ ID NO: 506 - DNA, 50 bp, homo sapiens
tcgctgtgaactttaaatccctcccatcatcttgccatacctttcctgcg

SEQ ID NO: 507 - DNA, 50 bp, homo sapiens
cgcttcctccacgcccattgggtcacgaaatccttgctaatcctggccgg

SEQ ID NO: 508 - DNA, 50 bp, homo sapiens
cgggctgtgtctcagcctcacagcactcctcccacgggcctgggaaggca

SEQ ID NO: 509 - DNA, 50 bp, homo sapiens
cgggcagccctgaagaggaacggtccctagagtccgtcccgcagctgctt

SEQ ID NO: 510 - DNA, 50 bp, homo sapiens
cgggaaggatcccatccaccagagctgccccacatggaccatggtcaggc

SEQ ID NO: 511 - DNA, 50 bp, homo sapiens
cgcctttgttgagaacagaacccccacaacatttaaggcaggccctgcac

SEQ ID NO: 512 - DNA, 50 bp, homo sapiens
cgcacacagaaatgagtcatatgttcagctttctgaaagtcaaagagaat

SEQ ID NO: 513 - DNA, 50 bp, homo sapiens
cgggccctcctgagcgggaggacgcagctttgtcttcagaccctgaggca

SEQ ID NO: 514 - DNA, 50 bp, homo sapiens
cggcaagagacagggaacagcatggagaactatgaaaaaataggcttcca

SEQ ID NO: 515 - DNA, 50 bp, homo sapiens
cggatgaagtgagagaagaatattccagcagagggagacaaggagactgc

SEQ ID NO: 516 - DNA, 50 bp, homo sapiens
cgcggcagccgcccctgcccagctcccctgctcagctcccctgctcagct

SEQ ID NO: 517 - DNA, 50 bp, homo sapiens
cgcctgcacaaagccggacgcctgcccgccccaccgtgggtcgatgcacc

SEQ ID NO: 518 - DNA, 50 bp, homo sapiens
tcaaattttcagtgatggaagaatcacacatcaccttgtggatttgaacg

SEQ ID NO: 519 - DNA, 50 bp, homo sapiens
aggagtcgggacttgtttcttctgtacccatacatcccctgtggcttccg

SEQ ID NO: 520 - DNA, 50 bp, homo sapiens
cgccgtgacatgtagttactgttttcatcttacagatgagggaaccaacg

SEQ ID NO: 521 - DNA, 50 bp, homo sapiens
cggaaaagtgattaaattcatctgccagggaattttaggctctctggtac

SEQ ID NO: 522 - DNA, 50 bp, homo sapiens
tctaggatatgctctttgaatgttattcctaacgtaatgcctttcattcg

SEQ ID NO: 523 - DNA, 50 bp, homo sapiens
tttctggttactaggattatggtggtatggttccctaatcattcctatcg

SEQ ID NO: 524 - DNA, 50 bp, homo sapiens
cggagtaggagtgaatcaatcgtacatccaagcagataacagctcaaatc

SEQ ID NO: 525 - DNA, 50 bp, homo sapiens
cgcagagttaagttcgaataagatggtcgtatttactttgccattctctc

SEQ ID NO: 526 - DNA, 50 bp, homo sapiens
tatttgacatcaaagacaggagatccagtggggaatgagacccacggccg

SEQ ID NO: 527 - DNA, 50 bp, homo sapiens
ctagttaatcctagttctgttgacattggaccaggctcagtaaataaacg

SEQ ID NO: 528 - DNA, 50 bp, homo sapiens
acggccgggcgtgggcagcgctggaggacaggctctcccagtgtgctgcg

SEQ ID NO: 529 - DNA, 50 bp, homo sapiens
cgccctgaggctccatgagtccatccagcccttgctaggtccacagcgac

SEQ ID NO: 530 - DNA, 50 bp, homo sapiens
cgtctgcagtcactctggaggtgacccaagcacctgggcccttagacagt

SEQ ID NO: 531 - DNA, 50 bp, homo sapiens
cccgctgggggacacgcctgagcatgtccacttccttcctgggggacacg

SEQ ID NO: 532 - DNA, 50 bp, homo sapiens
cgccccagccgcgcctgtagatgacgtcaccatcgtaagaccacaggacc

SEQ ID NO: 533 - DNA, 50 bp, homo sapiens
cggggtccattgccagtcacatagtgacttcaatgagcgttgctgccggc

SEQ ID NO: 534 - DNA, 50 bp, homo sapiens
ggagctgagcaggggagctgagcaggggagctgggcaggggcggctgccg

SEQ ID NO: 535 - DNA, 50 bp, homo sapiens
cgagaaccgtgcgacagtgcgtctgccaatacctggaatgtaggtgttcg

SEQ ID NO: 536 - DNA, 50 bp, homo sapiens
cggctggccagttctttttggtaggaaaatgtattgactatctttgtaaa

SEQ ID NO: 537 - DNA, 50 bp, homo sapiens
gcgggttggggaagctggcgaggccgagccccgctttgggcttctgggcg

SEQ ID NO: 538 - DNA, 50 bp, homo sapiens
cgtggccctgcagcaccaagaatatgcagggccttgcaatccagatggag

SEQ ID NO: 539 - DNA, 50 bp, homo sapiens
cttgttcctaggtgactccggcacataggctggaaacactacactcaccg

SEQ ID NO: 540 - DNA, 50 bp, homo sapiens
ccgggttccctggagatcctgtcttccacctagtgcagggcatgaggccg

SEQ ID NO: 541 - DNA, 50 bp, homo sapiens
gaataccctcagaggtgtttcggagtgggcaggagtggactgtgtctgcg

SEQ ID NO: 542 - DNA, 50 bp, homo sapiens
accatcacctgttagttcaggatgcaggtgtgaggcagcctgggggcgcg

SEQ ID NO: 543 - DNA, 50 bp, homo sapiens
cggcagtgagccgcaacaccagtcagccacgtgatcacaggggcaaacaa

SEQ ID NO: 544 - DNA, 50 bp, homo sapiens
cggttctcccctgagaggtctttcacagcaagggagtctgtatttttaga

SEQ ID NO: 545 - DNA, 50 bp, homo sapiens
cgccctcctgtccacctgctggcgcaacctgctcccccgtctctaggggt

SEQ ID NO: 546 - DNA, 50 bp, homo sapiens
cgcaaacaattcagagtgtgtgaggaaagttgatttcagagtccctgaaa

SEQ ID NO: 547 - DNA, 50 bp, homo sapiens
ctgtgatgtcactgatgaaacatctcaaaggaacaccctccgaaggttcg

SEQ ID NO: 548 - DNA, 50 bp, homo sapiens
gtttatttatgtggataaatcattcccatcaatgttacctacttttaacg

SEQ ID NO: 549 - DNA, 50 bp, homo sapiens
cggcaaaacagagcatgccacaccagggcatgggagccacaaaatgacag

SEQ ID NO: 550 - DNA, 50 bp, homo sapiens
cgaaggaagtgagcgcgttgatctggttgcggatggtgatgttgctcccg

SEQ ID NO: 551 - DNA, 50 bp, homo sapiens
tgagagctataaacaatgtgatactatgtcctgttaactcacagagaacg

SEQ ID NO: 552 - DNA, 50 bp, homo sapiens
cggttttaacacttggattacaggttcatctctccatgccagaagagagc

SEQ ID NO: 553 - DNA, 50 bp, homo sapiens
gtggtgctgagccatctctttcagggtctaaccttctgtggaaaagcacg

SEQ ID NO: 554 - DNA, 50 bp, homo sapiens
gaccttagaaggtcagaaatctttcaagccctgcaggaccgtaaaatgcg

SEQ ID NO: 555 - DNA, 50 bp, homo sapiens
gcttctgggtatgttgcaccttctgtaagaatttatttagtcacctgacg

SEQ ID NO: 556 - DNA, 50 bp, homo sapiens
cgcttctttaaatggcagagagaaggagaaaacttagatcttctgatacc

SEQ ID NO: 557 - DNA, 50 bp, homo sapiens
ctttcgtttcaacccatggtttatgtttgatctttactttgctgtcatcg

SEQ ID NO: 558 - DNA, 50 bp, homo sapiens
tgtagcatttagcaggtcaacaaaatgcactcccttattctttgtctccg

SEQ ID NO: 559 - DNA, 50 bp, homo sapiens
ctttgcccttcaagcctggctggaataattttacctaagtaggtttcacg

SEQ ID NO: 560 - DNA, 50 bp, homo sapiens
gcctaaataaactggatccaacttctcaggctggacttcttccagcttcg

SEQ ID NO: 561 - DNA, 50 bp, homo sapiens
tgctgggccacgctgaaaatttgtggctgagagctggaccctcgtcatcg

SEQ ID NO: 562 - DNA, 50 bp, homo sapiens
attagtctaagctggctcgtctcagccaatatctggcactcgcatacccg

SEQ ID NO: 563 - DNA, 50 bp, homo sapiens
cgtggctcaagtaacgaagtttcaagaaaaagagtcagagatctgaacta

SEQ ID NO: 564 - DNA, 50 bp, homo sapiens
cgcagcaggccacttaaactatctggtcagtttctttatcaacgaaatgg

SEQ ID NO: 565 - DNA, 50 bp, homo sapiens
gtcaaaaagttgccagagaggcgctttctcagcaaatctccctgagagcg

SEQ ID NO: 566 - DNA, 50 bp, homo sapiens
cgggaggcaaattttcagacatttgggtcctcggcagctgcagcagagtt

SEQ ID NO: 567 - DNA, 50 bp, homo sapiens
ataaaaccttgaatgcactgaaggaatgcataaaccgatttcacttagcg

SEQ ID NO: 568 - DNA, 50 bp, homo sapiens
ggtacacgcgagccttttggaaactattcccattcagtgccaactactcg

SEQ ID NO: 569 - DNA, 50 bp, homo sapiens
cgcagcagttgaaagtggtaaaaagctaagataattaaaatctctgccat

SEQ ID NO: 570 - DNA, 50 bp, homo sapiens
aggaggagctactgtgaaggtttctgcgtcttcaccctcactggctgtcg

SEQ ID NO: 571 - DNA, 50 bp, homo sapiens
tatattttaagcaaagcccagtgtctttaaagaacattctggtaatgtcg

SEQ ID NO: 572 - DNA, 50 bp, homo sapiens
agtggacctggccaacgtgtggcgcttcccctacctctgctacaagaacg

SEQ ID NO: 573 - DNA, 50 bp, homo sapiens
ctacacgtcggaccgggagaggcagctcttcagcatagatgccagtaccg

SEQ ID NO: 574 - DNA, 50 bp, homo sapiens
gagaacaccgcagctctgctaatcgagggacagccgcaacccaaagttcg

SEQ ID NO: 575 - DNA, 50 bp, homo sapiens
gctctcagggagatttgctgagaaagcgcctctctggcaactttttgacg

SEQ ID NO: 576 - DNA, 50 bp, homo sapiens
cgctccatccctgctggggcctcagggctcatgtctgggatttccccacc

SEQ ID NO: 577 - DNA, 50 bp, homo sapiens
ggccgcgttgctcaggacctgggactggagctggcggagctggtgcctcg

SEQ ID NO: 578 - DNA, 50 bp, homo sapiens
ggaaatgtttcttctttcagtgctgcctcctttcccttctaggatctccg

SEQ ID NO: 579 - DNA, 50 bp, homo sapiens
agttgggcgacatgctggccttgcctctgcagctgctgtgctctgctccg

SEQ ID NO: 580 - DNA, 50 bp, homo sapiens
cggcaggatgaatagccaagcttagttcgatacacttttgccctcagctg

SEQ ID NO: 581 - DNA, 50 bp, homo sapiens
cggcctgaggaggtcaggaaaggtattttagagaaactgatttcactata

SEQ ID NO: 582 - DNA, 50 bp, homo sapiens
cactcactatcaatgctccactacctagccagtgttattccagagaagcg

SEQ ID NO: 583 - DNA, 50 bp, homo sapiens
cggcctctttgttggtttaatcaggaggccttcgtgccacagtccaactt

SEQ ID NO: 584 - DNA, 50 bp, homo sapiens
gtccggagcatggtcctgggtcaccgttggtgtagcgtgttggtggaacg

SEQ ID NO: 585 - DNA, 50 bp, homo sapiens
gcaaggtacagttgagctgctgatcaccgtcctcgacgttaatgataacg

SEQ ID NO: 586 - DNA, 50 bp, homo sapiens
cgggtcacaacctcaaatgacctgctaccctgcccataaataacagagat

SEQ ID NO: 587 - DNA, 50 bp, homo sapiens
aaaaagaataagcaaatcatgcaaatcatcctgttctaaactctgaaccg

SEQ ID NO: 588 - DNA, 50 bp, homo sapiens
caagttggtgtccaccttcaagaattactattcattggtgctggacagcg

SEQ ID NO: 589 - DNA, 50 bp, homo sapiens
cgctatgtgccgtccttggcctcttccaggatggtgtagtggaactggtc

SEQ ID NO: 590 - DNA, 50 bp, homo sapiens
acataatttcagtcattaggcctcatagattggactaaataaatacctcg

SEQ ID NO: 591 - DNA, 50 bp, homo sapiens
gcactttactgttctcctgcaggggcttaaggtccagctcgaagtgctcg

SEQ ID NO: 592 - DNA, 50 bp, homo sapiens
cggcagagggtgtgctctgaggagggcccacctaagacggacctcatggc

SEQ ID NO: 593 - DNA, 50 bp, homo sapiens
ttctcttattcatatccattccatacccacaccaacttggataccatacg

SEQ ID NO: 594 - DNA, 50 bp, homo sapiens
tacagtctgggtggaaatcttggacaccaacgataactctcctgaagtcg

SEQ ID NO: 595 - DNA, 50 bp, homo sapiens
cgcagaaacctaggtaatactaatatttaatacataggcagatgaaaaaa

SEQ ID NO: 596 - DNA, 50 bp, homo sapiens
tgtgggggtttccacaattgctacttacggtttggagccacatgatgtcg

SEQ ID NO: 597 - DNA, 50 bp, homo sapiens
cggtgttagagggaacaaaagaaaatgtttgcaaatcattttataaacct

SEQ ID NO: 598 - DNA, 50 bp, homo sapiens
cggccagctccactactccgtctcggaggaggccaaacacggcaccttcg

SEQ ID NO: 599 - DNA, 50 bp, homo sapiens
ggcctcagggctcatgtctgggatttccccacctttgcggggcaggagcg

SEQ ID NO: 600 - DNA, 50 bp, homo sapiens
cgggtcacccgcgcactgaagacagcctctgtgctctcggaacggctctg

SEQ ID NO: 601 - DNA, 50 bp, homo sapiens
cccagggtgcctggacatgctgagcctccagggccagttcaccttcaccg

SEQ ID NO: 602 - DNA, 50 bp, homo sapiens
cgctagaactgagtttataggttaagatggaatttgagccaacatctgca

SEQ ID NO: 603 - DNA, 50 bp, homo sapiens
cttccacatgtttgtactgtggtttaactcatttaatccttgcaccatcg

SEQ ID NO: 604 - DNA, 50 bp, homo sapiens
gccagggcggggaccgcaaggtgggcgggaggcttggaggccgggatgcg

SEQ ID NO: 605 - DNA, 50 bp, homo sapiens
cggaggaataagagaagcagcaggactttaacagagactagaatatttaa

SEQ ID NO: 606 - DNA, 50 bp, homo sapiens
cggcttacagctggtggcaaagctgagcctggcctgacttgttcatgcct

SEQ ID NO: 607 - DNA, 50 bp, homo sapiens
cggaagatcttctaatggaaataaaaccagaggtatttgacatggtgttt

SEQ ID NO: 608 - DNA, 50 bp, homo sapiens
cggctgctccccagccagctcccgcttcaggttggcgctgaagagcagga

SEQ ID NO: 609 - DNA, 50 bp, homo sapiens
gattagggaagatgcaacaccccgcaccctctggcttagcagaacctacg

SEQ ID NO: 610 - DNA, 50 bp, homo sapiens
cggtgatcagcagctcaactgtaccttgcagctccggtttgcccccatca

SEQ ID NO: 611 - DNA, 50 bp, homo sapiens
cggatccgtttgaaacctgtccggtccctgctttctatgccaggcaaaaa

SEQ ID NO: 612 - DNA, 50 bp, homo sapiens
cgcgcccggcggtgggactctgatttcaacaccagattccaccaaagtcg

SEQ ID NO: 613 - DNA, 50 bp, homo sapiens
cgctggatgtaaatgatgatgcccagtgtttgacaaaggagtctatcatg

SEQ ID NO: 614 - DNA, 50 bp, homo sapiens
cgtgagcaccaacaggctggacaccgcgcagatggcaatgatcaagtaaa

SEQ ID NO: 615 - DNA, 50 bp, homo sapiens
cggtctgatccaaggacttcaacccactcaggccacttgatctcattaga

SEQ ID NO: 616 - DNA, 50 bp, homo sapiens
cgggaacctatctgactgacggacctggcgtccaagtggcctccaccaca

SEQ ID NO: 617 - DNA, 50 bp, homo sapiens
ggaatttgaaatctattgcctcatgtgcaaaaaaagcactcactaaaacg

SEQ ID NO: 618 - DNA, 50 bp, homo sapiens
gctccattcgatacctcgtgtttagagatcgttatcgcctcagataaacg

SEQ ID NO: 619 - DNA, 50 bp, homo sapiens
cgaaggcaagttttgctgactagaaaaacaagccaataatttgaaatggt

SEQ ID NO: 620 - DNA, 50 bp, homo sapiens
cgctgcatctcccgttgttcggcctttagagacagatgctgtcagagaaa

SEQ ID NO: 621 - DNA, 50 bp, homo sapiens
cgtccaactattcttggatctactcacatccctggatcaagtctggccgc

SEQ ID NO: 622 - DNA, 50 bp, homo sapiens
cgccaggatcccagctctcctcgccagtgataatccatgttaaatagtcg

SEQ ID NO: 623 - DNA, 50 bp, homo sapiens
cgcccaccttgcggtccccgccctggcgtctaggtggcaccagaatcccg

SEQ ID NO: 624 - DNA, 50 bp, homo sapiens
cgggcgcagctagatgtgctggaagcactgtttgccaagacccggtaccc

SEQ ID NO: 625 - DNA, 50 bp, homo sapiens
cggcactgcttttataagtaccatggtcaggaaatgacaagtgagacctg

SEQ ID NO: 626 - DNA, 50 bp, homo sapiens
tcctgcagaaaatatagcagaaagcggaatacctcttgcgaatccttccg

SEQ ID NO: 627 - DNA, 50 bp, homo sapiens
cgctgaagcgttcgcgtttacggttccaaaaccaagactgaaaggcagtc

SEQ ID NO: 628 - DNA, 50 bp, homo sapiens
cgcgccccccgcgcagccttatatctaacggtcaattcgtgcaatctgtc

SEQ ID NO: 629 - DNA, 50 bp, homo sapiens
ggagcacaccagagtgggctttaccaaactacatgcgccctcagaggacg

SEQ ID NO: 630 - DNA, 50 bp, homo sapiens
cgagttataggaagcgtttcagagagaggataacacatttgaatctattt

SEQ ID NO: 631 - DNA, 50 bp, homo sapiens
cggaggcagcagctcttccacgttccagcactaacttaaaaagagcacgg

SEQ ID NO: 632 - DNA, 50 bp, homo sapiens
cttcagccgcccccagagggctgtcagaagtctccaactcttgagttccg

SEQ ID NO: 633 - DNA, 50 bp, homo sapiens
agcaggagtgctgacctttcactcggggatgggggaactttgtctcctcg

SEQ ID NO: 634 - DNA, 50 bp, homo sapiens
attttcttctgttaaaaaaaatacctttcggccgggtgcggtggcactcg

SEQ ID NO: 635 - DNA, 50 bp, homo sapiens
cgctgttatttcttttgtggagaaagaccttgtggtggacagcgacatca

SEQ ID NO: 636 - DNA, 50 bp, homo sapiens
cgccagctccagccccaggtcctgcgcgatgcggcccacgaagttgccat

SEQ ID NO: 637 - DNA, 50 bp, homo sapiens
aaacttttagaggcagtcagtctgctaagaaggctccagcaagagaaacg

SEQ ID NO: 638 - DNA, 50 bp, homo sapiens
cgcagcatttgcctaataagctgacgctctcttgacagctcgattgcgtg

SEQ ID NO: 639 - DNA, 50 bp, homo sapiens
cggcttcccaaagtgccgggattacaaataggcatgagctaccgcacccg

SEQ ID NO: 640 - DNA, 50 bp, homo sapiens
cgcccgaggtcccgctccactgcccgcgccccccgcgcagccttatatct

SEQ ID NO: 641 - DNA, 50 bp, homo sapiens
cggctgaaagatggggcctttatctgccaaagaataatgcactactgatc

SEQ ID NO: 642 - DNA, 50 bp, homo sapiens
cgaccaactccccaccgcgctagaacacaccagcgtcggcttgccaggcg

SEQ ID NO: 643 - DNA, 50 bp, homo sapiens
cttcttttaacccccattgtaatttatttttacatcttttgtggttatcg

SEQ ID NO: 644 - DNA, 50 bp, homo sapiens
cggcacccatacagagagctcattcaaaagttcagctaaggatttgtcct

SEQ ID NO: 645 - DNA, 50 bp, homo sapiens
cgagccctaaaatggaagggcttcttatgacttgtccattgacaaaaatt

SEQ ID NO: 646 - DNA, 50 bp, homo sapiens
gactgacctgctgggctgagtcaggggctgtgggcagcattattcctgcg

SEQ ID NO: 647 - DNA, 50 bp, homo sapiens
ttcgctgtgggccacggccagtgtttctgtggaggtggctgatgtgaacg

SEQ ID NO: 648 - DNA, 50 bp, homo sapiens
gcgcggaccgtgggggaggtggcgggggcgaggccggcaagaggcggccg

SEQ ID NO: 649 - DNA, 50 bp, homo sapiens
cggagaagcgcatttgggacactatagtcaaatcccgtccaataaaaggt

SEQ ID NO: 650 - DNA, 50 bp, homo sapiens
cgaaagaggttggggtctgtctttatggttaatgtgaatccatttcctgg

SEQ ID NO: 651 - DNA, 50 bp, homo sapiens
cgcctcactatgactgctcacgaggtggcctctggagaactcatacctag

SEQ ID NO: 652 - DNA, 50 bp, homo sapiens
cgggccccgcctccgctcgtgccaccctgcgcctgctcccccaccgctca

SEQ ID NO: 653 - DNA, 50 bp, homo sapiens
cggaagtaattcatgtaatcattaccttttgaagccacatgatgtcgctg

SEQ ID NO: 654 - DNA, 50 bp, homo sapiens
ctaaatttgacctctttccataaattttctttgccccacttccaaatgcg

SEQ ID NO: 655 - DNA, 50 bp, homo sapiens
cggcttcctgctgctgctgccgctacagctccagggggagggaaaaggaa

SEQ ID NO: 656 - DNA, 50 bp, homo sapiens
tatgaagactgagtcatacttacacttacacgttcatgcgcatggtgtcg

SEQ ID NO: 657 - DNA, 50 bp, homo sapiens
ggaaactttaacctcctggttaaattctatttaaccaggacgccgattcg

SEQ ID NO: 658 - DNA, 50 bp, homo sapiens
cggtgagcctagattaggccactgcactacagcctgggagacagggtgaa

SEQ ID NO: 659 - DNA, 50 bp, homo sapiens
cgcagcccggcccgcggccgcctcttgccggcctcgcccccgccacctcc

SEQ ID NO: 660 - DNA, 50 bp, homo sapiens
gaggccaaagagagcgagcagtgagcgccggggcggacgagaagccagcg

SEQ ID NO: 661 - DNA, 50 bp, homo sapiens
cgcctcagataaacggggcagagaggtggggagataagcagtttaccctc

SEQ ID NO: 662 - DNA, 50 bp, homo sapiens
catgttgaaaaaacactcttcacgttcgctcggcctggtgaccagggtcg

SEQ ID NO: 663 - DNA, 50 bp, homo sapiens
cggtgttaaattttagagaggatgaagcaaagcagtatctgaatacgtaa

SEQ ID NO: 664 - DNA, 50 bp, homo sapiens
cggccagtgagagatgcatgagttaaacaagctttacttattctactgaa

SEQ ID NO: 665 - DNA, 50 bp, homo sapiens
cgcggtctatccgagaattcacaaacaaaatgccattctgcagatttacc

SEQ ID NO: 666 - DNA, 50 bp, homo sapiens
tgttcaaagtcttttctctgacagcatctgtctctaaaggccgaacaacg

SEQ ID NO: 667 - DNA, 50 bp, homo sapiens
acattgggtacatcaactaccccagacttaaaatgggagcatgatacccg

SEQ ID NO: 668 - DNA, 50 bp, homo sapiens
cgggggggaaatagctagatatgattctcaagaaagtctgtaataccacc

SEQ ID NO: 669 - DNA, 50 bp, homo sapiens
cggttttgatagttccttaaggacaagaatggctcccgtattataaatct

SEQ ID NO: 670 - DNA, 50 bp, homo sapiens
cgggcgcatcaagatcttccagctgggtacccccgatttgggccgacttt

SEQ ID NO: 671 - DNA, 50 bp, homo sapiens
cgagatcatttaggaacagctgaagaatcaaaaacagcaaagcacaatgg

SEQ ID NO: 672 - DNA, 50 bp, homo sapiens
ttctaaaataactccagctccaaatgaaggccagtggacccatgaacacg

SEQ ID NO: 673 - DNA, 50 bp, homo sapiens
agcaatggaagcagccgagcgtggcctcgcccatggtgaactggagaacg

SEQ ID NO: 674 - DNA, 50 bp, homo sapiens
cgccattaccaatcctcaatgttttgatcaaattagaagagcatgttttg

SEQ ID NO: 675 - DNA, 50 bp, homo sapiens
cggacagttaaaggagccaaggttgtgatcctgacacccagaattcaaag

SEQ ID NO: 676 - DNA, 50 bp, homo sapiens
aagatgtgggagtcaggtgaatagttcccatcgccagtaaccccagagcg

SEQ ID NO: 677 - DNA, 50 bp, homo sapiens
cagcttgaaagggacgtggggcgtcagggagcaggtgacatgtccattcg

SEQ ID NO: 678 - DNA, 50 bp, homo sapiens
gagaaaaaatacatccgcctacagaactccacgttcgcagaggggcatcg

SEQ ID NO: 679 - DNA, 50 bp, homo sapiens
cgggcatgggcccaacacgaggctgcatcctcggctcctcctcacagtcc

SEQ ID NO: 680 - DNA, 50 bp, homo sapiens
ttcgctgtgggccaccgccagcttgtctgtggaggtggccgacgtgaacg

SEQ ID NO: 681 - DNA, 50 bp, homo sapiens
cgaaagaatttgcggtaaagagcgacatcatgtggccatcatgtaagtaa

SEQ ID NO: 682 - DNA, 50 bp, homo sapiens
cggggctttctcccaacgataattaatagtcaaggccaacccttttggca

SEQ ID NO: 683 - DNA, 50 bp, homo sapiens
cgaatctgcaaattatttcccagatgctgagtaaagaggtgaggaggttc

SEQ ID NO: 684 - DNA, 50 bp, homo sapiens
tagagtgtccgtggaggtggccgacgtgaacgacaatgcgcctgcgttcg

SEQ ID NO: 685 - DNA, 50 bp, homo sapiens
taaatggacttactttgaggccatgatcaacatttaaatcttgctcttcg

SEQ ID NO: 686 - DNA, 50 bp, homo sapiens
tatcccaattctcagcctattctcaagagagtaagactgttagtatatcg

SEQ ID NO: 687 - DNA, 50 bp, homo sapiens
taacacttaataaagcattaaggacactgttccacctaatgatgtttccg

SEQ ID NO: 688 - DNA, 50 bp, homo sapiens
cgactgaaataataaataaaaccaagtagtttacagtagagtgtgtgggg

SEQ ID NO: 689 - DNA, 50 bp, homo sapiens
cggcctttagagacagatgctgtcagagaaaagactttgaacattgacca

SEQ ID NO: 690 - DNA, 50 bp, homo sapiens
cgacctgacatctgcatctggtgggaacagaaacgggttctttccaattg

SEQ ID NO: 691 - DNA, 50 bp, homo sapiens
tgggtgcaggccacgtggtggcgaaggtgcgcgcggtggacgccgattcg

SEQ ID NO: 692 - DNA, 50 bp, homo sapiens
agccactggactacgaggagctggagctgctgcagttctaggtgagagcg

SEQ ID NO: 693 - DNA, 50 bp, homo sapiens
agtgtcgaccctcttcaaaaaccacgcagcatttgcctaataagctgacg

SEQ ID NO: 694 - DNA, 50 bp, homo sapiens
gtctgttggtgctcacactgctgctatatactgctctgcggtgctccgcg

SEQ ID NO: 695 - DNA, 50 bp, homo sapiens
tgcaggttttccatgtggaggtggcagtgaaggacatcaatgacaatccg

SEQ ID NO: 696 - DNA, 50 bp, homo sapiens
cgaggtagcagagtttagatccagattggtctgatcccaaagcctgaatt

SEQ ID NO: 697 - DNA, 50 bp, homo sapiens
cgggtttttgttcttggggaaggcagattgtaattgctttcttcgggtac

SEQ ID NO: 698 - DNA, 50 bp, homo sapiens
cgggtgctgccagctgacagatgcagctgttgcgacaaccaggcgtcata

SEQ ID NO: 699 - DNA, 50 bp, homo sapiens
cgctggaatgagctcgaggtactctgcaaagagcctgggaaaatgacctt

SEQ ID NO: 700 - DNA, 50 bp, homo sapiens
gctgatgcctttgttctaaaaagctattttgtggacgacagcgacatccg

SEQ ID NO: 701 - DNA, 50 bp, homo sapiens
ttgcttagattccgcaatcagcagcttttgttctctgagcgagaacaccg

SEQ ID NO: 702 - DNA, 50 bp, homo sapiens
gtatgaccagctccccaccgcactggagcacaccagagtgggcttgcccg

SEQ ID NO: 703 - DNA, 50 bp, homo sapiens
cggaaactaaacgtagacaaaacgaataacattgtgttgctgctgcacgt

SEQ ID NO: 704 - DNA, 50 bp, homo sapiens
cgggttgaagtctgtcctgcaccctcttgtatcccaggagctgccaagag

SEQ ID NO: 705 - DNA, 50 bp, homo sapiens
cgagaatgcttggagaatgggacgtgggcctcaaagatcaactactcaca

SEQ ID NO: 706 - DNA, 50 bp, homo sapiens
ttggccgcatcgcgcaggacctagggctggagctggcggagctggtgccg

SEQ ID NO: 707 - DNA, 50 bp, homo sapiens
cagaggcaccggccccagccccactggatgtgtgtgggtcgaggtcttcg

SEQ ID NO: 708 - DNA, 50 bp, homo sapiens
cgaccccaaccaaatctgggggccaaaacgcaaagatcgcgggagaagcc

SEQ ID NO: 709 - DNA, 50 bp, homo sapiens
cgccagcaacttactatttcgcctgcaacttgcttttaagcctgccgccc

SEQ ID NO: 710 - DNA, 50 bp, homo sapiens
aggcagctacatctggaaaccatcttatccttctcttgttttacaaatcg

SEQ ID NO: 711 - DNA, 50 bp, homo sapiens
ttacatgatgtacacactcctagaccaggtagctgtagccatgaccttcg

SEQ ID NO: 712 - DNA, 50 bp, homo sapiens
caagctggtgtccacctacaagaactactactcattagtgctggacagcg

SEQ ID NO: 713 - DNA, 50 bp, homo sapiens
cgggactttttacaggcaccaaaagttattagcaacagaggggagtagga

SEQ ID NO: 714 - DNA, 50 bp, homo sapiens
tggggagtcagcgaggacggtggggccaggagcccttgggagggcctacg

SEQ ID NO: 715 - DNA, 50 bp, homo sapiens
cgaggctgccaaaaatgtctctggacattgcccagtgtccccggggaggc

SEQ ID NO: 716 - DNA, 50 bp, homo sapiens
cgagctgtcaagagagcgtcagcttattaggcaaatgctgcgtggttttt

SEQ ID NO: 717 - DNA, 50 bp, homo sapiens
agccagcgccaggctgcagttggcctccagcaggctgtggagcagtgccg

SEQ ID NO: 718 - DNA, 50 bp, homo sapiens
cgcatggaagacaatttttccacagattttggggtgggggtagtttcaag

SEQ ID NO: 719 - DNA, 50 bp, homo sapiens
cgccttctggtgctggtgaaggaccatggagagcccccgctgacagccac

SEQ ID NO: 720 - DNA, 50 bp, homo sapiens
atgacagacttcagactattaacccaggacttatattttgcctggggacg

SEQ ID NO: 721 - DNA, 50 bp, homo sapiens
cctgcgtttgggcagttcccgccctgcggctggctaggaggagctcctcg

SEQ ID NO: 722 - DNA, 50 bp, homo sapiens
tcagcagctttgtaccgctgggtccggaatcctctttactccctaaaccg

SEQ ID NO: 723 - DNA, 50 bp, homo sapiens
agcgcccttcataaaaattcttctggctgagggttctacgttggcatacg

SEQ ID NO: 724 - DNA, 50 bp, homo sapiens
cgcccagaaaataatctgagttcatccctagccctcaggtaacggatggg

SEQ ID NO: 725 - DNA, 50 bp, homo sapiens
cagaaggggccgaggggcctggacacggctgctctcgcttctgctcctcg

SEQ ID NO: 726 - DNA, 50 bp, homo sapiens
agcttgacccggctcagggcattcagagcgattacattcaagagacttcg

SEQ ID NO: 727 - DNA, 50 bp, homo sapiens
cggaataggatcgactccataatgactgtgatgctggaaaaaatttacta

SEQ ID NO: 728 - DNA, 50 bp, homo sapiens
cggggtcaggatttctcctgctcagctcgggactttttacaggcaccaaa

SEQ ID NO: 729 - DNA, 50 bp, homo sapiens
agggcagggccgagcgaggaatcctgactttcctgctctttaactttgcg

SEQ ID NO: 730 - DNA, 50 bp, homo sapiens
tgaatgccctgagccgggtcaagctggtgtctatgtttgtggctggagcg

SEQ ID NO: 731 - DNA, 50 bp, homo sapiens
cgagagtcaagcagtctggattcaggtattgagagcttttgttctgttac

SEQ ID NO: 732 - DNA, 50 bp, homo sapiens
tttttatggctatctctttatgatcctctcatttctgaacactgaactcg

SEQ ID NO: 733 - DNA, 50 bp, homo sapiens
cggtttagccacttcctgggtcactcttaattctaatgtttcgactttgg

SEQ ID NO: 734 - DNA, 50 bp, homo sapiens
acaactagttaaaagcctttcataccagtcagtattcccagccttgagcg

SEQ ID NO: 735 - DNA, 50 bp, homo sapiens
cgcgtttacggttccaaaaccaagactgaaaggcagtcttcacatattta

SEQ ID NO: 736 - DNA, 50 bp, homo sapiens
cggaacatacagtattttatctttatgcggaagatcttctaatggaaata

SEQ ID NO: 737 - DNA, 50 bp, homo sapiens
gcggggagcggggtcggctcagtggccctgagaccctagctctgctctcg

SEQ ID NO: 738 - DNA, 50 bp, homo sapiens
atcttatccttctcttgttttacaaatcgacgaatcagcggtctagaacg

SEQ ID NO: 739 - DNA, 50 bp, homo sapiens
cggcacagattatgatttttgtgactctgggaaaggttgtgctgtgtggg

SEQ ID NO: 740 - DNA, 50 bp, homo sapiens
cgcggtactagcttgttaactgcgcctcccgcgctgccagcctgagtcgc

SEQ ID NO: 741 - DNA, 50 bp, homo sapiens
cggttccaaaaccaagactgaaaggcagtcttcacatatttattgtttat

SEQ ID NO: 742 - DNA, 50 bp, homo sapiens
aatgctcaactgtctgcagatcagatgtcttgggatggaatccgttctcg

SEQ ID NO: 743 - DNA, 50 bp, homo sapiens
aggtcctgtctccatcagcatgcatttgcagggactggtctgtggtcacg

SEQ ID NO: 744 - DNA, 50 bp, homo sapiens
ggctgcgcggggaggctggtcccgggctgggcaggcgggctggcctcgcg

SEQ ID NO: 745 - DNA, 50 bp, homo sapiens
cggaatttaattgaagagatgaactccagtgtgccagaaaggacgtgaga

SEQ ID NO: 746 - DNA, 50 bp, homo sapiens
cgcgattccaccctggtgctgcagtttgtaaaagaagaaaaaatacctgt

SEQ ID NO: 747 - DNA, 50 bp, homo sapiens
gcggtctgtgggcgcgggccatgtggtggcgaaggtacgtgcagttgacg

SEQ ID NO: 748 - DNA, 50 bp, homo sapiens
aaaaaaatttcccctaaagtcttcagaagaaatgcggcccaggaaacacg

SEQ ID NO: 749 - DNA, 50 bp, homo sapiens
gtgagtgcgccctgagcgcgggacagggctaggtctgcctgggaggcccg

SEQ ID NO: 750 - DNA, 50 bp, homo sapiens
cggattccatcccaagacatctgatctgcagacagttgagcattcacagc

SEQ ID NO: 751 - DNA, 50 bp, artificial
  Probe Sequence
ccgaccaaaattaacaaaaatttcgtaacccaataaacgtaaaaaaaacg

SEQ ID NO: 752 - DNA, 50 bp, artificial
  Probe Sequence
aaacaactacgaaacgaactctaaaaaccgttcctcttcaaaactacccg

SEQ ID NO: 753 - DNA, 50 bp, artificial
  Probe Sequence
tacctcaaaatctaaaaacaaaactacgtcctcccgctcaaaaaaacccg

SEQ ID NO: 754 - DNA, 50 bp, artificial
  Probe Sequence
aactaaacaaaaaaactaaacaaaaaaactaaacaaaaacgactaccgcg

SEQ ID NO: 755 - DNA, 50 bp, artificial
  Probe Sequence
aatacatcgacccacgataaaacgaacaaacgtccgactttatacaaacg

SEQ ID NO: 756 - DNA, 50 bp, artificial
  Probe Sequence
aaaaaaataacaaaataaatacgaccatcttattcgaacttaactctacg

SEQ ID NO: 757 - DNA, 50 bp, artificial
  Probe Sequence
tatttaacatcaaaaacaaaaaatccaataaaaaataaaacccacgaccg

SEQ ID NO: 758 - DNA, 50 bp, artificial
  Probe Sequence
acgaccgaacgtaaacaacgctaaaaaacaaactctcccaatatactacg

SEQ ID NO: 759 - DNA, 50 bp, artificial
  Probe Sequence
cccgctaaaaaacacgcctaaacatatccacttccttcctaaaaaacacg

SEQ ID NO: 760 - DNA, 50 bp, artificial
  Probe Sequence
aatcctataatcttacgataataacgtcatctacaaacgcgactaaaacg

SEQ ID NO: 761 - DNA, 50 bp, artificial
  Probe Sequence
cgaaaaccgtacgacaatacgtctaccaatacctaaaatataaatattcg

SEQ ID NO: 762 - DNA, 50 bp, artificial
  Probe Sequence
acgaattaaaaaaactaacgaaaccgaaccccgctttaaacttctaaacg

SEQ ID NO: 763 - DNA, 50 bp, artificial
  Probe Sequence
aaataccctcaaaaatatttcgaaataaacaaaaataaactatatctacg

SEQ ID NO: 764 - DNA, 50 bp, artificial
  Probe Sequence
acccctaaaaacgaaaaaacaaattacgccaacaaataaacaaaaaaacg

SEQ ID NO: 765 - DNA, 50 bp, artificial
  Probe Sequence
cgaaaaaaataaacgcgttaatctaattacgaataataatattactcccg

SEQ ID NO: 766 - DNA, 50 bp, artificial
  Probe Sequence
gatacacgcgaaccttttaaaaactattcccattcaataccaactactcg

SEQ ID NO: 767 - DNA, 50 bp, artificial
  Probe Sequence
ctacacgtcgaaccgaaaaaaacaactcttcaacataaataccaataccg

SEQ ID NO: 768 - DNA, 50 bp, artificial
  Probe Sequence
cgaaaataccgtatttaacctcctccgaaacgaaataataaaactaaccg

SEQ ID NO: 769 - DNA, 50 bp, artificial
  Probe Sequence
caaaaccgttccgaaaacacaaaaactatcttcaatacgcgaataacccg

SEQ ID NO: 770 - DNA, 50 bp, artificial
  Probe Sequence
gccaaaacgaaaaccgcaaaataaacgaaaaacttaaaaaccgaaatacg

SEQ ID NO: 771 - DNA, 50 bp, artificial
  Probe Sequence
tttttacctaacataaaaaacaaaaaccgaacaaatttcaaacgaatccg

SEQ ID NO: 772 - DNA, 50 bp, artificial
  Probe Sequence
cgactttaataaaatctaatattaaaatcaaaatcccaccgccgaacgcg

SEQ ID NO: 773 - DNA, 50 bp, artificial
  Probe Sequence
actccattcgatacctcgtatttaaaaatcgttatcgcctcaaataaacg

SEQ ID NO: 774 - DNA, 50 bp, artificial
  Probe Sequence
cgactatttaacataaattatcactaacgaaaaaaactaaaatcctaacg

SEQ ID NO: 775 - DNA, 50 bp, artificial
  Probe Sequence
cgaaattctaataccacctaaacgccaaaacgaaaaccgcaaaataaacg

SEQ ID NO: 776 - DNA, 50 bp, artificial
  Probe Sequence
aaataccgaatcttaacaaacaatacttccaacacatctaactacgcccg

SEQ ID NO: 777 - DNA, 50 bp, artificial
  Probe Sequence
aactacctttcaatcttaattttaaaaccgtaaacgcgaacgcttcaacg

SEQ ID NO: 778 - DNA, 50 bp, artificial
  Probe Sequence
gacaaattacacgaattaaccgttaaatataaaactacgcgaaaaacgcg

SEQ ID NO: 779 - DNA, 50 bp, artificial
  Probe Sequence
ccgtactctttttaaattaatactaaaacgtaaaaaaactactacctccg

SEQ ID NO: 780 - DNA, 50 bp, artificial
  Probe Sequence
aacaaaaatactaacctttcactcgaaaataaaaaaactttatctcctcg

SEQ ID NO: 781 - DNA, 50 bp, artificial
  Probe Sequence
attttcttctattaaaaaaaatacctttcgaccgaatacgataacactcg

SEQ ID NO: 782 - DNA, 50 bp, artificial
  Probe Sequence
ataacaacttcgtaaaccgcatcgcgcaaaacctaaaactaaaactaacg

SEQ ID NO: 783 - DNA, 50 bp, artificial
  Probe Sequence
cgacttcccaaaataccgaaattacaaataaacataaactaccgcacccg

SEQ ID NO: 784 - DNA, 50 bp, artificial
  Probe Sequence
aaatataaaactacgcgaaaaacgcgaacaataaaacgaaacctcgaacg

SEQ ID NO: 785 - DNA, 50 bp, artificial
  Probe Sequence
cgcctaacaaaccgacgctaatatattctaacgcgataaaaaattaatcg

SEQ ID NO: 786 - DNA, 50 bp, artificial
  Probe Sequence
acgcgaaccgtaaaaaaaataacgaaaacgaaaccgacaaaaaacgaccg

SEQ ID NO: 787 - DNA, 50 bp, artificial
  Probe Sequence
taaacgataaaaaaacaaacgcaaaataacacgaacgaaaacgaaacccg

SEQ ID NO: 788 - DNA, 50 bp, artificial
  Probe Sequence
ttccttttccctccccctaaaactataacgacaacaacaacaaaaaaccg

SEQ ID NO: 789 - DNA, 50 bp, artificial
  Probe Sequence
aaaaataacgaaaacgaaaccgacaaaaaacgaccgcgaaccgaactacg

SEQ ID NO: 790 - DNA, 50 bp, artificial
  Probe Sequence
aaaaccaaaaaaaacgaacaataaacgccgaaacgaacgaaaaaccaacg

SEQ ID NO: 791 - DNA, 50 bp, artificial
  Probe Sequence
aaaaataaactacttatctccccacctctctaccccgtttatctaaaacg

SEQ ID NO: 792 - DNA, 50 bp, artificial
  Probe Sequence
catattaaaaaaacactcttcacgttcgctcgacctaataaccaaaatcg

SEQ ID NO: 793 - DNA, 50 bp, artificial
  Probe Sequence
aataaatctacaaaataacattttatttataaattctcgaataaaccgcg

SEQ ID NO: 794 - DNA, 50 bp, artificial
  Probe Sequence
aaaatcgacccaaatcgaaaatacccaactaaaaaatcttaatacgcccg

SEQ ID NO: 795 - DNA, 50 bp, artificial
  Probe Sequence
aaaaaaaaatacatccgcctacaaaactccacgttcgcaaaaaaacatcg

SEQ ID NO: 796 - DNA, 50 bp, artificial
  Probe Sequence
aaactataaaaaaaaaccgaaaatacaacctcgtattaaacccatacccg

SEQ ID NO: 797 - DNA, 50 bp, artificial
  Probe Sequence
ttcgctataaaccaccgccaacttatctataaaaataaccgacgtaaacg

SEQ ID NO: 798 - DNA, 50 bp, artificial
  Probe Sequence
taccaaaaaaattaaccttaactattaattatcgttaaaaaaaaaccccg

SEQ ID NO: 799 - DNA, 50 bp, artificial
  Probe Sequence
taaaatatccgtaaaaataaccgacgtaaacgacaatacgcctacgttcg

SEQ ID NO: 800 - DNA, 50 bp, artificial
  Probe Sequence
taaatacaaaccacgtaataacgaaaatacgcgcgataaacgccgattcg

SEQ ID NO: 801 - DNA, 50 bp, artificial
  Probe Sequence
atataaccaactccccaccgcactaaaacacaccaaaataaacttacccg

SEQ ID NO: 802 - DNA, 50 bp, artificial
  Probe Sequence
ttaaccgcatcgcgcaaaacctaaaactaaaactaacgaaactaataccg

SEQ ID NO: 803 - DNA, 50 bp, artificial
  Probe Sequence
caaaaacaccgaccccaaccccactaaatatatataaatcgaaatcttcg

SEQ ID NO: 804 - DNA, 50 bp, artificial
  Probe Sequence
aaacgacaaacttaaaaacaaattacaaacgaaataataaattactaacg

SEQ ID NO: 805 - DNA, 50 bp, artificial
  Probe Sequence
taaaaaatcaacgaaaacgataaaaccaaaaacccttaaaaaaacctacg

SEQ ID NO: 806 - DNA, 50 bp, artificial
  Probe Sequence
cctacgtttaaacaattcccgccctacgactaactaaaaaaaactcctcg

SEQ ID NO: 807 - DNA, 50 bp, artificial
  Probe Sequence
tcaacaactttataccgctaaatccgaaatcctctttactccctaaaccg

SEQ ID NO: 808 - DNA, 50 bp, artificial
  Probe Sequence
caaaaaaaaccgaaaaacctaaacacgactactctcgcttctactcctcg

SEQ ID NO: 809 - DNA, 50 bp, artificial
  Probe Sequence
ccaaaatcgaaacattaaaattaaaaataacccaaaaaataactaaaccg

SEQ ID NO: 810 - DNA, 50 bp, artificial
  Probe Sequence
taaatatataaaaactacctttcaatcttaattttaaaaccgtaaacgcg

SEQ ID NO: 811 - DNA, 50 bp, artificial
  Probe Sequence
tatttccattaaaaaatcttccgcataaaaataaaatactatatattccg

SEQ ID NO: 812 - DNA, 50 bp, artificial
  Probe Sequence
acgaaaaacgaaatcgactcaataaccctaaaaccctaactctactctcg

SEQ ID NO: 813 - DNA, 50 bp, artificial
  Probe Sequence
acgactcaaactaacaacgcgaaaaacgcaattaacaaactaataccgcg

SEQ ID NO: 814 - DNA, 50 bp, artificial
  Probe Sequence
aactacgcgaaaaaactaatcccgaactaaacaaacgaactaacctcgcg

SEQ ID NO: 815 - DNA, 50 bp, artificial
  Probe Sequence
gcgatctataaacgcgaaccatataataacgaaaatacgtacaattaacg

SEQ ID NO: 816 - DNA, 50 bp, artificial
  Probe Sequence
gtaaatacgccctaaacgcgaaacaaaactaaatctacctaaaaaacccg